(19) United States
(12) Patent Application Publication
(10) Pub. No.: US 2010/0292188 A1
(43) Pub. Date: Nov. 18, 2010

(54) Compounds Comprising A Cyclobutoxy Group

(75) Inventors: Frédéric Denonne, Bruxelles (BE); Sylvain Celanire, Reignier (FR); Anne Valade, Brussels (BE); Sabine Defays, Tubize (BE); Véronique Durieu, Sombreffe (BE)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: UCB PHARMA S.A., Brussels (BE)

(21) Appl. No.: 12/812,719

(22) PCT Filed: Jan. 22, 2009
(86) PCT No.: PCT/EP09/50719

§ 371 Date: Jul. 13, 2010

(30) Foreign Application Priority Data
Jan. 24, 2008 (EP) EP 08001308.9

(51) Int. Cl. A61K 31/675; C07D 513/04; A61K 31/5377; A61P 25/28; A61P 25/16; A61P 25/18; A61P 25/08; A61P 3/04; A61P 25/22; A61P 29/00; A61P 9/00; A61K 31/437; C07D 498/04; C07F 9/40; A61K 31/55

(52) U.S. Cl. 514/81; 544/127; 514/234.5; 546/114; 514/301; 546/115; 514/302; 546/23; 514/215; 540/578

(57) ABSTRACT

The present invention relates to compounds of formula (I) comprising a cyclobutoxy group, processes for preparing them, pharmaceutical compositions comprising said compounds and their use as pharmaceuticals.

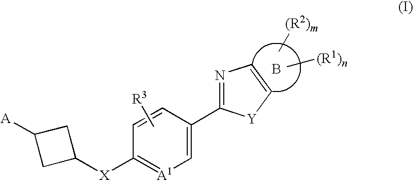

The present invention relates to compounds comprising a cyclobutoxy group, processes for preparing them, pharmaceutical compositions comprising said compounds and their use as pharmaceuticals.

The histamine H₃ receptor has been known for several years and identified pharmacologically in 1983 by Arrang, J. M. et al. (Nature 1983, 302, 832-837). Since the cloning of the human histamine H₃ receptor in 1999, histamine H₃ receptors have been successively cloned by sequence homology from a variety of species, including rat, guinea pig, mouse and monkey.

Histamine H₃-receptor agonists, antagonists and inverse agonists have shown potential therapeutic applications as described in the literature, for example by Stark, H. in Exp. Opin. Ther. Patents 2003, 13, 851-865, and by Leurs R. et al. in Nature Review Drug Discovery 2005, 4, 107-120.

The histamine H₃ receptor is predominantly expressed in the mammalian central nervous system but can also be found in the autonomic nervous system. Evidence has been shown that the histamine H₃ receptor displays high constitutive activity, which activity occurs in the absence of endogenous histamine or of a H₃-receptor agonist. Thus, a histamine H₃-receptor antagonist and/or inverse agonist could inhibit this activity.

The general pharmacology of histamine H₃ receptor, including H₃-receptor subtypes, has been reviewed by Hancock, A. A in Life Sci. 2003, 73, 3043-3072. The histamine H₃ receptor is not only considered as a presynaptic autoreceptor on histaminergic neurons, but also as a heteroreceptor on non-histaminergic neurons (Barnes, W. et al., Eur. J. Pharmacol. 2001, 431, 215-221). Indeed, the histamine H₃ receptor has been shown to regulate the release of histamine but also of other important neurotransmitters, including acetylcholine, dopamine, serotonin, norepinephrin and γ-aminobutyric acid (GABA).

Thus, the histamine H₃ receptor is of current interest for the development of new therapeutics and the literature suggests that novel histamine H₃-receptor antagonists or inverse agonists may be useful for the treatment and prevention of diseases or pathological conditions of the central nervous system including Mild Cognitive Impairment (MCI), Alzheimer's disease, learning and memory disorders, cognitive disorders, attention deficit disorder (ADD), attention-deficit hyperactivity disorder (ADHD), Parkinson's disease, schizophrenia, dementia, depression, epilepsy, seizures or convulsions, sleep/wake disorders, narcolepsy, pain and/or obesity.

H₃-receptor ligands alone or in combination with an acetylcholinesterase inhibitor may also be useful in the treatment of cholinergic-deficit disorders, Mild Cognitive Impairment and Alzheimer's disease as reported by Morisset, S. et al. in Eur. J. Pharmacol. 1996, 315, R1-R2.

H₃-receptor ligands, alone or in combination with a histamine H₁-receptor antagonist may be useful for the treatment of upper airway allergic disorders, as reported by McLeod, R. et al. in J. Pharmacol. Exp. Ther. 2003, 305, 1037-1044.

H₃-receptor ligands, alone or in combination with a serotonine reuptake inhibitor may be useful for the treatment of depression, as reported by Keith, J. M. et al in Bioorg. Med. Chem. Lett. 2007, 17, 702-706.

As described in international patent application WO 02/072093, H₃-receptor ligands alone or in combination with a muscarinic receptor ligand and particularly with a muscarinic M₂-receptor antagonist, may be useful for the treatment of cognitive disorders, Alzheimer's disease, attention-deficit hyperactivity disorder.

H₃-receptor ligands may also be useful in the treatment of sleep/wake and arousal/vigilance disorders such as hypersomnia, and narcolepsy according to Passani, M. B. et al. in Trends Pharmacol. Sci. 2004, 25(12), 618-625.

In general, H₃-receptor ligands, and particularly H₃-receptor antagonists or inverse agonists may be useful in the treatment of all types of cognitive-related disorders as reviewed by Hancock, A. A and Fox, G. B. in Expert Opin. Invest. Drugs 2004, 13, 1237-1248.

In particular, histamine H₃-receptor antagonists or inverse agonists may be useful in the treatment of cognitive dysfunctions in diseases such as Mild Cognitive Impairment, dementia, Alzheimer's disease, Parkinson's disease, Down's syndrome as well as in the treatment of attention-deficit hyperactivity disorder (ADHD) as non-psychostimulant agents (see for example Witkin, J. M. et al., Pharmacol. Ther. 2004, 103(1), 1-20).

H₃-receptor antagonists or inverse agonists may also be useful in the treatment of psychotic disorders such as schizophrenia, migraine, eating disorders such as obesity, inflammation, pain, anxiety, stress, depression and cardiovascular disorders, in particular acute myocardial infarction.

There is therefore a need to manufacture new compounds which can potentially act as H₃-receptor ligands.

Early literature reports (e.g. Ali, S. M. et al., J. Med. Chem. 1999, 42, 903-909 and Stark, H. et al., Drugs Fut. 1996, 21, 507-520) describe that an imidazole function is essential for high affinity histamine H₃-receptor ligands; this is confirmed, for example, by United States patents U.S. Pat. No. 6,506,756B2, U.S. Pat. No. 6,518,287B2, U.S. Pat. No. 6,528,522B2 and U.S. Pat. No. 6,762,186B2 which relate to substituted imidazole compounds that have H₃-receptor antagonist or dual histamine H₁-receptor and H₃-receptor antagonist activity.

International patent application WO 02/12214 relates to non-imidazole aryloxyalkylamines for the treatment of disorders and conditions mediated by the histamine receptor.

International patent application WO 02/074758 relates to bicyclic heterocyclic derivatives comprising an amine moiety and reported as H₃-receptor ligands.

International patent application WO 01/748810 relates to H₃-receptor antagonists comprising a benzoxazole or benzothiazole moiety.

International patent application WO 2006/103045 describes compounds comprising an oxazole or a thiazole moiety as H₃ receptor ligands.

International patent application WO 2006/136924 describes a class of phenoxycyclobutyl derivatives as H₃-receptor antagonists.

US patent application US 2005/171181 discloses cyclobutyl-arylamines as H₃-receptor modulators.

International patent application WO2006/097691 describes fused thiazole derivatives which display histamine H₃ receptor antagonist activity.

International patent applications WO 2006/132914 and WO 2007/038074 describe cyclobutyl amine derivatives as H₃-receptor modulators.

It has now surprisingly been found that compounds of formula (I) may act as H₃-receptor ligands and therefore may demonstrate therapeutic properties for one or more pathologies mentioned below.

The present invention relates to compounds of formula (I), geometrical isomers, enantiomers, diastereoisomers, pharmaceutically acceptable salts and all possible mixtures thereof,

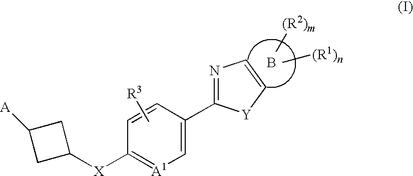

wherein

A is a substituted or unsubstituted aliphatic or cyclic amino group which is linked to the cyclobutyl group via an amino nitrogen;

A¹ is CH, C-halogen or N;

B is selected from the group consisting of heteroaryl, 5-8-membered heterocycloalkyl and 5-8-membered cycloalkyl;

X is O, S, NH or N(C₁₋₄ alkyl);

Y is O, S or NH;

R¹ is selected from the group comprising or consisting of sulfonyl, amino, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted heteroaryl, substituted or unsubstituted C₃₋₈ cycloalkyl, substituted or unsubstituted 3-8-membered heterocycloalkyl, acyl, substituted or unsubstituted C₁₋₆-alkyl aryl, substituted or unsubstituted C₁₋₆-alkyl heteroaryl, substituted or unsubstituted C₂₋₆-alkenyl aryl, substituted or unsubstituted C₂₋₆-alkenyl heteroaryl, substituted or unsubstituted C₂₋₆-alkynyl aryl, substituted or unsubstituted C₂₋₆-alkynyl heteroaryl, substituted or unsubstituted C₁₋₆-alkyl cycloalkyl, substituted or unsubstituted C₁₋₆-alkyl heterocycloalkyl, substituted or unsubstituted C₂₋₆-alkenyl cycloalkyl, substituted or unsubstituted C₂₋₆-alkenyl heterocycloalkyl, substituted or unsubstituted C₂₋₆-alkynyl cycloalkyl, substituted or unsubstituted C₂₋₆-alkynyl heterocycloalkyl, alkoxycarbonyl, aminocarbonyl, substituted or unsubstituted C₁₋₆-alkyl carboxy, substituted or unsubstituted C₁₋₆-alkyl acyl, substituted or unsubstituted aryl acyl, substituted or unsubstituted heteroaryl acyl, substituted or unsubstituted C₃₋₈-(hetero)cycloalkyl acyl, substituted or unsubstituted C₁₋₆-alkyl acyloxy, substituted or unsubstituted C₁₋₆-alkyl alkoxy, substituted or unsubstituted C₁₋₆-alkyl alkoxycarbonyl, substituted or unsubstituted C₁₋₆-alkyl aminocarbonyl, substituted or unsubstituted C₁₋₆-alkyl acylamino, acylamino, acylaminocarbonyl, ureido, substituted or unsubstituted C₁₋₆-alkyl ureido, substituted or unsubstituted C₁₋₆-alkyl carbamate, substituted or unsubstituted C₁₋₆-alkyl amino, substituted or unsubstituted C₁₋₆-alkyl sulfonyloxy, substituted or unsubstituted C₁₋₆-alkyl sulfonyl, substituted or unsubstituted C₁₋₆-alkyl sulfinyl, substituted or unsubstituted C₁₋₆-alkyl sulfanyl, substituted or unsubstituted C₁₋₆-alkyl sulfonylamino, aminosulfonyl, substituted or unsubstituted C₁₋₆-alkyl aminosulfonyl, hydroxy, substituted or unsubstituted C₁₋₆-alkyl hydroxy, phosphonate, substituted or unsubstituted C₁₋₆-alkyl phosphonate, substituted or unsubstituted C₁₋₆-alkyl phosphono, halogen, cyano, carboxy, oxo, thioxo;

n is equal to 0, 1, 2 or 3;

R² is selected from the group comprising or consisting of hydrogen, sulfonyl, amino, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted heteroaryl, substituted or unsubstituted C₃₋₈ cycloalkyl, substituted or unsubstituted 3-8-membered heterocycloalkyl, acyl, substituted or unsubstituted C₁₋₆-alkyl aryl, substituted or unsubstituted C₁₋₆-alkyl heteroaryl, substituted or unsubstituted C₂₋₆-alkenyl aryl, substituted or unsubstituted C₂₋₆-alkenyl heteroaryl, substituted or unsubstituted C₂₋₆-alkynyl aryl, substituted or unsubstituted C₂₋₆-alkynyl heteroaryl, substituted or unsubstituted C₁₋₆-alkyl cycloalkyl, substituted or unsubstituted C₁₋₆-alkyl heterocycloalkyl, substituted or unsubstituted C₂₋₆-alkenyl cycloalkyl, substituted or unsubstituted C₂₋₆-alkenyl heterocycloalkyl, substituted or unsubstituted C₂₋₆-alkynyl cycloalkyl, substituted or unsubstituted C₂₋₆-alkynyl heterocycloalkyl, alkoxycarbonyl, aminocarbonyl, substituted or unsubstituted C₁₋₆-alkyl carboxy, substituted or unsubstituted C₁₋₆-alkyl acyl, substituted or unsubstituted aryl acyl, substituted or unsubstituted heteroaryl acyl, substituted or unsubstituted C₃₋₈-(hetero)cycloalkyl acyl, substituted or unsubstituted C₁₋₆-alkyl acyloxy, substituted or unsubstituted C₁₋₆-alkyl alkoxy, substituted or unsubstituted C₁₋₆-alkyl alkoxycarbonyl, substituted or unsubstituted C₁₋₆-alkyl aminocarbonyl, substituted or unsubstituted C₁₋₆-alkyl acylamino, acylamino, acylaminocarbonyl, ureido, substituted or unsubstituted C₁₋₆-alkyl ureido, substituted or unsubstituted C₁₋₆-alkyl carbamate, substituted or unsubstituted C₁₋₆-alkyl amino, substituted or unsubstituted C₁₋₆-alkyl sulfonyloxy, substituted or unsubstituted C₁₋₆-alkyl sulfonyl, substituted or unsubstituted C₁₋₆-alkyl sulfinyl, substituted or unsubstituted C₁₋₆-alkyl sulfanyl, substituted or unsubstituted C₁₋₆-alkyl sulfonylamino, aminosulfonyl, substituted or unsubstituted C₁₋₆-alkyl aminosulfonyl, hydroxy, substituted or unsubstituted C₁₋₆-alkyl hydroxy, phosphonate, substituted or unsubstituted C₁₋₆-alkyl phosphonate, substituted or unsubstituted C₁₋₆-alkyl phosphono, halogen, cyano, carboxy, oxo, thioxo;

m is equal to 0 or 1; and

R³ is hydrogen or C₁₋₆ alkyl or halogen or C₁₋₆ alkoxy.

The term “alkyl”, as used herein, is a group which represents saturated, monovalent hydrocarbon radicals having straight (unbranched) or branched moieties, or combinations thereof, and containing 1-8 carbon atoms, preferably 1-6 carbon atoms; more preferably alkyl groups have 1-4 carbon atoms.

“Alkyl” groups according to the present invention may be unsubstituted or substituted. Preferred unsubstituted alkyl groups according to the present invention are methyl, ethyl, n-propyl, isopropyl and tert-butyl. “Alkyl” groups may be substituted by one or more substituents including halogen.

The term “halogen”, as used herein, represents a fluorine, chlorine, bromine, or iodine atom. Preferred halogen according to the present invention is fluorine.

The term “hydroxy”, as used herein, represents a group of formula —OH.

The term “C₁₋₆-alkyl hydroxy”, as used herein, refers to an alkyl as defined above substituted by one or more “hydroxy”. Preferred “C₁₋₆-alkyl hydroxy” groups according to the present invention are 2,3-dihydroxy-propyl, (2S)-2,3-dihydroxy-propyl, (2R)-2,3-dihydroxy-propyl and 2-hydroxyethyl.

The term “C₃₋₈ cycloalkyl”, as used herein, represents a monovalent group of 3 to 8 carbon atoms derived from a saturated or partially unsaturated cyclic hydrocarbon. Preferred C₃₋₈ cycloalkyl groups according to the present invention are cyclobutyl, cyclobutenyl and cyclopentenyl.

The C₃₋₈ cycloalkyl according to the invention may be substituted by a “hydroxy”, an “amino”, an “aminocarbonyl” or “oxo”. Example of such substituted C₃₋₈ cycloalkyl according to the present invention are 3-hydroxycyclobutyl, 1(aminocarbonyl)cyclopropyl, 1-hydroxycyclopropyl, 2-hydroxy-3,4-dioxocyclobut-1-en-1-yl, 3,4-dioxo-2-(propan-2-yloxy)cyclobut-1-en-1-yl and 2-amino-3,4-dioxocyclobut-1-en-1-yl.

The term “C₁₋₆-alkyl cycloalkyl”, as used herein, refers to a C₁₋₆ alkyl having a cycloalkyl substitutent as defined here above.

The term “alkylene”, as used herein, represents a group of formula —(CH₂)ₓ— in which x is comprised between 2 and 6, preferably comprised between 3 and 6.

The term “methylene” as used herein represents a group of formula —CH₂—.

The term “C₂₋₆ alkenyl” refers to alkenyl groups preferably having from 2 to 6 carbon atoms and having at least 1 or 2 sites of alkenyl unsaturation. The term “C₂₋₆ alkynyl” refers to alkynyl groups preferably having from 2 to 6 carbon atoms and having at least 1 to 2 sites of alkynyl unsaturation.

The term “aryl” as used herein, refers to an unsaturated aromatic carbocyclic group of from 6 to 14 carbon atoms having a single ring (e.g. phenyl) or multiple condensed rings (e.g. naphthyl). The “aryl” groups may be unsubstituted or substituted by 1 to 4 substituents independently selected from halogen, C₁₋₄ alkyl or C₁₋₄ alkoxy as defined herein.

The term “C₁₋₆-alkyl aryl”, as used herein, refers to a C₁₋₆ alkyl having an aryl substituent as defined hereabove.

The term “heteroaryl” as used herein represents an aryl group as defined here above wherein one or more of the carbon atoms have been replaced by one or more heteroatoms selected from O, S or N. Preferred heteroaryl according to the present invention is pyridyl and triazolyl.

The term “C₁₋₆-alkyl heteroaryl” refers to a C₁₋₆ alkyl having a heteroaryl substituent as defined here above.

The term “C₂₋₆-alkenyl aryl”, as used herein, refers to a C₂₋₆ alkenyl substituted by an aryl as defined here above.

The term “C₂₋₆-alkenyl heteroaryl”, as used herein, refers to a C₂₋₆ alkenyl substituted by a heteroaryl as defined here above.

The term “C₂₋₆-alkynyl aryl”, as used herein, refers to a C₂₋₆ alkynyl substituted by an aryl as defined here above.

The term “C₂₋₆-alkynyl heteroaryl”, as used herein, refers to a C₂₋₆ alkynyl substituted by a heteroaryl as defined here above.

The term “alkoxy”, as used herein, represents a group of formula —OR^{a} wherein R^{a} is an alkyl, acarboxyalkyl or an aryl group, as defined herein.

The term “C₁₋₆-alkyl alkoxy”, as used herein, refers to a C₁₋₆ alkyl group having an alkoxy substituent as defined hereabove.

The term “carbonyl”, as used herein represents a group of formula —C(═O)—.

The term “acyl”, as used herein, represents a group of formula —C(═O)R^{b} wherein R^{b} is C₁₋₆ alkyl, a C₁₋₆-alkyl alkoxy, a C₃₋₈ cycloalkyl optionally substituted by an hydroxy, an aminocarbonyl or oxo, a 3-8-membered heterocycloalkyl, a C₁₋₆-alkyl heterocycloalkyl, C₁₋₆-alkyl hydroxy, C₁₋₆-alkyl amino, C₁₋₆-alkyl acylamino, aminocarbonyl, C₁₋₆-alkyl aminocarbonyl, alkoxycarbonyl, a C₁₋₆-alkyl ureido or a heteroaryl as defined herein. Preferred acyl groups according to the invention are acetyl, methoxyacetyl, aminoacetyl, hydroxyacetyl, 3-amino-3-oxopropanoyl, 3,3,3-trifluoropropanoyl, (5-methyl-2H-1,2,3-triazol-4-yl)carbonyl, 4-(1-oxidothiomorpholin-4-yl)butanoyl, 3-(acetylamino)propanoyl, (carboxymethoxy)acetyl, 3,3,3-trifluoro-2-hydroxypropanoyl, tetrahydro-2H-pyran-4-ylcarbonyl, (1-hydroxycyclopropyl)carbonyl, [(1-aminocarbonyl)cylopropyl]carbonyl, ethoxy(oxo)acetyl, [(aminocarbonyl)amino]carbonyl, amino(oxo)acetyl, 2,3-dihydroxypropanoyl, (2S)-2,3-dihydroxypropanoyl and trifluoroacetyl

The term “C₁₋₆-alkyl acyl” as used herein refers to a C₁₋₆ alkyl having an acyl substituent as defined here above.

The term “3-8-membered heterocycloalkyl” as used herein represents a C₃₋₈ cycloalkyl as defined here above wherein one, two or three carbon atoms are replaced by one, two or three atoms selected from O, S or N. The heterocycloalkyl may be unsubstituted or substituted by any suitable group including, but not limited to, one or more, typically one, two or three, moieties selected from aminocarbonyl, C₁₋₆-alkyl aminocarbonyl, C₃₋₈ cycloalkyl, C₁₋₆-alkyl hydroxy, alkoxycarbonyl, C₁₋₆-alkyl alkoxycarbonyl, halogen, amino, oxo and C₁₋₆-alkyl as defined herein.

Examples of 3-8-membered heterocycloalkyl according to the present invention are piperidinyl, 4,4-difluoropiperidinyl, morpholin-4-yl, pyrrolidinyl, 4-isopropyl-piperazine, 3-(dimethylamino)pyrrolidinyl, azepanyl, (2S)-2-methylpyrrolidinyl, (2R)-2-methylpyrrolidinyl, 2-methylpyrrolidinyl, thiomorpholin-4-yl, 1,2,3,6-tetrahydropyridyl, 1,2,3,6-tetrahydropyridyl, 2,3,4,5-tetrahydro-1H-azepinyl, 1-oxidothiomorpholin-4-yl or tetrahydro-2H-pyran-4-yl.

The term “C₁₋₆-alkyl heterocycloalkyl”, as used herein, refers to a C₁₋₆ alkyl substituted by a heterocycloalkyl as defined here above.

The term “C₂₋₆-alkenyl cycloalkyl”, as used herein, refers to a C₂₋₆ alkenyl substituted by a cycloalkyl as defined here above.

The term “C₂₋₆-alkenyl heterocycloalkyl”, as used herein, refers to a C₂₋₆-alkenyl substituted by a heterocycloalkyl as defined here above.

The term “C₂₋₆-alkynyl cycloalkyl”, as used herein, refers to a C₂₋₆ alkynyl substituted by a cycloalkyl as defined here above.

The term “C₂₋₆-alkynyl heterocycloalkyl”, as used herein, refers to a C₂₋₆-alkynyl substituted by a heterocycloalkyl as defined here above.

The term “aryl acyl” as used herein refers to an aryl group having an acyl substituent as defined here above.

The term “heteroaryl acyl” as used herein refers to an heteroaryl group having an acyl substituent as defined here above.

The term “C₃₋₈-(hetero)cycloalkyl acyl” as used herein refers to a 3-8-membered heterocycloalkyl group having an acyl substituent as defined here above.

The term “amino”, as used herein, represents an aliphatic group of formula —NR^{c}R^{d} wherein R^{c} and R^{d} are independently hydrogen, “C₁₋₆ alkyl”, “C₂₋₆ alkenyl”, “C₂₋₆ alkynyl”, “C₃₋₈ cycloalkyl”, “heterocycloalkyl”, “aryl”, “heteroaryl”, “C₁₋₆-alkyl aryl”, “C₁₋₆-alkyl heteroaryl”, “C₁₋₆-alkyl cycloalkyl” or “C₁₋₆-alkyl heterocycloalkyl” groups; or a cyclic group of formula —NR^{c}R^{d} wherein R^{c} and R^{d} are linked together with N to form a 3 to 8 membered, preferably 5 to 7 membered heterocycloalkyl, as defined herein.

Examples of “amino” groups according to the present invention are amino, dimethylamino, piperidin-1-yl, 4,4-difluoropiperidin-1-yl, morpholin-4-yl, thiomorpholin-4-yl, pyrrolidin-1-yl, azepan-1-yl, 4-(isopropyl)piperazin-1-yl, 2-methylpyrrolidin-1-yl, (2S)-2-methylpyrrolidin-1-yl, (2R)-2-methylpyrrolidin-1-yl, (3R)-3-(dimethylamino)pyrrolydin-1-yl, 3-(dimethylamino)pyrrolydin-1-yl and 4-cyclopentyl-piperazin-1-yl.

The term “C₁₋₆-alkyl amino”, as used herein, represents a C₁₋₆ alkyl group substituted by an amino group as defined above.

The term “aminocarbonyl” as used herein refers to a group of formula —C(O)NR^{c}R^{d} wherein R^{c} and R^{d} are as defined here above for the amino group. Examples of “aminocarbonyl” according to the present invention include aminocarbonyl, morpholin-4-ylcarbonyl and (ethylamino)carbonyl.

The term “C₁₋₆-alkyl aminocarbonyl” as used herein, refers to a C₁₋₆ alkyl substituted by an aminocarbonyl as defined hereabove. An example of a C₁₋₆-alkyl aminocarbonyl according to the present invention is 2-amino-2-oxoethyl.

The term “C₃₋₈-cycloalkyl amino”, as used herein, represents a C₃₋₈ cycloalkyl group substituted by an amino group as defined above.

The term “acylamino”, as used herein refers to a group of formula —NR^{c}C(O)R^{d} wherein R^{c} and R^{d} are as defined hereabove for the amino group.

The term “C₁₋₆-alkyl acylamino”, as used herein refers to a C₁₋₆ alkyl substituted by an acylamino as defined hereabove.

The term “carboxy”, as used herein represents a group of formula —COOH.

The term “C₁₋₆-alkyl carboxy”, as used herein refers to a C₁₋₆ alkyl substituted by a carboxy group.

The term “cyano”, as used herein represents a group of formula —CN.

The term “alkoxycarbonyl” refers to the group —C(O)OR⁹ wherein R⁹ includes “C₁₋₆ alkyl”, “C₂₋₆ alkenyl”, “C₂₋₆ alkynyl”, “C₃₋₈ cycloalkyl”, “heterocycloalkyl”, “aryl”, “heteroaryl”, “C₁₋₆-alkyl aryl” or “C₁₋₆-alkyl heteroaryl”, “C₂₋₆-alkyl cycloalkyl”, “C₁₋₆-alkyl heterocycloalkyl”. Examples of alkoxycarbonyl according to the present invention are tert-butoxycarbonyl, methoxycarbonyl and ethoxycarbonyl.

The term “C₁₋₆-alkyl alkoxycarbonyl” refers to a C₁₋₆ alkyl having an alkoxycarbonyl as defined here above as substituent. Example of C₁₋₆-alkyl alkoxycarbonyl according to the present invention is 2-methoxy-2-oxoethyl.

The term “acyloxy” as used herein refers to a group of formula —OC(═O)R^{b} wherein R^{b} is as defined here above for acyl group.

The term “C₁₋₆-alkyl acyloxy” as used herein refers to a C₁₋₆ alkyl substituted by an acyloxy as defined here above.

The term “acylaminocarbonyl” refers to the group —C(O)NR^{h}C(O)Rⁱ wherein R^{h} and Rⁱ represent independently hydrogen, “C₁₋₆ alkyl”, “C₂₋₆ alkenyl”, “C₂₋₆ alkynyl”, “C₃₋₈ cycloalkyl”, “heterocycloalkyl”, “aryl”, “heteroaryl”, “C₁₋₆-alkyl aryl” or “C₁₋₆-alkyl heteroaryl”, “C₂₋₆-alkyl cycloalkyl”, “C₁₋₆-alkyl heterocycloalkyl”.

The term “ureido” as used herein refers to a group of formula —NRⁱC(O)NR^{c}R^{d} wherein Rⁱ is as defined here above for R^{c} or R^{d}, and R^{c} and R^{d} are as defined here above for the amino group. Rⁱ is typically hydrogen or C₁₋₄ alkyl.

The term “C₁₋₆-alkyl ureido” as used herein refers to a C₁₋₆ alkyl substituted by an ureido as defined here above. Example of C₁₋₆-alkyl ureido is [(aminocarbonyl)amino]methyl.

The term “carbamate”, as used herein, refers to a group of formula —NR^{c}C(O)OR^{d} wherein R^{c} and R^{d} are as defined here above for the amino group.

The term “C₁₋₆-alkyl carbamate” as used herein refers to a C₁₋₆ alkyl substituted by a carbamate as defined here above.

The term “oxo” as used herein refers to ═O.

The term “thioxo” as used herein refers to ═S.

The term “sulfonyl” as used herein refers to a group of formula “—SO₂—R^{k}” wherein R^{k} is selected from H, “aryl”, “heteroaryl”, “C₁₋₆ alkyl”, “C₁₋₆ alkyl” substituted with halogens, e.g., an —SO₂—CF₃ group, “C₂₋₆ alkenyl”, “C₂₋₆ alkynyl”, “C₃₋₈ cycloalkyl”, “heterocycloalkyl”, “aryl”, “heteroaryl”, “C₁₋₆-alkyl aryl” or “C₁₋₆-alkyl heteroaryl”, “C₂₋₆-alkenyl aryl”, “C₂₋₆-alkenyl heteroaryl”, “C₂₋₆-alkynyl aryl”, “C₂₋₆-alkynyl heteroaryl”, “C₁₋₆-alkyl cycloalkyl” or “C₁₋₆-alkyl heterocycloalkyl”.

The term “C₁₋₆-alkyl sulfonyl” as used herein refers to a C₁₋₆ alkyl substituted by a sulfonyl as defined here above.

The term “sulfonyloxy” as used herein refers to a group of formula “—OSO₂—R^{k}” wherein R^{k} is defined as here above for sulfonyl group.

The term “C₁₋₆-alkyl sulfonyloxy” as used herein refers to a C₁₋₆ alkyl substituted by a sulfonyloxy as defined here above.

The term “aminosulfonyl” as used herein refers to a group of formula —SO₂—NR^{c}R^{d} wherein R^{c} and R^{d} are as defined here above for the amino group. Example of an aminosulfonyl group according to the invention is morpholin-4-ylsulfonyl.

The term “C₁₋₆-alkyl aminosulfonyl” as used herein refers to a C₁₋₆ alkyl substituted by an aminosulfonyl as defined here above.

The term “sulfinyl” as used herein refers to a group “—S(O)—R^{k}” wherein R^{k} is as defined here above for sulfonyl group.

The term “C₁₋₆-alkyl sulfinyl” as used herein refers to a C₁₋₆ alkyl substituted by a sulfinyl as defined here above.

The term “sulfanyl” as used herein refers to a group of formula —S—R^{k} where R^{k} is as defined here above for sulfonyl group.

The term “C₁₋₆-alkyl sulfanyl” as used herein refers to a C₁₋₆ alkyl substituted by a sulfanyl as defined here above.

The term “sulfonylamino” as used herein refers to a group —NR^{c}SO₂—R^{k} wherein R^{k} is defined as here above for sulfonyl group and R^{c} is defined as here above for amino group.

The term “C₁₋₆-alkyl sulfonylamino” as used herein refers to a C₁₋₆ alkyl substituted by a sulfonylamino as defined here above.

The term “phosphonate” as used herein refers to a group of formula —P(O)—(OR^{m})₂ wherein R^{m} is an alkyl group as defined herein. The term “C₁₋₆-alkyl phosphonate” refers to a C₁₋₆ alkyl group substituted by a “phosphonate” as described here above. An example of a “C₁₋₆-alkyl phosphonate” according to the present invention is [bis(ethyloxy)phosphoryl]methyl.

The term “phosphono” as used herein refers to a group of formula —P(O)—(OH)₂.

The term “C₁₋₆-alkyl phosphono” refers to a C₁₋₆ alkyl group substituted by a “phosphono” as described herein. An example of “C₁₋₆-alkyl phosphono” according to the present invention is phosphonomethyl.

Unless otherwise constrained by the definition of the individual substituents, all the above set out groups may be “substituted” or unsubstituted”.

“Substituted or unsubstituted” as used herein, unless otherwise constrained by the definition of the individual substituents, shall mean that the above set out groups, like “C₁₋₆ alkyl”, “C₂₋₆ alkenyl”, “C₂₋₆ alkynyl”, “aryl” and “heteroaryl” etc. . . . may optionally be substituted with from 1 to 5 substituents selected from the group consisting of “C₁₋₆ alkyl”, “C₂₋₆ alkenyl”, “C₂₋₆ alkynyl”, “cycloalkyl”, “heterocycloalkyl”, “C₁₋₆-alkyl aryl”, “C₁₋₆-alkyl heteroaryl”, “C₁₋₆-alkyl cycloalkyl”, “C₁₋₆-alkyl heterocycloalkyl”, “C₁₋₆-alkyl hydroxy”, “amino”, “ammonium”, “acyl”, “acyloxy”, “acylamino”, “aminocarbonyl”, “alkoxycarbonyl”, “ureido”, “carbamate”, “aryl”, “heteroaryl”, “sulfinyl”, “sulfonyl”, “aminosulfonyl”, “alkoxy”, “sulfanyl”, “halogen”, “carboxy”, trihalomethyl, cyano, hydroxy, nitro, phosphonate and the like.

In one embodiment according to the present invention, A represents a group of formula —NR⁴R⁵ wherein R⁴ and R⁵ are independently substituted or unsubstituted C₁₋₆ alkyl, substituted or unsubstituted C₂₋₆ alkenyl, substituted or unsubstituted C₂₋₆ alkynyl, substituted or unsubstituted C₃₋₈ cycloalkyl, substituted or unsubstituted heterocycloalkyl, substituted or unsubstituted C₁₋₆-alkyl aryl, substituted or unsubstituted C₁₋₆-alkyl heteroaryl, substituted or unsubstituted C₁₋₆-alkyl cycloalkyl or substituted or unsubstituted C₁₋₆-alkyl heterocycloalkyl groups; or A is a 3 to 8 membered substituted or unsubstituted heterocycloalkyl linked to the cyclobutyl group via a nitrogen atom.

In another embodiment according to the present invention, A is a group —NR⁴R⁵ wherein R⁴ and R⁵ are independently substituted or unsubstituted C₁₋₆ alkyl; or A is a 3 to 8 membered substituted or unsubstituted heterocycloalkyl linked to the cyclobutyl group via a nitrogen atom.

In a particular embodiment according to the present invention, A is a 3 to 8 membered heterocycloalkyl linked to the cyclobutyl group via a nitrogen atom.

In another particular embodiment according to the present invention, A represents a 3 to 8 membered heterocycloalkyl linked to the cyclobutyl group via a nitrogen atom selected from the groups comprising or consisting of substituted or unsubstituted piperidin-1-yl, substituted or unsubstituted morpholin-4-yl, substituted or unsubstituted pyrrolidin-1-yl, substituted or unsubstituted piperazin-1-yl, substituted or unsubstituted azepan-1-yl or substituted or unsubstituted thiomorpholin-4-yl.

Typical examples of A according to the invention include pyrrolidin-1-yl, 2-methylpyrrolidin-1-yl, (2S)-2-methylpyrrolidin-1-yl, (2R)-2-methylpyrrolidin-1-yl, piperidin-1-yl, 4,4-difluoropiperidin-1-yl, morpholin-4-yl, (3R)-3-(dimethylamino)pyrrolidin-1-yl, 3-(dimethylamino)pyrrolidin-1-yl, azepan-1-yl, thiomorpholin-4-yl, 4-isopropylpiperazin-1-yl and 4-cyclopentylpiperazin-1-yl.

In one particular embodiment according to the present invention, A is selected from substituted or unsubstituted piperidin-1-yl, and substituted or unsubstituted pyrrolidin-1-yl. Examples of A according to this particular embodiment are piperidin-1-yl, 2-methylpyrrolidin-1-yl, (2S)-2-methylpyrrolidin-1-yl or (2R)-2-methylpyrrolidin-1-yl.

In another particular embodiment, A is piperidin-1-yl, (2S)-2-methylpyrrolidin-1-yl or (2R)-2-methylpyrrolidin-1-yl.

In a further particular embodiment, A is piperidin-1-yl.

In another particular embodiment, A is (2S)-2-methylpyrrolidin-1-yl.

In yet another particular embodiment, A is (2R)-2-methylpyrrolidin-1-yl. A₁ may be CH, C—F or N.

In one embodiment A¹ is CH or C—F.

In a particular embodiment according to the present invention, A¹ is CH.

In one embodiment according to the present invention, B is a substituted or unsubstituted 5, 6 or 7-membered cycloalkyl, a substituted or unsubstituted 5, 6 or 7-membered heterocycloalkyl, or a substituted or unsubstituted heteroaryl.

In another embodiment according to the present invention, B is substituted or unsubstituted 5, 6 or 7-membered cycloalkyl, a substituted or unsubstituted 5, 6 or 7-membered heterocycloalkyl, or a substituted or unsubstituted heteroaryl selected from the group comprising or consisting of a tetrahydropyridyl, a tetrahydro-1H-azepinyl, a cyclopentenyl or a pyridyl.

In a particular embodiment according to the present invention, B is a substituted or unsubstituted 5, 6 or 7-membered cycloalkyl, a substituted or unsubstituted 5, 6 or 7-membered heterocycloalkyl, or a substituted or unsubstituted heteroaryl which forms together with the oxazole, the thiazole or the imidazole ring fused heterocycles including 4,5,6,7-tetrahydro[1,3]thiazolopyridine, 4,5,6,7-tetrahydro[1,3]oxazolopyridine, 4,5,6,7-tetrahydro-1H-imidazopyridine, 5,6-dihydro-4H-cyclopenta[d][1,3]thiazole, 5,6,7,8-tetrahydro-4H-[1,3]thiazoloazepine, 5,6,7,8-tetrahydro-4H-[1,3]oxazoloazepine, 1H-imidazopyridine and [1,3]thiazolopyridine.

Examples of such heterocycles are 4,5,6,7-tetrahydro[1,3]thiazolo[5,4-c]pyridine, 4,5,6,7-tetrahydro[1,3]thiazolo[4,5-b]pyridine, 4,5,6,7-tetrahydro[1,3]thiazolo[4,5-c]pyridine, 4,5,6,7-tetrahydro[1,3]oxazolo[4,5-c]pyridine, 4,5,6,7-tetrahydro-3H-imidazo[4,5-c]pyridine, 5,6,7,8-tetrahydro-4H-[1,3]thiazolo[5,4-b]azepine, 5,6,7,8-tetrahydro-4H-[1,3]oxazolo[5,4-b]azepine, 5,6-dihydro-4H-cyclopenta[d][1,3]thiazole, 3H-imidazo[4,5-c]pyridine and [1,3]thiazolo[4,5-c]pyridine.

In a further particular embodiment B forms together with the thiazole ring a fused heterocycle including 4,5,6,7-tetrahydro[1,3]thiazolo[5,4-c]pyridine.

In a particular embodiment according of the present invention, X is O.

In another particular embodiment, X is S.

In a particular embodiment according to the present invention, Y is S.

In another particular embodiment, Y is O.

In a further embodiment, Y is NH.

In one embodiment according to the present invention, R¹ is selected from the group comprising or consisting of substituted or unsubstituted C₁₋₆ alkyl, substituted or unsubstituted aryl, substituted or unsubstituted heteroaryl, substituted or unsubstituted C₃₋₈ cycloalkyl, substituted or unsubstituted 3-8-membered heterocycloalkyl, acyl, substituted or unsubstituted C₁₋₆-alkyl cycloalkyl, substituted or unsubstituted C₁₋₆-alkyl heterocycloalkyl, alkoxycarbonyl, aminocarbonyl, substituted or unsubstituted C₁₋₆-alkyl alkoxycarbonyl, substituted or unsubstituted C₁₋₆-alkyl aminocarbonyl, hydroxy, halogen, cyano, carboxy, oxo, thioxo; and n is 0 or 1.

In another embodiment according to the present invention, R¹ is selected from the group comprising or consisting of substituted or unsubstituted C₁₋₆ alkyl, hydroxy, oxo; and n is 0 or 1.

In a particular embodiment according of the present invention, n is 0.

In one embodiment according to the present invention, R² is selected from the group consisting of hydrogen, carboxy, sulfonyl, amino, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted heteroaryl, substituted or unsubstituted C₃₋₈ cycloalkyl, substituted or unsubstituted 3-8-membered heterocycloalkyl, acyl, substituted or unsubstituted C₁₋₆-alkyl aryl, substituted or unsubstituted C₁₋₆-alkyl heteroaryl, substituted or unsubstituted C₂₋₆-alkenyl aryl, substituted or unsubstituted C₂₋₆-alkenyl heteroaryl, substituted or unsubstituted C₂₋₆-alkynyl aryl, substituted or unsubstituted C₂₋₆-alkynyl heteroaryl, substituted or unsubstituted C₁₋₆-alkyl cycloalkyl, substituted or unsubstituted C₁₋₆-alkyl heterocycloalkyl, substituted or unsubstituted C₂₋₆-alkenyl cycloalkyl, substituted or unsubstituted C₂₋₆-alkenyl heterocycloalkyl, substituted or unsubstituted C₂₋₆-alkynyl cycloalkyl, substituted or unsubstituted C₂₋₆-alkynyl heterocycloalkyl, alkoxycarbonyl, aminocarbonyl, substituted or unsubstituted C₁₋₆-alkyl carboxy, substituted or unsubstituted C₁₋₆-alkyl acyl, substituted or unsubstituted aryl acyl, substituted or unsubstituted heteroaryl acyl, substituted or unsubstituted C₃₋₈-(hetero)cycloalkyl acyl, substituted or unsubstituted C₁₋₆-alkyl acyloxy, substituted or unsubstituted C₁₋₆-alkyl alkoxy, substituted or unsubstituted C₁₋₆-alkyl alkoxycarbonyl, substituted or unsubstituted C₁₋₆-alkyl aminocarbonyl, substituted or unsubstituted C₁₋₆-alkyl acylamino, acylamino, ureido, substituted or unsubstituted C₁₋₆-alkyl ureido, substituted or unsubstituted C₁₋₆-alkyl carbamate, substituted or unsubstituted C₁₋₆-alkyl amino, aminosulfonyl, substituted or unsubstituted C₁₋₆-alkyl aminosulfonyl, hydroxy, substituted or unsubstituted C₁₋₆-alkyl hydroxy, phosphonate, substituted or unsubstituted C₁₋₆-alkyl phosphonate, substituted or unsubstituted C₁₋₆-alkyl phosphono, oxo and thioxo.

In another embodiment according to the present invention, R² is selected from the group consisting of hydrogen, carboxy, sulfonyl, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted heteroaryl, substituted or unsubstituted C₃₋₈ cycloalkyl, substituted or unsubstituted 3-8-membered heterocycloalkyl, acyl, substituted or unsubstituted C₁₋₆-alkyl cycloalkyl, substituted or unsubstituted C₁₋₆-alkyl heterocycloalkyl, alkoxycarbonyl, substituted or unsubstituted C₁₋₆-alkyl alkoxycarbonyl, aminocarbonyl, substituted or unsubstituted C₁₋₆-alkyl aminocarbonyl, acylamino, ureido, substituted or unsubstituted C₁₋₆-alkyl ureido, substituted or unsubstituted C₁₋₆-alkyl carbamate, amino, substituted or unsubstituted C₁₋₆-alkyl amino, aminosulfonyl, hydroxy, substituted or unsubstituted C₁₋₆-alkyl hydroxy, substituted or unsubstituted C₁₋₆-alkyl phosphonate, substituted or unsubstituted C₁₋₆-alkyl phosphono and oxo.

In a further embodiment, the present invention comprises compounds of formula (I) wherein R² is selected from the group consisting of hydrogen, carboxy, acyl, substituted or unsubstituted C₃₋₈ cycloalkyl, alkoxycarbonyl, substituted or unsubstituted C₁₋₆-alkyl alkoxycarbonyl, aminocarbonyl, substituted or unsubstituted C₁₋₆-alkyl aminocarbonyl, aminosulfonyl, substituted or unsubstituted C₁₋₆-alkyl hydroxy, substituted or unsubstituted C₁₋₆-alkyl phosphonate and substituted or unsubstituted C₁₋₆-alkyl phosphono.

Examples of R² according to this further embodiment are hydrogen, carboxy, methoxyacetyl, tert-butoxycarbonyl, acetyl, morpholin-4-ylcarbonyl, morpholin-4-ylsulfonyl, aminoacetyl, aminocarbonyl, hydroxyacetyl, 2,3-dihydroxypropyl, (2S)-2,3-dihydroxypropyl, (2R)-2,3-dihydroxypropyl, 2-amino-2-oxoethyl, (ethylamino)carbonyl, 3-hydroxycyclobutyl, 3-amino-3-oxopropanoyl, 2-methoxy-2-oxoethyl, [bis(ethyloxy)phosphoryl]methyl, 3,3,3-trifluoropropanoyl, phosphonomethyl, (5-methyl-2H-1,2,3-triazol-4-yl)carbonyl, 2-hydroxyethyl, 4-(1-oxidothiomorpholin-4-yl)butanoyl, 3-(acetylamino)propanoyl, (carboxymethoxy)acetyl, 3,3,3-trifluoro-2-hydroxypropanoyl, tetrahydro-2H-pyran-4-ylcarbonyl, (1-hydroxycyclopropyl)carbonyl, [(1-aminocarbonyl)cylopropyl]carbonyl, ethoxy(oxo)acetyl, [(aminocarbonyl)amino]carbonyl, amino(oxo)acetyl, 2,3-dihydroxypropanoyl, 2-hydroxy-3,4-dioxocyclobut-1-en-1-yl, 3,4-dioxo-2-(propan-2-yloxy)cyclobut-1-en-1-yl, 2-amino-3,4-dioxocyclobut-1-en-1-yl, (2S)-2,3-dihydroxypropanoyl and trifluoroacetyl.

In a particular embodiment, R² is selected from the group comprising or consisting of acetyl, aminoacetyl, aminocarbonyl, hydroxyacetyl, 2,3-dihydroxypropyl, (2S)-2,3-dihydroxypropyl, (2R)-2,3-dihydroxypropyl, 2-amino-2-oxoethyl, 3-hydroxycyclobutyl,3-amino-3-oxopropanoyl, (5-methyl-2H-1,2,3-triazol-4-yl)carbonyl, 2-hydroxyethyl, (carboxymethoxy)acetyl, tetrahydro-2H-pyran-4-ylcarbonyl, [(1-aminocarbonyl)cylopropyl]carbonyl, amino(oxo)acetyl, 2,3-dihydroxypropanoyl and 2-amino-3,4-dioxocyclobut-1-en-1-yl.

In another particular embodiment, R² is selected from the group comprising or consisting of acetyl, aminocarbonyl, hydroxyacetyl, 2-amino-2-oxoethyl and amino(oxo)acetyl.

In a particular embodiment according to the present invention, m is 1.

In one embodiment according to the present invention, R³ is hydrogen or halogen.

In another embodiment according to the present invention, R³ is hydrogen or fluorine.

In a particular embodiment according to the present invention, R³ is hydrogen.

In one embodiment the present invention relates to compounds of formula (I), geometrical isomers, enantiomers, diastereoisomers, pharmaceutically acceptable salts and all possible mixtures thereof,

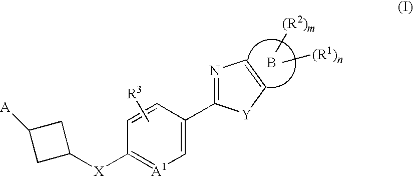

wherein

A is a group of formula —NR⁴R⁵ wherein R⁴ and R⁵ are independently substituted or unsubstituted C₁₋₆ alkyl, substituted or unsubstituted C₂₋₆ alkenyl, substituted or unsubstituted C₂₋₆ alkynyl, substituted or unsubstituted C₃₋₈ cycloalkyl, substituted or unsubstituted heterocycloalkyl, substituted or unsubstituted C₁₋₆-alkyl aryl, substituted or unsubstituted C₁₋₆-alkyl heteroaryl, substituted or unsubstituted C₁₋₆-alkyl cycloalkyl or substituted or unsubstituted C₁₋₆-alkyl heterocycloalkyl groups; or A is a 3 to 8 membered substituted or unsubstituted heterocycloalkyl linked to the cyclobutyl group via a nitrogen atom.

A¹ is CH, C-halogen or N;

B is a substituted or unsubstituted 5, 6 or 7-membered cycloalkyl, substituted or unsubstituted 5, 6 or 7-membered heterocycloalkyl, or substituted or unsubstituted heteroaryl;

X is O or S;

Y is O, S or NH;

n is equal to 0;

R² is selected from the group consisting of hydrogen, carboxy, sulfonyl, amino, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted heteroaryl, substituted or unsubstituted C₃₋₈ cycloalkyl, substituted or unsubstituted 3-8-membered heterocycloalkyl, acyl, substituted or unsubstituted C₁₋₆-alkyl aryl, substituted or unsubstituted C₁₋₆-alkyl heteroaryl, substituted or unsubstituted C₂₋₆-alkenyl aryl, substituted or unsubstituted C₂₋₆-alkenyl heteroaryl, substituted or unsubstituted C₂₋₆-alkynyl aryl, substituted or unsubstituted C₂₋₆-alkynyl heteroaryl, substituted or unsubstituted C₁₋₆-alkyl cycloalkyl, substituted or unsubstituted C₁₋₆-alkyl heterocycloalkyl, substituted or unsubstituted C₂₋₆-alkenyl cycloalkyl, substituted or unsubstituted C₂₋₆-alkenyl heterocycloalkyl, substituted or unsubstituted C₂₋₆-alkynyl cycloalkyl, substituted or unsubstituted C₂₋₆-alkynyl heterocycloalkyl, alkoxycarbonyl, aminocarbonyl, substituted or unsubstituted C₁₋₆-alkyl carboxy, substituted or unsubstituted C₁₋₆-alkyl acyl, substituted or unsubstituted aryl acyl, substituted or unsubstituted heteroaryl acyl, substituted or unsubstituted C₃₋₈-(hetero)cycloalkyl acyl, substituted or unsubstituted C₁₋₆-alkyl acyloxy, substituted or unsubstituted C₁₋₆-alkyl alkoxy, substituted or unsubstituted C₁₋₆-alkyl alkoxycarbonyl, substituted or unsubstituted C₁₋₆-alkyl aminocarbonyl, substituted or unsubstituted C₁₋₆-alkyl acylamino, acylamino, ureido, substituted or unsubstituted C₁₋₆-alkyl ureido, substituted or unsubstituted C₁₋₆-alkyl carbamate, substituted or unsubstituted C₁₋₆-alkyl amino, aminosulfonyl, substituted or unsubstituted C₁₋₆-alkyl aminosulfonyl, hydroxy, substituted or unsubstituted C₁₋₆-alkyl hydroxy, phosphonate, substituted or unsubstituted C₁₋₆-alkyl phosphonate, substituted or unsubstituted C₁₋₆-alkyl phosphono, oxo and thioxo;

m is equal to 0 or 1; and

R³ is hydrogen or halogen.

In another embodiment, the present invention relates to compounds of formula (I), geometrical isomers, enantiomers, diastereoisomers, pharmaceutically acceptable salts and all possible mixtures thereof,

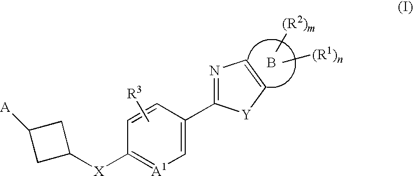

wherein

A is a group of formula —NR⁴R⁵ wherein R⁴ and R⁵ are independently substituted or unsubstituted C₁₋₆ alkyl; or A is a 3 to 8 membered substituted or unsubstituted heterocycloalkyl linked to the cyclobutyl group via a nitrogen atom.

A¹ is CH or C—F;

B is a substituted or unsubstituted 5, 6 or 7-membered cycloalkyl, substituted or unsubstituted 5, 6 or 7-membered heterocycloalkyl, or substituted or unsubstituted heteroaryl;

X is O or S;

Y is O, S or NH;

n is equal to 0;

R² is selected from the group consisting of hydrogen, carboxy, sulfonyl, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted heteroaryl, substituted or unsubstituted C₃₋₈ cycloalkyl, substituted or unsubstituted 3-8-membered heterocycloalkyl, acyl, substituted or unsubstituted C₁₋₆-alkyl cycloalkyl, substituted or unsubstituted C₁₋₆-alkyl heterocycloalkyl, alkoxycarbonyl, substituted or unsubstituted C₁₋₆-alkyl alkoxycarbonyl, aminocarbonyl, substituted or unsubstituted C₁₋₆-alkyl aminocarbonyl, acylamino, ureido, substituted or unsubstituted C₁₋₆-alkyl ureido, substituted or unsubstituted C₁₋₆-alkyl carbamate, amino, substituted or unsubstituted C₁₋₆-alkyl amino, aminosulfonyl, hydroxy, substituted or unsubstituted C₁₋₆-alkyl hydroxy, substituted or unsubstituted C₁₋₆-alkyl phosphonate, substituted or unsubstituted C₁₋₆-alkyl phosphono and oxo;

m is equal to 0 or 1; and

R³ is hydrogen or fluorine.

In a further embodiment, the present invention relates to compounds of formula (I), geometrical isomers, enantiomers, diastereoisomers, pharmaceutically acceptable salts and all possible mixtures thereof,

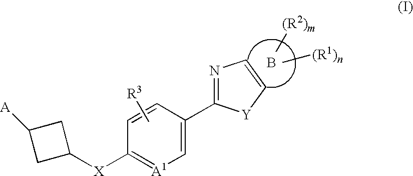

wherein

A is a 3 to 8 membered substituted or unsubstituted heterocycloalkyl linked to the cyclobutyl group via a nitrogen atom.

A¹ is CH;

B is a substituted or unsubstituted 5, 6 or 7-membered cycloalkyl, substituted or unsubstituted 5, 6 or 7-membered heterocycloalkyl, or a substituted or unsubstituted heteroaryl selected from the group comprising or consisting of tetrahydropyridyl, tetrahydro-1H-azepinyl, cyclopentenyl and pyridyl;

X is O or S;

Y is O, S or NH;

n is equal to 0;

R² is selected from the group comprising or consisting of hydrogen, carboxy, acyl, substituted or unsubstituted C₃₋₈ cycloalkyl, alkoxycarbonyl, substituted or unsubstituted C₁₋₆-alkyl alkoxycarbonyl, aminocarbonyl, substituted or unsubstituted C₁₋₆-alkyl aminocarbonyl, aminosulfonyl, substituted or unsubstituted C₁₋₆-alkyl hydroxy, substituted or unsubstituted C₁₋₆-alkyl phosphonate and substituted or unsubstituted C₁₋₆-alkyl phosphono;

m is equal to 0 or 1; and

R³ is hydrogen.

In a particular embodiment, the present invention relates to compounds of formula (I), geometrical isomers, enantiomers, diastereoisomers, pharmaceutically acceptable salts and all possible mixtures thereof,

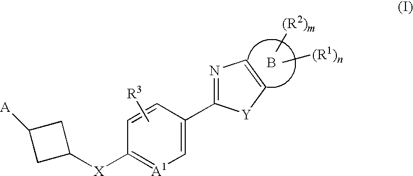

wherein

A is a 3 to 8 membered heterocycloalkyl linked to the cyclobutyl via a nitrogen atom selected from the group consisting of substituted or unsubstituted piperidin-1-yl, substituted or unsubstituted morpholin-4-yl, substituted or unsubstituted pyrrolidin-1-yl, substituted or unsubstituted piperazin-1-yl, substituted or unsubstituted azepanyl or substituted or unsubstituted thiomorpholin-4-yl;

A¹ is CH;

B is a substituted or unsubstituted 5, 6 or 7-membered cycloalkyl, a substituted or unsubstituted 5, 6 or 7-membered heterocycloalkyl, or a substituted or unsubstituted heteroaryl which forms together with the oxazole, the thiazole or the imidazole ring fused heterocycles including 4,5,6,7-tetrahydro[1,3]thiazolopyridine, 4,5,6,7-tetrahydro[1,3]oxazolopyridine, 4,5,6,7-tetrahydro-1H-imidazopyridine, 5,6-dihydro-4H-cyclopenta[d][1,3]thiazole, 5,6,7,8-tetrahydro-4H-[1,3]thiazoloazepine, 5,6,7,8-tetrahydro-4H-[1,3]oxazoloazepine, 1H-imidazopyridine and [1,3]thiazolopyridine.

X is O or S;

Y is O, S or NH;

n is equal to 0;

R² is selected from the group comprising or consisting of hydrogen, carboxy, acyl, substituted or unsubstituted C₃₋₈ cycloalkyl, alkoxycarbonyl, substituted or unsubstituted C₁₋₆-alkyl alkoxycarbonyl, aminocarbonyl, substituted or unsubstituted C₁₋₆-alkyl aminocarbonyl, aminosulfonyl, substituted or unsubstituted C₁₋₆-alkyl hydroxy, substituted or unsubstituted C₁₋₆-alkyl phosphonate and phosphonate and substituted or unsubstituted C₁₋₆-alkyl phosphono;

m is equal to 0 or 1; and

R³ is hydrogen.

In one specific embodiment, the present invention relates to compounds of formula (I), geometrical isomers, enantiomers, diastereoisomers, pharmaceutically acceptable salts and all possible mixtures thereof,

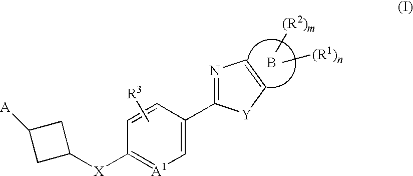

wherein

A is a 3 to 8 membered heterocycloalkyl selected from the group consisting of pyrrolidin-1-yl, 2-methylpyrrolidin-1-yl, (2S)-2-methylpyrrolidin-1-yl, (2R)-2-methylpyrrolidin-1-yl, piperidin-1-yl, 4,4-difluoropiperidin-1-yl, morpholin-4-yl, (3R)-3-(dimethylamino)pyrrolidin-1-yl, 3-(dimethylamino)pyrrolidin-1-yl, azepan-1-yl, thiomorpholin-4-yl, 4-isopropylpiperazin-1-yl and 4-cyclopentylpiperazin-1-yl;

A¹ is CH;

B is a substituted or unsubstituted 5, 6 or 7-membered cycloalkyl, a substituted or unsubstituted 5, 6 or 7-membered heterocycloalkyl, or a substituted or unsubstituted heteroaryl which forms together with the oxazole, the thiazole or the imidazole ring fused heterocycles including 4,5,6,7-tetrahydro[1,3]thiazolo[5,4-c]pyridine, 4,5,6,7-tetrahydro[1,3]thiazolo[4,5-b]pyridine, 4,5,6,7-tetrahydro[1,3]thiazolo[4,5-c]pyridine, 4,5,6,7-tetrahydro[1,3]oxazolo[4,5-c]pyridine, 4,5,6,7-tetrahydro-3H-imidazo[4,5-c]pyridine, 5,6,7,8-tetrahydro-4H-[1,3]thiazolo[5,4-b]azepine, 5,6,7,8-tetrahydro-4H-[1,3]oxazolo[5,4-b]azepine, 5,6-dihydro-4H-cyclopenta[d][1,3]thiazole, 3H-imidazo[4,5-c]pyridine and [1,3]thiazolo[4,5-c]pyridine;

X is O or S;

Y is O, S or NH;

n is equal to 0;

R² is selected from the group consisting of hydrogen, carboxy, methoxyacetyl, tert-butoxycarbonyl, acetyl, morpholin-4-ylcarbonyl, morpholin-4-ylsulfonyl, aminoacetyl, aminocarbonyl, hydroxyacetyl, 2,3-dihydroxypropyl, (2S)-2,3-dihydroxypropyl, (2R)-2,3-dihydroxypropyl, 2-amino-2-oxoethyl, (ethylamino)carbonyl, 3-hydroxycyclobutyl, 3-amino-3-oxopropanoyl, 2-methoxy-2-oxoethyl, [bis(ethyloxy)phosphoryl]methyl, 3,3,3-trifluoropropanoyl, phosphonomethyl, (5-methyl-2H-1,2,3-triazol-4-yl)carbonyl, 2-hydroxyethyl, 4-(1-oxidothiomorpholin-4-yl)butanoyl, 3-(acetylamino)propanoyl, (carboxymethoxy)acetyl, 3,3,3-trifluoro-2-hydroxypropanoyl, tetrahydro-2H-pyran-4-ylcarbonyl, (1-hydroxycyclopropyl)carbonyl, [(1-aminocarbonyl)cylopropyl]carbonyl, ethoxy(oxo)acetyl, [(aminocarbonyl)amino]carbonyl, amino(oxo)acetyl, 2,3-dihydroxypropanoyl, 2-hydroxy-3,4-dioxocyclobut-1-en-1-yl, 3,4-dioxo-2-(propan-2-yloxy)cyclobut-1-en-1-yl, 2-amino-3,4-dioxocyclobut-1-en-1-yl, (2S)-2,3-dihydroxypropanoyl and trifluoroacetyl;

m is equal to 0 or 1; and

R³ is hydrogen.

In another specific embodiment, the present invention relates to compounds of formula (I), geometrical isomers, enantiomers, diastereoisomers, pharmaceutically acceptable salts and all possible mixtures thereof,

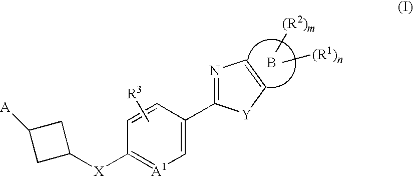

wherein

A is piperidin-1-yl, 2-methylpyrrolidin-1-yl, (2S)-2-methylpyrrolidin-1-yl or (2R)-2-methylpyrrolidin-1-yl;

A¹ is CH;

B forms together with the thiazole a 4,5,6,7-tetrahydro[1,3]thiazolo[5,4-c]pyridine;

X is O;

Y is S;

n is equal to 0;

R² is selected from the group consisting of acetyl, aminoacetyl, aminocarbonyl, hydroxyacetyl, 2,3-dihydroxypropyl, (2S)-2,3-dihydroxypropyl, (2R)-2,3-dihydroxypropyl, 2-amino-2-oxoethyl, 3-hydroxycyclobutyl, 3-amino-3-oxopropanoyl, (5-methyl-2H-1,2,3-triazol-4-yl)carbonyl, 2-hydroxyethyl, (carboxymethoxy)acetyl, tetrahydro-2H-pyran-4-ylcarbonyl, [(1-aminocarbonyl)cylopropyl]carbonyl, amino(oxo)acetyl, 2,3-dihydroxypropanoyl and 2-amino-3,4-dioxocyclobut-1-en-1-yl;

m is equal to 1; and

R³ is hydrogen.

In further specific embodiment, the present invention relates to compounds of formula (I), geometrical isomers, enantiomers, diastereoisomers, pharmaceutically acceptable salts and all possible mixtures thereof,

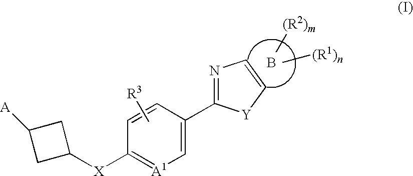

wherein

A is piperidin-1-yl, (2S)-2-methylpyrrolidin-1-yl or (2R)-2-methylpyrrolidin-1-yl;

A¹ is CH;

B forms together with the thiazole a 4,5,6,7-tetrahydro[1,3]thiazolo[5,4-c]pyridine;

X is O;

Y is S;

n is equal to 0;

R² is selected from the group consisting of acetyl, aminocarbonyl, hydroxyacetyl, 2-amino-2-oxoethyl and amino(oxo)acetyl;

m is equal to 1; and

R³ is hydrogen.

In one aspect, the present invention relates to compounds of formula (Ia), geometrical isomers, enantiomers, diastereoisomers, pharmaceutically acceptable salts and all possible mixtures thereof,

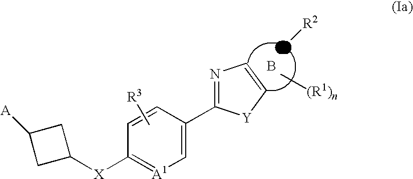

wherein A, A¹, X, Y, R¹, R², R³ and n are as herein defined and B is heteroaryl or 5-8-membered heterocycloalkyl.

Embodiments described hereinabove for A, A¹, X, Y, B, R¹, R², R³ and n in compounds of formula (I) also apply to A, A¹, X, Y, B, R¹, R², R³ and n in compounds of formula (Ia).

In another aspect, the present invention relates to compounds of formula (Ib), geometrical isomers, enantiomers, diastereoisomers, pharmaceutically acceptable salts and all possible mixtures thereof,

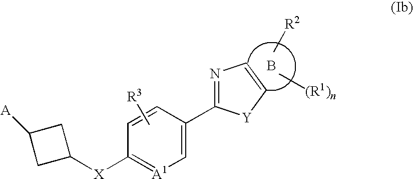

wherein A, A¹, X, Y, R¹, R², R³ and n are as herein defined and B is 5-8-membered cycloalkyl.

Embodiments described hereinabove for A, A¹, X, Y, B, R¹, R², R³ and n in compounds of formula (I) also apply to A, A¹, X, Y, B, R¹, R², R³ and n in compounds of formula (Ib).

In one aspect, the present invention relates to compounds of formula (Ic), geometrical isomers, enantiomers, diastereoisomers, pharmaceutically acceptable salts and all possible mixtures thereof,

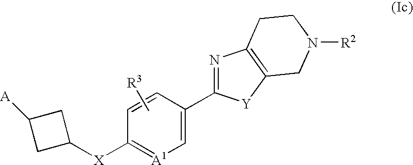

wherein A, A¹, X, Y, R² and R³ are as herein defined.

Embodiments described hereinabove for A, A¹, X, Y, R² and R³ in compounds of formula (I) also apply to A, A¹, X, Y, R² and R³ in compounds of formula (Ic).

In another aspect, the present invention relates to compounds of formula (Id), geometrical isomers, enantiomers, diastereoisomers, pharmaceutically acceptable salts and all possible mixtures thereof,

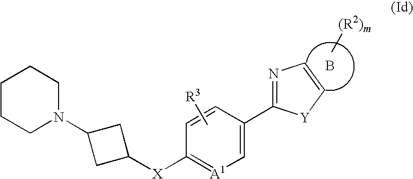

wherein A¹, X, Y, B, R², R³ and m are as herein defined.

Embodiments described hereinabove for A¹, X, Y, B, R² and R³ in compounds of formula (I) also apply to A¹, X, Y, B, R² and R³ in compounds of formula (Id).

In another aspect, the present invention relates to compounds of formula (Ie), geometrical isomers, enantiomers, diastereoisomers, pharmaceutically acceptable salts and all possible mixtures thereof,

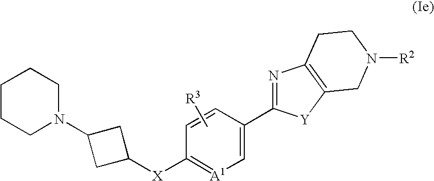

wherein X, Y, R² and R³ are as herein defined.

Embodiments described hereinabove for X, Y, R² and R³ in compounds of formula (I) also apply to X, Y, R² and R³ in compounds of formula (Ie).

In another aspect, the present invention relates to compounds of formula (If), geometrical isomers, enantiomers, diastereoisomers, pharmaceutically acceptable salts and all possible mixtures thereof,

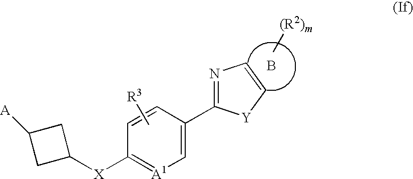

wherein A, A¹, B, Y, R², R³, m and n are as herein defined.

Embodiments described hereinabove for A, A¹, Y, B, R², R³, m and n in compounds of formula (I) also apply to A, A¹, X, Y, B, R², R³, m and n in compounds of formula (If).

In a particular embodiment, the present invention relates to a compound of formula (If) wherein

A is a 3 to 8 membered heterocycloalkyl linked to the cyclobutyl group via a nitrogen atom;

A1 is C—H;

Y is O, S or NH;

B is a substituted or unsubstituted 5, 6 or 7-membered cycloalkyl, a substituted or unsubstituted 5, 6 or 7-membered heterocycloalkyl, or a substituted or unsubstituted heteroaryl selected from the group comprising or consisting of a tetrahydropyridyl, a tetrahydro-1H-azepinyl, a cyclopentenyl or a pyridyl;

R² is selected from the group consisting of hydrogen, carboxy, acyl, substituted or unsubstituted C₃₋₈ cycloalkyl, alkoxycarbonyl, substituted or unsubstituted C₁₋₆-alkyl alkoxycarbonyl, aminocarbonyl, substituted or unsubstituted C₁₋₆ alkyl aminocarbonyl, aminosulfonyl, substituted or unsubstituted C₁₋₆-alkyl hydroxy, substituted or unsubstituted C₁₋₆-alkyl phosphonate and substituted or unsubstituted C₁₋₆-alkyl phosphono;

m is 1; and

R³ is hydrogen or halogen.

In a particular aspect, the present invention relates to compounds of formula (Ig), geometrical isomers, enantiomers, diastereoisomers, pharmaceutically acceptable salts and all possible mixtures thereof,

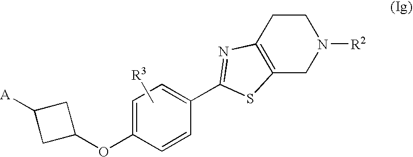

wherein A, R² and R³ are as herein defined.

According to a specific embodiment of compounds of formula (Ia), (Ib) and (Ic),

the A and X groups attached to the cyclobutyl in the A-cyclobutyl-X moiety are in trans configuration.

According to a specific embodiment of compounds of formula (Id) and (Ie), the piperidin-1-yl and X groups attached to the cyclobutyl in the (piperidin-1-yl)-cyclobutyl-X moiety are in trans configuration.

According to a specific embodiment of compounds of formula (If) and (Ig), the A and O groups attached to the cyclobutyl in the A-cyclobutyl-O moiety are in trans configuration.

Embodiments described hereinabove for R² and R³ in compounds of formula (I) also apply to R² and R³ in compounds of formula (If).

Examples of compounds according to the present invention are:
2-{4-[(trans-3-piperidin-1-ylcyclobutyl)oxy]phenyl}-5,6-dihydro-4H-cyclopenta[d][1,3]thiazole-5-carboxylic acid;
5-(methoxyacetyl)-2-{4-[(trans-3-piperidin-1-ylcyclobutyl)oxy]phenyl}-4,5,6,7-tetrahydro[1,3]thiazolo[5,4-c]pyridine;
tert-butyl 2-{4-[(trans-3-piperidin-1-ylcyclobutyl)oxy]phenyl}-6,7-dihydro[1,3]thiazolo[5,4-c]pyridine-5(4H)-carboxylate;
5-acetyl-2-{4-[(trans-3-piperidin-1-ylcyclobutyl)oxy]phenyl}-4,5,6,7-tetrahydro[1,3]thiazolo[5,4-c]pyridine;
2-{4-[(trans-3-piperidin-1-ylcyclobutyl)oxy]phenyl}-4,5,6,7-tetrahydro[1,3]thiazolo[5,4-c]pyridine;
5-(morpholin-4-ylcarbonyl)-2-{4-[(trans-3-piperidin-1-ylcyclobutyl)oxy]phenyl}-4,5,6,7-tetrahydro[1,3]thiazolo[5,4-c]pyridine;
5-(morpholin-4-ylsulfonyl)-2-{4-[(trans-3-piperidin-1-ylcyclobutyl)oxy]phenyl}-4,5,6,7-tetrahydro[1,3]thiazolo[5,4-c]pyridine;
5-acetyl-2-{4-[(trans-3-morpholin-4-ylcyclobutyl)oxy]phenyl}-4,5,6,7-tetrahydro[1,3]thiazolo[5,4-c]pyridine;
2-oxo-2-[2-{4-[(trans-3-piperidin-1-ylcyclobutyl)oxy]phenyl}-6,7-dihydro[1,3]thiazolo[5,4-c]pyridin-5(4H)-yl]ethanamine;
2-{4-[(trans-3-piperidin-1-ylcyclobutyl)oxy]phenyl}-6,7-dihydro[1,3]thiazolo[5,4-c]pyridine-5(4H)-carboxamide;
2-oxo-2-[2-{4-[(trans-3-piperidin-1-ylcyclobutyl)oxy]phenyl}-6,7-dihydro[1,3]thiazolo[5,4-c]pyridin-5(4H)-yl]ethanol;
5-acetyl-2-(4-{[trans-3-(4-isopropylpiperazin-1-yl)cyclobutyl]oxy}phenyl)-4,5,6,7-tetrahydro[1,3]thiazolo[5,4-c]pyridine;
5-acetyl-2-(4-{[trans-3-(4,4-difluoropiperidin-1-yl)cyclobutyl]oxy}phenyl)-4,5,6,7-tetrahydro[1,3]thiazolo[5,4-c]pyridine;
5-acetyl-2-{4-[(trans-3-pyrrolidin-1-ylcyclobutyl)oxy]phenyl}-4,5,6,7-tetrahydro[1,3]thiazolo[5,4-c]pyridine;
3-[2-{4-[(trans-3-piperidin-1-ylcyclobutyl)oxy}phenyl]-6,7-dihydro[1,3]thiazolo[5,4-c]pyridin-5(4H)-yl]propane-1,2-diol;
(2S)-3-[2-{4-[(trans-3-piperidin-1-ylcyclobutyl)oxy]phenyl}-6,7-dihydro[1,3]thiazolo[5,4-c]pyridin-5(4H)-yl]propane-1,2-diol;
(2R)-3-[2-{4-[(trans-3-piperidin-1-ylcyclobutyl)oxy]phenyl}-6,7-dihydro[1,3]thiazolo[5,4-c]pyridin-5(4H)-yl]propane-1,2-diol;
2-[2-{4-[(trans-3-piperidin-1-ylcyclobutyl)oxy]phenyl}-6,7-dihydro[1,3]thiazolo[5,4-c]pyridin-5(4H)-yl]acetamide;
5-acetyl-2-{4-[(trans-3-azepan-1-ylcyclobutyl)oxy]phenyl}-4,5,6,7-tetrahydro[1,3]thiazolo[5,4-c]pyridine;
(3R)-1-{trans-3-[4-(5-acetyl-4,5,6,7-tetrahydro[1,3]thiazolo[5,4-c]pyridin-2-yl)phenoxy]cyclobutyl}-N,N-dimethylpyrrolidin-3-amine;
N-ethyl-2-{4-[(trans-3-piperidin-1-ylcyclobutyl)oxy]phenyl}-6,7-dihydro[1,3]thiazolo[5,4-c]pyridine-5(4H)-carboxamide;
5-acetyl-2-{4-[(trans-3-thiomorpholin-4-ylcyclobutyl)oxy]phenyl}-4,5,6,7-tetrahydro[1,3]thiazolo[5,4-c]pyridine;
5-acetyl-2-{4-[(trans-3-piperidin-1-ylcyclobutyl)thio]phenyl}-4,5,6,7-tetrahydro[1,3]thiazolo[5,4-c]pyridine;
cis-3-[2-{4-[(trans-3-piperidin-1-ylcyclobutyl)oxy]phenyl}-6,7-dihydro[1,3]thiazolo[5,4-c]pyridin-5(4H)-yl]cyclobutanol;
3-oxo-3-[2-{4-[(trans-3-piperidin-1-ylcyclobutyl)oxy]phenyl}-6,7-dihydro[1,3]thiazolo[5,4-c]pyridin-5(4H)-yl]propanamide;
methyl[2-{4-[(trans-3-piperidin-1-ylcyclobutyl)oxy]phenyl}-6,7-dihydro[1,3]thiazolo[5,4-c]pyridin-5(4H)-yl]acetate;
diethyl {[2-{4-[(trans-3-piperidin-1-ylcyclobutyl)oxy]phenyl}-6,7-dihydro[1,3]thiazolo[5,4-c]pyridin-5(4H)-yl]methyl}phosphonate;
5-acetyl-2-{4-[(trans-3-piperidin-1-ylcyclobutyl)oxy]phenyl}-4,5,6,7-tetrahydro[1,3]oxazolo[4,5-c]pyridine;
5-acetyl-2-{4-[(trans-3-piperidin-1-ylcyclobutyl)oxy]phenyl}-4,5,6,7-tetrahydro[1,3]thiazolo[4,5-c]pyridine;
4-acetyl-2-{4-[(trans-3-piperidin-1-ylcyclobutyl)oxy]phenyl}-4,5,6,7-tetrahydro[1,3]thiazolo[4,5-b]pyridine;
4-acetyl-2-{4-[(trans-3-piperidin-1-ylcyclobutyl)oxy]phenyl}-5,6,7,8-tetrahydro-4H-[1,3]thiazolo[5,4-b]azepine;
2-{4-[(trans-3-piperidin-1-ylcyclobutyl)oxy]phenyl}[1,3]thiazolo[4,5-c]pyridine;
2-{4-[(trans-3-piperidin-1-ylcyclobutyl)oxy]phenyl}-5-(3,3,3-trifluoropropanoyl)-4,5,6,7-tetrahydro[1,3]thiazolo[5,4-c]pyridine;
{[2-{4-[(trans-3-piperidin-1-ylcyclobutyl)oxy]phenyl}-6,7-dihydro[1,3]thiazolo[5,4-c]pyridin-5(4H)-yl]methyl}phosphonic acid;
5-[(5-methyl-2H-1,2,3-triazol-4-yl)carbonyl]-2-{4-[(trans-3-piperidin-1-ylcyclobutyl)oxy]phenyl}-4,5,6,7-tetrahydro[1,3]thiazolo[5,4-c]pyridine;
5-acetyl-2-{2-fluoro-4-[(trans-3-piperidin-1-ylcyclobutyl)oxy]phenyl}-4,5,6,7-tetrahydro[1,3]thiazolo[5,4-c]pyridine;
2-[2-{4-[(trans-3-piperidin-1-ylcyclobutyl)oxy]phenyl}-6,7-dihydro[1,3]thiazolo[5,4-c]pyridin-5(4H)-yl]ethanol;
5-acetyl-2-{2,6-difluoro-4-[(trans-3-piperidin-1-ylcyclobutyl)oxy]phenyl}-4,5,6,7-tetrahydro[1,3]thiazolo[5,4-c]pyridine;
5-acetyl-2-{3-fluoro-4-[(trans-3-piperidin-1-ylcyclobutyl)oxy]phenyl}-4,5,6,7-tetrahydro[1,3]thiazolo[5,4-c]pyridine;
5-acetyl-2-{2,3-difluoro-4-[(trans-3-piperidin-1-ylcyclobutyl)oxy]phenyl}-4,5,6,7-tetrahydro[1,3]thiazolo[5,4-c]pyridine;
5-[4-(1-oxidothiomorpholin-4-yl)butanoyl]-2-{4-[(trans-3-piperidin-1-ylcyclobutyl)oxy]phenyl}-4,5,6,7-tetrahydro[1,3]thiazolo[5,4-c]pyridine;
N-{3-oxo-3-[2-{4-[(trans-3-piperidin-1-ylcyclobutyl)oxy]phenyl}-6,7-dihydro[1,3]thiazolo[5,4-c]pyridin-5(4H)-yl]propyl}acetamide;
{2-oxo-2-[2-{4-[(trans-3-piperidin-1-ylcyclobutyl)oxy]phenyl}-6,7-dihydro[1,3]thiazolo[5,4-c]pyridin-5(4H)-yl]ethoxy}acetic acid;
1,1,1-trifluoro-3-oxo-3-[2-{4-[(trans-3-piperidin-1-ylcyclobutyl)oxy]phenyl}-6,7-dihydro[1,3]thiazolo[5,4-c]pyridin-5(4H)-yl]propan-2-ol;
2-{4-[(trans-3-piperidin-1-ylcyclobutyl)oxy]phenyl}-5-(tetrahydro-2H-pyran-4-ylcarbonyl)-4,5,6,7-tetrahydro[1,3]thiazolo[5,4-c]pyridine;
1-{[2-{4-[(trans-3-piperidin-1-ylcyclobutyl)oxy]phenyl}-6,7-dihydro[1,3]thiazolo[5,4-c]pyridin-5(4H)-yl]carbonyl}cyclopropanol;
1-{[2-{4-[(trans-3-piperidin-1-ylcyclobutyl)oxy]phenyl}-6,7-dihydro[1,3]thiazolo[5,4-c]pyridin-5(4H)-yl]carbonyl}cyclopropanecarboxamide;
1-{[2-{4-[(trans-3-piperidin-1-ylcyclobutyl)oxy]phenyl}-6,7-dihydro[1,3]thiazolo[5,4-c]pyridin-5(4H)-yl]carbonyl}cyclopropanecarboxamide trifluoroacetate;
ethyl oxo[2-{4-[(trans-3-piperidin-1-ylcyclobutyl)oxy]phenyl}-6,7-dihydro[1,3]thiazolo[5,4-c]pyridin-5(4H)yl]acetate;
1-{trans-3-[4-(5-acetyl-4,5,6,7-tetrahydro[1,3]thiazolo[5,4-c]pyridin-2-yl)phenoxy]cyclobutyl}-N,N-dimethylpyrrolidin-3-amine;
5-acetyl-2-(4-{[trans-3-(4-cyclopentylpiperazin-1-yl)cyclobutyl]oxy}phenyl)-4,5,6,7-tetrahydro[1,3]thiazolo[5,4-c]pyridine;
1-{2-oxo-2-[2-{4-[(trans-3-piperidin-1-ylcyclobutyl)oxy]phenyl}-6,7-dihydro[1,3]thiazolo[5,4-c]pyridin-5(4H)-yl]ethyl}urea;
2-oxo-2-[2-{4-[(trans-3-piperidin-1-ylcyclobutyl)oxy]phenyl}-6,7-dihydro[1,3]thiazolo[5,4-c]pyridin-5(4H)-yl]acetamide;
3-oxo-3-[2-{4-[(trans-3-piperidin-1-ylcyclobutyl)oxy]phenyl}-6,7-dihydro[1,3]thiazolo[5,4-c]pyridin-5(4H)yl]propane-1,2-diol;
3-hydroxy-4-[2-{4-[(trans-3-piperidin-1-ylcyclobutyl)oxy]phenyl}-6,7-dihydro[1,3]thiazolo[5,4-c]pyridin-5(4H)-yl]cyclobut-3-ene-1,2-dione;
3-isopropoxy-4-[2-{4-[(trans-3-piperidin-1-ylcyclobutyl)oxy]phenyl}-6,7-dihydro[1,3]thiazolo[5,4-c]pyridin-5(4H)-yl]cyclobut-3-ene-1,2-dione;
5-acetyl-2-[4-({trans-3-[2-methylpyrrolidin-1-yl]cyclobutyl}oxy)phenyl]-4,5,6,7-tetrahydro[1,3]thiazolo[5,4-c]pyridine, isomer A;
3-amino-4-[2-{4-[(trans-3-piperidin-1-ylcyclobutyl)oxy]phenyl}-6,7-dihydro[1,3]thiazolo[5,4-c]pyridin-5(4H)-yl]cyclobut-3-ene-1,2-dione.1/2 trifluoroacetate;
5-acetyl-2-[4-({trans-3-[2-methylpyrrolidin-1-yl]cyclobutyl}oxy)phenyl]-4,5,6,7-tetrahydro[1,3]thiazolo[5,4-c]pyridine, isomer B;
(2S)-3-oxo-3-[2-{4-[(trans-3-piperidin-1-ylcyclobutyl)oxy]phenyl}-6,7-dihydro[1,3]thiazolo[5,4-c]pyridin-5(4H)-yl]propane-1,2-diol;
5-acetyl-2-{4-[(cis-3-piperidin-1-ylcyclobutyl)oxy]phenyl}-4,5,6,7-tetrahydro-3H-imidazo[4,5-c]pyridine;
5-acetyl-2-{4-[(trans-3-piperidin-1-ylcyclobutyl)oxy]phenyl}-4,5,6,7-tetrahydro-3H-imidazo[4,5-c]pyridine;
2-oxo-2-[2-{4-[(trans-3-piperidin-1-ylcyclobutyl)oxy]phenyl}-6,7-dihydro[1,3]thiazolo[4,5-c]pyridin-5(4H)-yl]ethanol;
2-{4-[(trans-3-piperidin-1-ylcyclobutyl)oxy]phenyl}-6,7-dihydro[1,3]thiazolo[4,5-c]pyridine-5(4H)-carboxamide;
3-oxo-3-[2-{4-[(trans-3-piperidin-1-ylcyclobutyl)oxy]phenyl}-6,7-dihydro[1,3]thiazolo[4,5-c]pyridin-5(4H)-yl]propanamide;
2-oxo-2-[2-{4-[(trans-3-piperidin-1-ylcyclobutyl)oxy]phenyl}-6,7-dihydro[1,3]thiazolo[4,5-b]pyridin-4(5H)-yl]ethanol;
4-acetyl-2-{4-[(trans-3-piperidin-1-ylcyclobutyl)oxy]phenyl}-5,6,7,8-tetrahydro-4H-[1,3]oxazolo[5,4-b]azepine;
2-{4-[(trans-3-piperidin-1-ylcyclobutyl)oxy]phenyl}-4-(trifluoroacetyl)-5,6,7,8-tetrahydro-4H-[1,3]oxazolo[5,4-b]azepine; and
2-{4-[(trans-3-piperidin-1-ylcyclobutyl)oxy]phenyl}-3H-imidazo[4,5-c]pyridine.

In a particular embodiment, the present invention relates to compounds of formula (I) selected from the group consisting of:
5-acetyl-2-{4-[(trans-3-piperidin-1-ylcyclobutyl)oxy]phenyl}-4,5,6,7-tetrahydro[1,3]thiazolo[5,4-c]pyridine;
2-{4-[(trans-3-piperidin-1-ylcyclobutyl)oxy]phenyl}-6,7-dihydro[1,3]thiazolo[5,4-c]pyridine-5(4H)-carboxamide;
2-oxo-2-[2-{4-[(trans-3-piperidin-1-ylcyclobutyl)oxy]phenyl}-6,7-dihydro[1,3]thiazolo[5,4-c]pyridin-5(4H)-yl]ethanol;
2-[2-{4-[(trans-3-piperidin-1-ylcyclobutyl)oxy]phenyl}-6,7-dihydro[1,3]thiazolo[5,4-c]pyridin-5(4H)-yl]acetamide;
2-oxo-2-[2-{4-[(trans-3-piperidin-1-ylcyclobutyl)oxy]phenyl}-6,7-dihydro[1,3]thiazolo[5,4-c]pyridin-5(4H)-yl]acetamide;
5-acetyl-2-[4-({trans-3-[2-methylpyrrolidin-1-yl]cyclobutyl}oxy)phenyl]-4,5,6,7-tetrahydro[1,3]thiazolo[5,4-c]pyridine and enantiomers; and
2-{4-[(trans-3-piperidin-1-ylcyclobutyl)oxy]phenyl}-6,7-dihydro[1,3]thiazolo[4,5-c]pyridine-5(4H)-carboxamide.

The compounds of the present invention are histamine H₃-receptor ligands. In one embodiment they are histamine H₃-receptor antagonists; in another embodiment they are histamine H₃-receptor inverse agonists.

In one embodiment, compounds of the present invention have particularly favorable drug properties, i.e. they have a good affinity to the H₃-receptor while having a low affinity towards other receptors or proteins; they have favorable pharmacokinetics and pharmacodynamics while having few side effects, e.g. toxicity such as cardiotoxicity. One of many methods known to determine the cardiovascular risk of drug compounds is to assess the binding of a test compound to hERG channels.

Compounds of the present invention display a particular low affinity on hERG channels.

Moreover, preferred compounds according to the present invention exhibit good brain H₃ receptor occupancy.

The “pharmaceutically acceptable salts” according to the invention include therapeutically active, non-toxic acid salt forms which the compounds of formula (I) are able to form.

The acid addition salt form of a compound of formula (I) that occurs in its free form as a base can be obtained by treating the free base with an appropriate acid such as an inorganic acid, for example, a hydrochloric, hydrobromic, sulfuric, nitric, phosphoric and the like; or an organic acid, such as, for example, acetic, trifluoroacetic, hydroxyacetic, propanoic, lactic, pyruvic, malonic, succinic, maleic, fumaric, malic, tartaric, citric, methanesulfonic, ethanesulfonic, benzenesulfonic, p-toluenesulfonic, cyclamic, salicylic, p-aminosalicylic, palmoic, and the like.

Conversely said salt forms can be converted into the free forms by treatment with an appropriate base.

Compounds of the formula (I) and their salts can be in the form of a solvate, which is included within the scope of the present invention. Such solvates include for example hydrates, alcoholates and the like.

Some of the compounds of formula (I) and some of their intermediates have at least one stereogenic center in their structure. This stereogenic center may be present in a R or a S configuration, said R and S notation is used in correspondence with the rules described in Pure Appl. Chem. 1976, 45, 11-30.

The invention also relates to all stereoisomeric forms such as enantiomeric and diastereomeric forms of the compounds of formula (I) or mixtures thereof (including all possible mixtures of stereoisomers).

With respect to the present invention reference to a compound or compounds is intended to encompass that compound in each of its possible isomeric forms and mixtures thereof, unless the particular isomeric form is referred to specifically.

The expression “enantiomerically pure” as used herein refers to compounds which have an enantiomeric excess (ee) greater 95%.

Compounds according to the present invention may exist in different polymorphic forms. Although not explicitly indicated in the above formula, such forms are included within the scope of the present invention.

The invention also includes within its scope pro-drug forms of the compounds of formula (I) and its various sub-scopes and sub-groups.

The term “prodrug” as used herein includes compound forms which are rapidly transformed in vivo to the parent compound according to the invention, for example, by hydrolysis in blood. Prodrugs are compounds bearing groups which are removed by biotransformation prior to exhibiting their pharmacological action. Such groups include moieties which are readily cleaved in vivo from the compound bearing it, which compound after cleavage remains or becomes pharmacologically active. Metabolically cleavable groups form a class of groups well known to practitioners of the art. They include, but are not limited to such groups as alkanoyl (i.e. acetyl, propionyl, butyryl, and the like), unsubstituted and substituted carbocyclic aroyl (such as benzoyl, substituted benzoyl and 1- and 2-naphthoyl), alkoxycarbonyl (such as ethoxycarbonyl), trialklysilyl (such as trimethyl- and triethylsilyl), monoesters formed with dicarboxylic acids (such as succinyl), phosphate, sulfate, sulfonate, sulfonyl, sulfinyl and the like. The compounds bearing the metabolically cleavable groups have the advantage that they may exhibit improved bioavailability as a result of enhanced solubility and/or rate of absorption conferred upon the parent compound by virtue of the presence of the metabolically cleavable group. T. Higuchi and V. Stella, “Pro-drugs as Novel Delivery System”, Vol. 14 of the A.C.S. Symposium Series; “Bioreversible Carriers in Drug Design”, ed. Edward B. Roche, American Pharmaceutical Association and Pergamon Press, 1987.

Compounds of formula (I) according to the invention may be prepared according to conventional methods known to the person skilled in the art of synthetic organic chemistry.

A. According to one embodiment, compounds of formula (I) wherein A¹ is CH, X is an oxygen, A, Y, B, R¹, R², R³, m and n having the same definition as described in the general formula above, may be prepared by reaction of a compound of formula (II) with a compound of formula (III) according to the equation:

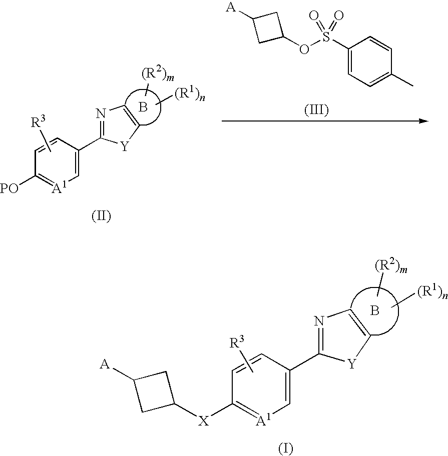

wherein A¹ is CH, P is a hydrogen, A, B, R¹, R², R³, Y, m and n having the same definition as in the general formula above for compound of formula (I).

This reaction may be carried out in the presence of a base, for example sodium hydride, in a solvent, for example N,N-dimethylacetamide, under an inert atmosphere, at a temperature ranging from 50 to 80° C. or in any other conditions that the man skilled in the art will deem appropriate, and according to conventional methods known to him.

In a particular embodiment, this method may be used for the synthesis of compounds of formula (Ia) as defined above.

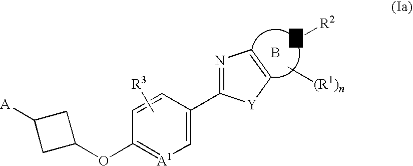

In another particular embodiment, this method may be used for the synthesis of compounds of formula (Ib) as defined above.

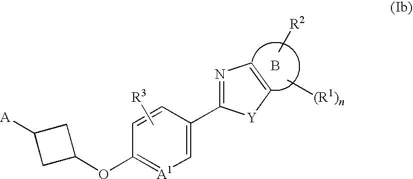

Compounds of formula (III) may be prepared by reaction of a compound of formula (IV) with p-toluenesulfonyl chloride or p-bromophenylsulfonyl chloride according to the equation:

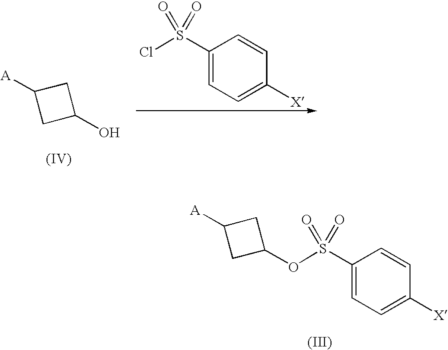

wherein A has the same definition as described above for compounds of formula (I) and X′ is methyl or bromide.

This reaction may be carried out using a base such as triethylamine or N-methylimidazole, in a solvent such as dichloromethane, at a temperature ranging from 0° C. to 25° C., under an inert atmosphere (argon or nitrogen), or according to any conventional method known by the man skilled in the art.

Compound of formula (IV) may be prepared from a compound of formula (V), according to the equation:

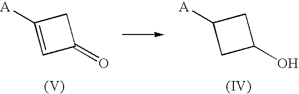

wherein A has the same definition as described above for compounds of formula I.

This reaction may be carried out using a reductive agent such as sodium borohydride, in a protic solvent such as ethanol, at a temperature ranging from 0° C. to 60° C., under an inert atmosphere (argon or nitrogen), or according to any conventional method known by the man skilled in the art.

Compound of formula (V) may be commercially available or prepared from cyclobutane-1,3-dione by reaction with an amine of formula AH, according to the equation:

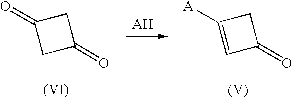

wherein A has the same definition as described above for compounds of formula I.

This reaction may be carried out in a solvent such as dioxane, at a temperature ranging from 0° C. to 30° C., under an inert atmosphere (argon or nitrogen), or according to any conventional method known by the man skilled in the art. Cyclobutan-1,3-dione is commercially available or may be prepared according to any conventional method known to the person skilled in the art.

Compounds of formula (II) may be prepared according to one of the following methods:

A.1. Some compounds of formula (II) wherein Y is a sulfur atom and P is hydrogen or a protecting group may be prepared by reaction of a compound of formula (VII) with a compound of formula (VIII) according to the equation:

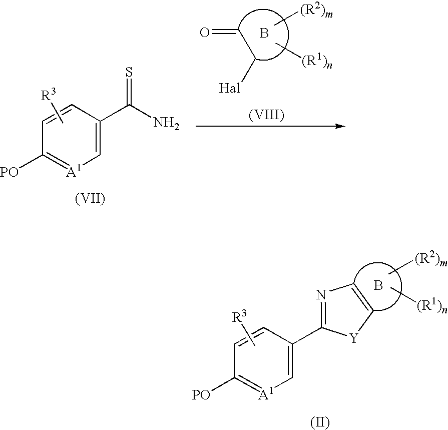

wherein P is hydrogen or a protecting group, Y is a sulfur, A¹, B, R¹, R², R³, m and n having the same definition as in the general formula above for compound of formula (I) and Hal is a leaving group, preferably a bromine atom.

Examples of protecting groups may be a benzyl group, a trialkylsilyl group, a tert-butoxy group, an acetyl group, an alkyl group or any other phenol-related protecting groups that the man skilled in the art will deem appropriate. Such protecting groups may be removed using any methodologies and experimental conditions that the man skilled in the art will deem appropriate, and according to conventional methods known to him.

This reaction may be carried out in the presence of a solvent, such as ethanol or iso-propanol, at a temperature ranging from 50° C. to 100° C., or according to the method described by Ashton, W. T. et al. in Bioorg. Med. Chem. Lett. 2005, 15, 2253, or according to any other conventional methods known to the man skilled in the art.

In a particular embodiment, this method may be used for the synthesis of compounds of formula (II), hereafter referenced as compounds (IIa), wherein P is hydrogen or a protecting group, Y is S, B is a 5-8-membered heterocycloalkyl group containing a nitrogen atom, m is 1 and R² is linked to the nitrogen atom, A1, R¹, R², R³ and n having the same definitions as described above for compounds of formula (II). Preferably, n is equal to 0.

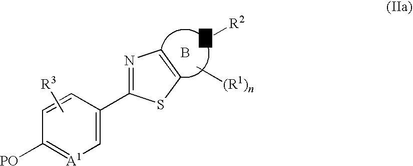

In another particular embodiment, the same method may be used for the synthesis of compounds of formula (II), hereafter referenced as compounds (IIb), wherein P is hydrogen or a protecting group, Y is S, B is a 5-8-membered heterocycloalkyl group containing a nitrogen atom, m is 1 and R² is linked to this nitrogen atom, one of the R¹ is an oxo group to form a lactam moiety, A1, R¹, R², n and R³ having the same definitions as described above for compounds of formula (II). Preferably, n is equal to 1.

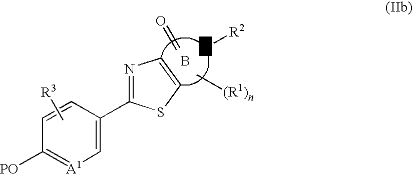

In another particular embodiment, the same method may be used for the synthesis of compounds of formula (II), hereafter referenced as compounds (IIc), wherein P is hydrogen or a protecting group, Y is S, B is a 5-8-membered cycloalkyl group, m is 1 and R² is alkoxycarbonyl, A¹, R¹, R³ and n having the same definitions as described above for compounds of formula (II). Preferably, n is equal to 0.

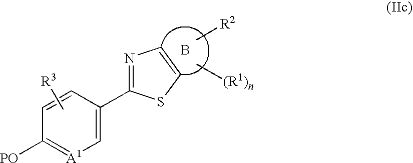

Compounds of formula (VII) may be commercially available or prepared according to any conventional method known to the person skilled in the art.

Compounds of formula (VIII) wherein Hal is a halogen atom, R¹, R², m and n being as defined in the specifications for compounds of general formula (I), may be prepared by reaction of a compound of formula (IX) with a halogen-releasing agent according to the equation:

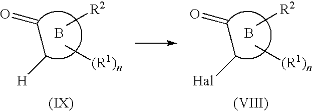

This reaction may be carried out using bromine (Br₂) or polymer-supported pyridinium tribromide, in a solvent such as dichloromethane or chloroform, at a temperature ranging from 0° C. to 25° C., according to the methods described by Marinko, P. et al. Eur. J. Med. Chem., 2004, 39, 257 or Habermann, J. et al. J. Chem. Soc., Perkin Trans. 1, 1999, 2425, or according to any conventional method known to him. Preferably, Hal is a bromine atom.

Alternatively, some compounds of formula (VIII) may be prepared in two steps according to the equation:

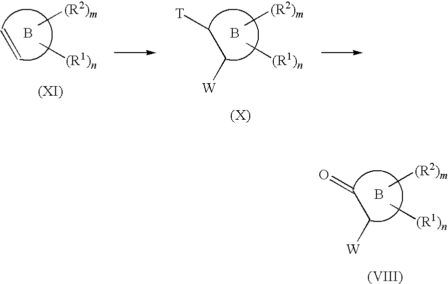

wherein W represents a halogen atom, preferably a bromine atom, T is hydroxy, B is a 5-8-membered heterocycloalkyl or a 5-8-membered cycloalkyl, R¹, R², m and n having the same definitions as described above.

Compounds of formula (VIII) may be prepared by reaction of a “halohydrine” of formula (X) with an oxidizing agent, such as Dess-Martin periodinane reagent or pyridinium chlorochromate, or according to any conventional methods known to the man skilled in the art.

Compounds of formula (X) may be commercially available. They may also be prepared by the reaction of a 5-8-membered cycloalkene or a 5-8-membered heterocycloalkene of formula (XI) with a halogen-releasing agent, such as N-bromosuccinimide, in the presence of water, according to the method described by Kim, W.-J. et al. in Heterocycles, 1995, 41, 1389; or according to any other conventional methods known to the man skilled in the art.

Compounds of formula (XI) may be commercially available or may be prepared according to any other conventional methods known to the man skilled in the art, For example, compounds (XI) may be prepared by intramolecular metathesis reaction of a di-alkene according to the method described by Yao, Q. et al. in Angew. Chem. Int. Ed., 2000, 39, 3896.

A.2. Some compounds of formula (II) wherein P is hydrogen, A¹, B, R¹, R², R³, m and n having the same definition as in the general formula above for compound of formula (I), may be prepared by deprotection of the corresponding compound of formula (II) wherein P is a protecting group. For example, when P is methyl or benzyl, this reaction may be carried out using boron tribromide in a solvent such as dichloromethane at room temperature, or using any other reagents and reaction conditions known to the man skilled in the art.

In a particular embodiment, compounds of formula (IIa) wherein P is hydrogen may be obtained by deprotection of the corresponding compound of formula (IIa) wherein P is a protecting group.

A.3. Some compounds of formula (IIa) may be obtained by reduction of the corresponding compounds of formula (IIb) according to the equation:

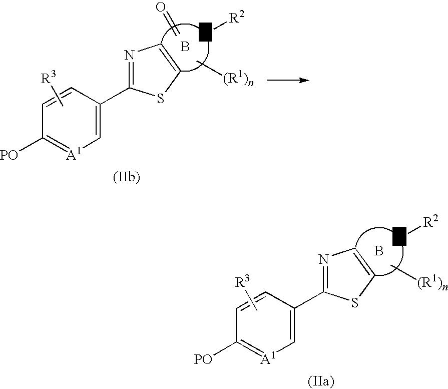

wherein P, A1, R¹, R², R³ and n have the same definitions as described above for compounds of formula (IIa). For example, this reaction may be carried out by the use of a reducing agent such as borane derivatives (e.g., borane-dimethyl sulfide complex) in a solvent such as THF or ether and at a temperature ranging from 0° C. to 100° C., preferably at room temperature. Alternatively, this reaction may be carried out using other experimental conditions that the man skilled in the art will deem appropriate, and according to conventional methods known to him.

A.4. Some compounds of formula (IIa) wherein P is a protecting group and R² is hydrogen may be prepared by cyclization of a compound of formula (XII) according to the equation:

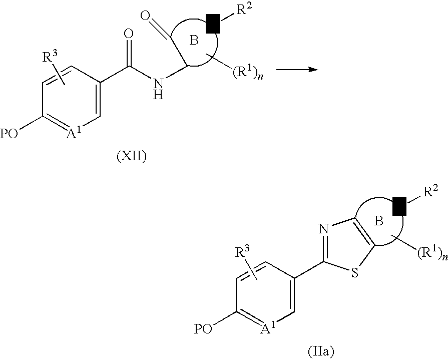

wherein P is a protecting group, R² is hydrogen, A¹, B, R¹, R³, Y, n having the same definition as described above for compounds of formula (IIa). This reaction may be carried out using Lawesson's reagent in a solvent such as pyridine at reflux temperature, or according to any other method known to the person skilled in the art.

Compounds of formula (XII) may be prepared by reaction of a compound of formula (XIII) with a compound of formula (XIV) according to the equation:

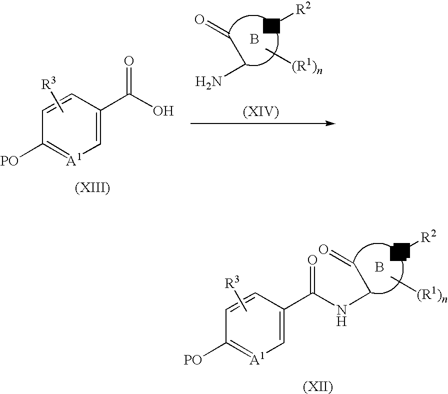

wherein P is a protecting group, A1, R¹, R², R³, n having the same definition as described above for compounds of formula (IIa). This reaction may be carried out using oxalyl chloride in a solvent such as dichloromethane at low temperature to form an intermediate acid chloride which is subsequently reacted with the compound of formula (XIV) in a solvent such as dichloromethane in the presence of a base such as triethylamine at room temperature or using any other reagents and reaction conditions known to the man skilled in the art.

Compounds of formula (XIII) and (XIV) may be commercially available or prepared according to any conventional methods known to the man skilled in the art.

A.5. Compounds of formula (IIa) wherein P is a protecting group and R² is hydrogen may alternatively be prepared by reduction of a compound of formula (XV) according to the equation:

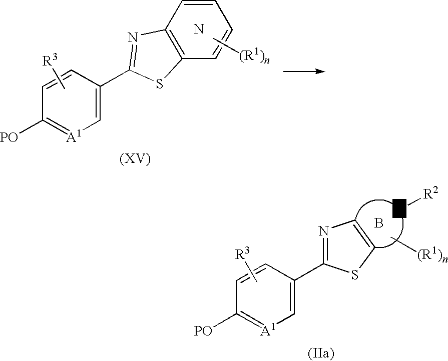

wherein P is a protecting group, R² is hydrogen, A¹, B, R¹, R³ and n having the same definition as described above for compound of formula (IIa). This reaction may be carried out using hydrogen in the presence of a suitable catalyst such as platinum dioxide in a solvent such as acetic acid or using lithium triethylborohydride in tetrahydrofuran or according to any conventional method known to the man skilled in the art.

Compounds of formula (XV) may be prepared by cyclization of a compound of formula (XVI) according to the equation:

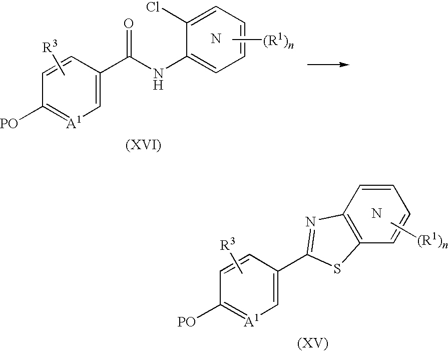

wherein P is a protecting group, R² is hydrogen, A¹, B, R¹, R³ and n having the same definition as described above for compounds of formula (IIa). For example, this reaction may be carried out using Lawesson's reagent in a solvent such as toluene at reflux temperature.

Compounds of formula (XVI) may be prepared by reaction of a compound of formula (XIII) with a compound of formula (XVII) according to the equation:

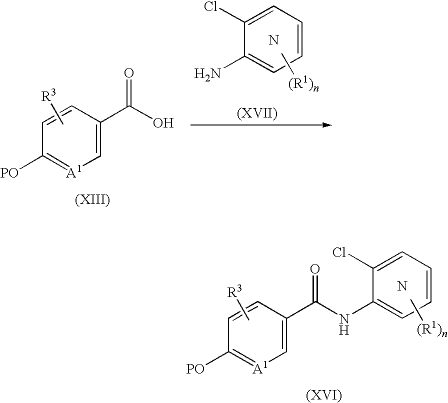

wherein P, A¹, R¹, R³ and n are defined as above. This reaction may be carried out using oxalyl chloride in a solvent such as dichloromethane, at low temperature to form an intermediate acid chloride which is subsequently reacted at room temperature with the compound of formula (XVII), pretreated with a strong base such as sodium hydride, in a solvent such as N,N-dimethylformamide or dichloromethane, or using any other reagents and reaction conditions known to the man skilled in the art.

Compounds of formula (XVII) may be commercially available or prepared according to any conventional methods known to the man skilled in the art.

A.6. Compounds of formula (II) wherein P is a protecting group and R² is an acyl group may be prepared by reaction of the corresponding compound of formula (II) wherein R² is hydrogen with acid chlorides or anhydrides in the presence of a base such as triethylamine or N,N-dimethylaminopyridine according to conventional methods known to the man skilled in the art.

A.7. Compounds of formula (II) wherein P is H, Y is oxygen, B is a 5-8-membered heterocycloalkyl group containing a nitrogen atom, R² is linked to the nitrogen atom and is hydrogen, hereafter referenced as compounds (II′a), may be prepared by deprotection of a compound of formula (II′b) according to the equation:

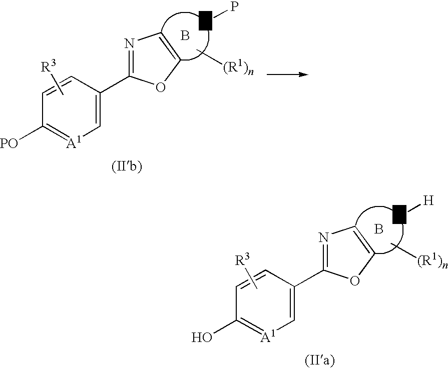

wherein P is a protecting group such as benzyl, B is a 5-8-membered heterocycloalkyl group containing a nitrogen atom, A¹, R¹, R³ and n having the same definitions as described above for compounds of formula (I). This reaction may be carried out using hydrogen as reducing agent in a solvent such as acetic acid in the presence of a suitable catalyst such as palladium acetate or using any other reagents and reaction conditions known to the man skilled in the art. Preferably, n is equal to 0.

Some compounds of formula (II′b) may be prepared by cyclisation of a compound of formula (XII) in the presence of a suitable activating agent, such as titanium (IV) chloride or any other reagents and reaction conditions known to the man skilled in the art.

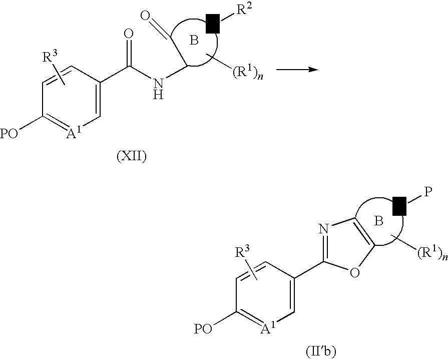

Some compounds of formula (II′b) may be prepared by reduction of a compound of formula (XVIII) according to the equation:

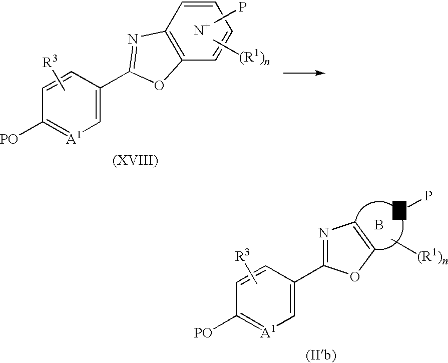

wherein P is a protecting group such as benzyl and n is preferably equal to 0, A¹, R¹, R³ and B having the same definitions as described above for compounds of formula (Ira). This reaction may be carried out using a reducing agent such as sodium borohydride in a solvent such as ethanol at a temperature ranging from 0° C. to 60° C. or using any other reagents and reaction conditions known to the man skilled in the art. Preferably, n is equal to 0.

Compounds of formula (XVIII) may be prepared by alkylation of compounds of formula (XIX) according to the equation:

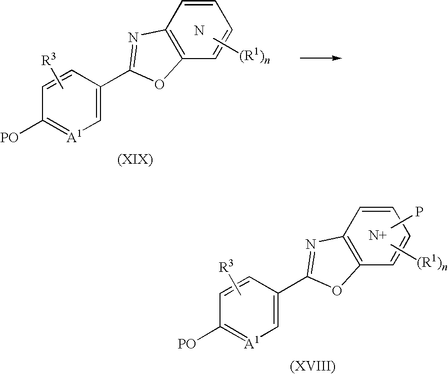

wherein P is a protecting group such as benzyl, A¹, R¹, R³ and n having the same definitions as described above for compounds of formula (Ira). This reaction may be carried out using an alkylating agent such as benzyl bromide according to any conventional methods known to the man skilled in the art. Preferably, n is equal to 0.

Compounds of formula (XIX) may be prepared by cyclization of a compound of formula (XX) according to the equation:

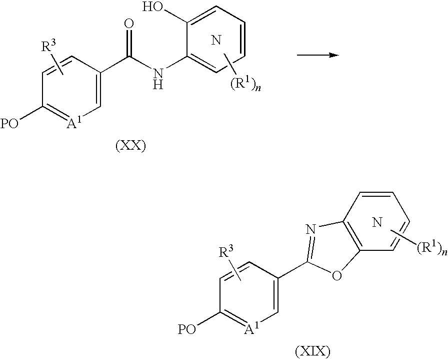

wherein P is a protecting group such as benzyl, A¹, R¹, R³ and n having the same definitions as described above for compounds of formula (II′a). This reaction may be carried out with triphenylphosphine and hexachloroethane in a solvent such as dichloromethane in the presence of a base such as triethylamine, at room temperature, according to the method described by Heuser, S. et al. Tetrahedron Lett., 2005, 46, 9001-9004. Preferably, n is equal to 0.

Compounds of formula (XX) may be prepared starting from the corresponding carboxylic acid (XIII) as indicated above for the preparation of compounds (XVI), or according to any method known to the man skilled in the art.

B. Compounds of formula (I) wherein A¹ is CH, X is a sulfur, Y is a sulfur, A, B, R¹, R², R³, m and n having the same definition as described above for the general formula (I), may be prepared by reaction of a compound of formula (XXI) with a compound of formula (VIII) according to the equation:

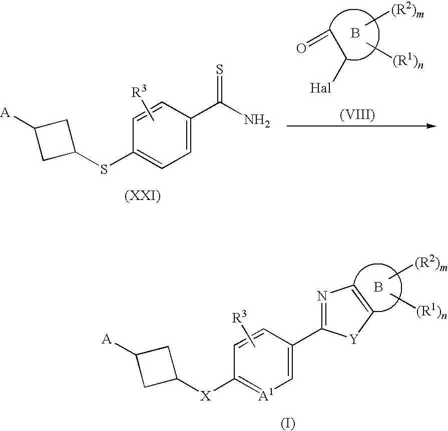

wherein A¹ is CH, X is a sulfur, Y is a sulfur, A, B, R¹, R², R³, m and n having the same definition as described in the general formula above and Hal is halogen, preferably a bromine atom.

This reaction may be carried out in the presence of a solvent, such as ethanol or iso-propanol, at a temperature ranging from 50° C. to 100° C., or according to the method described by Ashton, W. T. et al. in Bioorg. Med. Chem. Lett. 2005, 15, 2253, or according to any other conventional methods known to the man skilled in the art.

Compounds of formula (XXI) may be prepared from compounds of formula (XXII) according to the equation:

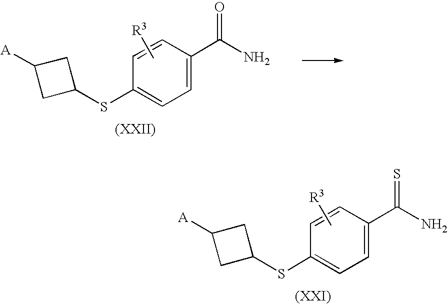

wherein A and R³ have the same definition as described above. For example, this reaction may be carried out using Lawesson's reagent in a solvent such as tetrahydrofuran at room temperature or according to any other conventional methods known to the man skilled in the art.

Compounds or formula (XXII) may be prepared by ammonolysis of compounds of formula (XXI) according to the equation:

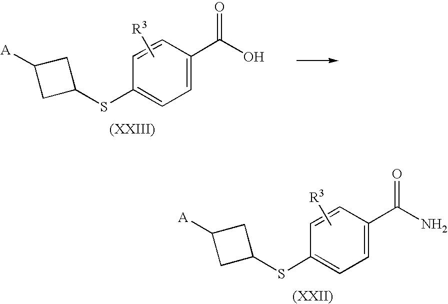

wherein A and R³ have the same definition as described above. This reaction may be carried out according to any conventional method known to the man skilled in the art.

Compounds of formula (XXIII) may be prepared by reaction of a compound of formula (XXIV) with a compound of formula (III) according to the equation:

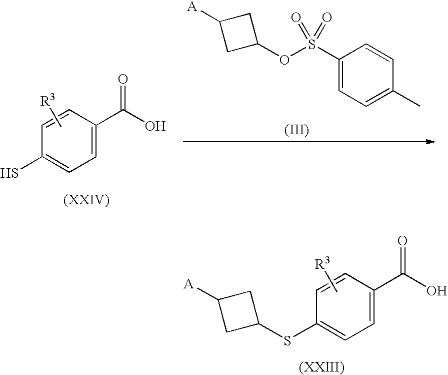

wherein A and R³ have the same definition as described above for compounds of formula (I).

This reaction may be carried out in the presence of a base, for example sodium hydride, in a solvent, for example N,N-dimethylacetamide, under an inert atmosphere, at a temperature ranging from 50° C. to 80° C. or in any other conditions that the man skilled in the art will deem appropriate, and according to conventional methods known to him.

Compounds of formula (XXIV) may be commercially available or prepared according to any conventional methods known to the man skilled in the art.

C. Some compounds of formula (I) wherein X is an oxygen, B is a heteroaryl, R² is H, m is 1, A, R¹, R³ and n having the same definition as described in the general formula (I) above may be prepared by cyclization of a compound of formula (XXV) wherein E is Cl or NH₂ according to the equation:

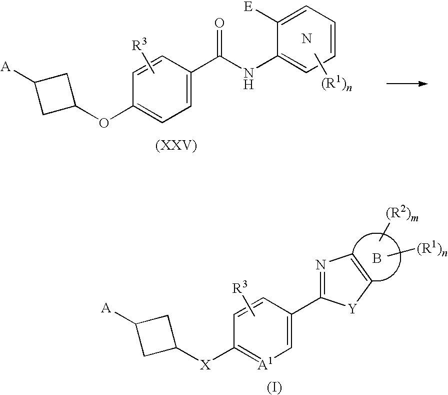

This reaction may be carried out in the presence of Lawesson's reagent in a solvent such as toluene at reflux (Y═S), or using hydrochloric acid in refluxing butanol (Y═N) or according to any other method known to the man skilled in the art.

Compound of formula (XXV) may be prepared by aminocarbonylation of a compound of formula (XXVI) according to the equation:

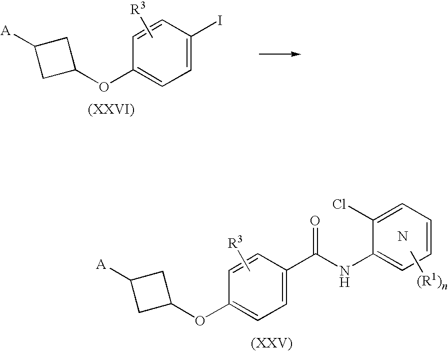

wherein A, R¹, R³ and n have the same definition as described in the general formula above. For example, this reaction may be carried out in the presence of a carbon monoxide source such as molybdenum hexacarbonyl, a suitable catalyst such as palladium acetate, and a base such as 1,8-diazabicyclo[5.4.0]undec-7-ene in a solvent such as dry tetrahydrofuran under microwave irradiation according to the method described by Letavic M. et al. Tetrahedron Lett., 2007, 48, 2339-2343, or according to any other method known to the man skilled in the art.

Compounds of formula (XXVI) may be prepared by reaction of a compound of formula (XXVII) with a compound of formula (III) according to the equation:

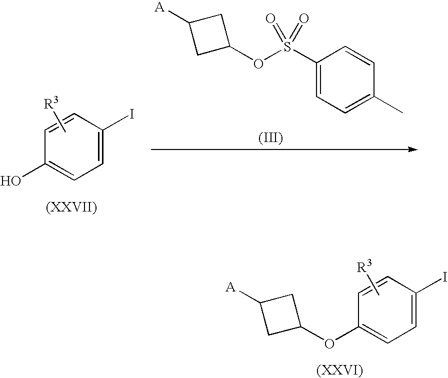

wherein R³ and A have the same definition as described above. This reaction may be carried out in the presence of a base, for example sodium hydride, in a solvent, for example N,N-dimethylacetamide, under an inert atmosphere, at a temperature ranging from 50° C. to 80° C., or in any other conditions that the man skilled in the art will deem appropriate, and according to conventional methods known to him.

Compounds of formula (XXVII) may be commercially available or prepared according to any conventional methods known to the man skilled in the art.

D. Some compounds of formula (I) may be prepared by classical transformation of other compounds of formula I as described hereafter:

Compounds of formula (Ia) wherein R² is hydrogen may be prepared by the deprotection of the corresponding compound of formula (Ia) wherein R² is t-butoxycarbonyl (Boc) using an acid such as trifluoroacetic acid according to conventional methods known to the man skilled in the art.

Compounds of formula (Ia) wherein R² is an acyl group may be prepared by reaction of the corresponding compound of formula (Ia) wherein R² is hydrogen with acid chlorides in the presence of a base such as triethylamine according to conventional methods known to the man skilled in the art. This reaction may also be carried out using a coupling agent, such as hydroxybenzotriazole, an activating agent, such as EDCI (1-(3-dimethylaminopropyl)-3-ethylcarbodiimide), in a solvent such as dichloromethane, or using any other reagents and reaction conditions known to the man skilled in the art.

Compounds of formula (Ia) wherein R² is aminocarbonyl may be prepared by reaction of the corresponding compound of formula (Ia) wherein R² is hydrogen with an isocyanate in the presence of a base such as triethylamine according to conventional methods known to the man skilled in the art or in any other reaction conditions that the man skilled in the art will deem appropriate, and according to conventional methods known to him.

Compounds of formula (Ia) wherein R² is an aminosulfonyl may be prepared from the corresponding compound of formula (Ia) wherein R² is hydrogen. For example, this reaction may be carried out using an aminosulfonyl chloride in the presence of a base such as triethylamine, in a solvent such as dichloromethane. Alternatively, this reaction may be performed according the method described by Beaudoin et al. in J. Org. Chem., 2003, 68, 115-119, or any modification of this present route.

Compounds of formula (Ia) wherein R² is an alkyl group may be prepared by reaction of the corresponding compounds of formula (Ia) wherein R² is hydrogen with alkyl halides in the presence of a base such as potassium carbonate in the presence of catalytic amount of sodium iodide according to conventional methods known to the man skilled in the art. Alternatively, this reaction may be performed by reductive amination using a reducing agent such as sodium borohydride and a carbonyl derivative in a solvent such as ethanol according to conventional methods known to the man skilled in the art.

Compounds of formula (Ia) wherein R² is a dialkylphosphonate may be prepared by reaction of the corresponding compound of formula (Ia) wherein R² is hydrogen with, firstly, benzotriazole and formaldehyde, in a solvent such as a mixture of methanol and water at room temperature, to generate a benzotriazolyl intermediate. This intermediate is directly reacted with triethylphosphite in the presence of a lewis acid such as zinc dibromide in a solvent such as dichloromethane according to the method described by Tiwari, R. K. et al. in Eur. J. Med. Chem., 2006, 41, 40-49, or any modification of this present route.

Compounds of formula (Ib) wherein R² is carboxy may be prepared by hydrolysis of the corresponding compound of formula (Ib) wherein R² is alkoxycarbonyl according to conventional methods known to the man skilled in the art.

Compounds of formula (Ia) wherein R² is a phosphonic acid can be prepared by dealkylation of the corresponding dialkylphosphonate of formula (Ia) in the presence of bromo-trimethylsilane in a solvent such as acetonitrile or according to any other method known to the man skilled in the art.

Compounds of formula (Ia) wherein R² is a cyclobutene-1,2-dione can be prepared by reaction of the corresponding compound of formula (Ia) wherein R² is hydrogen with 3,4-diisopropoxycyclobut-3-ene-1,2-dione in a solvent such as methanol or according to any other method known to the man skilled in the art. Further synthetic transformations may include hydrolysis in the presence of an aqueous acid or any other transformations known to the man skilled in the art.

In a further embodiment the present invention relates to synthetic intermediates of formula (II)

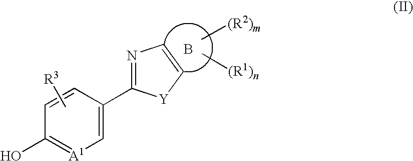

wherein

Y is S or O; and

A¹, B, R¹, R², R³, m and n having the same definition as in the general formula above for compound of formula (I).

In a particular embodiment, the present invention relates to compounds of formula (II) wherein

A¹ is CH;

B forms together with the thiazole a 4,5,6,7-tetrahydro[1,3]thiazolo[5,4-c]pyridine;

Y is S;

n is equal to 0;

R² is selected from the group consisting of acetyl, aminoacetyl, aminocarbonyl, hydroxyacetyl, 2,3-dihydroxypropyl, (2S)-2,3-dihydroxypropyl, (2R)-2,3-dihydroxypropyl, 2-amino-2-oxoethyl, 3-hydroxycyclobutyl,3-amino-3-oxopropanoyl, (5-methyl-2H-1,2,3-triazol-4-yl)carbonyl, 2-hydroxyethyl, (carboxymethoxy)acetyl, tetrahydro-2H-pyran-4-ylcarbonyl, [(1-aminocarbonyl)cylopropyl]carbonyl, amino(oxo)acetyl, 2,3-dihydroxypropanoyl and 2-amino-3,4-dioxocyclobut-1-en-1-yl;

m is 1; and

R³ is hydrogen.

Examples of compounds of formula (II) according to the present invention are:
4-(5-acetyl-4,5,6,7-tetrahydro[1,3]thiazolo[5,4-c]pyridin-2-yl)phenol;
tert-butyl 2-(4-hydroxyphenyl)-6,7-dihydro[1,3]thiazolo[5,4-c]pyridine-5(4H)-carboxylate;
2-(4-hydroxyphenyl)-6,7-dihydro[1,3]thiazolo[4,5-b]pyridin-5(4H)-one;
4-(4,5,6,7-tetrahydro[1,3]thiazolo[4,5-b]pyridin-2-yl)phenol;
4-(4-acetyl-5,6,7,8-tetrahydro-4H-[1,3]thiazolo[5,4-b]azepin-2-yl)phenol;
4-(4,5,6,7-tetrahydro[1,3]oxazolo[4,5-c]pyridin-2-yl)phenol;
4-(5-acetyl-4,5,6,7-tetrahydro[1,3]oxazolo[4,5-c]pyridin-2-yl)phenol;
4-(5-acetyl-4,5,6,7-tetrahydro[1,3]thiazolo[4,5-c]pyridin-2-yl)phenol;
ethyl 2-(4-hydroxyphenyl)-5,6-dihydro-4H-cyclopenta[d][1,3]thiazole-5-carboxylate;
1-[2-(2-fluoro-4-hydroxyphenyl)-6,7-dihydro[1,3]thiazolo[5,4-c]pyridin-5(4H)-yl]ethanone;
1-[2-(3-fluoro-4-hydroxyphenyl)-6,7-dihydro[1,3]thiazolo[5,4-c]pyridin-5(4H)-yl]ethanone;
1-[2-(2,6-difluoro-4-hydroxyphenyl)-6,7-dihydro[1,3]thiazolo[5,4-c]pyridin-5(4H)-yl]ethanone; and
1-[2-(2,3-difluoro-4-hydroxyphenyl)-6,7-dihydro[1,3]thiazolo[5,4-c]pyridin-5(4H)-yl]ethanone.

In a further embodiment, the present invention relates to synthetic intermediates of formula (III),

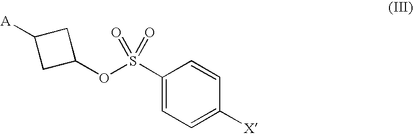

wherein

A is a substituted or unsubstituted aliphatic or cyclic amino group which is linked to the cyclobutyl group via an amino nitrogen; and

X′ is methyl or bromine.

Examples of compound of formula (III) according to the present invention are:
cis-3-morpholin-4-ylcyclobutyl 4-methylbenzenesulfonate;
cis-3-(4-isopropylpiperazin-1-yl)cyclobutyl 4-methylbenzenesulfonate;
cis-3-(4,4-difluoropiperidin-1-yl)cyclobutyl 4-methylbenzenesulfonate;
cis-3-pyrrolidin-1-ylcyclobutyl 4-methylbenzenesulfonate;
cis-3-azepan-1-ylcyclobutyl 4-methylbenzenesulfonate;
cis-3-[(3R)-3-(dimethylamino)pyrrolidin-1-yl]cyclobutyl 4-methylbenzenesulfonate;
cis-3-thiomorpholin-4-ylcyclobutyl 4-methylbenzenesulfonate;
cis-3-piperidin-1-ylcyclobutyl 4-methylbenzenesulfonate;
cis-3-(2-methylpyrrolidin-1-yl)cyclobutyl 4-methylbenzenesulfonate;
cis-3-[3-(dimethylamino)pyrrolidin-1-yl]cyclobutyl 4-bromobenzenesulfonate;
cis-3-(4-cyclopentylpiperazin-1-yl)cyclobutyl-4-bromobenzenesulfonate; and
cis-3-(piperidin-1-yl)cyclobutyl-4-bromobenzenesulfonate.

According to a specific embodiment of compounds of formula (III), the A and O groups attached to the cyclobutyl in the A-cyclobutyl-O moiety are in cis configuration.

In a further embodiment, the present invention relates to synthetic intermediates of formula (XXI),

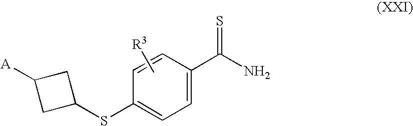

wherein

A is a substituted or unsubstituted aliphatic or cyclic amino group which is linked to the cyclobutyl group via an amino nitrogen;

R³ is hydrogen or C₁₋₆ alkyl or halogen or C₁₋₆ alkoxy.

An example of compound of formula (XXI) is 4-[(trans-3-piperidin-1-ylcyclobutyl)sulfanyl]benzenecarbothioamide.

In another embodiment, the present invention relates to synthetic intermediates of formula (XXII),

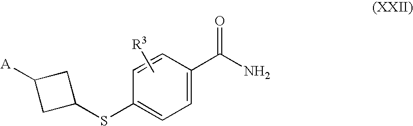

wherein

A is a substituted or unsubstituted aliphatic or cyclic amino group which is linked to the cyclobutyl group via an amino nitrogen;

R³ is hydrogen or C₁₋₆ alkyl or halogen or C₁₋₆ alkoxy.

An example of compound of formula (XXII) is 4-[(trans-3-piperidin-1-ylcyclobutyl)sulfanyl]benzamide.

In another embodiment, the present invention relates to synthetic intermediates of formula (XXIII),

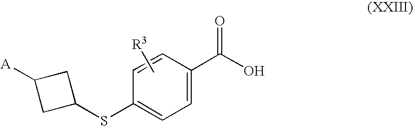

wherein

A is a substituted or unsubstituted aliphatic or cyclic amino group which is linked to the cyclobutyl group via an amino nitrogen;

R³ is hydrogen or C₁₋₆ alkyl or halogen or C₁₋₆ alkoxy.

An example of compound of formula (XXIII) is 4-[(trans-3-piperidin-1-ylcyclobutyl)sulfanyl]benzoic acid.

In another embodiment, the present invention relates to synthetic intermediates of formula (XXV),

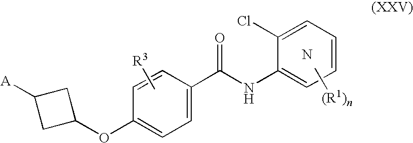

wherein

A is a substituted or unsubstituted aliphatic or cyclic amino group which is linked to the cyclobutyl group via an amino nitrogen;

R¹ is selected from the group comprising or consisting of sulfonyl, amino, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted heteroaryl, substituted or unsubstituted C₃₋₈ cycloalkyl, substituted or unsubstituted 3-8-membered heterocycloalkyl, acyl, substituted or unsubstituted C₁₋₆-alkyl aryl, substituted or unsubstituted C₁₋₆-alkyl heteroaryl, substituted or unsubstituted C₂₋₆-alkenyl aryl, substituted or unsubstituted C₂₋₆-alkenyl heteroaryl, substituted or unsubstituted C₂₋₆-alkynyl aryl, substituted or unsubstituted C₂₋₆-alkynyl heteroaryl, substituted or unsubstituted C₁₋₆-alkyl cycloalkyl, substituted or unsubstituted C₁₋₆-alkyl heterocycloalkyl, substituted or unsubstituted C₂₋₆-alkenyl cycloalkyl, substituted or unsubstituted C₂₋₆-alkenyl heterocycloalkyl, substituted or unsubstituted C₂₋₆-alkynyl cycloalkyl, substituted or unsubstituted C₂₋₆-alkynyl heterocycloalkyl, alkoxycarbonyl, aminocarbonyl, substituted or unsubstituted C₁₋₆-alkyl carboxy, substituted or unsubstituted C₁₋₆-alkyl acyl, substituted or unsubstituted aryl acyl, substituted or unsubstituted heteroaryl acyl, substituted or unsubstituted C₃₋₈-(hetero)cycloalkyl acyl, substituted or unsubstituted C₁₋₆-alkyl acyloxy, substituted or unsubstituted C₁₋₆-alkyl alkoxy, substituted or unsubstituted C₁₋₆-alkyl alkoxycarbonyl, substituted or unsubstituted C₁₋₆-alkyl aminocarbonyl, substituted or unsubstituted C₁₋₆-alkyl acylamino, acylamino, acylaminocarbonyl, ureido, substituted or unsubstituted C₁₋₆-alkyl ureido, substituted or unsubstituted C₁₋₆-alkyl carbamate, substituted or unsubstituted C₁₋₆-alkyl amino, substituted or unsubstituted C₁₋₆-alkyl sulfonyloxy, substituted or unsubstituted C₁₋₆-alkyl sulfonyl, substituted or unsubstituted C₁₋₆-alkyl sulfinyl, substituted or unsubstituted C₁₋₆-alkyl sulfanyl, substituted or unsubstituted C₁₋₆-alkyl sulfonylamino, aminosulfonyl, substituted or unsubstituted C₁₋₆-alkyl aminosulfonyl, hydroxy, substituted or unsubstituted C₁₋₆-alkyl hydroxy, phosphonate, substituted or unsubstituted C₁₋₆-alkyl phosphonate, substituted or unsubstituted C₁₋₆-alkyl phosphono, halogen, cyano, carboxy, oxo and thioxo;

n is equal to 0, 1, 2 or 3;

R³ is hydrogen or C₁₋₆ alkyl or halogen or C₁₋₆ alkoxy.

An example of compound of formula (XXV) is N-(4-chloropyridin-3-yl)-4-[(trans-3-piperidin-1-ylcyclobutyl)oxy]benzamide.

Examples of synthetic intermediates used for the synthesis of compounds of formula (I) according to the present invention are:
3-morpholin-4-ylcyclobut-2-en-1-one;
3-(4-isopropylpiperazin-1-yl)cyclobut-2-en-1-one;
3-(4,4-difluoropiperidin-1-yl)cyclobut-2-en-1-one;
3-azepan-1-ylcyclobut-2-en-1-one;
3-[(3R)-3-(dimethylamino)pyrrolidin-1-yl]cyclobut-2-en-1-one;
3-thiomorpholin-4-ylcyclobut-2-en-1-one;
cis-3-morpholin-4-ylcyclobutanol;
cis-3-(4-isopropylpiperazin-1-yl)cyclobutanol;
cis-3-(4,4-difluoropiperidin-1-yl)cyclobutanol;
cis-3-pyrrolidin-1-ylcyclobutanol;
cis-3-azepan-1-ylcyclobutanol;
cis-3-[(3R)-3-(dimethylamino)pyrrolidin-1-yl]cyclobutanol;
cis-3-thiomorpholin-4-ylcyclobutanol;
cis-3-piperidin-1-ylcyclobutanol;
cis-3-morpholin-4-ylcyclobutyl 4-methylbenzenesulfonate;
cis-3-(4-isopropylpiperazin-1-yl)cyclobutyl 4-methylbenzenesulfonate;
cis-3-(4,4-difluoropiperidin-1-yl)cyclobutyl 4-methylbenzenesulfonate;
cis-3-pyrrolidin-1-ylcyclobutyl 4-methylbenzenesulfonate;
cis-3-azepan-1-ylcyclobutyl 4-methylbenzenesulfonate;
cis-3-[(3R)-3-(dimethylamino)pyrrolidin-1-yl]cyclobutyl 4-methylbenzenesulfonate;
cis-3-thiomorpholin-4-ylcyclobutyl 4-methylbenzenesulfonate;
cis-3-piperidin-1-ylcyclobutyl 4-methylbenzenesulfonate;
4-(5-acetyl-4,5,6,7-tetrahydro[1,3]thiazolo[5,4-c]pyridin-2-yl)phenol;
tert-butyl 2-(4-hydroxyphenyl)-6,7-dihydro[1,3]thiazolo[5,4-c]pyridine-5(4H)-carboxylate;
3-methyl-1-(morpholin-4-ylsulfonyl)-1H-imidazol-3-ium trifluoromethanesulfonate;
tert-butyl {2-oxo-2-[2-{4-[(trans-3-piperidin-1-ylcyclobutyl)oxy]phenyl}-6,7-dihydro[1,3]thiazolo[5,4-c]pyridin-5(4H)-yl]ethyl}carbamate;
methyl 3-oxo-3-[2-{4-[(trans-3-piperidin-1-ylcyclobutyl)oxy]phenyl}-6,7-dihydro[1,3]thiazolo[5,4-c]pyridin-5(4H)-yl]propanoate;
3-[2-{4-[(trans-3-piperidin-1-ylcyclobutyl)oxy]phenyl}-6,7-dihydro[1,3]thiazolo[5,4-c]pyridin-5(4H)-yl]cyclobut-2-en-1-one;
2-(4-hydroxyphenyl)-6,7-dihydro[1,3]thiazolo[4,5-b]pyridin-5(4H)-one;
4-(4,5,6,7-tetrahydro[1,3]thiazolo[4,5-b]pyridin-2-yl)phenol;
2-{4-[(trans-3-piperidin-1-ylcyclobutyl)oxy]phenyl}-4,5,6,7-tetrahydro[1,3]thiazolo[4,5-b]pyridine;
4-[(trans-3-piperidin-1-ylcyclobutyl)sulfanyl]benzoic acid;
4-[(trans-3-piperidin-1-ylcyclobutyl)sulfanyl]benzamide;
4-[(trans-3-piperidin-1-ylcyclobutyl)sulfanyl]benzenecarbothioamide;
4-(benzyloxy)-N-(2-oxoazepan-3-yl)benzamide;
2-[4-(benzyloxy)phenyl]-5,6,7,8-tetrahydro-4H-[1,3]thiazolo[5,4-b]azepine hydrochloride;
4-acetyl-2-[4-(benzyloxy)phenyl]-5,6,7,8-tetrahydro-4H-[1,3]thiazolo[5,4-b]azepine;
4-(4-acetyl-5,6,7,8-tetrahydro-4H-[1,3]thiazolo[5,4-b]azepin-2-yl)phenol;
4-(benzyloxy)-N-(4-hydroxypyridin-3-yl)benzamide;
2-[4-(benzyloxy)phenyl][1,3]oxazolo[4,5-c]pyridine;
5-benzyl-2-[4-(benzyloxy)phenyl][1,3]oxazolo[4,5-c]pyridin-5-ium;
5-benzyl-2-[4-(benzyloxy)phenyl]-4,5,6,7-tetrahydro[1,3]oxazolo[4,5-c]pyridine;
4-(4,5,6,7-tetrahydro[1,3]oxazolo[4,5-c]pyridin-2-yl)phenol;
4-(5-acetyl-4,5,6,7-tetrahydro[1,3]oxazolo[4,5-c]pyridin-2-yl)phenyl acetate;
4-(5-acetyl-4,5,6,7-tetrahydro[1,3]oxazolo[4,5-c]pyridin-2-yl)phenol;
4-(benzyloxy)-N-(4-chloropyridin-3-yl)benzamide;
2-[4-(benzyloxy)phenyl][1,3]thiazolo[4,5-c]pyridine;
2-[4-(benzyloxy)phenyl]-4,5,6,7-tetrahydro[1,3]thiazolo[4,5-c]pyridine;
5-acetyl-2-[4-(benzyloxy)phenyl]-4,5,6,7-tetrahydro[1,3]thiazolo[4,5-c]pyridine;
4-(5-acetyl-4,5,6,7-tetrahydro[1,3]thiazolo[4,5-c]pyridin-2-yl)phenol;
methyl 3-bromo-4-hydroxycyclopentanecarboxylate;
methyl 3-bromo-4-oxocyclopentanecarboxylate;
ethyl 2-(4-hydroxyphenyl)-5,6-dihydro-4H-cyclopenta[d][1,3]thiazole-5-carboxylate;
ethyl 2-{4-[(trans-3-piperidin-1-ylcyclobutyl)oxy]phenyl}-5,6-dihydro-4H-cyclopenta[d][1,3]thiazole-5-carboxylate;
1-[trans-3-(4-iodophenoxy)cyclobutyl]piperidine;
N-(4-chloropyridin-3-yl)-4-[(trans-3-piperidin-1-ylcyclobutyl)oxy]benzamide;
3-(4-cyclopentylpiperazin-1-yl)cyclobut-2-en-1-one;
3-[3-(dimethylamino)pyrrolidin-1-yl]cyclobut-2-en-1-one;
3-(2-methylpyrrolidin-1-yl)cyclobut-2-en-1-one;
cis-3-(4-cyclopentylpiperazin-1-yl)cyclobutanol;
cis-3-[3-(dimethylamino)pyrrolidin-1-yl]cyclobutanol;
cis-3-(2-methylpyrrolidin-1-yl)cyclobutanol;
cis-3-(2-methylpyrrolidin-1-yl)cyclobutyl 4-methylbenzenesulfonate;
2-(benzyloxy)-1-[2-(4-{[trans-3-(piperidin-1-yl)cyclobutyl]oxy}phenyl)-6,7-dihydro[1,3]thiazolo[4,5-b]pyridin-4(5H)-yl]ethanone;
2-fluoro-4-hydroxybenzenecarbothioamide;
3-fluoro-4-(4,5,6,7-tetrahydro[1,3]thiazolo[5,4-c]pyridin-2-yl)phenol;
4-(5-acetyl-4,5,6,7-tetrahydro[1,3]thiazolo[5,4-c]pyridin-2-yl)-3-fluorophenyl acetate;
1-[2-(2-fluoro-4-hydroxyphenyl)-6,7-dihydro[1,3]thiazolo[5,4-c]pyridin-5(4H)-yl]ethanone;
3-fluoro-4-methoxybenzenecarbothioamide;
2,6-difluoro-4-methoxybenzenecarbothioamide;
2,3-difluoro-4-methoxybenzenecarbothioamide;
2-(3-fluoro-4-methoxyphenyl)-4,5,6,7-tetrahydro[1,3]thiazolo[5,4-c]pyridine;
2-(2,6-difluoro-4-methoxyphenyl)-4,5,6,7-tetrahydro[1,3]thiazolo[5,4-c]pyridine;
2-(2,3-difluoro-4-methoxyphenyl)-4,5,6,7-tetrahydro[1,3]thiazolo[5,4-c]pyridine;
1-[2-(3-fluoro-4-methoxyphenyl)-6,7-dihydro[1,3]thiazolo[5,4-c]pyridin-5(4H)-yl]ethanone;
1-[2-(2,6-difluoro-4-methoxyphenyl)-6,7-dihydro[1,3]thiazolo[5,4-c]pyridin-5(4H)-yl]ethanone;
1-[2-(2,3-difluoro-4-methoxyphenyl)-6,7-dihydro[1,3]thiazolo[5,4-c]pyridin-5(4H)-yl]ethanone;
1-[2-(3-fluoro-4-hydroxyphenyl)-6,7-dihydro[1,3]thiazolo[5,4-c]pyridin-5(4H)-yl]ethanone;
1-[2-(2,6-difluoro-4-hydroxyphenyl)-6,7-dihydro[1,3]thiazolo[5,4-c]pyridin-5(4H)-yl]ethanone;
1-[2-(2,3-difluoro-4-hydroxyphenyl)-6,7-dihydro[1,3]thiazolo[5,4-c]pyridin-5(4H)-yl]ethanone;
4-(5-acetyl-4,5,6,7-tetrahydro[1,3]thiazolo[5,4-c]pyridin-2-yl)-2-fluorophenyl acetate;
4-(5-acetyl-4,5,6,7-tetrahydro[1,3]thiazolo[5,4-c]pyridin-2-yl)-3,5-difluorophenyl acetate;
4-(5-acetyl-4,5,6,7-tetrahydro[1,3]thiazolo[5,4-c]pyridin-2-yl)-2,3-difluorophenyl acetate;
N-(2-oxoazepan-3-yl)-4-{[trans-3-(piperidin-1-yl)cyclobutyl]oxy}benzamide;
N-(3-aminopyridin-4-yl)-4-{[trans-3-(piperidin-1-yl)cyclobutyl]oxy}benzamide;
cis-3-[3-(dimethylamino)pyrrolidin-1-yl]cyclobutyl 4-bromobenzenesulfonate;
cis-3-(4-cyclopentylpiperazin-1-yl)cyclobutyl 4-bromobenzenesulfonate;
cis-3-(piperidin-1-yl)cyclobutyl 4-bromobenzenesulfonate;
N,N-dimethyl-1-{trans-3-[4-(4,5,6,7-tetrahydro[1,3]thiazolo[5,4-c]pyridin-2-yl)phenoxy]cyclobutyl}pyrrolidin-3-amine;
2-(4-{[trans-3-(4-cyclopentylpiperazin-1-yl)cyclobutyl]oxy}phenyl)-4,5,6,7-tetrahydro[1,3]thiazolo[5,4-c]pyridine;
2-(4-{[trans-3-(2-methylpyrrolidin-1-yl)cyclobutyl]oxy}phenyl)-4,5,6,7-tetrahydro[1,3]thiazolo[5,4-c]pyridine;
1-[2-(4-{[trans-3-(2-methylpyrrolidin-1-yl)cyclobutyl]oxy}phenyl)-6,7-dihydro[1,3]thiazolo[5,4-c]pyridin-5(4H)-yl]ethanone; and
2-(4-{[trans-3-(piperidin-1-yl)cyclobutyl]oxy}phenyl)-4,5,6,7-tetrahydro[1,3]thiazolo[4,5-c]pyridine.

It has now been found that compounds of formula (I) according to the present invention and their pharmaceutically acceptable salts are useful in a variety of medical disorders.

For example, the compounds according to the invention are useful for the treatment and prevention of diseases or pathological conditions of the central nervous system including mild-cognitive impairments, Alzheimer's disease, learning and memory disorders, cognitive disorders, attention deficit disorder, attention-deficit hyperactivity disorder, Parkinson's disease, schizophrenia, dementia, depression, epilepsy, seizures, convulsions, sleep/wake and arousal/vigilance disorders such as hypersomnia and narcolepsy, pain and/or obesity.

Furthermore, compounds according to the invention alone or in combination with an antiepileptic drug (AED) may be useful in the treatment of epilepsy, seizure or convulsions. It is known from literature that the combination of H₃-receptor ligands with an AED may produce additive synergistic effects on efficacy with reduced side-effects such as decreased vigilance, sedation or cognitive problems.

Furthermore, compounds of general formula (I) alone or in combination with a histamine H₁ antagonist may also be used for the treatment of upper airway allergic disorders.

In a particular embodiment of the present invention, compounds of general formula (I), alone or in combination with muscarinic receptor ligands and particularly with a muscarinic M₂ antagonist, may be useful for the treatment of cognitive disorders, Alzheimer's disease, and attention-deficit hyperactivity disorder.

Particularly, compounds of general formula (I) displaying NO-donor properties, alone or in combination with a nitric oxide (NO) releasing agent may be useful in the treatment of cognitive dysfunctions.

Compounds of general formula (I) may also be used in the treatment and prevention of multiple sclerosis (MS).

Usually, compounds of general formula (I) may be used in the treatment and prevention of all types of cognitive-related disorders.

In one embodiment, compounds of general formula (I) may be used for the treatment and prevention of cognitive dysfunctions in diseases such as mild cognitive impairment, dementia, Alzheimer's disease, Parkinson's disease, Down's syndrome as well as for the treatment of attention-deficit hyperactivity disorder.

In another embodiment, compounds of general formula (I) may also be used for the treatment and prevention of psychotic disorders, such as schizophrenia; or for the treatment of eating disorders, such as obesity; or for the treatment of inflammation and pain disorders; or for the treatment of anxiety, stress and depression; or for the treatment of cardiovascular disorders, for example, myocardial infarction; or for the treatment and prevention of multiple sclerosis (MS).

Pain disorders include neuropathic pain, such as associated with diabetic neuropathy, post-herpetic neuralgia; trigeminal neuralgia, posttraumatic peripheral neuropathy, phantom limb pain, with cancer and neuropathies induced by treatment with antineoplastic agents, pain due to nerve damage associated with demyelinating disease such as multiple sclerosis, neuropathy associated with HIV, post-operative pain; corneal pain, obstetrics pain (pain relief during delivery or after caesarean section), visceral pain, inflammatory pain such as associated to rheumatoid arthritis; low-back pain/sciatica; carpal tunnel syndrome, allodynic pain such as fibromyalgia; chronic pain associated with Complex Regional Pain Syndrome (CRPS) and chronic muscle pain such as, yet not limited to, that associated with back spasm.

In a particular embodiment, compounds of formula (I) may be used for the treatment and prevention neuropathic pain.

In one embodiment, compounds of formula (I) according to the present invention may be used as a medicament.

In another embodiment, compounds of formula (I) according to the present invention may be used for the treatment or prevention of mild-cognitive impairement, Alzheimer's disease, learning and memory disorders, attention-deficit hyperactivity disorder, Parkinson's disease, schizophrenia, dementia, depression, epilepsy, seizures, convulsions, sleep/wake disorders, cognitive dysfunctions, narcolepsy, hypersomnia, obesity, upper airway allergic disorders, Down's syndrome, anxiety, stress, cardiovascular disorders, inflammation, pain disorders, particularly neuropathic pain, or multiple sclerosis.

In a particular embodiment, compounds of formula (I) according to the present invention may be used for the treatment of mild cognitive impairment, dementia, Alzheimer's disease, Parkinson's disease, Down's syndrome as well as for the treatment of attention-deficit hyperactivity disorder.

In a further embodiment, the present invention concerns the use of a compound of formula (I) or a pharmaceutically acceptable salt thereof or of a pharmaceutical composition comprising an effective amount of said compound for the manufacture of a medicament for the treatment and prevention of mild-cognitive impairement, Alzheimer's disease, learning and memory disorders, attention-deficit hyperactivity disorder, Parkinson's disease, schizophrenia, dementia, depression, epilepsy, seizures, convulsions, sleep/wake disorders, cognitive dysfunctions, narcolepsy, hypersomnia, obesity, upper airway allergic disorders, Down's syndrome, anxiety, stress, cardiovascular disorders, inflammation, pain disorders, particularly neuropathic pain, or multiple sclerosis.

In another embodiment, the present invention concerns the use of a compound of formula (I) or a pharmaceutically acceptable salt thereof or a pharmaceutical composition comprising an effective amount of said compound for the manufacture of a medicament for the treatment of cognitive dysfunctions in diseases such as mild cognitive impairment, dementia, Alzheimer's disease, Parkinson's disease, Down's syndrome as well as for the treatment of attention-deficit hyperactivity disorder.

The methods of the invention comprise administration to a mammal (preferably human) suffering from above mentioned conditions or disorders, of a compound according to the invention in an amount sufficient to alleviate or prevent the disorder or condition.

The compound is conveniently administered in any suitable unit dosage form, including but not limited to one containing 3 to 3000 mg of active ingredient per unit dosage form.

The term “treatment” as used herein includes curative treatment and prophylactic treatment.

By “curative” is meant efficacy in treating a current symptomatic episode of a disorder or condition.

By “prophylactic” is meant prevention of the occurrence or recurrence of a disorder or condition.

The term “cognitive disorders” as used herein refers to disturbances of cognition, which encompasses perception, learning and reasoning or in other terms the physiological (mental/neuronal) process of selectively acquiring, storing, and recalling information.

The term “attention-deficit hyperactivity disorder” (ADHD) as used herein refers to a problem with inattentiveness, over-activity, impulsivity, or a combination of these. For these problems to be diagnosed as ADHD, they must be out of the normal range for the child's age and development. The term “attention-deficit disorder” (ADD) is also commonly used for the same disorder.

The term “Alzheimer's disease” (AD) as used herein refers to a progressive, neurodegenerative disease characterized in the brain by abnormal clumps (amyloid plaques) and tangled bundles of fibers (neurofibrillary tangles) composed of misplaced proteins. Age is the most important risk factor for AD; the number of people with the disease doubles every 5 years beyond age 65. Three genes have been discovered that cause early onset (familial) AD. Other genetic mutations that cause excessive accumulation of amyloid protein are associated with age-related (sporadic) AD. Symptoms of AD include memory loss, language deterioration, impaired ability to mentally manipulate visual information, poor judgment, confusion, restlessness, and mood swings. Eventually AD destroys cognition, personality, and the ability to function. The early symptoms of AD, which include forgetfulness and loss of concentration, are often missed because they resemble natural signs of aging.

The term “Parkinson's disease” (PD) as used herein refers to a group of conditions called motor system disorders, which are the result of the loss of dopamine-producing brain cells. The four primary symptoms of PD are tremor, or trembling in hands, arms, legs, jaw, and face; rigidity, or stiffness of the limbs and trunk; bradykinesia, or slowness of movement; and postural instability, or impaired balance and coordination. As these symptoms become more pronounced, patients may have difficulty walking, talking, or completing other simple tasks. PD usually affects people over the age of 50. Early symptoms of PD are subtle and occur gradually. In some people the disease progresses more quickly than in others. As the disease progresses, the shaking, or tremor, which affects the majority of PD patients may begin to interfere with daily activities. Other symptoms may include depression and other emotional changes; difficulty in swallowing, chewing, and speaking; urinary problems or constipation; skin problems; and sleep disruptions.

The term “Down's syndrome” as used herein refers to a chromosome abnormality, usually due to an extra copy of the 21^{st} chromosome. This syndrome, usually but not always results in mental retardation and other conditions. The term “mental retardation” refers to a below-average general intellectual function with associated deficits in adaptive behavior that occurs before age 18.

The term “mild-cognitive impairment” as used herein refers to a transitional stage of cognitive impairment between normal aging and early Alzheimer's disease. It refers particularly to a clinical state of individuals who are memory impaired but are otherwise functioning well and do not meet clinical criteria for dementia.

The term “obesity” as used herein refers to a body mass index (BMI) which is greater than 30 kg/m².

The term “dementia” as used herein refers to a group of symptoms involving progressive impairment of brain function. American Geriatrics Society refers to dementia as a condition of declining mental abilities, especially memory. The person will have problems doing things he or she used to be able to do, like keep the check book, drive a car safely, or plan a meal. He or she will often have problems finding the right words and may become confused when given too many things to do at once. The person with dementia may also change in personality, becoming aggressive, paranoid, or depressed.

The term “schizophrenia” as used herein refers to a group of psychotic disorders characterized by disturbances in thought, perception, attention, affect, behavior, and communication that last longer than 6 months. It is a disease that makes it difficult for a person to tell the difference between real and unreal experiences, to think logically, to have normal emotional responses to others, and to behave normally in social situations.

The term “anxiety” as used herein refers to a feeling of apprehension or fear. Anxiety is often accompanied by physical symptoms, including twitching or trembling, muscle tension, headaches, sweating, dry mouth, difficulty swallowing and/or abdominal pain.

The term “narcolepsy” as used herein refers to a sleep disorder associated with uncontrollable sleepiness and frequent daytime sleeping.

The term “depression” as used herein refers to a disturbance of mood and is characterized by a loss of interest or pleasure in normal everyday activities. People who are depressed may feel “down in the dumps” for weeks, months, or even years at a time. Some of the following symptoms may be symptoms of depression: persistent sad, anxious, or “empty” mood; feelings of hopelessness, pessimism; feelings of guilt, worthlessness, helplessness; loss of interest or pleasure in hobbies and activities that were once enjoyed, including sex; decreased energy, fatigue, being “slowed down”; difficulty concentrating, remembering, making decisions; insomnia, early-morning awakening, or oversleeping; appetite and/or weight loss or overeating and weight gain; thoughts of death or suicide; suicide attempts; restlessness, irritability; persistent physical symptoms that do not respond to treatment, such as headaches, digestive disorders, and chronic pain.

The term “epilepsy” as used herein refers a brain disorder in which clusters of nerve cells, or neurons, in the brain sometimes signal abnormally. In epilepsy, the normal pattern of neuronal activity becomes disturbed, causing strange sensations, emotions, and behavior or sometimes convulsions, muscle spasms, and loss of consciousness. Epilepsy is a disorder with many possible causes. Anything that disturbs the normal pattern of neuron activity—from illness to brain damage to abnormal brain development—can lead to seizures. Epilepsy may develop because of an abnormality in brain wiring, an imbalance of nerve signaling chemicals called neurotransmitters, or some combination of these factors. Having a seizure does not necessarily mean that a person has epilepsy. Only when a person has had two or more seizures is he or she considered to have epilepsy.

The term “seizure” as used herein refers to a transient alteration of behaviour due to the disordered, synchronous, and rhythmic firing of populations of brain neurones.

The term “migraine” as used herein means a disorder characterised by recurrent attacks of headache that vary widely in intensity, frequency, and duration. The pain of a migraine headache is often described as an intense pulsing or throbbing pain in one area of the head. It is often accompanied by extreme sensitivity to light and sound, nausea, and vomiting. Some individuals can predict the onset of a migraine because it is preceded by an “aura,” visual disturbances that appear as flashing lights, zig-zag lines or a temporary loss of vision. People with migraine tend to have recurring attacks triggered by a lack of food or sleep, exposure to light or hormonal irregularities (only in women). Anxiety, stress, or relaxation after stress can also be triggers. For many years, scientists believed that migraines were linked to the dilation and constriction of blood vessels in the head. Investigators now believe that migraine is caused by inherited abnormalities in genes that control the activities of certain cell populations in the brain. The International Headache Society (IHS, 1988) classifies migraine with aura (classical migraine) and migraine without aura (common migraine) as the major types of migraine.

The term “multiple sclerosis” (MS) as used herein is a chronic disease of the central nervous system in which gradual destruction of myelin occurs in patches throughout the brain or spinal cord or both, interfering with the nerve pathways. As more and more nerves are affected, a patient experiences a progressive interference with functions that are controlled by the nervous system such as vision, speech, walking, writing, and memory.

Activity in any of the above-mentioned indications can of course be determined by carrying out suitable clinical trials in a manner known to a person skilled in the relevant art for the particular indication and/or in the design of clinical trials in general.

For treating diseases, compounds of formula (I) or their pharmaceutically acceptable salts may be employed at an effective daily dosage and administered in the form of a pharmaceutical composition.

Therefore, another embodiment of the present invention concerns a pharmaceutical composition comprising an effective amount of a compound of formula (I) or a pharmaceutically acceptable salt thereof in combination with a pharmaceutically acceptable diluent or carrier.

To prepare a pharmaceutical composition according to the invention, one or more of the compounds of formula (I) or a pharmaceutically acceptable salt thereof is intimately admixed with a pharmaceutical diluent or carrier according to conventional pharmaceutical compounding techniques known to the skilled practitioner.

Suitable diluents and carriers may take a wide variety of forms depending on the desired route of administration, e.g., oral, rectal, parenteral or intranasal.

Pharmaceutical compositions comprising compounds according to the invention can, for example, be administered orally, parenterally, i.e., intravenously, intramuscularly or subcutaneously, intrathecally, by inhalation or intranasally.

Pharmaceutical compositions suitable for oral administration can be solids or liquids and can, for example, be in the form of tablets, pills, dragees, gelatin capsules, solutions, syrups, chewing-gums and the like.

To this end the active ingredient may be mixed with an inert diluent or a non-toxic pharmaceutically acceptable carrier such as starch or lactose. Optionally, these pharmaceutical compositions can also contain a binder such as microcrystalline cellulose, gum tragacanth or gelatine, a disintegrant such as alginic acid, a lubricant such as magnesium stearate, a glidant such as colloidal silicon dioxide, a sweetener such as sucrose or saccharin, or colouring agents or a flavouring agent such as peppermint or methyl salicylate.

The invention also contemplates compositions which can release the active substance in a controlled manner. Pharmaceutical compositions which can be used for parenteral administration are in conventional form such as aqueous or oily solutions or suspensions generally contained in ampoules, disposable syringes, glass or plastics vials or infusion containers.

In addition to the active ingredient, these solutions or suspensions can optionally also contain a sterile diluent such as water for injection, a physiological saline solution, oils, polyethylene glycols, glycerine, propylene glycol or other synthetic solvents, antibacterial agents such as benzyl alcohol, antioxidants such as ascorbic acid or sodium bisulphite, chelating agents such as ethylene diamine-tetra-acetic acid, buffers such as acetates, citrates or phosphates and agents for adjusting the osmolarity, such as sodium chloride or dextrose.

These pharmaceutical forms are prepared using methods which are routinely used by pharmacists.

The amount of active ingredient in the pharmaceutical compositions can fall within a wide range of concentrations and depends on a variety of factors such as the patient's sex, age, weight and medical condition, as well as on the method of administration. Thus the quantity of compound of formula (I) in compositions for oral administration is at least 0.5% by weight and can be up to 80% by weight with respect to the total weight of the composition.

For the preferred oral compositions, the daily dosage is in the range 3 to 3000 milligrams (mg) of compounds of formula (I).

In compositions for parenteral administration, the quantity of compound of formula (I) present is at least 0.5% by weight and can be up to 33% by weight with respect to the total weight of the composition. For the preferred parenteral compositions, the dosage unit is in the range 3 mg to 3000 mg of compounds of formula (I).

The daily dose can fall within a wide range of dosage units of compound of formula (I) and is generally in the range 3 to 3000 mg. However, it should be understood that the specific doses can be adapted to particular cases depending on the individual requirements, at the physician's discretion.

The following examples illustrate how the compounds covered by formula (I) may be synthesized. They are provided for illustrative purposes only and are not intended, nor should they be construed, as limiting the invention in any manner. Those skilled in the art will appreciate that routine variations and modifications of the following examples can be made without exceeding the spirit or scope of the invention.

NMR spectra are recorded on a BRUKER AVANCE 400 NMR Spectrometer fitted with a Linux workstation running XWIN NMR 3.5 software and a 5 mm inverse ¹H/BB probehead, or BRUKER DRX 400 NMR fitted with a SG Fuel running XWIN NMR 2.6 software and a 5 mm inverse geometry ¹H/¹³C/¹⁹F triple probehead. The compound is studied in d₆-dimethylsulfoxide (or d₃-chloroform) solution at a probe temperature of 313 K or 300 K and at a concentration of 10 mg/ml. The instrument is locked on the deuterium signal of d₆-dimethylsulfoxide (or d₃-chloroform). Chemical shifts are given in ppm downfield from TMS (tetramethylsilane) taken as internal standard.

HPLC analyses are performed using one of the following systems:
an Agilent 1100 series HPLC system mounted with an INERTSIL ODS 3 C18, DP 5 μm, 250×4.6 mm column. The gradient runs from 100% solvent A (acetonitrile, water, phosphoric acid (5/95/0.001, v/v/v)) to 100% solvent B (acetonitrile, water, phosphoric acid (95/5/0.001, v/v/v)) in 6 min with a hold at 100% B of 4 min. The flow rate is set at 2.5 ml/min. The chromatography is carried out at 35° C.
a HP 1090 series HPLC system mounted with a HPLC Waters Symetry C18, 250×4.6 mm column. The gradient runs from 100% solvent A (methanol, water, phosphoric acid (15/85/0.001M, v/v/M)) to 100% solvent B (methanol, water, phosphoric acid (85/15/0.001 M, v/v/M)) in 10 min with a hold at 100% B of 10 min. The flow rate is set at 1 ml/min. The chromatography is carried out at 40° C.

Mass spectrometric measurements in LC/MS mode are performed as follows:

HPLC Conditions

Analyses are performed using a WATERS Alliance HPLC system mounted with an INERTSIL ODS 3, DP 5 μm, 250×4.6 mm column.

The gradient runs from 100% solvent A (acetonitrile, water, trifluoroacetic acid (10/90/0.1, v/v/v)) to 100% solvent B (acetonitrile, water, trifluoroacetic acid (90/10/0.1, v/v/v)) in 7 min with a hold at 100% B of 4 min. The flow rate is set at 2.5 ml/min and a split of 1/25 is used just before API source.

MS Conditions

Samples are dissolved in acetonitrile/water, 70/30, v/v at the concentration of about 250 μg/ml. API spectra (+ or −) are performed using a FINNIGAN LCQ ion trap mass spectrometer. APCI source operated at 450° C. and the capillary heater at 160° C. ESI source operated at 3.5 kV and the capillary heater at 210° C.

Mass spectrometric measurements in DIP/EI mode are performed as follows: samples are vaporized by heating the probe from 50° C. to 250° C. in 5 min. EI (Electron Impact) spectra are recorded using a FINNIGAN TSQ 700 tandem quadrupole mass spectrometer. The source temperature is set at 150° C.

Mass spectrometric measurements on a TSQ 700 tandem quadrupole mass spectrometer (Finnigan MAT) in GC/MS mode are performed with a gas chromatograph model 3400 (Varian) fitted with a split/splitless injector and a DB-5MS fused-silica column (15 m×0.25 mm I.D., 1 μm) from J&W Scientific. Helium (purity 99.999%) is used as carrier gas. The injector (CTC A200S autosampler) and the transfer line operate at 290 and 250° C., respectively. Sample (1 μl) is injected in splitless mode and the oven temperature is programmed as follows: 50° C. for 5 min., increasing to 280° C. (23° C./min) and holding for 10 min. The TSQ 700 spectrometer operates in electron impact (EI) or chemical ionization (CI/CH₄) mode (mass range 33-800, scan time 1.00 sec). The source temperature is set at 150° C.

Specific rotation is recorded on a Perkin-Elmer 341 polarimeter. The angle of rotation is recorded at 25° C. on 1% solutions in methanol, at 589 nm.

Melting points are determined on a Büchi 535 or 545 Tottoli-type fusionometre, and are not corrected, or by the onset temperature on a Perkin Elmer DSC 7.

Preparative chromatographic separations are performed on silicagel 60 Merck, particle size 15-40 μm, reference 1.15111.9025, using Novasep axial compression columns (80 mm i.d.), flow rates between 70 and 150 ml/min. Amount of silicagel and solvent mixtures as described in individual procedures.

Preparative Chiral Chromatographic separations are performed on a DAICEL Chiralpak AD 20 μm, 100*500 mm column using an in-house build instrument with various mixtures of lower alcohols and C5 to C8 linear, branched or cyclic alkanes at ±350 ml/min. Solvent mixtures as described in individual procedures.

### EXAMPLE 1

### Synthesis of 5-acetyl-2-{4-[(trans-3-morpholin-4-ylcyclobutyl)oxy]phenyl}-4,5,6,7-tetrahydro[1,3]thiazolo[5,4-c]pyridine 8

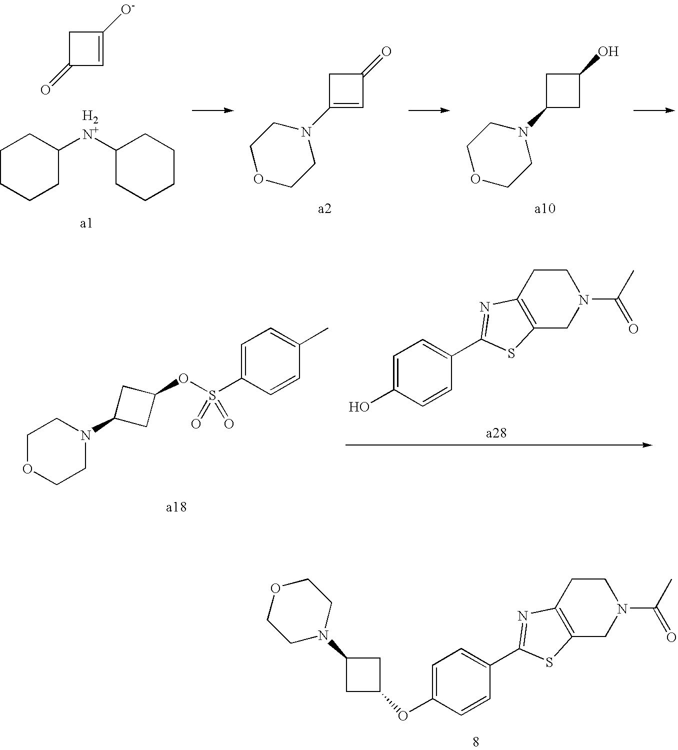

### 1.1 Synthesis of 3-morpholin-4-ylcyclobut-2-en-1-one a2

Trifluoroacetic acid (0.924 ml, 12.43 mmol, 1.1 eq) is added over 10 minutes to a stirred suspension of N-cyclohexylcyclohexanaminium 3-oxocyclobut-1-en-1-olate a1 (3 g, 11.30 mmol, 1 eq) in dioxane (15 ml). After 12 hours at room temperature, the resulting suspension is filtered and washed with dioxane (3 ml). The filtrate is then stirred at room temperature and treated dropwise with morpholine (1.29 ml, 14.69 mmol, 1.3 eq) while maintaining the temperature below 20° C. throughout the addition (20 minutes) with a water bath. The mixture is stirred overnight at room temperature. Dioxane is then removed under reduced pressure. The resulting oil (2.4 g) is purified by chromatography over silicagel, (eluent: dichloromethane/methanol/ammonia 98:1.8:0.2 then 97:2.7:0.3) to afford 1.1 g of 3-morpholin-4-ylcyclobut-2-en-1-one a2.

Yield: 64%.

LC-MS (MH⁺): 154.

The following compounds may be synthesized according to the same method:

| | | |
|---|---|---|
| a3 | 3-(4-isopropylpiperazin-1-yl)cyclobut-2-en-1- | LC-MS (MH⁺): 195 |
| | one | |
| a4 | 3-(4,4-difluoropiperidin-1-yl)cyclobut-2-en-1- | LC-MS (MH⁺): 188 |
| | one | |
| a5 | 3-pyrrolidin-1-ylcyclobut-2-en-1-one | LC-MS (MH⁺): 138 |
| a6 | 3-azepan-1-ylcyclobut-2-en-1-one | LC-MS (MH⁺): 166 |
| a7 | 3-[(3R)-3-(dimethylamino)pyrrolidin-1- | LC-MS (MH⁺): 181 |
| | yl]cyclobut-2-en-1-one | |
| a8 | 3-thiomorpholin-4-ylcyclobut-2-en-1-one | LC-MS (MH⁺): 170 |
| a9 | 3-piperidin-1-ylcyclobut-2-en-1-one | ¹H NMR (CDCl₃) δ: 4.47 (s, |
| | | 1 H), 3.22 (m, 4 H), 2.95 (s, |
| | | 2 H), 1.53 (m, 6 H) |
| a70 | 3-(4-cyclopentylpiperazin-1-yl)cyclobut-2-en- | LC-MS (MH⁺): 221 |
| | 1-one | |
| a71 | 3-[3-(dimethylamino)pyrrolidin-1-yl]cyclobut-2- | LC-MS (MH⁺): 181 |
| | en-1-one | |
| a72 | 3-(2-methylpyrrolidin-1-yl)cyclobut-2-en-1-one | LC-MS (MH⁺): 152 |

### 1.2 Synthesis of cis-3-morpholin-4-ylcyclobutanol a10

A solution of 3-morpholin-4-ylcyclobut-2-en-1-one a2 (1.1 g, 7.18 mmol, 1 eq) in ethanol (18 ml) is treated with portions of sodium borohydride (0.951 g, 25.13 mmol, 3.5 eq). At the end of the addition, the mixture is stirred at 50° C. for 12 h, cooled down to 20° C. and treated with acetone (2.3 ml). The solvents are removed under reduced pressure to leave a yellow solid that is then taken up in dichloromethane. This organic layer is filtered over celite and concentrated under reduced pressure to afford 1.5 g of cis-3-morpholin-4-ylcyclobutanol a10 as a yellow oil which is directly used in the next step without further purification.

Yield: 100%.

LC-MS (MH⁺): 158.

The following compounds may be synthesized according to the same method:

| | | |
|---|---|---|
| a11 | cis-3-(4-isopropylpiperazin-1-yl)cyclobutanol | LC-MS (MH⁺): 199 |
| a12 | cis-3-(4,4-difluoropiperidin-1-yl)cyclobutanol | LC-MS (MH⁺): 192 |
| a13 | cis-3-pyrrolidin-1-ylcyclobutanol | LC-MS (MH⁺): 142 |
| a14 | cis-3-azepan-1-ylcyclobutanol | LC-MS (MH⁺): 170 |
| a15 | cis-3-[(3R)-3-(dimethylamino)pyrrolidin-1- | LC-MS (MH⁺): 185 |
| | yl]cyclobutanol | |
| a16 | cis-3-thiomorpholin-4-ylcyclobutanol | LC-MS (MH⁺): 174 |
| a17 | cis-3-piperidin-1-ylcyclobutanol | ¹H NMR (CDCl₃) δ: 3.81 (m, |
| | | 3 H), 2.38 (m, 2 H), 2.06 (m, 4 |
| | | H), 1.69 (m, 2 H), 1.43 (m, 4 |
| | | H), 1.29 (bs, 2 H) |
| a73 | cis-3-(4-cyclopentylpiperazin-1- | LC-MS (MH⁺): 225 |
| | yl)cyclobutanol | |
| a74 | cis-3-[3-(dimethylamino)pyrrolidin-1- | LC-MS (MH⁺): 185 |
| | yl]cyclobutanol | |
| a75 | cis-3-(2-methylpyrrolidin-1-yl)cyclobutanol | LC-MS (MH⁺): 156 |

### 1.3 Synthesis of cis-3-morpholin-4-ylcyclobutyl 4-methylbenzenesulfonate a18

A solution of cis-3-morpholin-4-ylcyclobutanol a10 (1.5 g, 9.54 mmol, 1.0 eq) and N-methylimidazole (0.84 ml, 10.50 mmol, 1.1 eq) in ethyl acetate (15 ml) is treated with p-toluenesulfonyl chloride (2.0 g, 10.50 mmol, 1.1 eq). The mixture is stirred at 20° C. for 1 h. The mixture is washed with water, dried over magnesium sulfate and concentrated under vacuum to afford 2.6 g of yellow oil. This oil is purified by flash chromatography over silicagel (dichloromethane/methanol 100:0 to 90:10) to yield 0.61 g of cis-3-morpholin-4-ylcyclobutyl 4-methylbenzenesulfonate a18 as an orange oil.

Yield: 21%.

LC-MS (MH⁺): 312.

The following compounds may be synthesized according to the same method:

| | | |
|---|---|---|
| a19 | cis-3-(4-isopropylpiperazin-1-yl)cyclobutyl | LC-MS (MH⁺): 353 |
| | 4-methylbenzenesulfonate | |
| a20 | cis-3-(4,4-difluoropiperidin-1-yl)cyclobutyl | LC-MS (MH⁺): 346 |
| | 4-methylbenzenesulfonate | |
| a21 | cis-3-pyrrolidin-1-ylcyclobutyl 4- | LC-MS (MH⁺): 296 |
| | methylbenzenesulfonate | |
| a22 | cis-3-azepan-1-ylcyclobutyl 4- | LC-MS (MH⁺): 324 |
| | methylbenzenesulfonate | |
| a23 | cis-3-[(3R)-3-(dimethylamino)pyrrolidin-1- | LC-MS (MH⁺): 339 |
| | yl]cyclobutyl 4-methylbenzenesulfonate | |
| a24 | cis-3-thiomorpholin-4-ylcyclobutyl 4- | LC-MS (MH⁺): 328 |
| | methylbenzenesulfonate | |
| a25 | cis-3-piperidin-1-ylcyclobutyl 4- | LC-MS (MH⁺): 310 |
| | methylbenzenesulfonate | |
| a76 | cis-3-(2-methylpyrrolidin-1-yl)cyclobutyl 4- | LC-MS (MH⁺): 310 |
| | methylbenzenesulfonate | |

### 1.4 Synthesis of 4-(5-acetyl-4,5,6,7-tetrahydro[1,3]thiazolo[5,4-c]pyridin-2-yl)phenol a28

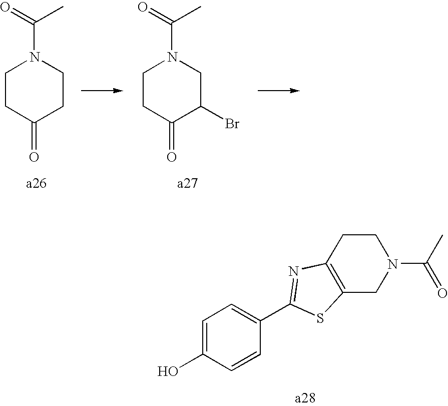

### 1.4.1 Synthesis of 1-acetyl-3-bromopiperidin-4-one a27.

A solution of bromine (12.24 g, 76.60 mmol, 1.1 eq) in chloroform (10 ml) is added dropwise to a solution of 1-acetylpiperidin-4-one a26 (9.83 g, 69.63 mmol, 1 eq) in chloroform (160 ml) at 0° C. The mixture is left to warm up to 20° C. The white solid that forms is filtered and washed with water to give 14.9 g of 1-acetyl-3-bromopiperidin-4-one a27.

Yield: 97%.

GC-MS (M^{+•}): 219/221.

### 1.4.2 Synthesis of 4-(5-acetyl-4,5,6,7-tetrahydro[1,3]thiazolo[5,4-c]pyridin-2-yl)phenol a28

A solution of 1-acetyl-3-bromopiperidin-4-one a27 (14.37 g, 65.27 mmol, 1 eq) in isopropanol (220 ml) is treated with 4-hydroxybenzenecarbothioamide (10 g, 65.27 mmol, 1 eq) and the mixture is stirred at 60° C. for 2 hours. The mixture is then concentrated under reduced pressure. The crude product is taken up in dichloromethane-methanol (90:10) and washed with water. The organic layer is dried over magnesium sulfate and concentrated in vacuo. The residue is purified over silica gel (eluent: dichloromethane/methanol 90:10). The product is taken up with a 1:1 mixture of ethyl acetate and water and the solid obtained is filtered and dried to afford 4.61 g of 4-(5-acetyl-4,5,6,7-tetrahydro[1,3]thiazolo[5,4-c]pyridin-2-yl)phenol a28 as a white solid.

Yield: 26%.

¹H NMR (DMSO): δ 9.97 (m, 1H), 7.71 (m, 2H), 6.85 (d, J=8.6 Hz, 2H), 4.72 (m, 2H), 3.78 (m, 2H), 2.81 (m, 2H), 2.11 (m, 3H).

Tert-butyl 2-(4-hydroxyphenyl)-6,7-dihydro[1,3]thiazolo[5,4-c]pyridine-5(4H)-carboxylate a29 may be synthesized according to the same method.

LC-MS (MH⁺): 333.

### 1.5 Synthesis of 5-acetyl-2-{4-[(trans-3-morpholin-4-ylcyclobutyl)oxy]phenyl}-4,5,6,7-tetrahydro[1,3]thiazolo[5,4-c]pyridine 8

A solution of 4-(5-acetyl-4,5,6,7-tetrahydro[1,3]thiazolo[5,4-c]pyridin-2-yl)phenol a28 (0.404 g, 1.47 mmol, 1 eq) in dry N,N-dimethylacetamide (6 ml) is treated with sodium hydride (60% dispersion in mineral oil, 72 mg, 1.8 mmol, 1.1 eq) under an argon atmosphere. After 30 minutes, cis-3-morpholin-4-ylcyclobutyl 4-methylbenzenesulfonate a18 (0.51 g, 1.64 mmol, 1 eq) is added and the mixture is stirred at 70° C. overnight. The mixture is poured onto an aqueous saturated sodium chloride solution and extracted with ethyl acetate. The organic layer is dried over magnesium sulfate and concentrated under reduced pressure. The residue is purified by chromatography over silicagel (eluent: ethyl acetate/methanol 90:10) to afford 0.175 g of 5-acetyl-2-{4-[(trans-3-morpholin-4-ylcyclobutyl)oxy]phenyl}-4,5,6,7-tetrahydro[1,3]thiazolo[5,4-c]pyridine 8 as a yellow solid.

Yield: 29%.

LC-MS (MH⁺): 414.

Compounds 4, 12, 13, 14, 19, 20 and 22 may be synthesized according to the same method.

### EXAMPLE 2

### Synthesis of 5-acetyl-2-{4-[(trans-3-piperidin-1-ylcyclobutyl)oxy]phenyl}-4,5,6,7-tetrahydro[1,3]thiazolo[5,4-c]pyridine 4

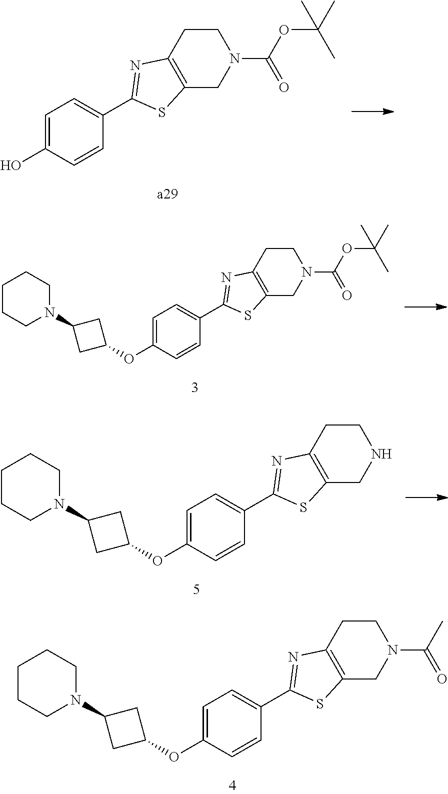

### 2.1 Synthesis of tert-butyl 2-{4-[(trans-3-piperidin-1-ylcyclobutyl)oxy]phenyl}-6,7-dihydro[1,3]thiazolo[5,4-c]pyridine-5(4H)-carboxylate 3

Sodium hydride 60% (89 mg, 2.22 mmol, 2 eq) is added to a solution of tert-butyl 2-(4-hydroxyphenyl)-6,7-dihydro[1,3]thiazolo[5,4-c]pyridine-5(4H)-carboxylate a29 (370 mg, 1.11 mmol, 1 eq) in dry N,N-dimethylformamide (15 ml) at 0° C. The mixture is stirred at room temperature for 30 minutes then cis-3-piperidin-1-ylcyclobutyl 4-methylbenzenesulfonate a25 (344 mg, 1.11 mmol, 1 eq) is added and the mixture is heated at 80° C. After 3 days, sodium hydride (135 mg, 3.33 mmol, 3 eq) and cis-3-piperidin-1-ylcyclobutyl 4-methylbenzenesulfonate a25 (525 mg, 1.66 mmol, 1.5 eq) are added and the mixture is heated at 80° C. for 4 more days. The mixture is then concentrated to dryness. The residue is dissolved in ethyl acetate and washed with a saturated solution of sodium hydrogenocarbonate. The aqueous phase is extracted with ethyl acetate, the combined organic phases are dried over magnesium sulfate and concentrated in vacuo to give 618 mg of a crude solid that is purified by chromatography over silicagel (eluent: ethyl acetate 100%) to afford 335 mg of tert-butyl 2-{4-[(trans-3-piperidin-1-ylcyclobutyl)oxy]phenyl}-6,7-dihydro[1,3]thiazolo[5,4-c]pyridine-5(4H)-carboxylate 3 as an orange solid.

Yield: 64%.

LC-MS (MH⁺): 470.

### 2.2 Synthesis of 2-{4-[(trans-3-piperidin-1-ylcyclobutyl)oxy]phenyl}-4,5,6,7-tetrahydro[1,3]thiazolo[5,4-c]pyridine 5

Trifluoroacetic acid (1.5 ml) is added to a solution of tert-butyl 2-{4-[(trans-3-piperidin-1-ylcyclobutyl)oxy]phenyl}-6,7-dihydro[1,3]thiazolo[5,4-c]pyridine-5(4H)-carboxylate 3 (280 mg, 0.59 mmol, 1 eq) in dichloromethane (8 ml) and the mixture is stirred at room temperature for 2 h. The mixture is concentrated to dryness. The residue is dissolved in water, brought to pH 9 with a saturated solution of potassium carbonate and extracted twice with dichloromethane. The combined organic layers are dried over magnesium sulfate and concentrated under vacuum to afford 180 mg of 2-{4-[(trans-3-piperidin-1-ylcyclobutyl)oxy]phenyl}-4,5,6,7-tetrahydro[1,3]thiazolo[5,4-c]pyridine 5 as an orange solid.

Yield: 83%.

LC-MS (MH⁺): 370.

### 2.3 Synthesis of 5-acetyl-2-{4-[(trans-3-piperidin-1-ylcyclobutyl)oxy]phenyl}-4,5,6,7-tetrahydro[1,3]thiazolo[5,4-c]pyridine 4

Triethylamine (91 μl, 0.65 mmol, 1.52 eq) and acetyl chloride (41 mg, 0.52 mmol, 1.2 eq) are added to a solution of 2-{4-[(trans-3-piperidin-1-ylcyclobutyl)oxy]phenyl}-4,5,6,7-tetrahydro[1,3]thiazolo[5,4-c]pyridine 5 (160 mg, 0.43 mmol, 1 eq) in dichloromethane (10 ml) at 0° C. The mixture is stirred 3 h at 20° C. Dichloromethane is added and the organic layer is successively washed with a saturated solution of sodium hydrogenocarbonate and with brine. The organic layer is dried over magnesium sulfate and concentrated under reduced pressure to afford 176 mg of a yellow solid. The crude mixture is purified by chromatography over silica gel (dichloromethane/methanol/ammonia 96:4:0.4) to afford 95 mg of 5-acetyl-2-{4-[(trans-3-piperidin-1-ylcyclobutyl)oxy]phenyl}-4,5,6,7-tetrahydro[1,3]thiazolo[5,4-c]pyridine 4 as a white solid.

Yield: 54%.

LC-MS (MH⁺): 412.

### EXAMPLE 3

### Synthesis of 2-{4-[(trans-3-piperidin-1-ylcyclobutyl)oxy]phenyl}-6,7-dihydro[1,3]thiazolo[5,4-c]pyridine-5(4H)-carboxamide 10

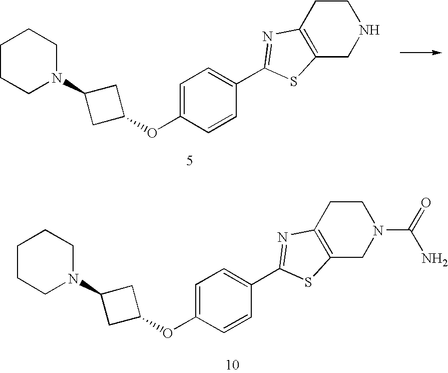

A solution of 2-{4-[(trans-3-piperidin-1-ylcyclobutyl)oxy]phenyl}-4,5,6,7-tetrahydro[1,3]thiazolo[5,4-c]pyridine 5 (1.0 g, 2.7 mmol, 1 eq) in dichloromethane (32 ml) is treated with trimethylsilylisocyanate (400 μl, 2.9 mmol, 1.1 eq). The mixture is stirred overnight at room temperature, quenched with water and extracted with dichloromethane. The organic layer is dried over magnesium sulfate, concentrated under reduced pressure and purified by chromatography over silicagel (dichloromethane/methanol/ammonia 96:4:1) to afford 410 mg of 2-{4-[(trans-3-piperidin-1-ylcyclobutyl)oxy]phenyl}-6,7-dihydro[1,3]thiazolo[5,4-c]pyridine-5(4H)-carboxamide 10 as a white solid.

Yield: 36%.

LC-MS (MH⁺): 413.

N-ethyl-2-{4-[(trans-3-piperidin-1-ylcyclobutyl)oxy]phenyl}-6,7-dihydro[1,3]thiazolo[5,4-c]pyridine-5(4H)-carboxamide 21 may be synthesized according to the same method.

### EXAMPLE 4

### Synthesis of 5-(morpholin-4-ylcarbonyl)-2-{4-[(trans-3-piperidin-1-ylcyclobutyl)oxy]phenyl}-4,5,6,7-tetrahydro[1,3]thiazolo[5,4-c]pyridine 6

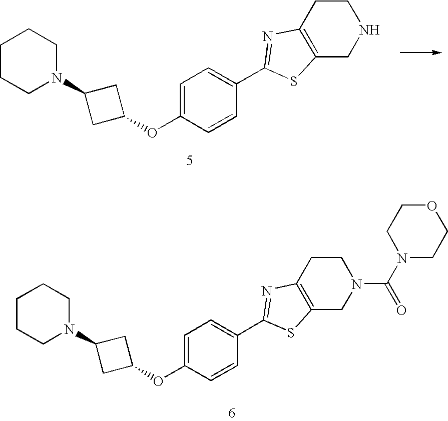

Triphosgene (0.064 g, 0.225 mmol, 0.37 eq) is added to a solution of 2-{4-[(trans-3-piperidin-1-ylcyclobutyl)oxy]phenyl}-4,5,6,7-tetrahydro[1,3]thiazolo[5,4-c]pyridine 5 (0.225 g, 0.61 mmol, 1 eq) in dichloromethane (10 ml) at 0° C. The mixture is stirred 1 h at room temperature, cooled down to 0° C., then morpholine (0.053 ml, 0.61 mmol, 1 eq) and triethylamine (0.085 ml, 0.61 mmol, 1 eq) are added. The mixture is stirred overnight at room temperature and washed twice with a saturated aqueous solution of sodium hydrogenocarbonate. The organic layer is dried over magnesium sulfate and concentrated under reduced pressure. The residue is purified by chromatography over silicagel (dichloromethane/ethanol/ammonia 95:5:0.5) to yield 0.135 g of 5-(morpholin-4-ylcarbonyl)-2-{4-[(trans-3-piperidin-1-ylcyclobutyl)oxy]phenyl}-4,5,6,7-tetrahydro[1,3]thiazolo[5,4-c]pyridine 6 as a yellow solid.

Yield: 46%.

LC-MS (MH⁺): 483.

### EXAMPLE 5

### Synthesis of 5-(morpholin-4-ylsulfonyl)-2-{4-[(trans-3-piperidin-1-ylcyclobutyl)oxy]phenyl}-4,5,6,7-tetrahydro[1,3]thiazolo[5,4-c]pyridine 7

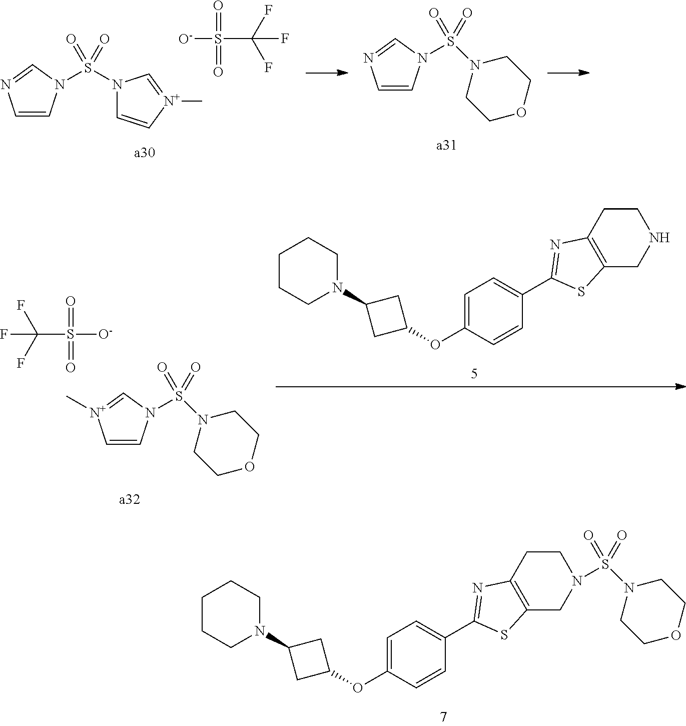

### 5.1 Synthesis of 4-(1H-imidazol-1-ylsulfonyl)morpholine a31

Morpholine (0.35 g, 3.92 mmol, 1 eq) is added to a solution of 1-(1H-imidazol-1-ylsulfonyl)-3-methyl-1H-imidazol-3-ium trifluoromethanesulfonate a30 (1.7 g, 4.7 mmol, 1.2 eq) (obtained as described in J. Org. Chem. 2003, 68, 115-199) in acetonitrile (70 ml) and the mixture is stirred overnight at room temperature. The solvent is removed under reduced pressure to lead to a residue which is then diluted with ethyl acetate and washed twice with a saturated aqueous solution of sodium hydrogen carbonate. The organic layer is then dried over magnesium sulfate and concentrated under reduced pressure. The residue is purified by chromatography over silicagel (dichloromethane/methanol 99:1) to yield 0.49 g of 4-(1H-imidazol-1-ylsulfonyl)morpholine a31.

Yield: 48%.

LC-MS (MH⁺): 218.

### 5.2 Synthesis of 3-methyl-1-(morpholin-4-ylsulfonyl)-1H-imidazol-3-ium trifluoromethanesulfonate a32

Methyl trifluoromethanesulfonate (0.25 ml, 2.2 mmol, 1 eq) is added dropwise to a stirred suspension of 4-(1H-imidazol-1-ylsulfonyl)morpholine a31 (0.48 g, 2.2 mmol, 1 eq) in dichloromethane (15 ml) at 0° C. under argon atmosphere. The mixture is stirred for 2 hours at 0° C., the resulting suspension is filtered and washed with dichloromethane to afford 0.6 g of 3-methyl-1-(morpholin-4-ylsulfonyl)-1H-imidazol-3-ium trifluoromethanesulfonate a32 as a white solid which is directly used in the next step without any other purification.

Yield: 73%.

LC-MS (MH⁺): 382.

### 5.3 Synthesis of 5-(morpholin-4-ylsulfonyl)-2-{4-[(trans-3-piperidin-1-ylcyclobutyl)oxy]phenyl}-4,5,6,7-tetrahydro[1,3]thiazolo[5,4-c]pyridine 7

3-methyl-1-(morpholin-4-ylsulfonyl)-1H-imidazol-3-ium trifluoromethanesulfonate a32 (0.595 g, 1.56 mmol, 1.2 eq) is added to a solution of 2-{4-[(trans-3-piperidin-1-ylcyclobutyl)oxy]phenyl}-4,5,6,7-tetrahydro[1,3]thiazolo[5,4-c]pyridine 5 (0.48 g, 1.3 mmol, 1 eq) in acetonitrile (20 ml) and the mixture is stirred overnight at room temperature. The solvent is removed under reduced pressure to give a residue which is then diluted with ethyl acetate and washed twice with a saturated aqueous solution of sodium hydrogenocarbonate. The organic layer is then dried over magnesium sulfate and concentrated under reduced pressure. The residue is purified by chromatography over silicagel (eluent: dichloromethane/methanol 97:3) to yield 0.16 g of 5-(morpholin-4-ylsulfonyl)-2-{4-[(trans-3-piperidin-1-ylcyclobutyl)oxy]phenyl}-4,5,6,7-tetrahydro[1,3]thiazolo[5,4-c]pyridine 7 as a yellow solid.

Yield: 24%.

LC-MS (MH⁺): 519.

### EXAMPLE 6

### Synthesis of 2-oxo-2-[2-{4-[(trans-3-piperidin-1-ylcyclobutyl)oxy]phenyl}-6,7-dihydro[1,3]thiazolo[5,4-c]pyridin-5(4H)-yl]ethanol 11

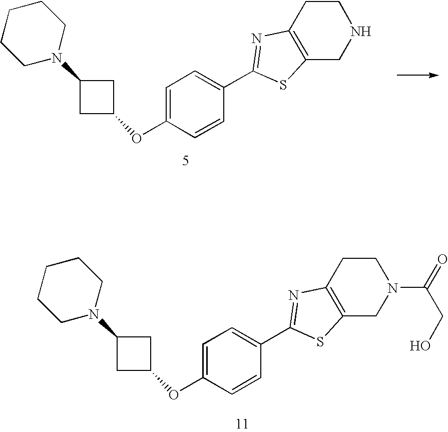

A solution of 2-{4-[(trans-3-piperidin-1-ylcyclobutyl)oxy]phenyl}-4,5,6,7-tetrahydro[1,3]thiazolo[5,4-c]pyridine 5 (1.2 g, 3.2 mmol, 1 eq), in dichloromethane (18 ml) is treated with 1-(3-dimethylaminopropyl)-3-ethylcarbodiimide hydrochloride (2.2 g, 11.4 mmol, 3.5 eq), 1-hydroxybenzotriazole (0.9 g, 6.5 mmol, 2.0 eq) and N,N-dimethylaminopyridine (0.4 g, 3.2 mmol, 1.0 eq). The mixture is stirred 30 minutes at room temperature and glycolic acid (300 mg, 3.9 mmol, 1.2 eq) is added. The resulting mixture is stirred overnight at room temperature, quenched with a 0.5 N aqueous hydrogen chloride solution and extracted with dichloromethane. The organic layer is washed with an aqueous saturated sodium bicarbonate solution, dried over magnesium sulfate and evaporated to dryness. The residue is purified by chromatography over silicagel (dichloromethane/methanol/ammonia 96:4:0.4) to afford 415 mg of 2-oxo-2-[2-{4-[(trans-3-piperidin-1-ylcyclobutyl)oxy]phenyl}-6,7-dihydro[1,3]thiazolo[5,4-c]pyridin-5(4H)-yl]ethanol 11 as a yellow solid.

Yield: 30%.

LC-MS (MH⁺): 428.

Compounds 41, 42, 53, 60, 63 and 65 may be synthesized according to the same method.

### EXAMPLE 7

### Synthesis of 2-oxo-2-[2-{4-[(trans-3-piperidin-1-ylcyclobutyl)oxy]phenyl}-6,7-dihydro[1,3]thiazolo[5,4-c]pyridin-5(4H)-yl]ethanamine 9

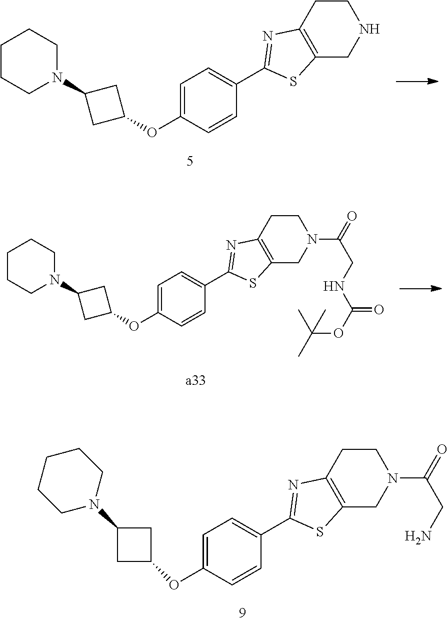

### 7.1 Synthesis of tert-butyl {2-oxo-2-[2-{4-[(trans-3-piperidin-1-ylcyclobutyl)oxy]phenyl}-6,7-dihydro[1,3]thiazolo[5,4-c]pyridin-5(4H)-yl]ethyl}carbamate a33

A solution of 2-{4-[(trans-3-piperidin-1-ylcyclobutyl)oxy]phenyl}-4,5,6,7-tetrahydro[1,3]thiazolo[5,4-c]pyridine 5 (500 mg, 1.4 mmol, 1.0 eq) in dichloromethane (8 ml) is treated with 1-(3-dimethylaminopropyl)-3-ethylcarbodiimide hydrochloride (0.9 g, 4.9 mmol, 3.5 eq), 1-hydroxybenzotriazole (380 mg, 2.8 mmol, 2.0 eq) and 4-(N,N-dimethylamino)-pyridine (175 mg, 1.4 mmol, 1.0 eq). The mixture is stirred 10 minutes at 0° C. and N-(tert-butoxycarbonyl)glycine (270 mg, 1.5 mmol, 1.1 eq) is added. The resulting mixture is stirred overnight at room temperature, then quenched with a 0.5 N aqueous hydrogen chloride solution and extracted with dichloromethane. The organic layer is washed with an aqueous saturated sodium bicarbonate solution, dried over magnesium sulfate and evaporated to dryness to afford tert-butyl {2-oxo-2-[2-{4-[(trans-3-piperidin-1-ylcyclobutyl)oxy]phenyl}-6,7-dihydro[1,3]thiazolo[5,4-c]pyridin-5(4H)-yl]ethyl}carbamate a33, which is used in the next step without any further purification.

LC-MS (MH⁺): 527.

### 7.2 Synthesis of 2-oxo-2-[2-{4-[(trans-3-piperidin-1-ylcyclobutyl)oxy]phenyl}-6,7-dihydro[1,3]thiazolo[5,4-c]pyridin-5(4H)-yl]ethanamine 9

Trifluoroacetic acid (3.0 ml, 4.5 mmol, 30 eq) is added dropwise at 0° C. to a solution of tert-butyl {2-oxo-2-[2-{4-[(trans-3-piperidin-1-ylcyclobutyl)oxy]phenyl}-6,7-dihydro[1,3]thiazolo[5,4-c]pyridin-5(4H)-yl]ethyl}carbamate a33 (7.4 g, 1.4 mmol, 1.0 eq) in dichloromethane (18 ml) and the mixture is stirred overnight at room temperature. The reaction mixture is treated with an aqueous saturated potassium carbonate solution and extracted with dichloromethane. The organic layer is dried over magnesium sulfate and concentrated to dryness. The residue is purified by chromatography over silicagel (dichloromethane/methanol/ammonia 94:6) to afford 59 mg of 2-oxo-2-[2-{4-[(trans-3-piperidin-1-ylcyclobutyl)oxy]phenyl}-6,7-dihydro[1,3]thiazolo[5,4-c]pyridin-5(4H)-yl]ethanamine 9 as a yellow solid.

Yield: 10%.

LC-MS (MH⁺): 427.

### EXAMPLE 8

### Synthesis of 5-(methoxyacetyl)-2-{4-[(trans-3-piperidin-1-ylcyclobutyl)oxy]phenyl}-4,5,6,7-tetrahydro[1,3]thiazolo[5,4-c]pyridine 2

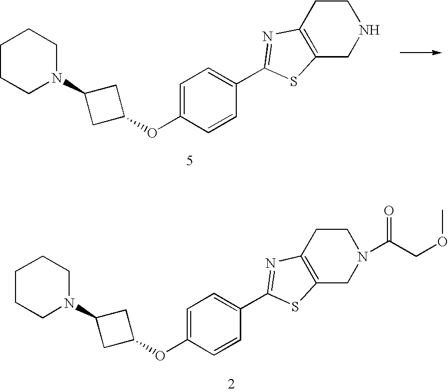

Methoxyacetyl chloride (0.066 ml, 0.61 mmol, 1 eq) is added to a solution of 2-{4-[(trans-3-piperidin-1-ylcyclobutyl)oxy]phenyl}-4,5,6,7-tetrahydro[1,3]thiazolo[5,4-c]pyridine 5 (0.225 g, 0.61 mmol, 1 eq) and triethylamine (0.085 ml, 0.61 mmol, 1 eq) in dichloromethane (10 ml) at 0° C. The mixture is stirred overnight at room temperature then washed twice with an aqueous saturated solution of sodium hydrogencarbonate. The organic layer is dried over magnesium sulfate and concentrated under reduced pressure. The residue is purified by chromatography over silicagel (dichloromethane/methanol 97:3) to yield 0.152 g of 5-(methoxyacetyl)-2-{4-[(trans-3-piperidin-1-ylcyclobutyl)oxy]phenyl}-4,5,6,7-tetrahydro[1,3]thiazolo[5,4-c]pyridine 2 as a white solid.

Yield: 56%.

LC-MS (MH⁺): 442.

### EXAMPLE 9

### Synthesis of 3-oxo-3-[2-{4-[(trans-3-piperidin-1-ylcyclobutyl)oxy]phenyl}-6,7-dihydro[1,3]thiazolo[5,4-c]pyridin-5(4H)-yl]propanamide 25

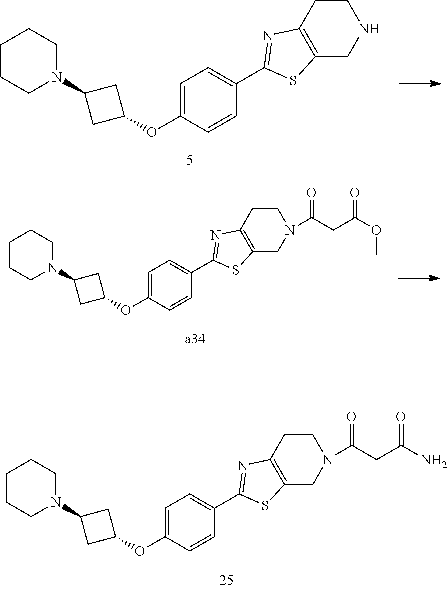

### 9.1 Synthesis of methyl 3-oxo-3-[2-{4-[(trans-3-piperidin-1-ylcyclobutyl)oxy]phenyl}-6,7-dihydro[1,3]thiazolo[5,4-c]pyridin-5(4H)-yl]propanoate a34

Methyl-3-chloro-3-oxopropionate (0.44 g, 3.2 mmol, 1.2 eq) is added to a suspension of 2-{4-[(trans-3-piperidin-1-ylcyclobutyl)oxy]phenyl}-4,5,6,7-tetrahydro[1,3]thiazolo[5,4-c]pyridine 5 (1 g, 2.7 mmol, 1 eq) and triethylamine (0.75 ml, 5.4 mmol, 2 eq) in dichloromethane (30 ml). The mixture is stirred overnight at room temperature, successively washed with water and with brine. The organic layer is dried over magnesium sulfate and concentrated under reduced pressure to yield 1.26 g of methyl 3-oxo-3-[2-{4-[(trans-3-piperidin-1-ylcyclobutyl)oxy]phenyl}-6,7-dihydro[1,3]thiazolo[5,4-c]pyridin-5(4H)-yl]propanoate a34 which is directly used in the next step without any other purification.

Yield: >95%.

LC-MS (MH+): 470.

### 9.2 Synthesis of 3-oxo-3-[2-{4-[(trans-3-piperidin-1-ylcyclobutyl)oxy]phenyl}-6,7-dihydro[1,3]thiazolo[5,4-c]pyridin-5(4H)-yl]propanamide 25

A 7 N solution of ammonia in methanol (25 ml) is added to a solution of methyl 3-oxo-3-[2-{4-[(trans-3-piperidin-1-ylcyclobutyl)oxy]phenyl}-6,7-dihydro[1,3]thiazolo[5,4-c]pyridin-5(4H)-yl]propanoate a34 (1.1 g, 2.34 mmol, 1 eq) in methanol (20 ml) and the mixture is stirred for 48 h at 70° C. in a sealed vessel. The solvent is removed under reduced pressure to give a residue which is then diluted with dichloromethane and washed twice with water. The organic layer is dried over magnesium sulfate and concentrated under reduced pressure. The residue is purified by chromatography over silicagel (dichloromethane/methanol/ammonia 98:2:0.2) to yield 0.189 g of 3-oxo-3-[2-{4-[(trans-3-piperidin-1-ylcyclobutyl)oxy]phenyl}-6,7-dihydro[1,3]thiazolo[5,4-c]pyridin-5(4H)-yl]propanamide 25 as a beige solid.

Yield: 18%.

LC-MS (MH⁺): 455.

### EXAMPLE 10

### Synthesis of methyl[2-{4-[(trans-3-piperidin-1-ylcyclobutyl)oxy]phenyl}-6,7-dihydro[1,3]thiazolo[5,4-c]pyridin-5(4H)-yl]acetate 26

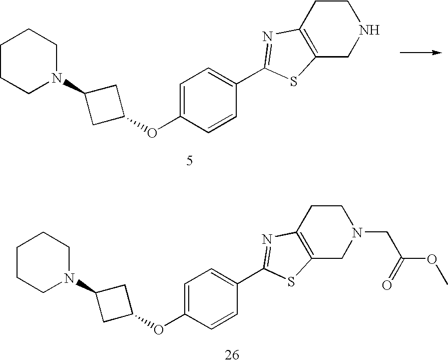

Methyl bromoacetate (460 μl, 4.9 mmol, 1.2 eq) is added to a mixture of 2-{4-[(trans-3-piperidin-1-ylcyclobutyl)oxy]phenyl}-4,5,6,7-tetrahydro[1,3]thiazolo[5,4-c]pyridine 5 (1.5 g, 4.1 mmol, 1.0 eq), potassium phosphate (2.7 g, 12.7 mmol, 2.6 eq) and sodium iodide (189 mg, 1.3 mmol, 0.3 eq) in dimethylformamide/acetonitrile (2:1 v/v, 60 ml). The mixture is stirred at 40° C. for 1 hour, then water is added. The reaction mixture is extracted with dichloromethane. The organic layer is dried over magnesium sulfate and evaporated to dryness. The residue is purified by chromatography over silicagel (eluent: dichloromethane/methanol/ammonia 96:4) to afford 540 mg of methyl [2-{4-[(trans-3-piperidin-1-ylcyclobutyl)oxy]phenyl}-6,7-dihydro[1,3]thiazolo[5,4-c]pyridin-5(4H)-yl]acetate 26 as a yellow solid.

Yield: 30%.

LC-MS (MH⁺): 442.

### EXAMPLE 11

### Synthesis of (2S)-3-[2-{4-[(trans-3-piperidin-1-ylcyclobutyl)oxy]phenyl}-6,7-dihydro[1,3]thiazolo[5,4-c]pyridin-5(4H)-yl]propane-1,2-diol 16

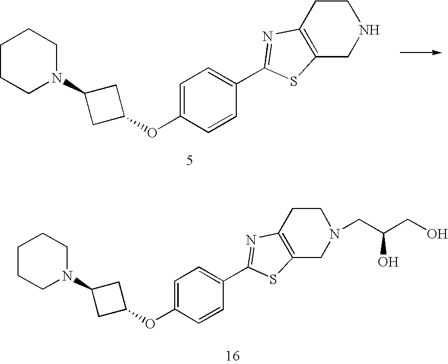

(2R)-3-chloropropane-1,2-diol (0.28 g, 2.53 mmol, 1.2 eq) is added to a suspension of 2-{4-[(trans-3-piperidin-1-ylcyclobutyl)oxy]phenyl}-4,5,6,7-tetrahydro[1,3]thiazolo[5,4-c]pyridine 5 (0.78 g, 2.11 mmol, 1 eq) and potassium carbonate (0.58 g, 4.22 mmol, 2 eq) in acetonitrile (35 ml) with a catalytic quantity of sodium iodide. The mixture is stirred for 54 h under reflux. The solvent is removed under reduced pressure and the residue is diluted with ethyl acetate. This organic layer is washed twice with an aqueous saturated solution of sodium hydrogen carbonate, dried over magnesium sulfate and concentrated under reduced pressure. The residue is purified by chromatography over silicagel (gradient: dichloromethane/methanol/ammonia 100:0:0 to 95:5:0.5) to yield 0.197 g of (2S)-3-[2-{4-[(trans-3-piperidin-1-ylcyclobutyl)oxy]phenyl}-6,7-dihydro[1,3]thiazolo[5,4-c]pyridin-5(4H)-yl]propane-1,2-diol 16 as a beige solid.

Yield: 21%.

LC-MS (MH⁺): 444.

Chiral chromatography (Chiralcel OD-H, iso-hexane/n-propanol/diethylamine 50:50:0.1): t_{R}=5′49 (ee: 94.8%).

Compounds 15 and 17 may be synthesized according to the same method by using respectively 3-chloropropane-1,2-diol and (2S)-3-chloropropane-1,2-diol as reactive.

Chiral chromatography for compound 17 (Chiralcel OD-H, iso-hexane/n-propanol/diethylamine 50:50:0.1): t_{R}=4′85 (ee: 94%).

Compound 18 may be synthesized using 2-bromoacetamide. Compound 37 may be synthesized using 2-bromoethanol.

### EXAMPLE 12

### Synthesis of cis-3-[2-{4-[(trans-3-piperidin-1-ylcyclobutyl)oxy]phenyl}-6,7-dihydro[1,3]thiazolo[5,4-c]pyridin-5(4H)-yl]cyclobutanol 24

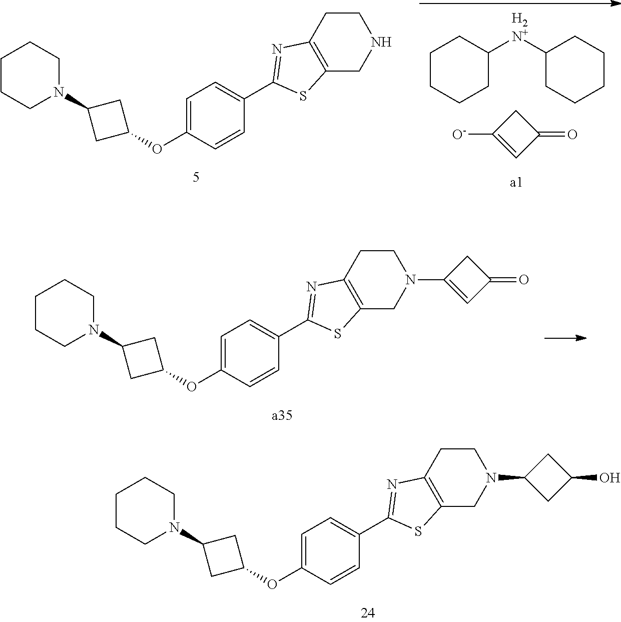

### 12.1 Synthesis of 3-[2-{4-[(trans-3-piperidin-1-ylcyclobutyl)oxy]phenyl}-6,7-dihydro[1,3]thiazolo[5,4-c]pyridin-5(4H)-yl]cyclobut-2-en-1-one a35

Trifluoroacetic acid (0.22 ml, 2.97 mmol, 1.1 eq) is added to a stirred suspension of N-cyclohexylcyclohexanaminium 3-oxocyclobut-1-en-1-olate a1 (0.71 g, 2.7 mmol, 1 eq) and 2-{4-[(trans-3-piperidin-1-ylcyclobutyl)oxy]phenyl}-4,5,6,7-tetrahydro[1,3]thiazolo[5,4-c]pyridine 5 (1.0 g, 2.7 mmol, 1 eq) in dioxane (40 ml). The reaction mixture is stirred overnight at 20° C. The solvent is then removed under reduced pressure, the residue is taken up with ethyl acetate and the organic layer is washed twice with an aqueous saturated solution of sodium hydrogen carbonate, dried over magnesium sulfate and concentrated under vacuum to yield 1.25 g of 3-[2-{4-[(trans-3-piperidin-1-ylcyclobutyl)oxy]phenyl}-6,7-dihydro[1,3]thiazolo[5,4-c]pyridin-5(4H)-yl]cyclobut-2-en-1-one a35 which is directly used in the next step without any other purification.

Yield: >95%.

LC-MS (MH⁺): 436.

### 12.2 Synthesis of cis-3-[2-{4-[(trans-3-piperidin-1-ylcyclobutyl)oxy]phenyl}-6,7-dihydro[1,3]thiazolo[5,4-c]pyridin-5(4H)-yl]cyclobutanol 24

A solution of 3-[2-{4-[(trans-3-piperidin-1-ylcyclobutyl)oxy]phenyl}-6,7-dihydro[1,3]thiazolo[5,4-c]pyridin-5(4H)-yl]cyclobut-2-en-1-one a35 (1.2 g, 2.75 mmol, 1 eq) in ethanol (35 ml) is treated with portions of sodium borohydride (0.52 g, 13.77 mmol, 3.5 eq). At the end of the addition, the mixture is stirred overnight at 70° C. The solvent is removed under reduced pressure, then the residue is diluted with dichloromethane. This organic layer is washed with water, dried over magnesium sulfate and concentrated under reduced pressure. The residue is purified by chromatography over silicagel (dichloromethane/heptane/methanol/ammonia 48.5:48.5:3:0.3) to afford 0.271 g of cis-3-[2-{4-[(trans-3-piperidin-1-ylcyclobutyl)oxy]phenyl}-6,7-dihydro[1,3]thiazolo[5,4-c]pyridin-5(4H)-yl]cyclobutanol 24 as a beige solid.

Yield: 22%.

LC-MS (MH⁺): 440.

### EXAMPLE 13

### Synthesis of 4-acetyl-2-{4-[(trans-3-piperidin-1-ylcyclobutyl)oxy]phenyl}-4,5,6,7-tetrahydro[1,3]thiazolo[4,5-b]pyridine 30

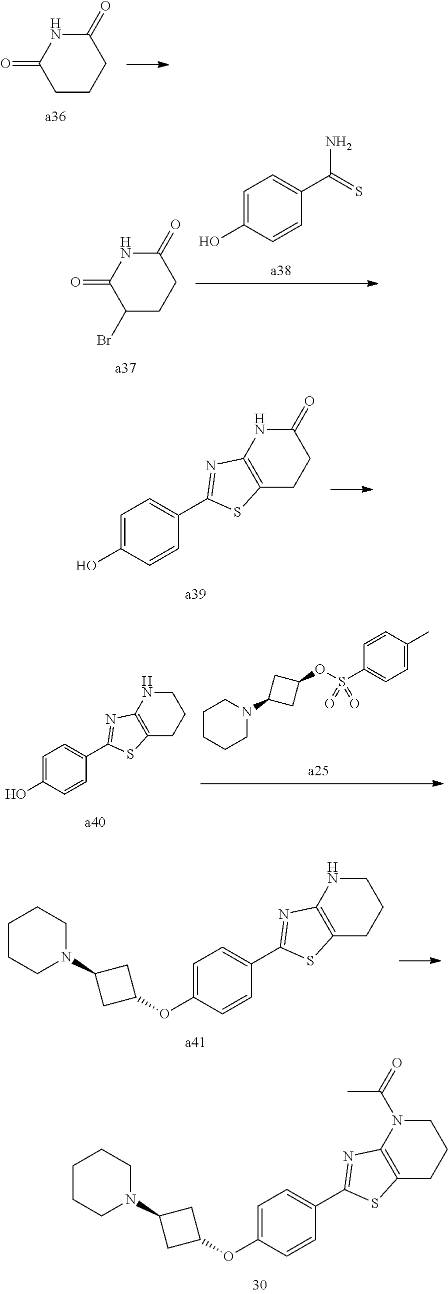

### 13.1 Synthesis of 3-bromopiperidine-2,6-dione a37

Bromine (4.5 ml, 87.8 mmol) is added to a suspension of piperidine-2,6-dione a36 (10.2 g, 50.3 mmol) suspended in chloroform (20 ml) and the mixture is stirred in a closed vessel for 90 minutes at a bath temperature of 110° C. After cooling, the vessel is opened and stirring is continued until no more hydrogen bromide escapes. The reaction mixture is evaporated in vacuo. The residue is dissolved in ethanol and evaporated to afford 17.1 g of 3-bromopiperidine-2,6-dione a37 as white crystals.

Yield: 99%.

LC-MS (MH⁺): 193.

### 13.2 Synthesis of 2-(4-hydroxyphenyl)-6,7-dihydro[1,3]thiazolo[4,5-b]pyridin-5(4H)-one a39

A mixture of 4-hydroxythiobenzamide a38 (50.0 g, 0.33 mmol, 1 eq) and 3-bromopiperidine-2,6-dione a37 (69.0 g, 0.36 mmol, 1.1 eq) in 2-propanol (500 ml) is heated under reflux for 2 h. The solid is dissolved on reaching circa 60° C. before the product starts to precipitate out. The resulting yellow suspension is cooled to 20° C., slowly filtered, and washed with fresh 2-propanol (2×100 ml). The crude product (70.5 g) is taken up with 2:1 ethanol/water (3.7 l) at 70° C. A remaining undissolved impurity is filtered off. The filtrate is heated at reflux for 30 minutes, then the clear solution is allowed to drift slowly to room temperature overnight while stirring. The sandy crystals are collected, reslurried in ethanol (200 ml) for 1 hour, then re-isolated and dried in vacuo at 50-80° C. to afford 38.7 g of 2-(4-hydroxyphenyl)-6,7-dihydro[1,3]thiazolo[4,5-b]pyridin-5(4H)-one a39 as a pale yellow-green powder.

Yield: 48%.

¹H NMR (DMSO) δ 10.62 (s, 1H), 10.01 (s, 1H), 7.67 (d, J=7 Hz, 2H), 6.83 (d, J=7 Hz, 2H), 2.94 (t, J=7.3 Hz, 2H), 2.59 (t, J=7.3 Hz, 2H).

### 13.3 Synthesis of 2-(4-hydroxyphenyl)-4,5,6,7-tetrahydro[1,3]thiazolo[4,5-b]pyridine a40

A suspension of 2-(4-hydroxyphenyl)-6,7-dihydro[1,3]thiazolo[4,5-b]pyridin-5(4H)-one a39 (23.9 g, 97 mmol) in tetrahydrofuran (500 ml) is cooled to 0-5° C. under nitrogen. Borane-dimethyl sulfide complex (22.8 g, 28.5 ml, 300 mmol) is added drop-wise over circa 30 minutes at 4-6° C., followed by a tetrahydrofuran line-wash (2×50 mL). Gas evolution and a mild exotherm are noted during the early stages of the step. After stirring at 5° C. for another hour, the preparation is allowed to warm to 20-25° C. overnight. The reaction is then quenched by adding methanol (250 ml) cautiously at <10° C. (NB exotherm & gas evolution). The resulting solution is concentrated by distillation at atmospheric pressure (740 ml solvent removed). Methanol (500 ml) is charged and the operation is resumed until a further 260 ml distillate has been collected. The residual cloudy solution is cooled slowly to 0-5° C. and the pale yellow crystals that form are filtered off, washed with methanol (2×20 ml), and dried in vacuo at 50° C. to afford 9.1 g of 2-(4-hydroxyphenyl)-4,5,6,7-tetrahydro[1,3]thiazolo[4,5-b]pyridine a40 as a yellow powder.

Yield: 40%.

¹H NMR (DMSO) δ 9.88 (s, 1H), 7.6 (d, 2H), 6.82 (d, 2H), 5.69 (s, 1H), 3.18 (m, 2H), 2.69 (t, 2H), 1.86 (m, 2H).

### 13.4 Synthesis of 2-{4-[(trans-3-piperidin-1-ylcyclobutyl)oxy]phenyl}-4,5,6,7-tetrahydro[1,3]thiazolo[4,5-b]pyridine a41

A solution of 2-(4-hydroxyphenyl)-4,5,6,7-tetrahydro[1,3]thiazolo[4,5-b]pyridine a40 (3 g, 12.9 mmol, 1 eq) in dry N,N-dimethylacetamide (90 ml) is treated with sodium hydride (60% dispersion in mineral oil, 0.77 g, 19.35 mmol, 1.5 eq) under an argon atmosphere. After 15 minutes, cis-3-piperidin-1-ylcyclobutyl 4-methylbenzenesulfonate a25 (3.19 g, 10.32 mmol, 0.8 eq) is added and the mixture is stirred at 70° C. for 60 hours. The mixture is concentrated under reduced pressure, diluted with ethyl acetate and washed twice with water. The organic layer is then dried over magnesium sulfate and concentrated under reduced pressure. The residue is purified by chromatography over silicagel (gradient: dichloromethane/ethanol 100:0 to 80:20) to afford 1.24 g of 2-{4-[(trans-3-piperidin-1-ylcyclobutyl)oxy]phenyl}-4,5,6,7-tetrahydro[1,3]thiazolo[4,5-b]pyridine a41.

Yield: 32%.

LC-MS (MH⁺): 370

### 13.5 Synthesis of 4-acetyl-2-{4-[(trans-3-piperidin-1-ylcyclobutyl)oxy]phenyl}-4,5,6,7-tetrahydro[1,3]thiazolo[4,5-b]pyridine 30

Acetyl chloride (0.049 ml, 0.62 mmol, 1.2 eq) is added to a suspension of 2-{4-[(trans-3-piperidin-1-ylcyclobutyl)oxy]phenyl}-4,5,6,7-tetrahydro[1,3]thiazolo[4,5-b]pyridine a41 (0.19 g, 0.51 mmol, 1 eq) and triethylamine (0.086 ml, 0.62 mmol, 1.2 eq) in dichloromethane (10 ml). The mixture is stirred overnight at room temperature then washed twice with an aqueous saturated solution of ammonium chloride. The organic layer is dried over magnesium sulfate and concentrated under reduce pressure. The residue is purified by chromatography over silicagel (eluent: dichloromethane/ethanol/ammonia 95:5:0.5) to yield 0.03 g of 4-acetyl-2-{4-[(trans-3-piperidin-1-ylcyclobutyl)oxy]phenyl}-4,5,6,7-tetrahydro[1,3]thiazolo[4,5-b]pyridine 30 as a beige solid.

Yield: 15%.

LC-MS (MH⁺): 412.

2-(benzyloxy)-1-[2-(4-{[trans-3-(piperidin-1-yl)cyclobutyl]oxy}phenyl)-6,7-dihydro[1,3]thiazolo[4,5-b]pyridin-4(5H)-yl]ethanone a77 may be synthesized according to the same method (LC-MS (MH⁺): 518).

### EXAMPLE 14

### Synthesis of 5-acetyl-2-{4-[(trans-3-piperidin-1-ylcyclobutyl)thio]phenyl}-4,5,6,7-tetrahydro[1,3]thiazolo[5,4-c]pyridine 23

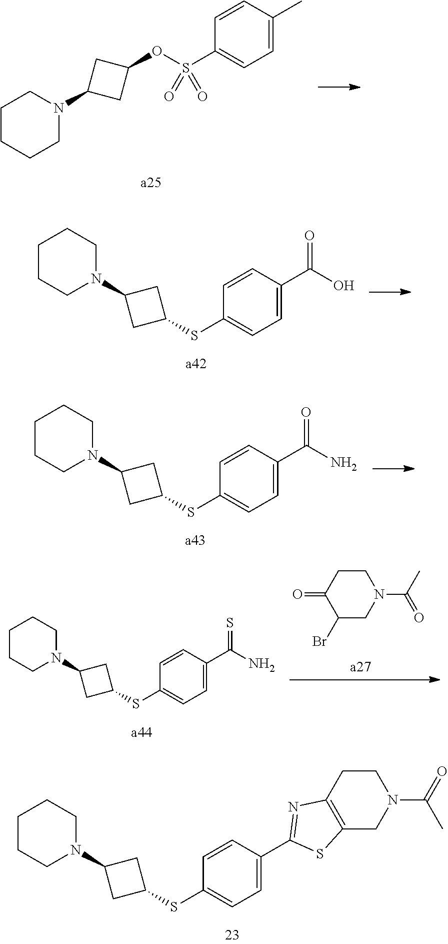

### 14.1 Synthesis of 4-[(trans-3-piperidin-1-ylcyclobutyl)sulfanyl]benzoic acid a42

Sodium hydride (60% in oil, 520 mg, 12.9 mmol, 1.0 eq) is added at 0° C. to a solution of 4-mercaptobenzoic acid (3.0 g, 12.9 mmol, 1.0 eq) in dimethylformamide (20 ml). After 15 minutes, a solution of cis-3-piperidin-1-ylcyclobutyl 4-methylbenzenesulfonate a25 (4.0 g, 12.9 mmol, 1.0 eq) in dimethylformamide (30 ml) is added. The reaction mixture is stirred for 6 days at 40° C. Methanol is added at room temperature. The mixture is concentrated under reduced pressure. The resulting orange solid is filtered off, rinsed with ethanol and recrystallised from a mixture ethanol/water to afford 638 mg of a mixture of 4-[(trans-3-piperidin-1-ylcyclobutyl)sulfanyl]benzoic acid a42 as the main product and the disulfide derivative of 4-mercaptobenzoic acid as a by-product (¹H NMR ratio: 5:1). This mixture is directly used in the following step without any further purification.

¹H NMR (DMSO) δ 7.84 (d, J=8.2 Hz, 2H), 7.23 (d, J=8.2 Hz, 2H), 3.96 (m, 1H), 3.15 (m, 1H), 2.54 (m, 2H), 2.33 (m, 4H), 2.04 (m, 2H), 1.52 (m, 4H), 1.40 (m, 2H).

### 14.2 Synthesis of 4-[(trans-3-piperidin-1-ylcyclobutyl)sulfanyl]benzamide a43

Ammonium bicarbonate (450 mg, 5.7 mmol, 2.6 eq) is added to a mixture of 4-[(trans-3-piperidin-1-ylcyclobutyl)sulfanyl]benzoic acid a42 (638 mg, 2.2 mmol, 1.0 eq), di-tert-butyl dicarbonate (1.2 g, 5.7 mmol, 2.6 eq) and pyridine (230 μl, 2.8 mmol, 1.3 eq) in dimethylformamide (6.4 ml). The reaction mixture is stirred overnight at room temperature, then a solution of 10% n-propanol in ethyl acetate (20 ml) is added. The mixture is washed twice with water. The organic layer is dried over magnesium sulfate and concentrated to dryness to afford 680 mg of a mixture of 4-[(trans-3-piperidin-1-ylcyclobutyl)sulfanyl]benzamide a43 as the main product and the disulfide derivative of 4-mercaptobenzamide as a by-product (LC-MS ratio: 4:1). This mixture is directly used in the following step.

LC-MS (MH⁺): 442.

### 14.3 Synthesis of 4-[(trans-3-piperidin-1-ylcyclobutyl)sulfanyl]benzenecarbothioamide a44

2,4-bis(4-methoxyphenyl)-2,4-dithioxo-1,3,2,4-dithiadiphosphetane (Lawesson's reagent) (2.6 g, 6.5 mmol, 1.5 eq) is added to a mixture of 4-[(trans-3-piperidin-1-ylcyclobutyl)sulfanyl]benzamide a43 (1.2 g, 4.3 mmol, 1.0 eq) in tetrahydrofuran (24 ml). The resulting mixture is stirred at room temperature for 3 hours, then the yellow solid is filtered and rinsed with tetrahydrofuran. The filtrate is concentrated to dryness to afford crude 4-[(trans-3-piperidin-1-ylcyclobutyl)sulfanyl]benzenecarbothioamide a44 that is directly used in the following step.

LC-MS (MH⁺): 307.

### 14.4 Synthesis of 5-acetyl-2-{4-[(trans-3-piperidin-1-ylcyclobutyl)thio]phenyl}-4,5,6,7-tetrahydro[1,3]thiazolo[5,4-c]pyridine 23

A solution of 1-acetyl-3-bromopiperidin-4-one a27 (1.0 g, 4.5 mmol, 1.1 eq) in isopropanol (25 ml) is treated with 4-[(trans-3-piperidin-1-ylcyclobutyl)sulfanyl]benzenecarbothioamide a44 (1.2 g, 4.0 mmol, 1 eq) and the mixture is stirred at 60° C. for 5 days, then 1-acetyl-3-bromo-piperidin-4-one hydrobromide salt (2.0 g, 4.0 mmol, 1.0 eq) is added. The mixture is stirred at 60° C. overnight, then cooled to room temperature. Ethyl acetate is added and the mixture is washed with an aqueous saturated solution of sodium bicarbonate. The organic layer is dried over magnesium sulfate and concentrated under vacuum. The residue is purified over silica gel (dichloromethane/methanol/ammonia 96:4:1). The product is triturated with ethanol and the solid obtained is filtered and dried to afford 100 mg of 5-acetyl-2-{4-[(trans-3-piperidin-1-ylcyclobutyl)thio]phenyl}-4,5,6,7-tetrahydro[1,3]thiazolo[5,4-c]pyridine 23 as a beige solid.

Yield (for 4 steps): 6%.

LC-MS (MH⁺): 428.

### EXAMPLE 15

### Synthesis of 4-acetyl-2-{4-[(trans-3-piperidin-1-ylcyclobutyl)oxy]phenyl}-5,6,7,8-tetrahydro-4H-[1,3]thiazolo[5,4-b]azepine 31

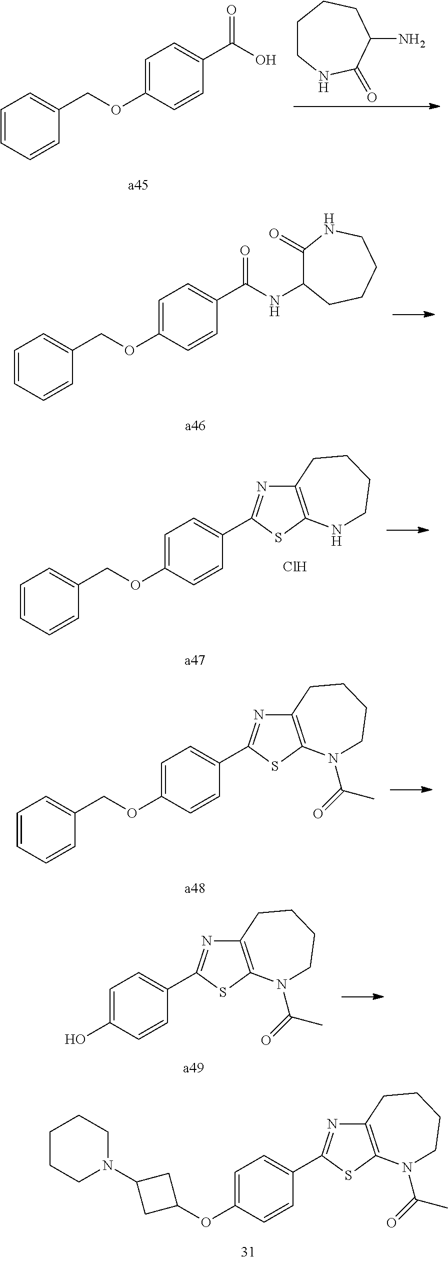

### 15.1 Synthesis of 4-(benzyloxy)-N-(2-oxoazepan-3-yl)benzamide a46

A suspension of 4-(benzyloxy)benzoic acid a45 (5.0 g, 21.93 mmol, 1 eq) and N,N-dimethylformamide (0.5 ml) in dichloromethane (300 ml) at 0° C. is treated with oxalyl chloride (2.83 ml, 26.32 mmol, 1.2 eq). The mixture is left to warm up to room temperature and stirred until gas evolution has ceased. Half of the solvent is removed under reduced pressure and the resulting solution is added to a mixture of DL-α-amino-ε-caprolactam (3.37 g, 26.32 mmol, 1.2 eq) and triethylamine (6.11 ml, 43.86 mmol, 2 eq) in dichloromethane (300 ml). After 1 h stirring at 20° C., water (200 ml) is added and the organic layer is dried over magnesium sulfate and concentrated. The residue is taken up with ethyl acetate, the resulting suspension is filtered and the solid dried at 40° C. under reduced pressure to afford 5.9 g of 4-benzyloxy-N-(2-oxoazepan-3-yl)benzamide a46.

Yield: 80%.

LC-MS (MH⁺): 339.

### 15.2 Synthesis of 2-[4-(benzyloxy)phenyl]-5,6,7,8-tetrahydro-4H-[1,3]thiazolo[5,4-b]azepine hydrochloride a47

A suspension of 4-benzyloxy-N-(2-oxoazepan-3-yl)benzamide a46 (2.0 g, 5.91 mmol, 1 eq) in pyridine (20 ml) is treated with Lawesson's reagent (1.43 g, 3.55 mmol, 0.6 eq) and the mixture is stirred at 100° C. for 20 h. After cooling down to room temperature, the mixture is poured on an aqueous saturated solution of sodium hydrogenocarbonate (150 ml) and the aqueous layer is extracted with dichloromethane (2×100 ml). The combined organic layers are dried over magnesium sulfate and concentrated under reduced pressure. The residue is taken up with a 1:1 mixture of ethyl acetate and dichloromethane (50 ml) and the uncyclised thioamide is filtered off. The organic layer is concentrated and the residue is purified by chromatography over silicagel (heptane/ethyl acetate 3:1). The main fraction from chromatography is concentrated under reduced pressure. The residue is dissolved in a 1:5 mixture of methanol/diethyl ether (10 ml) and treated with a 2 M solution of hydrogen chloride in diethyl ether (2 ml). The obtained solid is dried at 40° C. under vacuum to afford 900 mg of 2-[4-(benzyloxy)phenyl]-5,6,7,8-tetrahydro-4H-[1,3]thiazolo[5,4-b]azepine hydrochloride a47.

Yield: 23%.

LC-MS (MH⁺): 337.

### 15.3 Synthesis of 4-acetyl-2-[4-(benzyloxy)phenyl]-5,6,7,8-tetrahydro-4H-[1,3]thiazolo[5,4-b]azepine a48

A suspension of 2-[4-(benzyloxy)phenyl]-5,6,7,8-tetrahydro-4H-[1,3]thiazolo[5,4-b]azepine hydrochloride a47 (0.9 g, 2.68 mmol, 1 eq) in dichloromethane (40 ml) is treated with triethylamine (1.12 ml, 8.04 mmol, 3 eq) and a solution of acetyl chloride (0.23 ml, 3.21 mmol, 1.2 eq) in dichloromethane (5 ml). After 2 h stirring at 20° C., water (20 ml) is added. The organic layer is dried over magnesium sulfate and concentrated under reduced pressure. Chromatography over silicagel (dichloromethane/methanol/ammonia 95:5:0.5) affords 210 mg of 4-acetyl-2-[4-(benzyloxy)phenyl]-5,6,7,8-tetrahydro-4H-[1,3]thiazolo[5,4-b]azepine a48.

Yield: 64%.

LC-MS (MH⁺): 379.

### 15.4 Synthesis of 4-(4-acetyl-5,6,7,8-tetrahydro-4H-[1,3]thiazolo[5,4-b]azepin-2-yl)phenol a49

A solution of 4-acetyl-2-[4-(benzyloxy)phenyl]-5,6,7,8-tetrahydro-4H-[1,3]thiazolo[5,4-b]azepine a48 (0.6 g, 1.5 mmol, 1 eq) in dichloromethane (10 ml) is treated with a 1 M solution of boron tribromide in dichloromethane (8 ml, 8.0 mmol, 5 eq). The mixture is left to stir overnight at 20° C. before addition of water (10 ml). The organic layer is dried over magnesium sulfate and concentrated under reduced pressure. Chromatography over silicagel (dichloromethane/methanol/ammonia 95:5:0.5) affords 360 mg of 4-(4-acetyl-5,6,7,8-tetrahydro-4H-[1,3]thiazolo[5,4-b]azepin-2-yl)phenol a49.

Yield: 79%.

LC-MS (MH⁺): 289.

### 15.5 Synthesis of 4-acetyl-2-{4-[(trans-3-piperidin-1-ylcyclobutyl)oxy]phenyl}-5,6,7,8-tetrahydro-4H-[1,3]thiazolo[5,4-b]azepine 31

A solution of 4-(4-acetyl-5,6,7,8-tetrahydro-4H-[1,3]thiazolo[5,4-b]azepin-2-yl)phenol a49 (0.16 g, 0.55 mmol, 1 eq) in dry N,N-dimethylacetamide (7 ml) is treated with sodium hydride (60% dispersion in mineral oil, 0.03 g, 0.83 mmol, 1.5 eq) (under an argon atmosphere). After 15 minutes, cis-3-piperidin-1-ylcyclobutyl 4-methylbenzenesulfonate a25 (0.19 g, 0.61 mmol, 1.1 eq) is added and the mixture is stirred at 60° C. during 60 hours. The mixture is concentrated under reduced pressure, diluted with ethyl acetate (20 ml) and washed with brine (10 ml). The organic layer is dried over magnesium sulfate and concentrated under reduced pressure. The residue is purified by chromatography over silicagel (dichloromethane/methanol/ammonia 98:2:0.2). The solid obtained after evaporation of solvent is dried at 50° C. under vacuum overnight to afford 67 mg of 4-acetyl-2-{4-[(trans-3-piperidin-1-ylcyclobutyl)oxy]phenyl}-5,6,7,8-tetrahydro-4H-[1,3]thiazolo[5,4-b]azepine 31 as a brown solid.

Yield: 36%.

LC-MS (MH⁺): 426.

### EXAMPLE 16

### Synthesis of 5-acetyl-2-{4-[(trans-3-piperidin-1-ylcyclobutyl)oxy]phenyl}-4,5,6,7-tetrahydro[1,3]oxazolo[4,5-c]pyridine 28

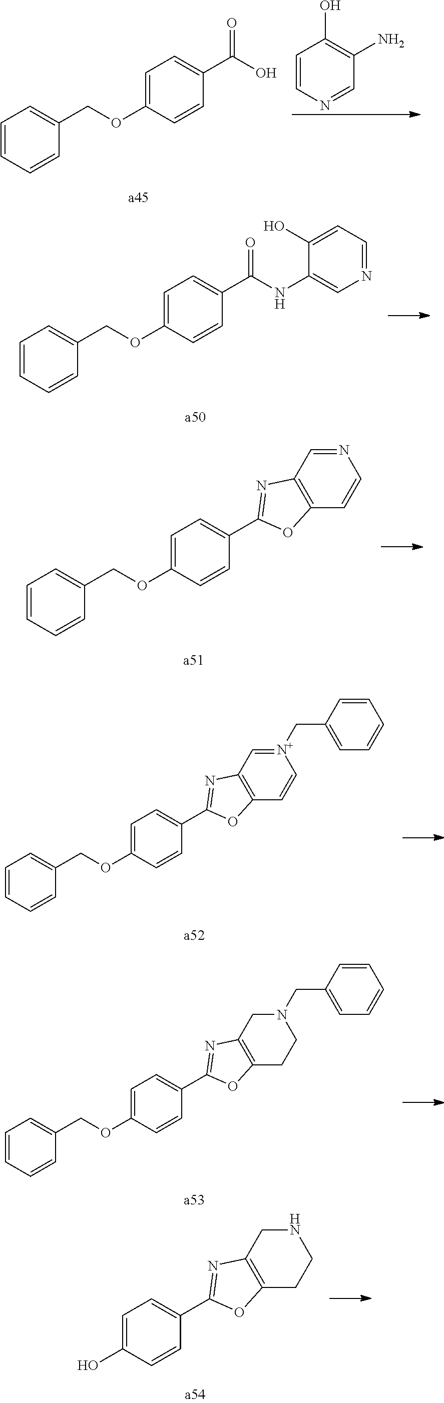

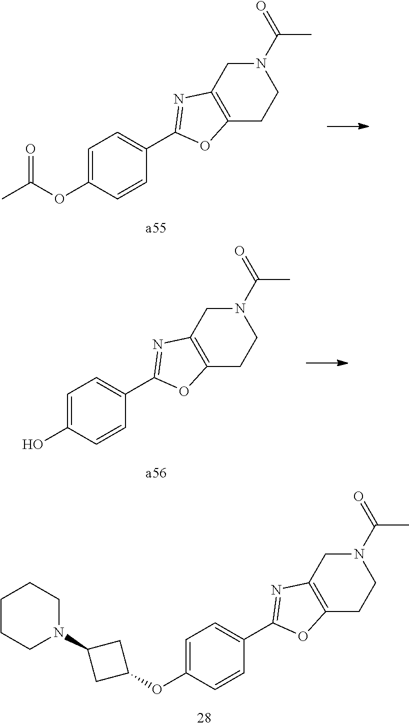

### 16.1 Synthesis of 4-(benzyloxy)-N-(4-hydroxypyridin-3-yl)benzamide a50

A suspension of 4-(benzyloxy)benzoic acid a45 (10.0 g, 43.81 mmol, 1 eq) and N,N-dimethylformamide (0.5 ml) in dichloromethane at 0° C. is treated with oxalyl chloride (5.18 ml, 48.19 mmol, 1.1 eq). The mixture is left to warm up to room temperature. When gas evolution has stopped, half of the solvent is removed under reduced pressure, and the solution is added dropwise to a solution of 3-aminopyridin-4-ol (4.82 g, 43.81 mmol, 1 eq) and triethylamine (12.15 ml, 87.62 mmol, 2 eq) in dichloromethane (300 ml). The mixture is stirred at 20° C. for 24 h and water (200 ml) is added. The aqueous phase is extracted with a 9:1 mixture of dichloromethane and methanol (2×300 ml). The combined organic layers are dried over magnesium sulfate and concentrated under reduced pressure. The residue is triturated with ethyl acetate (50 ml) and the resulting suspension is filtered off. The solid is dried at 40° C. in vacuo to yield 10 g of 4-(benzyloxy)-N-(4-hydroxypyridin-3-yl)benzamide a50.

Yield: 71%.

LC-MS (MH⁺): 321.

### 16.2 Synthesis of 2-[4-(benzyloxy)phenyl][1,3]oxazolo[4,5-c]pyridine a51

A solution of hexachloroethane (16.81 g, 71.02 mmol, 2.5 eq) in dry dichloromethane (300 ml) is treated with triphenylphosphine (22.35 g, 85.22 mmol, 3 eq) and triethylamine (31.68 ml, 227.25 mmol, 8 eq). After 10 minutes stirring at 20° C., 4-(benzyloxy)-N-(4-hydroxypyridin-3-yl)benzamide a50 (9.1 g, 28.41 mmol, 1 eq) is added in several portions. The suspension is stirred overnight at 20° C. and filtered. This solid is triturated with a 1 M aqueous solution of hydrogen chloride (50 ml) and this suspension is filtered off. The solid is rinsed with diethyl ether and dried at 40° C. in vacuo to yield 7.9 g of 2-[4-(benzyloxy)phenyl][1,3]oxazolo[4,5-c]pyridine a51.

Yield: 92%.

LC-MS (MH⁺): 303.

### 16.3 Synthesis of 5-benzyl-2-[4-(benzyloxy)phenyl][1,3]oxazolo[4,5-c]pyridin-5-ium a52

A solution of 2-[4-(benzyloxy)phenyl][1,3]oxazolo[4,5-c]pyridine a51 (2.0 g, 6.62 mmol, 1 eq) in N,N-dimethylformamide (10 ml) is treated with benzyl bromide (0.9 ml, 7.28 mmol, 1.1 eq) and stirred at 20° C. for one hour, then at 60° C. overnight. After cooling to 20° C., ethyl acetate (50 ml) is added to the mixture. The resulting suspension is filtered off and the solid is dried at 40° C. in vacuo to yield 2.6 g of 5-benzyl-2-[4-(benzyloxy)phenyl][1,3]oxazolo[4,5-c]pyridin-5-ium a52.

Yield: 100%.

LC-MS (MH⁺): 393.

### 16.4 Synthesis of 5-benzyl-2-[4-(benzyloxy)phenyl]-4,5,6,7-tetrahydro[1,3]oxazolo[4,5-c]pyridine a53

A solution 5-benzyl-2-[4-(benzyloxy)phenyl][1,3]oxazolo[4,5-c]pyridin-5-ium a52 (2.6 g, 6.61 mmol, 1 eq) in ethanol (150 ml) is treated with portions of sodium borohydride (1.0 g, 26.43 mmol, 4 eq) and the mixture is stirred at 60° C. for 2 hours then overnight at 20° C. Water (1 ml) is added and the mixture is concentrated under reduced pressure. The mixture is taken up with a 1:1 mixture of dichloromethane and water (40 ml). The organic layer is dried over magnesium sulfate and concentrated under reduced pressure. Chromatography over silicagel (dichloromethane/methanol/ammonia 90:9:1) affords 0.97 g of 5-benzyl-2-[4-(benzyloxy)phenyl]-4,5,6,7-tetrahydro[1,3]oxazolo[4,5-c]pyridine a53.

Yield: 46%.

LC-MS (MH⁺): 397.

### 16.5 Synthesis of 4-(4,5,6,7-tetrahydro[1,3]oxazolo[4,5-c]pyridin-2-yl)phenol a54

A solution 5-benzyl-2-[4-(benzyloxy)phenyl]-4,5,6,7-tetrahydro[1,3]oxazolo[4,5-c]pyridine a53 (0.97 g, 2.4 mmol, 1 eq) in a 1:1 mixture of ethyl acetate and acetic acid (10 ml) is stirred overnight in an autoclave at 70° C. and under a hydrogen (20 bar). The mixture is filtered and the filtrate is concentrated to dryness to afford 0.5 g of 4-(4,5,6,7-tetrahydro[1,3]oxazolo[4,5-c]pyridin-2-yl)phenol a54.

Yield: 100%.

LC-MS (MH⁺): 217.

### 16.6 Synthesis of 4-(5-acetyl-4,5,6,7-tetrahydro[1,3]oxazolo[4,5-c]pyridin-2-yl)phenyl acetate a55

A solution of 4-(4,5,6,7-tetrahydro[1,3]oxazolo[4,5-c]pyridin-2-yl)phenol a54 (0.6 g, 2.77 mmol, 1 eq) in dichloromethane (10 ml) is treated with triethylamine (0.85 ml, 6.1 mmol, 2.2 eq) and acetyl chloride (0.48 g, 6.1 mmol, 2.2 eq). The mixture is stirred overnight and water (10 ml) is added. The organic layer is dried over magnesium sulfate and concentrated in vacuo. Chromatography over silicagel (dichloromethane/methanol/ammonia 97:3:0.3) affords 0.3 g of 4-(5-acetyl-4,5,6,7-tetrahydro[1,3]oxazolo[4,5-c]pyridin-2-yl)phenyl acetate a55.

Yield: 36%.

LC-MS (MH⁺): 301.

### 16.7 Synthesis of 4-(5-acetyl-4,5,6,7-tetrahydro[1,3]oxazolo[4,5-c]pyridin-2-yl)phenol a56

A solution of 4-(5-acetyl-4,5,6,7-tetrahydro[1,3]oxazolo[4,5-c]pyridin-2-yl)phenyl acetate a55 (0.3 g, 1.16 mmol, 1 eq) in tetrahydrofuran (10 ml) is treated with a solution of lithium hydroxide (50 mg, 1.16 mmol, 1 eq) in water (0.5 ml). After stirring at 70° C. for 2 hours, the mixture is concentrated under reduced pressure. The residue is taken up with water (10 ml) and a 1 M aqueous solution of hydrochloric acid (2.0 ml). This aqueous layer is extracted with dichloromethane (2×10 ml). The organic layer is dried over magnesium sulfate and concentrated in vacuo to afford 230 mg of 4-(5-acetyl-4,5,6,7-tetrahydro[1,3]oxazolo[4,5-c]pyridin-2-yl)phenol a56.

Yield: 85%.

LC-MS (MH⁺): 259.

### 16.8 Synthesis of 5-acetyl-2-{4-[(trans-3-piperidin-1-ylcyclobutyl)oxy]phenyl}-4,5,6,7-tetrahydro[1,3]oxazolo[4,5-c]pyridine 28

A solution of 4-(5-acetyl-4,5,6,7-tetrahydro[1,3]oxazolo[4,5-c]pyridin-2-yl)phenol a56 (0.2 g, 0.78 mmol, 1 eq) in dry N,N-dimethylformamide (10 ml) is treated with potassium tert-butoxyde (0.27 g, 2.34 mmol, 3 eq) under an argon atmosphere. After 15 minutes, cis-3-piperidin-1-ylcyclobutyl 4-methylbenzenesulfonate a25 (0.23 g, 0.78 mmol, 1 eq) is added. The mixture is stirred at 80° C. overnight, concentrated under reduced pressure, diluted with ethyl acetate (20 ml) and washed twice with brine (2×20 ml). The organic layer is dried over magnesium sulfate and concentrated under reduced pressure. The residue is purified by reversed-phase chromatography (acetonitrile/water/trifluoroacetic acid 5/95/0.1 to 35/65/0.1). After concentration under reduced pressure, the residue is taken up with dichloromethane (20 ml), washed with an aqueous saturated solution of sodium hydrogenocarbonate, dried over magnesium sulfate and concentrated to dryness to afford 20 mg of 5-acetyl-2-{4-[(trans-3-piperidin-1-ylcyclobutyl)oxy]phenyl}-4,5,6,7-tetrahydro[1,3]oxazolo[4,5-c]pyridine 28 as a white solid.

Yield: 7%.

LC-MS (MH⁺): 396.

### EXAMPLE 17

### Synthesis of 5-acetyl-2-{4-[(trans-3-piperidin-1-ylcyclobutyl)oxy]phenyl}-4,5,6,7-tetrahydro[1,3]thiazolo[4,5-c]pyridine 29

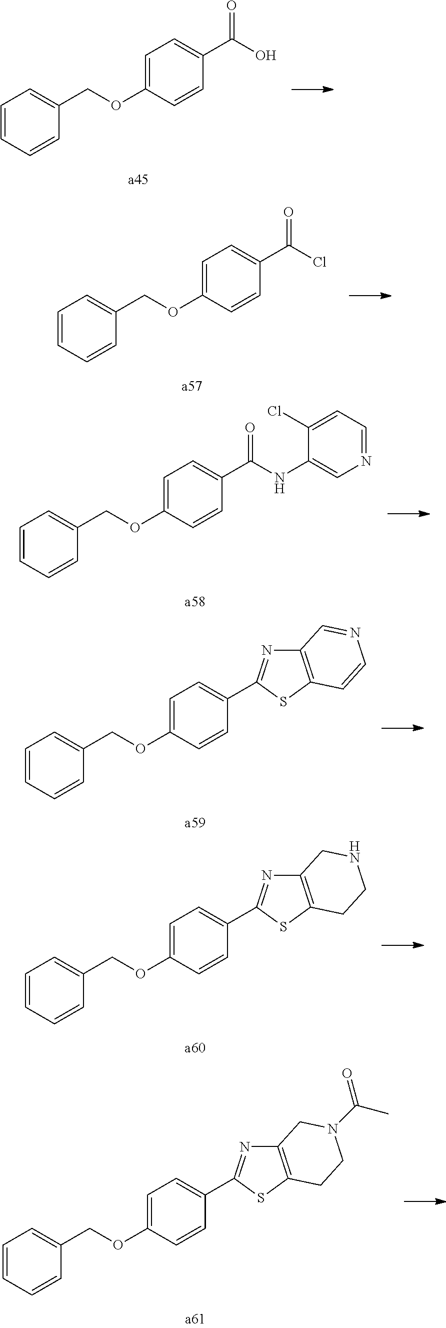

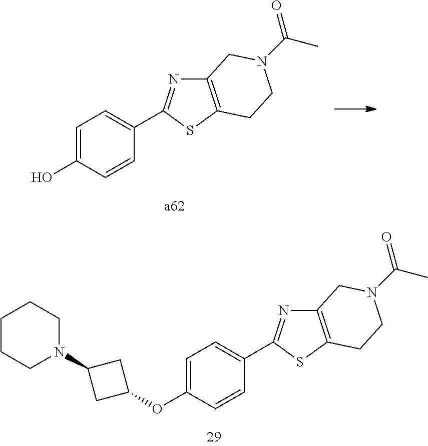

### 17.1 Synthesis of 4-(benzyloxy)benzoyl chloride a57

A solution of 4-(benzyloxy)benzoic acid a45 (17.76 g, 77.8 mmol, 1 eq) in dichloromethane (700 ml) and N,N-dimethylformamide (400 μl) is treated with oxalyl chloride (9.2 ml, 85.58 mmol, 1.1 eq). The mixture is stirred for 20 h at 20° C. The mixture is then concentrated under reduced pressure and used as such in the next step.

### 17.2 Synthesis of 4-(benzyloxy)-N-(4-chloropyridin-3-yl)benzamide a58

A solution of 3-amino-4-chloropyridine (10 g, 77.8 mmol, 1 eq) in N,N-dimethylformamide (300 ml) is treated with sodium hydride (60% dispersion in mineral oil, 6.85 g, 171 mmol, 2.2 eq). After 1 h stirring at 20° C., the resulting solution is treated dropwise with a solution of 4-(benzyloxy)benzoyl chloride a57 (19.193 g, 77.8 mmol, 1 eq) in dichloromethane (300 ml). The mixture is stirred for 24 h at 20° C. The mixture is concentrated under reduced pressure. The residue is dissolved in ethyl acetate and the organic layer is washed with water then with brine, dried over magnesium sulfate and concentrated under reduced pressure. The residue is taken up with methanol (700 ml) and treated with a suspension of sodium hydride (60% dispersion in mineral oil, 3.1 g, 124 mmol, 1.6 eq). After 2 h stirring at 20° C., the mixture is concentrated under reduced pressure. The residue is taken up and sonicated in ethyl acetate (400 ml). The solid that settles (4-(benzyloxy)benzoic acid) is filtered off and the resulting solution is concentrated under reduced pressure. The residue is taken up with ethyl acetate (400 ml) and left to stand overnight. The suspension is again filtered and the solid is washed with ethyl acetate and dried. The filtrate is concentrated and the residue is purified by chromatography over silicagel (dichloromethane/methanol 99:1). The two batches of solid are pooled together to yield a combined 10.8 g of 4-(benzyloxy)-N-(4-chloropyridin-3-yl)benzamide a58.

Yield: 41%.

LC-MS (MH⁺): 339/341.

### 17.3 Synthesis of 2-[4-(benzyloxy)phenyl][1,3]thiazolo[4,5-c]pyridine a59

A solution of 4-(benzyloxy)-N-(4-chloropyridin-3-yl)benzamide a58 (6.9 g, 20.37 mmol, 1 eq) in toluene (200 ml) is treated with 2,4-bis(4-methoxyphenyl)-2,4-dithioxo-1,3,2,4-dithiadiphosphetane (Lawesson's reagent, 5.766 g, 14.26 mmol, 0.7 eq) and the mixture is stirred at 110° C. for 20 h. After cooling down to room temperature, water (400 ml) is added. The aqueous layer is extracted with toluene (400 ml). The organic layer is washed with an aqueous saturated solution of sodium hydrogenocarbonate, dried over magnesium sulfate and concentrated under reduced pressure. The residue is taken up in ethyl acetate (400 ml) and sonicated. The resulting suspension is filtered, washed with ethyl acetate and dried under vacuum to give a first batch of solid. The filtrate is concentrated under reduced pressure and purified by chromatography over silicagel (dichloromethane/methanol 99:1) to give a second batch. The two batches of solid are combined to yield 2.54 g of 2-[4-(benzyloxy)phenyl][1,3]thiazolo[4,5-c]pyridine a59.

Yield: 40%.

LC-MS (MH⁺): 319.

### 17.4 Synthesis of 2-[4-(benzyloxy)phenyl]-4,5,6,7-tetrahydro[1,3]thiazolo[4,5-c]pyridine a60

A suspension of 2-[4-(benzyloxy)phenyl][1,3]thiazolo[4,5-c]pyridine a59 (1 g, 3.14 mmol, 1 eq) and platinum (IV) oxide (142.6 mg, 0.63 mmol, 0.2 eq) in acetic acid (150 ml) is stirred overnight at 70° C. under a hydrogen atmosphere (50 bar) in an autoclave. The mixture is then filtered over Celite and concentrated to dryness to afford 2-[4-(benzyloxy)phenyl]-4,5,6,7-tetrahydro[1,3]thiazolo[4,5-c]pyridine a60 which is directly used in the next step without any further purification.

Yield: 100%

LC-MS (MH⁺): 323.

### 17.5 Synthesis of 5-acetyl-2-[4-(benzyloxy)phenyl]-4,5,6,7-tetrahydro[1,3]thiazolo[4,5-c]pyridine a61

A solution of 2-[4-(benzyloxy)phenyl]-4,5,6,7-tetrahydro[1,3]thiazolo[4,5-c]pyridine a60 (2.028 g, 6.29 mmol, 1 eq), acetic anhydride (96 mg, 9.44 mmol, 1.5 eq) and 4-(N,N-dimethylamino)pyridine (77 mg, 0.63 mmol, 0.1 eq) in dichloromethane (60 ml) is stirred for 1 h 30 at 40° C. Water is then added, the organic layer is collected, dried over magnesium sulfate and concentrated under reduced pressure. The resulting material is purified by chromatography over silicagel (dichloromethane/methanol 99:1) to yield 450 mg of pure 5-acetyl-2-[4-(benzyloxy)phenyl]-4,5,6,7-tetrahydro[1,3]thiazolo[4,5-c]pyridine a61.

Yield: 50%.

LC-MS (MH⁺): 365.

### 17.6 Synthesis of 4-(5-acetyl-4,5,6,7-tetrahydro[1,3]thiazolo[4,5-c]pyridin-2-yl)phenol a62

A solution 5-acetyl-2-[4-(benzyloxy)phenyl]-4,5,6,7-tetrahydro[1,3]thiazolo[4,5-c]pyridine a61 (1.1 g, 3.02 mmol, 1 eq) in dichloromethane (30 ml) is cooled to 0° C. The mixture is treated dropwise with a 1M solution of boron tribromide in dichloromethane (18 ml, 18.11 mmol, 6 eq). The mixture is left to warm to room temperature and is stirred for 2 h. Water is added and the mixture is extracted with a 90:10 mixture of dichloromethane/methanol. The organic layer is washed with an aqueous saturated solution of sodium hydrogenocarbonate, dried over magnesium sulfate and concentrated under reduced pressure to afford 370 mg of 4-(5-acetyl-4,5,6,7-tetrahydro[1,3]thiazolo[4,5-c]pyridin-2-yl)phenol a62, which is directly used in the next step without any further purification.

Yield: 45%

LC-MS (MH⁺): 275.

### 17.7 Synthesis of 5-acetyl-2-{4-[(trans-3-piperidin-1-ylcyclobutyl)oxy]phenyl}-4,5,6,7-tetrahydro[1,3]thiazolo[4,5-c]pyridine 29

A solution of 4-(5-acetyl-4,5,6,7-tetrahydro[1,3]thiazolo[4,5-c]pyridin-2-yl)phenol a62 (370 mg, 1.02 mmol, 1 eq) in N,N-dimethylacetamide (10 ml) is treated with sodium hydride (60% dispersion in mineral oil, 81.2 mg, 2 mmol, 2 eq). The mixture is stirred for 10 min at room temperature. Cis-3-piperidin-1-ylcyclobutyl 4-methylbenzenesulfonate a25 (527 mg, 1.5 mmol, 1.5 eq) is added and the mixture is heated at 70° C. for 3 days. After cooling down to room temperature, brine is added and the aqueous phase is extracted with ethyl acetate. The organic layer are washed with brine, dried over magnesium sulfate and concentrated under reduced pressure. Chromatography over silicagel (dichloromethane/methanol 95:5 to 90:10) affords 246 mg of 5-acetyl-2-{4-[(trans-3-piperidin-1-ylcyclobutyl)oxy]phenyl}-4,5,6,7-tetrahydro[1,3]thiazolo[4,5-c]pyridine 29 as a beige solid.

Yield: 67%.

LC-MS (MH⁺): 412.

### EXAMPLE 18

### Synthesis of 2-{4-[(3-piperidin-1-ylcyclobutyl)oxy]phenyl}-5,6-dihydro-4H-cyclopenta[d][1,3]thiazole-5-carboxylic acid 1

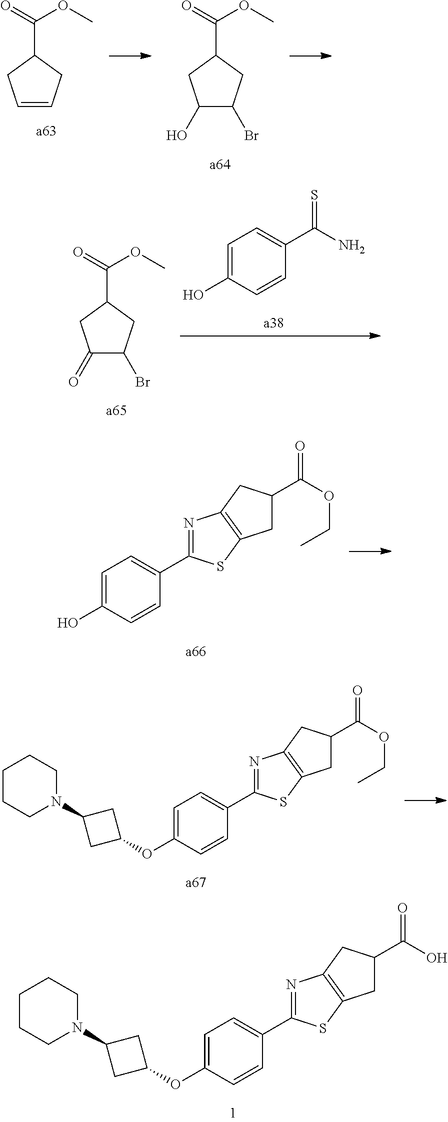

### 18.1 Synthesis of methyl 3-bromo-4-hydroxycyclopentanecarboxylate a64

A solution of methyl cyclopent-3-ene-1-carboxylate a63 (6.69 g, 53 mmol, 1 eq) in acetonitrile (70 ml) is treated with calcium carbonate (5.3 g, 53 mmol, 1 eq) in water (18 ml). The mixture is cooled to 0° C. and a solution of N-bromosuccinimide (9.44 g, 53 mmol, 1 eq) in acetonitrile (70 ml) is added slowly. The mixture is stirred at room temperature for 4 hours, filtered and concentrated under vacuum. Water is then added and the product is extracted 3 times with ethyl acetate. The combined organic layers are washed with brine, dried over magnesium sulfate and concentrated under reduced pressure. The resulting orange solid is taken up with dichloromethane. The solid is then filtered, rinsed with dichloromethane and the filtrate is concentrated under vacuum to afford 10.71 g of methyl 3-bromo-4-hydroxycyclopentanecarboxylate a64 as an orange oil.

Yield: 90%.

GC-MS (M^{+•}): 222/224.

### 18.2. Synthesis of methyl 3-bromo-4-oxocyclopentanecarboxylate a65

A solution of methyl 3-bromo-4-hydroxycyclopentanecarboxylate a64 (5.58 g, 25 mmol, 1 eq) in dichloromethane (200 ml) is cooled to 0° C. and treated with a 15% solution of 1,1,1-triacetoxy-1,1-dihydro-1,2-benziodoxol-3(1H)-one (Dess-Martin's reagent) in dichloromethane (130 ml). The mixture is stirred at room temperature for 48 hours. A saturated solution of sodium thiosulfate is added and the mixture is stirred for one hour. Water is added, the two layers are separated and the organic layer is successively washed with an aqueous saturated solution of sodium hydrogenocarbonate and brine. It is dried over magnesium sulfate and concentrated under reduced pressure. The resulting brown oil is taken up with dichloromethane, heated and the solid is filtered off. The dichloromethane solution is concentrated under vacuum, the residue is taken up with diethyl ether, sonicated, the solid is filtered off and the solution is concentrated under reduced pressure. Methyl 3-bromo-4-oxocyclopentanecarboxylate a65 (4.57 g) is obtained as an orange oil and directly used in the next step without any further purification.

Yield: 83%.

GC-MS (M^{+•}): 220/222.

### 18.3. Synthesis of ethyl 2-[4-hydroxyphenyl]-5,6-dihydro-4H-cyclopenta[d][1,3]thiazole-5-carboxylate a66

A solution of methyl 3-bromo-4-oxocyclopentanecarboxylate a65 (4.1 g, 18.5 mmol, 1 eq) in ethanol (40 ml) is treated with 4-hydroxythiobenzamide a38 (2.8 g, 18.5 mmol, 1 eq). The reaction is stirred overnight under reflux. The mixture is then concentrated and the residue taken up with ethyl acetate. The organic layer is washed with a 1N aqueous solution of sodium hydroxide, neutralized with a 1N aqueous solution of HCl, dried over magnesium sulfate and concentrated under vacuum to afford 2.7 g of ethyl 2-[4-hydroxyphenyl]-5,6-dihydro-4H-cyclopenta[d][1,3]thiazole-5-carboxylate a66.

Yield: 51%.

LC-MS (MH⁺): 290.

### 18.4 Synthesis of ethyl 2-{4-[(trans-3-piperidin-1-ylcyclobutyl)oxy]phenyl}-5,6-dihydro-4H-cyclopenta[d][1,3]thiazole-5-carboxylate a67

To a solution of ethyl 2-(4-hydroxyphenyl)-5,6-dihydro-4H-cyclopenta[d][1,3]thiazole-5-carboxylate a66 (540 mg, 1.99 mmol, 1 eq) in N,N-dimethylformamide (20 ml) at 0° C. is added sodium hydride 60% (159 mg, 3.98 mmol, 2 eq). The mixture is stirred at room temperature for 30 minutes then cis-3-piperidin-1-ylcyclobutyl 4-methylbenzenesulfonate a25 (615 mg, 1.99 mmol, 1 eq) is added and the mixture is heated at 80° C. After one night sodium hydride (80 mg, 1.99 mmol, 1 eq) and cis-3-piperidin-1-ylcyclobutyl 4-methylbenzenesulfonate a25 (310 mg, 1 mmol, 0.5 eq) are added and the mixture is heated at 80° C. for one more night. The mixture is then concentrated to dryness. The residue is dissolved in ethyl acetate and washed with an aqueous saturated solution of ammonium chloride. The aqueous phase is extracted with ethyl acetate, the combined organic phases are dried over magnesium sulfate and concentrated under vacuum to give 580 mg of a red oil. The crude mixture is purified by chromatography over silica gel (dichloromethane, then dichloromethane/methanol/ammonia 85:15:1.5) to give two fractions. The first one, an orange oil, corresponds to ethyl 2-{4-[(trans-3-piperidin-1-ylcyclobutyl)oxy]phenyl}-5,6-dihydro-4H-cyclopenta[d][1,3]thiazole-5-carboxylate a67 (155 mg, yield: 18%, LC-MS (MH⁺): 427). The second one, a red solid, corresponds to crude 2-{4-[(3-piperidin-1-ylcyclobutyl)oxy]phenyl}-5,6-dihydro-4H-cyclopenta[d][1,3]thiazole-5-carboxylic acid (100 mg, yield: 13%, LC-MS (MH⁺): 399).

### 18.5 Synthesis of 2-{4-[(3-piperidin-1-ylcyclobutyl)oxy]phenyl}-5,6-dihydro-4H-cyclopenta[d][1,3]thiazole-5-carboxylic acid 1

A solution of lithium hydroxide monohydrate (30 mg, 0.70 mmol, 2 eq) in water (1.4 ml) is added to a solution of ethyl 2-{4-[(trans-3-piperidin-1-ylcyclobutyl)oxy]phenyl}-5,6-dihydro-4H-cyclopenta[d][1,3]thiazole-5-carboxylate a67 (150 mg, 0.35 mmol, 1 eq) in tetrahydrofuran (7 ml) and the mixture is heated at reflux overnight. Water (15 ml) is added and the aqueous phase is washed with ethyl acetate, acidified to pH 6 with a 1 N aqueous solution of hydrochloric acid and extracted three times with ethyl acetate. The combined organic layers are dried over magnesium sulfate and concentrated under vacuum to afford 40 mg of crude product. Most of the product stays in the aqueous layer. Crude product, aqueous layer and second fraction resulting from the previous step are combined and purified by reverse phase chromatography (acetonitrile/water/trifluoroacetic acid 5:95:0.1 to 95:5:0.1) to afford 100 mg of 2-{4-[(3-piperidin-1-ylcyclobutyl)oxy]phenyl}-5,6-dihydro-4H-cyclopenta[d][1,3]thiazole-5-carboxylic acid 1 as an orange solid.

Yield: 19%.

LC-MS (MH⁺): 399.

### EXAMPLE 19

### Synthesis of diethyl {[2-{4-[(trans-3-piperidin-1-ylcyclobutyl)oxy]phenyl}-6,7-dihydro[1,3]thiazolo[5,4-c]pyridin-5(4H)-yl]methyl}phosphonate 27

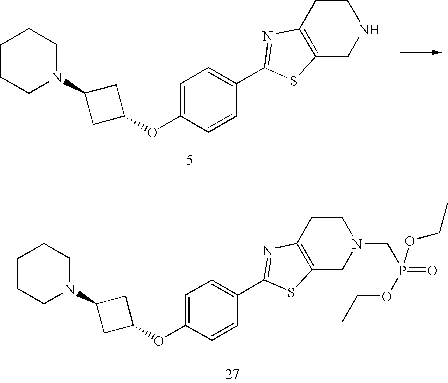

A mixture of benzotriazole (0.16 g, 1.35 mmol, 1 eq) and 2-{4-[(trans-3-piperidin-1-ylcyclobutyl)oxy]phenyl}-4,5,6,7-tetrahydro[1,3]thiazolo[5,4-c]pyridine 5 (0.5 g, 1.35 mmol, 1 eq) in a mixture of methanol (8 ml) and water (0.4 ml) is stirred at 25° C. for 20 minutes. The mixture is vigorously stirred and formaldehyde (37% aqueous solution, 1.28 ml, 1.49 mmol, 1.1 eq) is added. After 4 h, the suspension is filtered and the precipitate is washed with cold methanol (2 ml). To the benzotriazolyl intermediate in dry dichloromethane (30 ml) at 0° C. are successively added zinc dibromide (0.3 g, 1.3 mmol, 1.2 eq) and triethylphosphite (0.22 ml, 1.3 mmol, 1.2 eq). The reaction mixture is stirred at 0° C. for 2 h, then at 20° C. for 20 h, and the reaction is quenched with water (10 ml). After extraction with dichloromethane, the combined organic layers are successively washed with 1 N aqueous solution of sodium hydroxide (20 ml) and brine (20 ml), and dried over magnesium sulfate. After removal of the solvent under vacuum, the residue crystallizes to afford 0.68 g of {[2-{4-[(trans-3-piperidin-1-ylcyclobutyl)oxy]phenyl}-6,7-dihydro[1,3]thiazolo[5,4-c]pyridin-5(4H)-yl]methyl}phosphonate 27 as a yellow sticky solid.

Yield: 96%.

LC-MS (MH⁺): 520.

### EXAMPLE 20

### Synthesis of 2-{4-[(trans-3-piperidin-1-ylcyclobutyl)oxy]phenyl}[1,3]thiazolo[4,5-c]pyridine 32

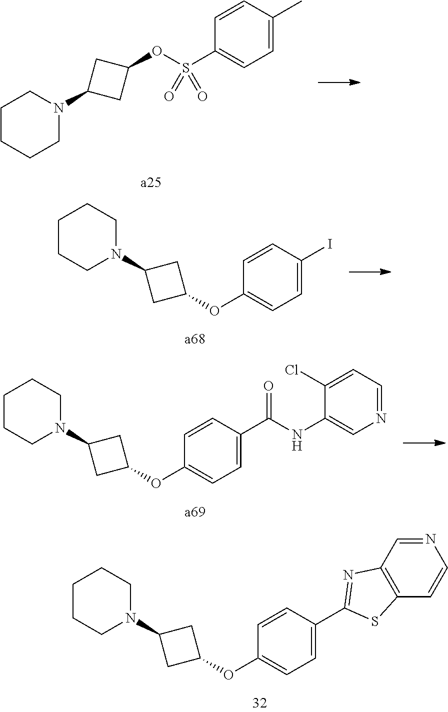

### 20.1 Synthesis of 1-[trans-3-(4-iodophenoxy)cyclobutyl]piperidine a68

A solution of 4-iodophenol (15.4 g, 70.3 mmol, 1.5 eq) in dry N,N-dimethylformamide (65 ml) is treated with sodium hydride (60% dispersion in mineral oil, 2.0 g, 84.3 mmol, 1.8 eq) under an argon atmosphere. After 30 minutes, cis-3-piperidin-1-ylcyclobutyl 4-methylbenzenesulfonate a25 (14.5 g, 46.9 mmol, 1 eq) is added and the mixture is stirred at 70° C. for 2 days. The mixture is diluted with ethyl acetate and washed with brine. The organic layer is then dried over magnesium sulfate and concentrated under reduced pressure. The residue is purified by chromatography over silicagel (dichloromethane 100% to dichloromethane/ethanol/ammonia 97:2.7:0.3) to afford 1-[trans-3-(4-iodophenoxy)cyclobutyl]piperidine a68 as an orange solid (11.5 g).

Yield: 69%.

LC-MS (MH⁺): 358.

### 20.2 Synthesis of N-(4-chloropyridin-3-yl)-4-[(trans-3-piperidin-1-ylcyclobutyl)oxy]benzamide a69

A process vial is charged with 1-[3-(4-Iodophenoxy)-cyclobutyl]-piperidine a68 (2.8 g, 7.8 mmol, 1 eq) and palladium (II) acetate (352 mg, 1.6 mmol, 0.2 eq), molybdenum hexacarbonyl (2277 mg, 8.6 mmol, 1.1 eq) and dry tetrahydrofuran (36 ml). The vial is capped with a Teflon septum under argon and the mixture is cooled to 0° C. with an ice bath. 1,8-Diazabicyclo[5.4.0]undec-7-ene (2.98 g, 19.6 mmol, 2.5 eq) is added. The vial is stirred under microwave irradiation at 125° C. for 20 min. After cooling, the reaction mixture is filtered through a short Celite pad. The filtrate is concentrated under reduced pressure. The residue is purified by silica gel flash chromatography (dichloromethane/methanol 98:2) to yield 960 mg of pure N-(4-chloropyridin-3-yl)-4-[(trans-3-piperidin-1-ylcyclobutyl)oxy]benzamide a69.

Yield: 31%.

LC-MS (MH⁺): 386/388.

### 20.3 Synthesis of 2-{4-[(trans-3-piperidin-1-ylcyclobutyl)oxy]phenyl}[1,3]thiazolo[4,5-c]pyridine 32

N-(4-chloropyridin-3-yl)-4-[(trans-3-piperidin-1-ylcyclobutyl)oxy]benzamide a69 (300 mg, 0.78 mmol, 1 eq) in toluene (10 ml) is treated with 2,4-bis(4-methoxyphenyl)-2,4-dithioxo-1,3,2,4-dithiadiphosphetane (Lawesson's reagent, 220 mg, 0.54 mmol, 0.7 eq) and the mixture is stirred at 110° C. for 20 h. After cooling down to room temperature, the solvant is concentrated under reduced pressure. The residue is purified by silica gel flash chromatography (dichloromethane/methanol 90:10) to yield 205 mg of pure 2-{4-[(trans-3-piperidin-1-ylcyclobutyl)oxy]phenyl}[1,3]thiazolo[4,5-c]pyridine 32.

Yield: 72%.

LC-MS (MH⁺): 366.

### EXAMPLE 21

### Synthesis of 2-{4-[(trans-3-piperidin-1-ylcyclobutyl)oxy]phenyl}-5-(3,3,3-trifluoropropanoyl)-4,5,6,7-tetrahydro[1,3]thiazolo[5,4-c]pyridine 33

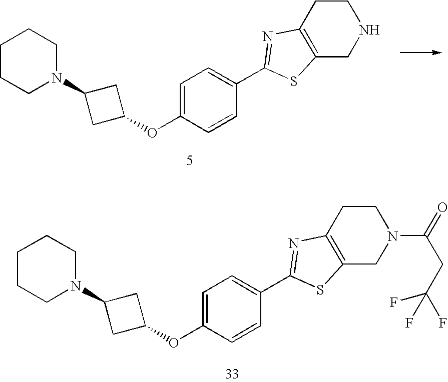

Oxalyl chloride (0.16 ml, 1.49 mmol, 1.1 eq) and N,N-dimethylformamide (0.1 ml) are added to a solution of 3,3,3-trifluoropropanoic acid (0.19 g, 1.49 mmol, 1.1 eq) in dichloromethane at 0° C. The reaction mixture is allowed to warm at 25° C. and is concentrated. The residue is added to a solution of 2-{4-[(trans-3-piperidin-1-ylcyclobutyl)oxy]phenyl}-4,5,6,7-tetrahydro[1,3]thiazolo[5,4-c]pyridine 5 (0.5 g, 1.35 mmol, 1 eq) and triethylamine (0.47 ml, 3.38 mmol, 2.5 eq) in dichloromethane (20 ml). The mixture is stirred at 25° C. overnight, then washed with an aqueous solution of potassium hydrogen sulfate, dried over magnesium sulfate and concentrated under reduced pressure. The residue is purified by chromatography over silicagel (gradient: dichloromethane/methanol/ammonia 98:2:0.2 to 90:10:1) to yield 270 mg of 2-{4-[(trans-3-piperidin-1-ylcyclobutyl)oxy]phenyl}-5-(3,3,3-trifluoropropanoyl)-4,5,6,7-tetrahydro[1,3]thiazolo[5,4-c]pyridine 33.

Yield: 40%

LC-MS (MH⁺): 480.

Ethyl oxo[2-{4-[(trans-3-piperidin-1-ylcyclobutyl)oxy]phenyl}-6,7-dihydro[1,3]thiazolo[5,4-c]pyridin-5(4H)-yl]acetate 49 may be synthesized according to the same method.

### EXAMPLE 22

### Synthesis of 5-[(5-methyl-2H-1,2,3-triazol-4-yl)carbonyl]-2-{4-[(trans-3-piperidin-1-ylcyclobutyl)oxy]phenyl}-4,5,6,7-tetrahydro[1,3]thiazolo[5,4-c]pyridine 35

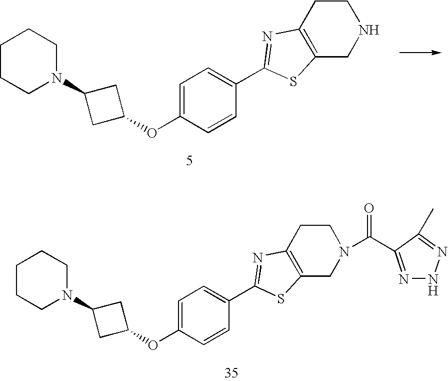

2-{4-[(trans-3-piperidin-1-ylcyclobutyl)oxy]phenyl}-4,5,6,7-tetrahydro[1,3]thiazolo[5,4-c]pyridine 5 (0.5 g, 1.35 mmol, 1 eq) is added to a solution of 5-methyl-2H-1,2,3-triazole-4-carboxylic acid (0.21 g, 1.62 mmol, 1.2 eq) and hydroxybenzotriazole (0.22 g, 1.62 mmol, 1.2 eq) in N,N-dimethylformamide (15 ml). 1-(3-dimethylaminopropyl)-3-ethylcarbodiimide hydrochloride (0.31 g, 1.62 mmol, 1.2 eq) is added to the mixture and stirred for 30 minutes at 25° C. Then, triethylamine (0.73 ml, 1.62 mmol, 1.2 eq) is added and the mixture is stirred at 25° C. for 20 hours. The solvent is concentrated under reduced pressure and the residue is taken up in dichloromethane/methanol 90:10, washed with water, dried over magnesium sulfate and concentrated under reduced pressure to give 0.7 g of crude product. The residue is purified by chromatography over silicagel (gradient: acetonitrile/water/ammonia 5:95:0.1 to 60:40:0.1) to yield 0.35 g of 5-[(5-methyl-2H-1,2,3-triazol-4-yl)carbonyl]-2-{4-[(trans-3-piperidin-1-ylcyclobutyl)oxy]phenyl}-4,5,6,7-tetrahydro[1,3]thiazolo[5,4-c]pyridine 35.

Yield: 54%.

LC-MS (MH⁺): 479.

Compounds 43, 44, 45, 46, 47, 52 and 54 may be synthesized according to the same method.

### EXAMPLE 23

### Synthesis of 3-isopropoxy-4-[2-{4-[(trans-3-piperidin-1-ylcyclobutyl)oxy]phenyl}-6,7-dihydro[1,3]thiazolo[5,4-c]pyridin-5(4H)-yl]cyclobut-3-ene-1,2-dione 56 and 3-hydroxy-4-[2-{4-[(trans-3-piperidin-1-ylcyclobutyl)oxy]phenyl}-6,7-dihydro[1,3]thiazolo[5,4-c]pyridin-5(4H)-yl]cyclobut-3-ene-1,2-dione 55

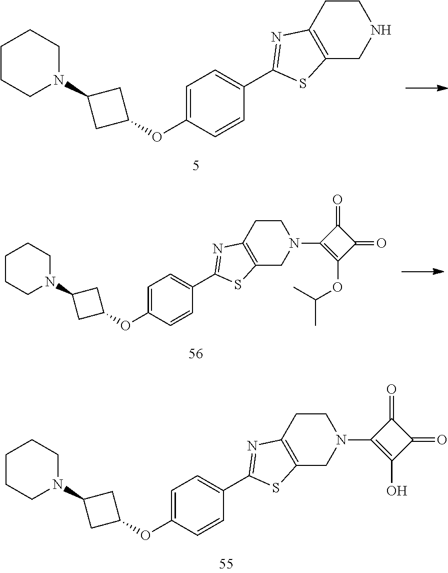

### 23.1 Synthesis of 3-isopropoxy-4-[2-{4-[(trans-3-piperidin-1-ylcyclobutyl)oxy]phenyl}-6,7-dihydro[1,3]thiazolo[5,4-c]pyridin-5(4H)-yl]cyclobut-3-ene-1,2-dione 56

3,4-diisopropoxycyclobut-3-ene-1,2-dione (0.72 g, 3.66 mmol, 1.5 eq) is added to a solution of 2-{4-[(trans-3-piperidin-1-ylcyclobutyl)oxy]phenyl}-4,5,6,7-tetrahydro[1,3]thiazolo[5,4-c]pyridine 5 (900 mg, 2.44 mmol, 1 eq) in methanol (10 ml). The mixture is stirred at room temperature for 60 hours, then concentrated under reduced pressure to afford 2 g of the crude product. The residue is purified by chromatography over silicagel (gradient: acetonitrile/water/ammonia 5:95:0.1 to 60:40:0.1). The obtained oil is triturated in dry ether to yield 660 mg of 3-isopropoxy-4-[2-{4-[(trans-3-piperidin-1-ylcyclobutyl)oxy]phenyl}-6,7-dihydro[1,3]thiazolo[5,4-c]pyridin-5(4H)-yl]cyclobut-3-ene-1,2-dione 56 as a white solid.

Yield: 56%.

LC-MS (MH⁺): 508.

3-amino-4-[2-{4-[(trans-3-piperidin-1-ylcyclobutyl)oxy]phenyl}-6,7-dihydro[1,3]thiazolo[5,4-c]pyridin-5(4H)-yl]cyclobut-3-ene-1,2-dione.1/2 trifluoroacetate 58 may be synthesized according to the same method.

### 23.2 Synthesis of 3-hydroxy-4-[2-{4-[(trans-3-piperidin-1-ylcyclobutyl)oxy]phenyl}-6,7-dihydro[1,3]thiazolo[5,4-c]pyridin-5(4H)-yl]cyclobut-3-ene-1,2-dione 55

HCl 6N (1 ml) is added to a solution of 3-isopropoxy-4-[2-{4-[(trans-3-piperidin-1-ylcyclobutyl)oxy]phenyl}-6,7-dihydro[1,3]thiazolo[5,4-c]pyridin-5(4H)-yl]cyclobut-3-ene-1,2-dione 56 (330 mg, 0.65 mmol, 1 eq) in ethanol (10 ml), methanol (10 ml) and dichloromethane (10 ml) and the mixture is stirred for 4 days. HCl 6N (5 ml) is added and the mixture stirred for another 5 hours. The mixture is filtered over celite, concentrated. The residue is purified by chromatography over silicagel (gradient: acetonitrile/water/ammonia 5:95:0.1 to 60:40:0.1) to give 300 mg of 3-hydroxy-4-[2-{4-[(trans-3-piperidin-1-ylcyclobutyl)oxy]phenyl}-6,7-dihydro[1,3]thiazolo[5,4-c]pyridin-5(4H)-yl]cyclobut-3-ene-1,2-dione 55 as a white solid.

Yield: 100%.

LC-MS (MH⁺): 466.

### EXAMPLE 24

### Synthesis of {[2-{4-[(trans-3-piperidin-1-ylcyclobutyl)oxy]phenyl}-6,7-dihydro[1,3]thiazolo[5,4-c]pyridin-5(4H)-yl]methyl}phosphonic acid 34

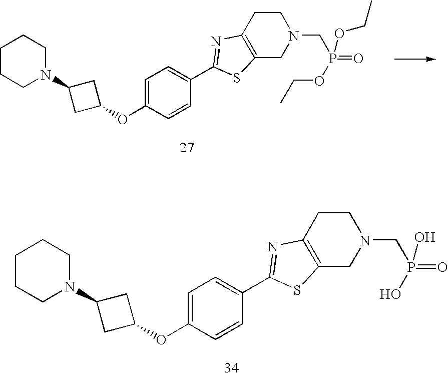

Bromotrimethylsilane (0.7 ml, 5.3 mmol, 4.45 eq) is added to a solution of diethyl {[2-{4-[(trans-3-piperidin-1-ylcyclobutyl)oxy]phenyl}-6,7-dihydro[1,3]thiazolo[5,4-c]pyridin-5(4H)-yl]methyl}phosphonate 27 (620 mg, 1.19 mmol, 1 eq) in acetonitrile (10 ml). The mixture is stirred at room temperature overnight. Water is added to the mixture, then concentrated under reduced pressure. The crude product is purified by chromatography over silicagel (gradient: acetonitrile/water/ammonia 5:95 to 30:70) to afford 53 mg of {[2-{4-[(trans-3-piperidin-1-ylcyclobutyl)oxy]phenyl}-6,7-dihydro[1,3]thiazolo[5,4-c]pyridin-5(4H)-yl]methyl}phosphonic acid 34 as a yellow powder.

Yield: 10%.

LC-MS (MH⁺): 464.

### EXAMPLE 25

### Synthesis of 2-oxo-2-[2-{4-[(trans-3-piperidin-1-ylcyclobutyl)oxy]phenyl}-6,7-dihydro[1,3]thiazolo[4,5-b]pyridin-4(5H)-yl]ethanol 66

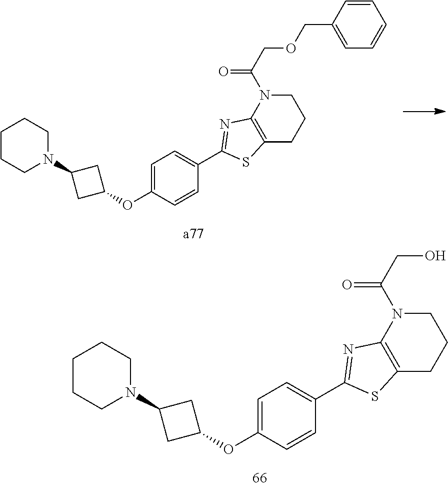

A 1M solution of boron tribromide in dichloromethane (41.7 ml, 41.7 mmol, 6 eq) is added dropwise to a solution of 2-(benzyloxy)-1-[2-(4-{[trans-3-(piperidin-1-yl)cyclobutyl]oxy}phenyl)-6,7-dihydro[1,3]thiazolo[4,5-b]pyridin-4(5H)-yl]ethanone a77 (3.6 g, 6.95 mmol, 1 eq) in dichloromethane (80 ml). The mixture is stirred at 25° C. for 1.5 hour and poured onto crushed ice and water. The layers are separated and the organic layer is washed once with water and once with a saturated aqueous solution of ammonium chloride, then dried over magnesium sulfate and concentrated under reduced pressure. The residue is purified by chromatography over silicagel (eluent: dichloromethane/methanol/ammonia 96:4:0.4) to afford 165 mg of 2-oxo-2-[2-{4-[(trans-3-piperidin-1-ylcyclobutyl)oxy]phenyl}-6,7-dihydro[1,3]thiazolo[4,5-b]pyridin-4(5H)-yl]ethanol 66 as a beige solid.

Yield: 5.6%.

LC-MS (MH⁺): 428.

### EXAMPLE 26

### Synthesis of 5-acetyl-2-{2-fluoro-4-[(trans-3-piperidin-1-ylcyclobutyl)oxy]phenyl}-4,5,6,7-tetrahydro[1,3]thiazolo[5,4-c]pyridine 36

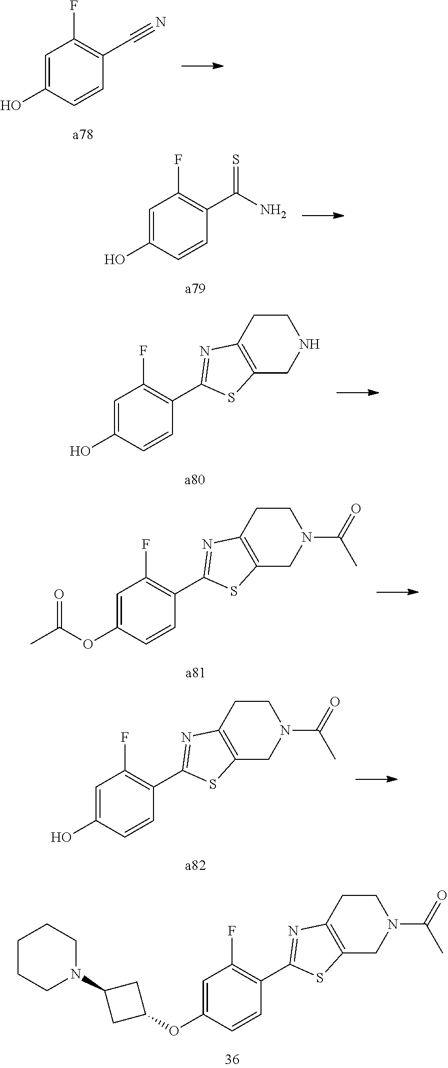

### 26.1 Synthesis of 2-fluoro-4-hydroxybenzenecarbothioamide a79

A solution of phosphorus hemipentasulfide (22 g, 49.6 mmol, 2 eq) in ethanol (50 ml), under an argon atmosphere, is treated with 2-fluoro-4-hydroxybenzonitrile a78 (3.4 g, 24.8 mmol, 1 eq) at 25° C. The mixture is stirred overnight at 80° C., then diluted with diethyl ether and carefully washed with a saturated aqueous solution of sodium hydrogenocarbonate. The organic layer is dried over magnesium sulfate and concentrated under reduced pressure. The residue is taken up in hexane and the precipitate is filtered, taken up in N,N-dimethylformamide and dried to afford 4.24 g of 2-fluoro-4-hydroxybenzenecarbothioamide a79 as an orange solid.

Yield: 100%.

LC-MS (MH⁺): 172.

### 26.2 Synthesis of 3-fluoro-4-(4,5,6,7-tetrahydro[1,3]thiazolo[5,4-c]pyridin-2-yl)phenol a80

3-bromo-piperidin-4-one hydrobromide (6.42 g, 24.8 mmol, 1 eq) is added to a solution of 2-fluoro-4-hydroxybenzenecarbothioamide a79 (4.24 g, 24.8 mmol, 1 eq) in N,N-dimethylformamide (80 ml) under an argon atmosphere. The mixture is stirred at 50° C. overnight, then is concentrated under reduced pressure (m=6.2 g). 3-fluoro-4-(4,5,6,7-tetrahydro[1,3]thiazolo[5,4-c]pyridin-2-yl)phenol a80 is used in the next step without any further purification.

Yield: 100%.

LC-MS (MH⁺): 251.

### 26.3 Synthesis of 4-(5-acetyl-4,5,6,7-tetrahydro[1,3]thiazolo[5,4-c]pyridin-2-yl)-3-fluorophenyl acetate a81

3-fluoro-4-(4,5,6,7-tetrahydro[1,3]thiazolo[5,4-c]pyridin-2-yl)phenol a80 (6.2 g, 24.8 mmol, 1 eq) in dichloromethane (100 ml) is treated with acetic anhydride (11.5 ml, 122.23 mmol, 4.92 eq) and 4-dimethylaminopyridine (1 g, 8.18 mmol, 0.33 eq). The mixture is stirred at 40° C. for 60 hours, diluted in dichloromethane and washed with a saturated aqueous solution of sodium hydrogenocarbonate. The organic phase is dried over magnesium sulfate and concentrated under reduced pressure. The residue is purified by chromatography over silicagel (eluent: dichloromethane/methanol/ammonia 99:0.9:0.1, then heptane/dichloromethane/methanol/ammonia 49:49:1.8:0.2) to afford 1.2 g of 4-(5-acetyl-4,5,6,7-tetrahydro[1,3]thiazolo[5,4-c]pyridin-2-yl)-3-fluorophenyl acetate a81.

Yield: 14%.

LC-MS (MH⁺): 335.

### 26.4 Synthesis of 1-[2-(2-fluoro-4-hydroxyphenyl)-6,7-dihydro[1,3]thiazolo[5,4-c]pyridin-5(4H)-yl]ethanone a82

Sodium (approximatively 100 mg) is added to a solution of 4-(5-acetyl-4,5,6,7-tetrahydro[1,3]thiazolo[5,4-c]pyridin-2-yl)-3-fluorophenyl acetate a81 (1.2 g, 3.59 mmol, 1 eq) in methanol (10 ml) and the mixture is stirred at 25° C. for 3 hours. The mixture is concentrated under reduced pressure to give 1.105 g of crude 1-[2-(2-fluoro-4-hydroxyphenyl)-6,7-dihydro[1,3]thiazolo[5,4-c]pyridin-5(4H)-yl]ethanone a82 as a solid which is used in the next step without any further purification.

Yield: 100%.

LC-MS (MH⁺): 293.

### 26.5 Synthesis of 5-acetyl-2-{2-fluoro-4-[(trans-3-piperidin-1-ylcyclobutyl)oxy]phenyl}-4,5,6,7-tetrahydro[1,3]thiazolo[5,4-c]pyridine 36

A solution of 1-[2-(2-fluoro-4-hydroxyphenyl)-6,7-dihydro[1,3]thiazolo[5,4-c]pyridin-5(4H)-yl]ethanone a82 (1.05 g, 3.59 mmol, 1 eq) in tetrahydrofuran (50 ml) is treated under an argon atmosphere with molecular sieves (600 mg), 15-crown-5 (1.28 ml, 7.18 mmol, 2 eq) and the mixture is stirred at 30° C. for 30 minutes. Sodium hydride (60% in mineral oil, 287 mg, 7.18 mmol, 2 eq) is added and the mixture is stirred at 60° C. for 1 hour. Then, 4-bromo-benzenesulfonic acid 3-piperidin-1-yl-cyclobutyl ester (1.48 g, 3.95 mmol, 1.1 eq) are added and the mixture is heated at 60° C. for 17 days. The mixture is taken up in water, extracted 3 times with ethyl acetate, dried over magnesium sulfate and concentrated under reduced pressure. The residue is purified by flash chromatography over alumina (gradient: dichloromethane/hexane 60:40 to 100:0) to afford an orange solid. T is solid is triturated in diethyl ether to afford 270 mg of 5-acetyl-2-{2-fluoro-4-[(trans-3-piperidin-1-ylcyclobutyl)oxy]phenyl}-4,5,6,7-tetrahydro[1,3]thiazolo[5,4-c]pyridine 36 as a beige solid.

Yield: 16%.

LC-MS (MH⁺): 430.

### EXAMPLE 27

### Synthesis of 5-acetyl-2-{3-fluoro-4-[(trans-3-piperidin-1-ylcyclobutyl)oxy]phenyl}-4,5,6,7-tetrahydro[1,3]thiazolo[5,4-c]pyridine 39

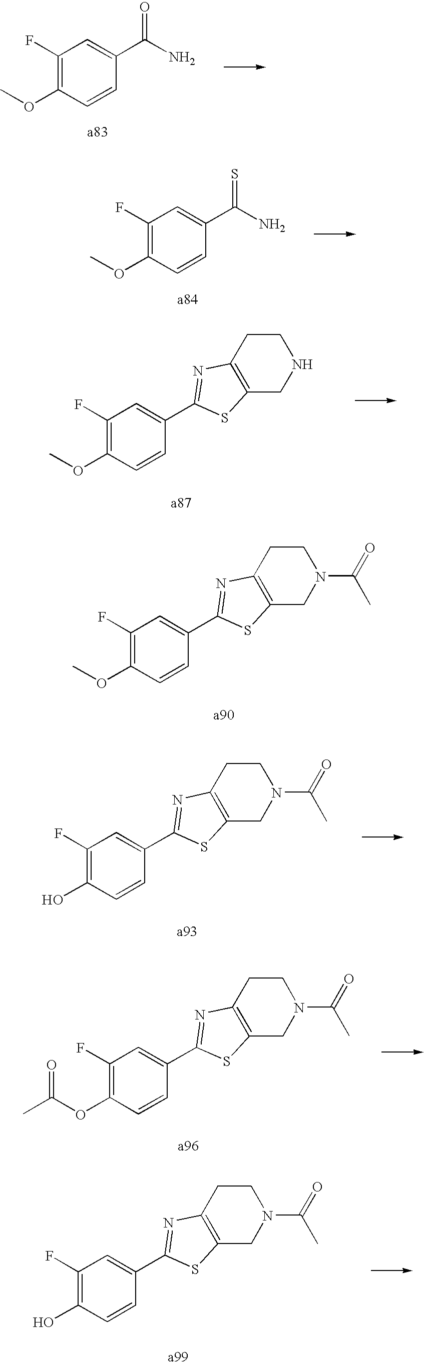

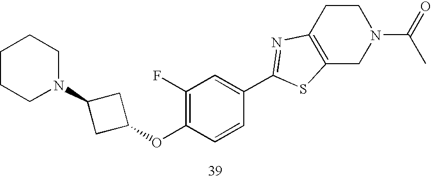

### 27.1 Synthesis of 3-fluoro-4-methoxybenzenecarbothioamide a84

A solution of 3-fluoro-4-methoxybenzamide a83 (5.09 g, 30.1 mmol, 1 eq) in tetrahydrofuran (220 ml), under an argon atmosphere, is treated with Lawesson's reagent (17.05 g, 42.1 mmol, 1.4 eq) at 0° C. The mixture is stirred at 25° C. overnight. The precipitate is filtered and the filtrate is concentrated under reduced pressure. The residue is taken up in dichloromethane and the precipitate is filtered to give 4.51 g of 3-fluoro-4-methoxybenzenecarbothioamide a84 as a yellow solid.

Yield: 81%.

LC-MS (MH⁺): 186.

The following compounds may be synthesized according to the same method:

| | | |
|---|---|---|
| a85 | 2,6-difluoro-4- | LC-MS (MH⁺): 204 |
| | methoxybenzenecarbothioamide | |
| a86 | 2,3-difluoro-4-methoxy-thiobenzamide | LC-MS (MH⁺): 204 |

### 27.2 Synthesis of 2-(3-fluoro-4-methoxyphenyl)-4,5,6,7-tetrahydro[1,3]thiazolo[5,4-c]pyridine a87

3-bromo-piperidin-4-one hydrobromide (6.21 g, 24 mmol, 1 eq) is added to a solution of 3-fluoro-4-methoxybenzenecarbothioamide a84 (4.44 g, 24 mmol, 1 eq) in N,N-dimethylformamide (80 ml) under an argon atmosphere. The mixture is stirred at 50° C. for 7 days, then concentrated under reduced pressure and purified by chromatography over silicagel (gradient: dichloromethane/methanol/ammonia 98:2:0.2 to 97:3:0.3) to afford 2.73 g of 2-(3-fluoro-4-methoxyphenyl)-4,5,6,7-tetrahydro[1,3]thiazolo[5,4-c]pyridine a87 as a yellow solid.

Yield: 43%.

LC-MS (MH⁺): 265.

The following compounds may be synthesized according to the same method:

| | | |
|---|---|---|
| a88 | 2-(2,6-difluoro-4-methoxyphenyl)-4,5,6,7- | LC-MS (MH⁺): 283 |
| | tetrahydro[1,3]thiazolo[5,4-c]pyridine | |
| a89 | 2-(2,3-difluoro-4-methoxyphenyl)-4,5,6,7- | LC-MS (MH⁺): 283 |
| | tetrahydro[1,3]thiazolo[5,4-c]pyridine | |

### 27.3 Synthesis of 1-[2-(3-fluoro-4-methoxyphenyl)-6,7-dihydro[1,3]thiazolo[5,4-c]pyridin-5(4H)-yl]ethanone a90

2-(3-fluoro-4-methoxyphenyl)-4,5,6,7-tetrahydro[1,3]thiazolo[5,4-c]pyridine a87 (2.7 g, 10.2 mmol, 1 eq) in dichloromethane (35 ml) is treated with acetic anhydride (1.45 ml, 15.3 mmol, 1.5 eq) and 4-dimethylaminopyridine (0.13 g, 1.06 mmol, 0.1 eq). The mixture is stirred at 40° C. for 3 hours. The mixture is diluted in dichloromethane and washed with water. The organic phase is dried over magnesium sulfate and concentrated under reduced pressure to give 3.19 g of 1-[2-(3-fluoro-4-methoxyphenyl)-6,7-dihydro[1,3]thiazolo[5,4-c]pyridin-5(4H)-yl]ethanone a90 as a beige solid.

Yield: 100%.

LC-MS (MH⁺): 307.

The following compounds may be synthesized according to the same method:

| | | |
|---|---|---|
| a91 | 1-[2-(2,6-difluoro-4-methoxyphenyl)-6,7- | LC-MS (MH⁺): 325 |
| | dihydro[1,3]thiazolo[5,4-c]pyridin-5(4H)- | |
| | yl]ethanone | |
| a92 | 1-[2-(2,3-difluoro-4-methoxyphenyl)-6,7- | LC-MS (MH⁺): 325 |
| | dihydro[1,3]thiazolo[5,4-c]pyridin-5(4H)- | |
| | yl]ethanone | |

### 27.4 Synthesis of 1-[2-(3-fluoro-4-hydroxyphenyl)-6,7-dihydro[1,3]thiazolo[5,4-c]pyridin-5(4H)-yl]ethanone a93

Dry lithium iodide (1.36 g, 10.18 mmol, 1 eq) is added to a solution of 1-[2-(3-fluoro-4-methoxyphenyl)-6,7-dihydro[1,3]thiazolo[5,4-c]pyridin-5(4H)-yl]ethanone a90 (3.1 g, 10.18 mmol, 1 eq) in 2,6-lutidine (30 ml). The mixture is stirred under an argon atmosphere overnight at 125° C. The mixture is taken up in ethyl acetate and washed with water. The aqueous phase is extracted with dichloromethane. The aqueous phase is concentrated under reduced pressure to give a brown solid. This solid is dried under reduced pressure with P₂O₅ to afford 3.5 g of crude 1-[2-(3-fluoro-4-hydroxyphenyl)-6,7-dihydro[1,3]thiazolo[5,4-c]pyridin-5(4H)-yl]ethanone a93.

Yield: 100%.

LC-MS (MH⁺): 293

The following compounds may be synthesized according to the same method:

| | | |
|---|---|---|
| a94 | 1-[2-(2,6-difluoro-4-hydroxyphenyl)-6,7- | LC-MS (MH⁺): 311 |
| | dihydro[1,3]thiazolo[5,4-c]pyridin-5(4H)- | |
| | yl]ethanone | |
| a95 | 1-[2-(2,3-difluoro-4-hydroxyphenyl)-6,7- | LC-MS (MH⁺): 311 |
| | dihydro[1,3]thiazolo[5,4-c]pyridin-5(4H)- | |
| | yl]ethanone | |

### 27.5 Synthesis of 4-(5-acetyl-4,5,6,7-tetrahydro[1,3]thiazolo[5,4-c]pyridin-2-yl)-2-fluorophenyl acetate a96

Acetic anhydride (1.9 ml, 20.36 mmol, 2 eq) and 4-dimethymaminopyridine (245 mg, 2 mmol, 0.2 eq) are added to a solution of 1-[2-(3-fluoro-4-hydroxyphenyl)-6,7-dihydro[1,3]thiazolo[5,4-c]pyridin-5(4H)-yl]ethanone a93 (2.98 g, 10.18 mmol, 1 eq) in dichloromethane (30 ml). The mixture is stirred at 40° C. for 5 hours. The mixture is taken up in dichloromethane and washed with water. The organic phase is dried over magnesium sulfate and concentrated under reduced pressure. The residue is purified by flash chromatography over silicagel (gradient: dichloromethane/methanol 100:0 to 0:100) to afford 2.5 g of crude 4-(5-acetyl-4,5,6,7-tetrahydro[1,3]thiazolo[5,4-c]pyridin-2-yl)-2-fluorophenyl acetate a96.

Yield: 40%.

LC-MS (MH⁺): 335.

The following compounds may be synthesized according to the same method:

| a97 | 4-(5-acetyl-4,5,6,7-tetrahydro[1,3]thiazolo[5,4- | LC-MS (MH⁺): |
|---|---|---|
| | c]pyridin-2-yl)-3,5-difluorophenyl acetate | 353 |
| a98 | 4-(5-acetyl-4,5,6,7-tetrahydro[1,3]thiazolo[5,4- | LC-MS (MH⁺): |
| | c]pyridin-2-yl)-2,3-difluorophenyl acetate | 353 |

### 27.6 Synthesis of 1-[2-(3-fluoro-4-hydroxyphenyl)-6,7-dihydro[1,3]thiazolo[5,4-c]pyridin-5(4H)-yl]ethanone a99

A solution of 4-(5-acetyl-4,5,6,7-tetrahydro[1,3]thiazolo[5,4-c]pyridin-2-yl)-2-fluorophenyl acetate a96 (2.5 g, 7.48 mmol, 1 eq) in methanol (150 ml) is treated under an argon atmosphere with sodium and the mixture is stirred at 25° C. overnight. The mixture is concentrated under reduced pressure. The residue is purified by reverse phase chromatography over silicagel (gradient: acetonitrile/water 5:95 to 70:30) to afford 0.9 g of 1-[2-(3-fluoro-4-hydroxyphenyl)-6,7-dihydro[1,3]thiazolo[5,4-c]pyridin-5(4H)-yl]ethanone a99 as a beige solid.

Yield: 40%.

LC-MS (MH⁺): 293.

The following compounds may be synthesized according to the same method:

| | | |
|---|---|---|
| a100 | 1-[2-(2,6-difluoro-4-hydroxyphenyl)-6,7- | LC-MS (MH⁺): 311 |
| | dihydro[1,3]thiazolo[5,4-c]pyridin-5(4H)- | |
| | yl]ethanone | |
| a101 | 1-[2-(2,3-difluoro-4-hydroxyphenyl)-6,7- | LC-MS (MH⁺): 311 |
| | dihydro[1,3]thiazolo[5,4-c]pyridin-5(4H)- | |
| | yl]ethanone | |

### 27.7 Synthesis of 5-acetyl-2-{3-fluoro-4-[(trans-3-piperidin-1-ylcyclobutyl)oxy]phenyl}-4,5,6,7-tetrahydro[1,3]thiazolo[5,4-c]pyridine 39

A solution of 1-[2-(3-fluoro-4-hydroxyphenyl)-6,7-dihydro[1,3]thiazolo[5,4-c]pyridin-5(4H)-yl]ethanone a99 (0.9 g, 3.07 mmol, 1 eq) in tetrahydrofuran (50 ml) is treated under an argon atmosphere with molecular sieves (600 mg), 15-crown-5 (1.1 ml, 6.14 mmol, 2 eq) and the mixture is stirred at 30° C. for 30 minutes. Sodium hydride (60% in mineral oil, 246 mg, 6.14 mmol, 2 eq) is added and the mixture is stirred at 60° C. for 1 hour. Then, cis-3-(piperidin-1-yl)cyclobutyl 4-bromobenzenesulfonate a106 (1.26 g, 3.37 mmol, 1.1 eq) is added and the mixture is heated at 60° C. for 12 days. The mixture is taken up in water, extracted with ethyl acetate 3 times, dried over magnesium sulfate and concentrated under reduced pressure. The residue is purified by flash chromatography over alumine (gradient: dichloromethane/hexane 90:10 to 100:0) to afford 60 mg of 5-acetyl-2-{3-fluoro-4-[(trans-3-piperidin-1-ylcyclobutyl)oxy]phenyl}-4,5,6,7-tetrahydro[1,3]thiazolo[5,4-c]pyridine 39 as an orange solid.

Yield: 4%.

LC-MS (MH⁺): 430.

Compounds 38 and 40 may be synthesized according to the same method.

### EXAMPLE 28

### Synthesis of 4-acetyl-2-{4-[(trans-3-piperidin-1-ylcyclobutyl)oxy]phenyl}-5,6,7,8-tetrahydro-4H-[1,3]oxazolo[5,4-b]azepine 67

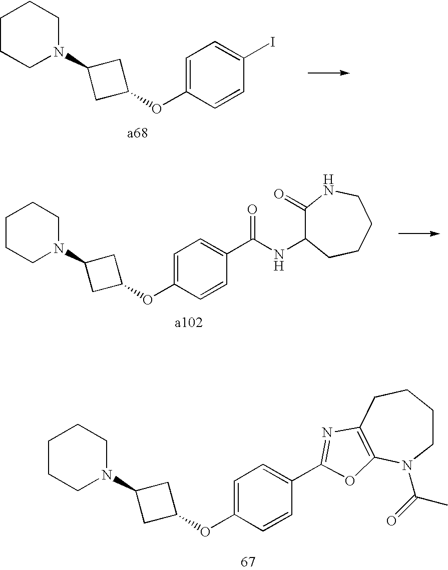

### 28.1 Synthesis of N-(2-oxoazepan-3-yl)-4-{[trans-3-(piperidin-1-yl)cyclobutyl]oxy}benzamide a102

A thick-walled vial is charged with 3-amino-azepan-2-one (0.86 g, 6.69 mmol, 3.15 eq), palladium (II) acetate (48 mg, 0.21 mmol, 0.1 eq), molybdenum hexacarbonyl (574 mg, 2.18 mmol, 1.024 eq) 1-[trans-3-(4-iodophenoxy)cyclobutyl]piperidine a68 (0.76 g, 2.12 mmol, 1 eq) and dry tetrahydrofuran (7.5 ml). The vial is capped with a Teflon septum under argon atmosphere and the mixture is cooled to 0° C. with an ice bath. 1,8-Diazabicyclo[5.4.0]undec-7-ene (1.1 ml, 7.22 mmol, 3.4 eq) is added through the septum. The vial is stirred under microwave irradiation at 125° C. for 20 minutes. After cooling, the reaction mixture is filtered through a short Celite pad, and the filtrate is taken up with ethyl acetate, washed with water and brine. The organic phase is dried over magnesium sulfate and concentrated under reduced pressure. The residue is purified by flash chromatography over silicagel (gradient: dichloromethane/methanol/ammonia 100:0:0 to 90:10:0.1) to afford N-(2-oxoazepan-3-yl)-4-{[trans-3-(piperidin-1-yl)cyclobutyl]oxy}benzamide a102.

Yield: 46%.

LC-MS (MH⁺): 386.

### 28.2 Synthesis of 4-acetyl-2-{4-[(trans-3-piperidin-1-ylcyclobutyl)oxy]phenyl}-5,6,7,8-tetrahydro-4H-[1,3]oxazolo[5,4-b]azepine 67

A thick-walled vial is charged with N-(2-oxo-azepan-3-yl)-4-(3-piperidin-1-yl-cyclobutoxy)-benzamide (0.58 g, 1.5 mmol, 1 eq), acetic anhydride (1.4 ml, 14.81 mmol, 9.8 eq) and titanium tetrachloride (2.2 ml, 19.8 mmol, 13.2 eq) in chloroform (22 ml). The vial is sealed with a Teflon septum and the mixture is stirred at 100° C. for 1 hour under microwave irradiation. The mixture is taken up in dichloromethane (50 ml) and a saturated aqueous solution of sodium hydrogenocarbonate is added to reach pH 9. A solid precipitates and is filtered off. The organic and aqueous phase are separated. The aqueous phase is extracted with dichloromethane, the combined organic phases are dried over magnesium sulfate and concentrated under reduced pressure to give a brown oil. The residue is purified by flash chromatography over silicagel (gradient: dichloromethane/methanol/ammonia 95:5:0.5 to 90:10:0.1) to afford 0.343 g of 4-acetyl-2-{4-[(trans-3-piperidin-1-ylcyclobutyl)oxy]phenyl}-5,6,7,8-tetrahydro-4H-[1,3]oxazolo[5,4-b]azepine 67.

Yield: 50%.

LC-MS (MH⁺): 410.

2-{4-[(trans-3-piperidin-1-ylcyclobutyl)oxy]phenyl}-4-(trifluoroacetyl)-5,6,7,8-tetrahydro-4H-[1,3]oxazolo[5,4-b]azepine 68 may be synthesized according to the same method.

### EXAMPLE 29

### Synthesis of 2-{4-[(trans-3-piperidin-1-ylcyclobutyl)oxy]phenyl}-3H-imidazo[4,5-c]pyridine 69, 5-acetyl-2-{4-[(cis-3-piperidin-1-ylcyclobutyl)oxy]phenyl}-4,5,6,7-tetrahydro-3H-imidazo[4,5-c]pyridine 61 and 5-acetyl-2-{4-[(trans-3-piperidin-1-ylcyclobutyl)oxy]phenyl}-4,5,6,7-tetrahydro-3H-imidazo[4,5-c]pyridine 62

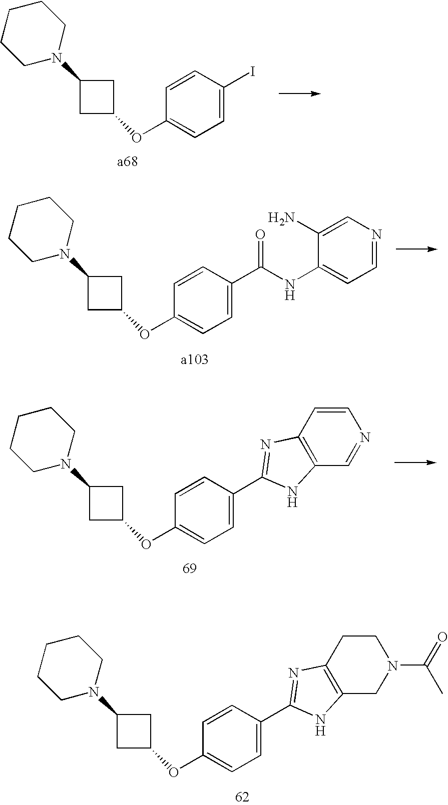

### 29.1 Synthesis of N-(3-aminopyridin-4-yl)-4-{[trans-3-(piperidin-1-yl)cyclobutyl]oxy}benzamide a103

Pyridine-3,4-diamine (5.08 g, 46.54 mmol, 3.3 eq), palladium acetate (0.596 g, 2.65 mmol, 0.19 eq) and molecular sieves are added to a solution of 1-[trans-3-(4-iodophenoxy)cyclobutyl]piperidine a68 (5.05 g, 14.14 mmol, 1 eq) in tetrahydrofuran (150 ml) in a sealed vessel. The mixture is stirred at 70° C. under 22 bars of carbon oxide during 54 hours. The mixture is then taken up in ethyl acetate (300 ml) and washed with water (2×100 ml). It is further washed with water at 35° C. until the pH of the aqueous phase reaches pH 7.5. The organic phase is then washed with brine (100 ml), dried over magnesium sulfate and concentrate under reduced pressure to afford 2.3 g of crude product. The aqueous phase is also concentrated, taken up in 60 ml of water, extracted with ethyl acetate (3×40 ml), dried over magnesium sulfate and concentrated under reduced pressure to give another 0.4 g of crude product.

The first 2.3 g of crude product is purified by reverse phase chromatography over silicagel (eluant: methanol/water/ammonia 5:95:0.1) to afford 0.777 g of N-(3-aminopyridin-4-yl)-4-{[trans-3-(piperidin-1-yl)cyclobutyl]oxy}benzamide a103.

Yield: 15%.

LC-MS (MH⁺): 367.

### 29.2 Synthesis of 2-{4-[(trans-3-piperidin-1-ylcyclobutyl)oxy]phenyl}-3H-imidazo[4,5-c]pyridine 69

N-(3-aminopyridin-4-yl)-4-{[trans-3-(piperidin-1-yl)cyclobutyl]oxy}benzamide a103 (0.39 g, 1.06 mmol, 1 eq) in butanol (12 ml) is treated with hydrochloric acid 37% (0.45 ml) in a sealed tube and the mixture is stirred under microwave irradiation at 120° C. for 80 minutes. The vessel is washed with water and the aqueous phase is basified with pellets of sodium hydroxide to reach pH 10. The aqueous phase is extracted with ethyl acetate (3×25 ml). The organic phase is washed with water, brine and dried over magnesium sulfate, then concentrated under reduced pressure. The residue is purified by preparative TLC over silicagel (eluent: dichloromethane/methanol/ammonia 87.5:12.5:1.25) to afford 2-{4-[(trans-3-piperidin-1-ylcyclobutyl)oxy]phenyl}-3H-imidazo[4,5-c]pyridine 69.

Yield: 5%.

LC-MS (MH⁺): 349.

### 29.3 Synthesis of 5-acetyl-2-{4-[(cis-3-piperidin-1-ylcyclobutyl)oxy]phenyl}-4,5,6,7-tetrahydro-3H-imidazo[4,5-c]pyridine 61 and 5-acetyl-2-{4-[(trans-3-piperidin-1-ylcyclobutyl)oxy]phenyl}-4,5,6,7-tetrahydro-3H-imidazo[4,5-c]pyridine 62

A solution of 2-{4-[(trans-3-piperidin-1-ylcyclobutyl)oxy]phenyl}-3H-imidazo[4,5-c]pyridine 69 (0.5 g, 1.43 mmol, 1 eq) in acetic acid (100 ml) and platinum dioxide (0.149 g, 0.66 mmol, 0.46 eq) is treated under 75 bars of hydrogen at 100° C. overnight. The mixture is filtered over Celite and concentrated to dryness. The residue is purified by chromatography over silicagel (gradient: dichloromethane/methanol/ammonia 100:0:0 to 85:15:1.5) then purified by reverse phase chromatography (eluent: acetonitrile/water/trifluoroacetic acid 5:95:0.1) to afford 5-acetyl-2-{4-[(trans-3-piperidin-1-ylcyclobutyl)oxy]phenyl}-4,5,6,7-tetrahydro-3H-imidazo[4,5-c]pyridine 62 and 5-acetyl-2-{4-[(cis-3-piperidin-1-ylcyclobutyl)oxy]phenyl}-4,5,6,7-tetrahydro-3H-imidazo[4,5-c]pyridine 61.

Yield: 17% (compound 62) and 2.5% (compound 61).

LC-MS (MH⁺): 395.

### EXAMPLE 30

### Synthesis of 1-{trans-3-[4-(5-acetyl-4,5,6,7-tetrahydro[1,3]thiazolo[5,4-c]pyridin-2-yl)phenoxy]cyclobutyl}-N,N-dimethylpyrrolidin-3-amine 50

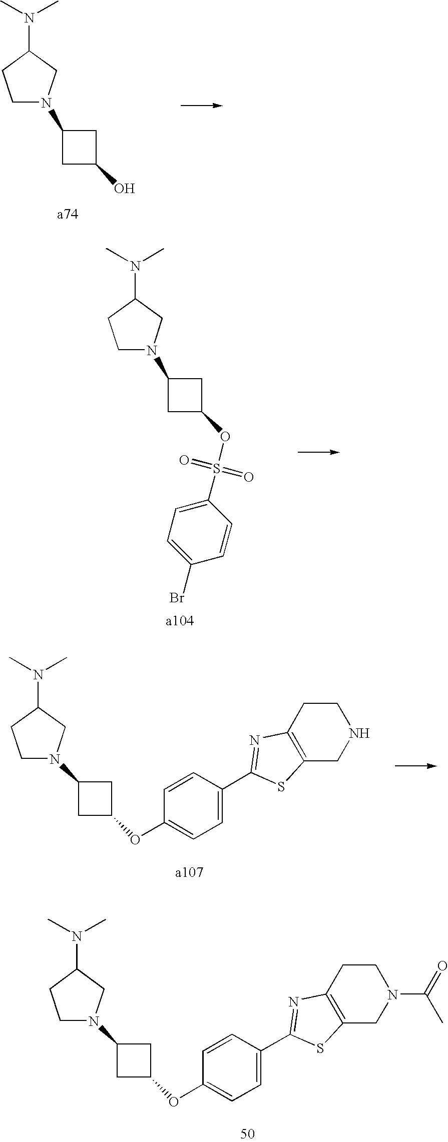

### 30.1 Synthesis of cis-3-[3-(dimethylamino)pyrrolidin-1-yl]cyclobutyl 4-bromobenzenesulfonate a104

A solution of cis-3-[3-(dimethylamino)pyrrolidin-1-yl]cyclobutanol a74 (4.95 g, 26.86 mmol, 1 eq) in ethyl acetate (130 ml) is treated with N-methylimidazole (2.36 ml, 29.55 mmol, 1.1 eq) and 4-bromo-benzenesulfonyl chloride (8.24 g, 32.23 mmol, 1.2 eq) at 25° C. After 4 hours, the mixture is washed with a saturated aqueous solution of sodium hydrogenocarbonate, dried over magnesium sulfate and concentrated under reduced pressure. The residue is purified by flash chromatography over silicagel (gradient: dichloromethane/methanol/ammonia 100:0:0 to 90:10:0.1) to give 3.24 g of cis-3-[3-(dimethylamino)pyrrolidin-1-yl]cyclobutyl 4-bromobenzenesulfonate a104 as a black oil.

Yield: 30%.

LC-MS (MH⁺): 403/405.

Cis-3-(4-cyclopentylpiperazin-1-yl)cyclobutyl 4-bromobenzenesulfonate a105 (LC-MS (MH⁺): 443/445) may be synthesized according to the same method.

Alternative method: synthesis of cis-3-(piperidin-1-yl)cyclobutyl 4-bromobenzenesulfonate a106.

A solution of cis-3-piperidin-1-ylcyclobutanol a17 (310 mg, 2 mmol, 1 eq.) in ethyl acetate (10 ml) is treated with 4-bromobenzenesulfonyl chloride (613 mg, 2.4 mmol, 1.2 eq) and N-methylimidazole (240 μl, 3 mmol, 1.5 eq). The mixture is stirred for 12 h at room temperature. The reaction mixture is filtered and the precipitate is rinsed with ethyl acetate. The solid is dissolved in ethyl acetate and washed with saturated sodium hydrogencarbonate and saturated ammonium chloride. The organic phase is dried over magnesium sulphate to yield 543 mg of cis-3-(piperidin-1-yl)cyclobutyl 4-bromobenzenesulfonate a106 as a yellow oil.

Yield: 72%.

LC-MS (MH⁺): 374/376.

### 30.2 Synthesis of N,N-dimethyl-1-{trans-3-[4-(4,5,6,7-tetrahydro[1,3]thiazolo[5,4-c]pyridin-2-yl)phenoxy]cyclobutyl}pyrrolidin-3-amine a107

A solution of 4-(4,5,6,7-tetrahydro-thiazolo[5,4-c]pyridin-2-yl)-phenol (3.34 g, 14.39 mmol, 1 eq) in dry tetrahydrofuran (28 ml), 15-crown-5 (5.6 ml, 28.77 mmol, 2 eq) and molecular sieves (20 g) is stirred at 40° C. for 20 minutes. Sodium hydride (60% mineral oil, 1.151 g, 28.77 mmol, 2 eq) is added, and the mixture is stirred at 60° C. for 2 hours. Cis-3-[3-(dimethylamino)pyrrolidin-1-yl]cyclobutyl 4-bromobenzenesulfonate a104 (7 g, 18.7 mmol, 1.3 eq) is then added and the mixture is stirred at 70° C. overnight. The mixture is taken up in ethyl acetate and washed with water, dried over magnesium sulfate and concentrated under reduced pressure to give an orange oil. This oil is taken up in dichloromethane and washed with brine, dried over magnesium sulfate and concentrated under reduced pressure to give 1.68 g of N,N-dimethyl-1-{trans-3-[4-(4,5,6,7-tetrahydro[1,3]thiazolo[5,4-c]pyridin-2-yl)phenoxy]cyclobutyl}pyrrolidin-3-amine a107 as an orange oil used without any further purification.

Yield: 53%.

LC-MS (MH⁺): 399.

The following compounds may be synthesized according to the same method:

| | | |
|---|---|---|
| a108 | 2-(4-{[trans-3-(4-cyclopentylpiperazin-1- | LC-MS (MH⁺): 439 |
| | yl)cyclobutyl]oxy}phenyl)-4,5,6,7- | |
| | tetrahydro[1,3]thiazolo[5,4-c]pyridine | |
| a109 | 2-(4-{[trans-3-(2-methylpyrrolidin-1- | LC-MS (MH⁺): 370 |
| | yl)cyclobutyl]oxy}phenyl)-4,5,6,7- | |
| | tetrahydro[1,3]thiazolo[5,4-c]pyridine | |

### 30.3 Synthesis of 1-{trans-3-[4-(5-acetyl-4,5,6,7-tetrahydro[1,3]thiazolo[5,4-c]pyridin-2-yl)phenoxy]cyclobutyl}-N,N-dimethylpyrrolidin-3-amine 50

A solution of N,N-dimethyl-1-{trans-3-[4-(4,5,6,7-tetrahydro[1,3]thiazolo[5,4-c]pyridin-2-yl)phenoxy]cyclobutyl}pyrrolidin-3-amine a107 (1.68 g, 4.22 mmol, 1 eq) in dichloromethane (65 ml) is treated with 4-dimethylaminopyridine (86 mg, 0.42 mmol, 0.1 eq) and acetic anhydride (0.6 ml, 6.32 mmol, 1.5 eq). The mixture is stirred at 40° C. overnight. The mixture is washed with water and brine, dried over magnesium sulfate and concentrated under reduced pressure to give 860 mg of crude product. The residue is purified by reverse phase chromatography over silicagel (gradient: acetonitrile/water/ammonia 5:95:0.1 to 50:50:0.1) to yield 70 mg of 1-{trans-3-[4-(5-acetyl-4,5,6,7-tetrahydro[1,3]thiazolo[5,4-c]pyridin-2-yl)phenoxy]cyclobutyl}-N,N-dimethylpyrrolidin-3-amine 50 as a white sticky solid.

Yield: 4%.

LC-MS (MH⁺): 441.

5-acetyl-2-(4-{[trans-3-(4-cyclopentylpiperazin-1-yl)cyclobutyl]oxy}phenyl)-4,5,6,7-tetrahydro[1,3]thiazolo[5,4-c]pyridine 51 and 1-[2-(4-{[trans-3-(2-methylpyrrolidin-1-yl)cyclobutyl]oxy}phenyl)-6,7-dihydro[1,3]thiazolo[5,4-c]pyridin-5(4H)-yl]ethanone a110 (LC-MS (MH⁺): 412) may be synthesized according to the same method.

Compound a110 is separated by chiral chromatography (phase: chiralpak IA; 30° C., column 50*500 mm; eluent: ethanol/heptane/diethylamine 50:50:0.1) to give 5-acetyl-2-[4-({trans-3-[2-methylpyrrolidin-1-yl]cyclobutyl}oxy)phenyl]-4,5,6,7-tetrahydro[1,3]thiazolo[5,4-c]pyridine, isomer A 57 and 5-acetyl-2-[4-({trans-3-[2-methylpyrrolidin-1-yl]cyclobutyl}oxy)phenyl]-4,5,6,7-tetrahydro[1,3]thiazolo[5,4-c]pyridine, isomer B 59.

### EXAMPLE 31

### Synthesis of 2-{4-[(trans-3-piperidin-1-ylcyclobutyl)oxy]phenyl}-6,7-dihydro[1,3]thiazolo[4,5-c]pyridine-5(4H)-carboxamide 64

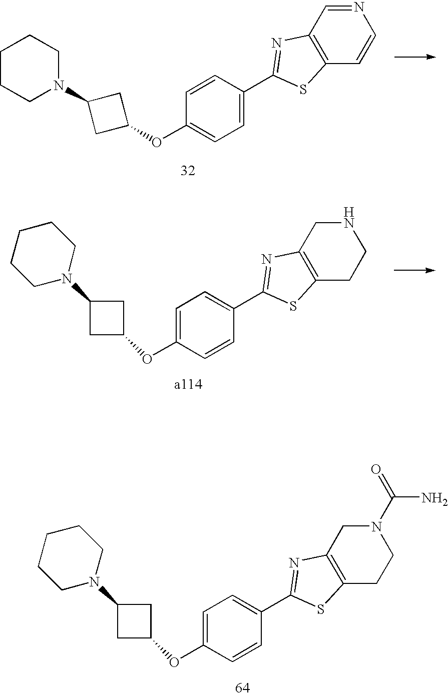

### 31.1 Synthesis of 2-(4-{[trans-3-(piperidin-1-yl)cyclobutyl]oxy}phenyl)-4,5,6,7-tetrahydro[1,3]thiazolo[4,5-c]pyridine a111

A solution of 2-{4-[(trans-3-piperidin-1-ylcyclobutyl)oxy]phenyl}[1,3]thiazolo[4,5-c]pyridine 32 (915 mg, 2.5 mmol, 1 eq) in dry tetrahydrofuran (45 ml) is treated with a 1M solution of lithium triethylborohydride in tetrahydrofuran (10.5 ml, 4.2 eq). The mixture is stirred one hour at 20° C. 1 N hydrogen chloride and ethyl acetate are added and the phases are separated. The aqueous phase is brought to pH 9 with solid potassium carbonate and further extracted twice with ethyl acetate. These last organic phases are dried over magnesium sulphate and concentrated in vacuo to afford 399 mg of crude 2-(4-{[trans-3-(piperidin-1-yl)cyclobutyl]oxy}phenyl)-4,5,6,7-tetrahydro[1,3]thiazolo[4,5-c]pyridine a111 as a yellow oil which is used without further purification.

Yield: 43%.

LC-MS (MH⁺): 370.

### 31.3 Synthesis of 2-{4-[(trans-3-piperidin-1-ylcyclobutyl)oxy]phenyl}-6,7-dihydro[1,3]thiazolo[4,5-c]pyridine-5(4H)-carboxamide 64

Compound 64 may be obtained as described in example 3.

LC-MS (MH⁺): 413.

Table I indicates the IUPAC name of the compound, the ion peak observed in mass spectrometry, the ¹H NMR description and melting point.

TABLE 1
Physical Characterisation of Example Compounds.

| n^{o} | IUPAC name | MH⁺ | ¹H NMR | MP ° C. |
|---|---|---|---|---|
| 1 | 2-{4-[(trans-3-piperidin-1-ylcyclobutyl)oxy]phenyl}- | 399 | (DMSO) 7.82 (m, 2 H), 6.94 (m, 2 H), 4.92 (m, 1 H), 3.94 (m, | — |
| | 5,6-dihydro-4H-cyclopenta[d][1,3]thiazole-5- | | 1 H), 3.75 (m, 1 H), 3.20 (m, 2 H), 3.10 (m, 2 H), 3.00 (m, 2 | |
| | carboxylic acid trifluoroacetate | | H), 2.79 (m, 4 H), 2.45 (m, 2 H), 1.83 (m, 2 H), 1.72 (m, 1 H), | |
| | | | 1.64 (m, 2 H), 1.38 (m, 1 H) | |
| 2 | 5-(methoxyacetyl)-2-{4-[(trans-3-piperidin-1- | 442 | 7.79 (d, J = 8.5 Hz, 2 H), 6.81 (d, J = 8.8 Hz, 2 H), 4.79 (m, 3 | 128 |
| | ylcyclobutyl)oxy]phenyl}-4,5,6,7- | | H), 4.21 (m, 2 H), 3.97 (t, J = 5.3 Hz, 1 H), 3.83 (t, J = 5.5 Hz, | |
| | tetrahydro[1,3]thiazolo[5,4-c]pyridine | | 1 H), 3.44 (m, 3 H), 2.99 (m, 3 H), 2.41 (m, 2 H), 2.31 (m, 5 | |
| | | | H), 1.70 (d, J = 0.5 Hz, 1 H), 1.61 (m, 4 H), 1.47 (m, 2 H) | |
| 3 | tert-butyl 2-{4-[(trans-3-piperidin-1- | 470 | 7.79 (m, 2 H), 6.81 (m, 2 H), 4.78 (m, 1 H), 4.62 (m, 2 H), | 162.3 |
| | ylcyclobutyl)oxy]phenyl}-6,7- | | 3.76 (m, 2 H), 3.00 (quint, J = 7.3 Hz, 1 H), 2.91 (s, 2 H), 2.33 (m, 8 | |
| | dihydro[1,3]thiazolo[5,4-c]pyridine-5(4H)- | | H), 1.60 (m, 4 H), 1.50 (s, 9 H), 1.46 (m, 2 H) | |
| | carboxylate | | | |
| 4 | 5-acetyl-2-{4-[(trans-3-piperidin-1- | 412 | 7.80 (m, 2 H), 6.82 (m, 2 H), 4.82 (s, 1 H), 4.78 (m, 1 H), | 170.3 |
| | ylcyclobutyl)oxy]phenyl}-4,5,6,7- | | 4.69 (m, 1 H), 3.96 (m, 1 H), 3.81 (m, 1 H), 2.98 (m, 3 H), 2.41 (m, | |
| | tetrahydro[1,3]thiazolo[5,4-c]pyridine | | 2 H), 2.31 (m, 5 H), 2.22 (s, 2 H), 2.19 (s, 1 H), 1.71 (s, 1 H), | |
| | | | 1.60 (m, 4 H), 1.45 (m, 2 H) | |
| 5 | 2-{4-[(trans-3-piperidin-1-ylcyclobutyl)oxy]phenyl}- | 370 | 7.80 (m, 2 H), 6.80 (m, 2 H), 4.81 (m, 1 H), 4.07 (m, 2 H), | 144.5 |
| | 4,5,6,7-tetrahydro[1,3]thiazolo[5,4-c]pyridine | | 3.21 (t, J = 6.0 Hz, 2 H), 3.07 (m, 1 H), 2.86 (m, 2 H), 2.50 (m, 2 | |
| | | | H), 2.35 (m, 6 H), 1.65 (m, 4 H), 1.48 (s, 2 H) | |
| 6 | 5-(morpholin-4-ylcarbonyl)-2-{4-[(trans-3-piperidin- | 483 | 7.79 (d, J = 8.8 Hz, 2 H), 6.81 (d, J = 8.8 Hz, 2 H), 4.79 (m, 1 | 149.3 |
| | 1-ylcyclobutyl)oxy]phenyl}-4,5,6,7- | | H), 4.49 (s, 2 H), 3.72 (m, 4 H), 3.61 (t, J = 5.5 Hz, 2 H), | |
| | tetrahydro[1,3]thiazolo[5,4-c]pyridine | | 3.33 (m, 4 H), 3.00 (m, 3 H), 2.40 (m, 2 H), 2.31 (m, 6 H), 1.61 (m, | |
| | | | 4 H), 1.47 (d, J = 4.5 Hz, 2 H) | |
| 7 | 5-(morpholin-4-ylsulfonyl)-2-{4-[(trans-3-piperidin- | 519 | 7.79 (d, J = 8.6 Hz, 2 H), 6.81 (d, J = 8.6 Hz, 2 H), 4.81 (m, 1 | 178 |
| | 1-ylcyclobutyl)oxy]phenyl}-4,5,6,7- | | H), 4.55 (s, 2 H), 3.70 (m, 6 H), 3.23 (m, 4 H), 3.08 (m, 1 H), | |
| | tetrahydro[1,3]thiazolo[5,4-c]pyridine | | 3.01 (t, J = 5.7 Hz, 2 H), 2.50 (m, 3 H), 2.35 (m, 7 H), 1.65 (m, | |
| | | | 4 H) | |
| 8 | 5-acetyl-2-{4-[(trans-3-morpholin-4- | 414 | (DMSO) 7.79 (m, 2 H), 6.92 (d, J = 8.4 Hz, 2 H), 4.81 (m, 1 | 161.6-162.2 |
| | ylcyclobutyl)oxy]phenyl}-4,5,6,7- | | H), 4.73 (m, 2 H), 3.79 (m, 2 H), 3.60 (t, J = 4.0 Hz, 4 H), | |
| | tetrahydro[1,3]thiazolo[5,4-c]pyridine | | 2.91 (m, 2 H), 2.77 (m, 1 H), 2.41 (m, 2 H), 2.30 (s, 4 H), 2.13 (m, 5 | |
| | | | H) | |
| 9 | 2-oxo-2-[2-{4-[(trans-3-piperidin-1- | 427 | (CDCl3 + CD3OD) 7.78 (d, J = 8.8 Hz, 2 H), 6.90 (d, J = 8.8 Hz, | 199 |
| | ylcyclobutyl)oxy]phenyl}-6,7- | | 2 H), 4.68-4.81 (m, 3 H), 3.69-3.88 (m, 2 H), 3.52 & | |
| | dihydro[1,3]thiazolo[5,4-c]pyridin-5(4H)- | | 3.59 (2s, 2 H), 2.76-2.93 (m, 3 H), 2.08-2.42 (m, 8H), 1.49 (m, 4 | |
| | yl]ethanamine | | H), 1.39 (m, 2 H) | |
| 10 | 2-{4-[(trans-3-piperidin-1-ylcyclobutyl)oxy]phenyl}- | 413 | 7.78 (d, J = 8.8 Hz, 2H), 6.80 (d, J = 8.5 Hz, 2H), 4.78 (m, 1 | 199 |
| | 6,7-dihydro[1,3]thiazolo[5,4-c]pyridine-5(4H)- | | H), 4.68 (m, 2 H), 3.73 (t, J = 5.8 Hz, 2 H), 2.93-3.04 (m, 3 H), | |
| | carboxamide | | 2.20-2.46 (m, 8H), 1.86 (bs, 2 H), 1.60 (m, 4 H), 1.46 (m, 2 H) | |
| 11 | 2-oxo-2-[2-{4-[(trans-3-piperidin-1- | 428 | 7.78 (d, J = 8.8 Hz, 2 H), 6.81 (d, J = 8.8 Hz, 2 H), 4.79 (m, 1 | 177 |
| | ylcyclobutyl)oxy]phenyl}-6,7- | | H), 4.51 & 4.88 (2s, 2 H), 4.25 & 4.31 (2s, 2 H), 4.03 (t, J = 5.8 Hz, | |
| | dihydro[1,3]thiazolo[5,4-c]pyridin-5(4H)-yl]ethanol | | 1 H), 3.63 (t, J = 5.8 Hz, 2 H), 2.94-3.05 (m, 3 H), | |
| | | | 2.19-2.46 (m, 8H), 1.60 (m, 4 H), 1.47 (m, 2H) | |
| 12 | 5-acetyl-2-(4-{[trans-3-(4-isopropylpiperazin-1- | 455 | (DMSO) 7.79 (m, 2 H), 6.91 (d, J = 8.8 Hz, 2 H), 4.76 (m, 3 | 109 |
| | yl)cyclobutyl]oxy}phenyl)-4,5,6,7- | | H), 3.79 (m, 2 H), 2.84 (m, 3 H), 2.63 (m, 2 H), 2.33 (m, 9 H), | |
| | tetrahydro[1,3]thiazolo[5,4-c]pyridine | | 2.15 (m, 5 H), 0.97 (d, J = 6.5 Hz, 6 H) | |
| 13 | 5-acetyl-2-(4-{[trans-3-(4,4-difluoropiperidin-1- | 448 | (DMSO) 7.79 (m, 2 H), 6.91 (d, J = 8.8 Hz, 2 H), 4.76 (m, 3 | 175 |
| | yl)cyclobutyl]oxy}phenyl)-4,5,6,7- | | H), 3.78 (m, 2 H), 3.03 (m, 1 H), 2.89 (t, J = 5.5 Hz, 1 H), | |
| | tetrahydro[1,3]thiazolo[5,4-c]pyridine | | 2.76 (t, J = 5.5 Hz, 1 H), 2.39 (m, 6 H), 2.20 (m, 5 H), 1.96 (m, 4 H) | |
| 14 | 5-acetyl-2-{4-[(trans-3-pyrrolidin-1- | 398 | 7.78 (d, J = 8.8 Hz, 2 H), 6.82 (m, 2 H), 4.91 (m, 1 H), 4.82 (s, | |
| | ylcyclobutyl)oxy]phenyl}-4,5,6,7- | | 1 H), 4.69 (s, 1 H), 3.96 (t, J = 5.8 Hz, 1 H), 3.80 (t, J = 5.8 Hz, | |
| | tetrahydro[1,3]thiazolo[5,4-c]pyridine | | 1 H), 3.13 (m, 1 H), 2.98 (t, J = 5.8 Hz, 1 H), 2.93 (t, J = 5.8 Hz, | |
| | | | 1 H), 2.53 (m, 6 H), 2.34 (m, 2 H), 2.21 (m, 3 H), | |
| | | | 1.85 (m, 4 H) | |
| 15 | 3-[2-{4-[(trans-3-piperidin-1- | 444 | (DMSO) 7.78 (m, 2 H), 6.91 (d, J = 7.3 Hz, 2 H), 4.81 (s, 1 H), | 169 |
| | ylcyclobutyl)oxy]phenyl}-6,7- | | 4.52 (s, 1 H), 3.77 (s, 2 H), 3.69 (s, 1 H), 3.36 (d, J = 5.0 Hz, 4 | |
| | dihydro[1,3]thiazolo[5,4-c]pyridin-5(4H)- | | H), 2.88 (s, 2 H), 2.79 (s, 2 H), 2.64 (dd, J = 12.7, 3.3 Hz, 2 | |
| | yl]propane-1,2-diol | | H), 2.47 (d, J = 7.2 Hz, 6 H), 2.23 (d, J = 0.6 Hz, 2 H), 1.56 (s, | |
| | | | 4 H), 1.44 (d, J = 1.1 Hz, 2 H) | |
| 16 | (2S)-3-[2-{4-[(trans-3-piperidin-1- | 444 | 7.79 (d, J = 8.8 Hz, 2 H), 6.80 (d, J = 8.8 Hz, 2 H), 4.79 (m, 1 | 175 |
| | ylcyclobutyl)oxy]phenyl}-6,7- | | H), 3.90 (m, 2 H), 3.78 (m, 2 H), 3.54 (dd, J = 11.3, 4.3 Hz, 1 | |
| | dihydro[1,3]thiazolo[5,4-c]pyridin-5(4H)- | | H), 3.01 (m, 6 H), 2.76 (dd, J = 12.8, 9.8 Hz, 1 H), 2.65 (dd, | |
| | yl]propane-1,2-diol | | J = 12.5, 3.8 Hz, 1 H), 2.40 (m, 3 H), 2.31 (m, 6 H), 1.60 (m, 4 | |
| | | | H), 1.46 (d, J = 4.5 Hz, 2 H) | |
| 17 | (2R)-3-[2-{4-[(trans-3-piperidin-1- | 444 | 7.79 (d, J = 8.8 Hz, 2 H), 6.80 (d, J = 8.8 Hz, 2 H), 4.79 (m, 1 | 172 |
| | ylcyclobutyl)oxy]phenyl}-6,7- | | H), 3.90 (m, 2 H), 3.78 (m, 2 H), 3.54 (dd, J = 11.3, 4.3 Hz, 1 | |
| | dihydro[1,3]thiazolo[5,4-c]pyridin-5(4H)- | | H), 3.01 (m, 6 H), 2.76 (dd, J = 12.8, 9.8 Hz, 1 H), 2.65 (dd, | |
| | yl]propane-1,2-diol | | J = 12.5, 3.8 Hz, 1 H), 2.40 (m, 3 H), 2.31 (m, 6 H), 1.60 (m, 4 | |
| | | | H), 1.46 (d, J = 4.5 Hz, 2 H) | |
| 18 | 2-[2-{4-[(trans-3-piperidin-1- | 427 | (DMSO) 7.87 (d, J = 8.8 Hz, 2 H), 6.98 (d, J = 9.0 Hz, 2 H), | 231 |
| | ylcyclobutyl)oxy]phenyl}-6,7- | | 4.93 (t, J = 6.8 Hz, 1 H), 4.66 (m, 2 H), 4.14 (s, 2 H), | |
| | dihydro[1,3]thiazolo[5,4-c]pyridin-5(4H)- | | 3.95 (quint, J = 7.5 Hz, 1 H), 3.71 (s, 2 H), 3.41 (d, J = 11.8 Hz, | |
| | yl]acetamide | | 2 H), 3.14 (s, 2 H), 2.80 (m, 4 H), 2.46 (m, 2 H), 1.86 (m, 2 H), | |
| | | | 1.68 (m, 3 H), 1.40 (m, 1 H) | |
| 19 | 5-acetyl-2-{4-[(trans-3-azepan-1- | 426 | 7.77 (d, J = 8.3 Hz, 2 H), 6.84 (d, J = 8.3 Hz, 2 H), | 135 |
| | ylcyclobutyl)oxy]phenyl}-4,5,6,7- | | 4.70-4.82 (m, 3 H), 3.76-3.90 (m, 2 H), 3.28 (m, 1 H), 2.78-2.96 (m, | |
| | tetrahydro[1,3]thiazolo[5,4-c]pyridine | | 2 H), 2.54 (s, 4 H), 2.35 (m, 2 H), 2.24 (m, 2 H), 2.13 & 2.16 (2s, | |
| | | | 3 H), 1.53-1.69 (m, 8 H) | |
| 20 | (3R)-1-{trans-3-[4-(5-acetyl-4,5,6,7- | 441 | (DMSO) 7.78 (m, 2 H), 6.91 (d, J = 8.5 Hz, 2 H), 4.83 (quint, | |
| | tetrahydro[1,3]thiazolo[5,4-c]pyridin-2- | | J = 5.8 Hz, 1 H), 4.73 (m, 2 H), 3.79 (m, 2 H), 2.98 (m, 1 H), | |
| | yl)phenoxy]cyclobutyl}-N,N-dimethylpyrrolidin-3- | | 2.89 (t, J = 5.5 Hz, 1 H), 2.69 (m, 4 H), 2.44 (m, 2 H), 2.35 (m, | |
| | amine | | 1 H), 2.14 (m, 12 H), 1.87 (m, 1 H), 1.61 (m, 1 H) | |
| 21 | N-ethyl-2-{4-[(trans-3-piperidin-1- | 441 | (DMSO) 7.78 (d, J = 8.8 Hz, 2 H), 6.90 (d, J = 8.8 Hz, 2 H), | 200 |
| | ylcyclobutyl)oxy]phenyl}-6,7- | | 6.70 (t, J = 5.3 Hz, 1 H), 4.78 (m, 1 H), 4.56 (s, 2 H), 3.67 (t, | |
| | dihydro[1,3]thiazolo[5,4-c]pyridine-5(4H)- | | J = 5.5 Hz, 2H), 3.07 (m, 2 H), 2.88 (m, 1 H), 2.75 (m, 2 H), | |
| | carboxamide | | 2.35 (m, 2 H), 2.16 (m, 6 H), 1.50 (m, 4 H), 1.39 (m, 2 H), | |
| | | | 1.02 (t, J = 7.0 Hz, 3 H) | |
| 22 | 5-acetyl-2-{4-[(trans-3-thiomorpholin-4- | 430 | (DMSO) 7.81 (m, 2 H), 6.92 (d, J = 8.5 Hz, 2 H), 4.89 (t, J = 6.0 Hz, | 196 |
| | ylcyclobutyl)oxy]phenyl}-4,5,6,7- | | 1 H), 4.72 (m, 2 H), 4.05 (t, J = 8.0 Hz, 1 H), 3.79 (m, | |
| | tetrahydro[1,3]thiazolo[5,4-c]pyridine | | 2 H), 3.64 (s, 2 H), 2.89 (m, 10 H), 2.45 (m, 2 H), 2.10 (m, 3 | |
| | | | H) | |
| 23 | 5-acetyl-2-{4-[(trans-3-piperidin-1- | 428 | 7.77 (d, J = 8.3 Hz), 7.19 (d, J 8.3 Hz), 4.70 & 4.83 (2s, 2 H), | |
| | ylcyclobutyl)thio]phenyl}-4,5,6,7- | | 3.88 (m, 3 H), 3.02 (m, 3 H), 2.53 (m, 2 H), 2.25 (m, 9 H), | |
| | tetrahydro[1,3]thiazolo[5,4-c]pyridine | | 1.60 (m, 4 H), 1.45 (m, 2 H) | |
| 24 | cis-3-[2-{4-[(trans-3-piperidin-1- | 440 | 7.79 (m, 2 H), 6.80 (m, 2 H), 4.78 (m, 1 H), 4.04 (m, 1 H), | 169 |
| | ylcyclobutyl)oxy]phenyl}-6,7- | | 3.62 (s, 2 H), 3.00 (m, 1 H), 2.94 (m, 2 H), 2.77 (t, J = 5.8 Hz, 2 H), | |
| | dihydro[1,3]thiazolo[5,4-c]pyridin-5(4H)- | | 2.60 (m, 3 H), 2.35 (m, 9 H), 1.88 (m, 2 H), 1.60 (m, 4 H), | |
| | yl]cyclobutanol | | 1.46 (d, J = 3.8 Hz, 2 H) | |
| 25 | 3-oxo-3-[2-{4-[(trans-3-piperidin-1- | 455 | 7.79 (m, 2 H), 7.34 (m, 1 H), 6.81 (d, J = 8.5 Hz, 2 H), | 176 |
| | ylcyclobutyl)oxy]phenyl}-6,7- | | 5.67 (m, 1 H), 4.84 (s, 1 H), 4.79 (m, 2 H), 3.99 (t, J = 5.8 Hz, 1 H), | |
| | dihydro[1,3]thiazolo[5,4-c]pyridin-5(4H)- | | 3.89 (t, J = 5.8 Hz, 1 H), 3.48 (s, 1 H), 3.44 (s, 1 H), 2.98 (m, 3 | |
| | yl]propanamide | | H), 2.41 (m, 2 H), 2.31 (m, 6 H), 1.60 (m, 4 H), 1.46 (d, J = 4.5 Hz, | |
| | | | 2 H) | |
| 26 | methyl [2-{4-[(trans-3-piperidin-1- | 442 | 7.80 (d, J = 8.8 Hz, 2 H), 6.79 (d, J = 8.8 Hz, 2 H), 5.01 (m, 1 | 221 |
| | ylcyclobutyl)oxy]phenyl}-6,7- | | H), 3.92 (s, 2 H), 3.76 (s, 3 H), 3.64 (m, 1 H), 3.50 (m, 3 H), | |
| | dihydro[1,3]thiazolo[5,4-c]pyridin-5(4H)-yl]acetate | | 3.36 (m, 2 H), 3.05 (m, 2 H), 2.97 (m, 2 H), 2.23-2.53 (m, 6 | |
| | | | H), 1.77-2.00 (m, 4 H), 1.42 (m, 1 H) | |
| 27 | diethyl {[2-{4-[(trans-3-piperidin-1- | 520 | 7.79 (d, J = 8.8 Hz, 2 H), 6.80 (d, J = 8.8 Hz, 2 H), 4.78 (m, 1 | |
| | ylcyclobutyl)oxy]phenyl}-6,7- | | H), 4.19 (m, 4 H), 3.95 (m, 2 H), 3.11 (t, J = 6.0 Hz, 2 H), | |
| | dihydro[1,3]thiazolo[5,4-c]pyridin-5(4H)- | | 3.01 (m, 3 H), 2.95 (m, 2 H), 2.40 (d, J = 6.3 Hz, 2 H), 2.26 (m, 6 | |
| | yl]methyl}phosphonate | | H), 1.61 (m, 4 H), 1.46 (m, 2 H), 1.35 (t, J = 7.0 Hz, 6 H) | |
| 28 | 5-acetyl-2-{4-[(trans-3-piperidin-1- | 396 | 7.90 (d, J = 8.8 Hz, 2 H), 6.83 (m, 2 H), 4.79 (m, 1 H), | 141.9 |
| | ylcyclobutyl)oxy]phenyl}-4,5,6,7- | | 4.57 (m, 2 H), 3.88 (m, 2 H), 3.02 (m, 1 H), 2.83 (m, 2 H), 2.45 | |
| | tetrahydro[1,3]oxazolo[4,5-c]pyridine | | (d, J = 6.3 Hz,2 H), 2.32 (m, 6H), 2.20 (m, 3 H), 1.61 (m, 4H), | |
| | | | 1.44 (m, 2 H) | |
| 29 | 5-acetyl-2-{4-[(trans-3-piperidin-1- | 412 | 7.79 (m, 2 H), 6.82 (m, 2 H), 4.86-4.66 (m, 3 H), 3.99-3.73 (m, | 154.2 |
| | ylcyclobutyl)oxy]phenyl}-4,5,6,7- | | 2 H), 3.06-2.83 (m, 3 H), 2.46-2.27 (m, 8 H), 2.21 (m, 3 H), | |
| | tetrahydro[1,3]thiazolo[4,5-c]pyridine | | 1.61 (m, 4 H), 1.47 (d, J = 4.5 Hz, 2 H) | |
| 30 | 4-acetyl-2-{4-[(trans-3-piperidin-1- | 412 | (DMSO) 7.78 (d, J = 8.8 Hz, 2 H), 6.81 (d, J = 8.8 Hz, 2 H), | 146 |
| | ylcyclobutyl)oxy]phenyl}-4,5,6,7- | | 4.79 (m, 1 H), 3.95 (m, 2 H), 2.99 (m, 1 H), 2.85 (t, J = 6.5 Hz, | |
| | tetrahydro[1,3]thiazolo[4,5-b]pyridine | | 2 H), 2.62 (s, 3 H), 2.41 (m, 2 H), 2.31 (m, 4 H), 2.03 (m, 2 H), | |
| | | | 1.79 (s, 2 H), 1.61 (m, 4 H), 1.47 (d, J = 4.0 Hz, 2 H) | |
| 31 | 4-acetyl-2-{4-[(trans-3-piperidin-1- | 426 | (DMSO) 7.75 (dd, J = 15.8, 8.5 Hz, 2 H), 6.89 (m, 2 H), | 49 |
| | ylcyclobutyl)oxy]phenyl}-5,6,7,8-tetrahydro-4H- | | 4.77 (m, 1 H), 3.81 (m, 1 H), 3.60 (m, 1H), 2.89 (m, 3 H), 2.35 (m, 2 | |
| | [1,3]thiazolo[5,4-b]azepine | | H), 2.25-2.10 (m, 7 H), 2.06 (s, 2 H), 1.94 (m, 1 H), 1.78 (d, | |
| | | | J = 4.8 Hz, 2 H), 1.59 (m, 1 H), 1.50 (m, 4 H), 1.39 | |
| | | | (d, J = 4.5 Hz, 2 H) | |
| 32 | 2-{4-[(trans-3-piperidin-1- | 366 | 9.48 (m, 1 H), 8.70 (m, 1 H), 8.47 (d, J = 6.3 Hz, 1 H), 8.10 (d, | 65 |
| | ylcyclobutyl)oxy]phenyl}[1,3]thiazolo[4,5-c]pyridine | | J = 8.8 Hz, 2 H), 6.97 (m, 2 H), 4.99 (m, 1 H), 3.90 (s, 1 H), | |
| | | | 3.68 (m, 2 H), 3.05 (m, 2 H), 2.64 (m, 4 H), 1.80-2.07 (m, 5 | |
| | | | H), 1.48 (m, 1 H) | |
| 33 | 2-{4-[(trans-3-piperidin-1-ylcyclobutyl)oxy]phenyl}- | 480 | 7.31 (m, 2 H), 6.34 (d, J = 8.4 Hz, 2 H), 4.29 (m, 3 H), | 167-168 |
| | 5-(3,3,3-trifluoropropanoyl)-4,5,6,7- | | 3.41 (m, 2 H), 2.88 (m, 2 H), 2.51 (m, 3 H), 1.92 (m, 2 H), 1.78 (m, | |
| | tetrahydro[1,3]thiazolo[5,4-c]pyridine | | 6 H), 1.13 (m, 4 H), 0.99 (d, J = 3.9 Hz, 2 H) | |
| 34 | {[2-{4-[(trans-3-piperidin-1- | 464 | 7.63 (d, J = 8.5 Hz, 2 H), 6.74 (d, J = 8.5 Hz, 2 H), 4.69 (s, 1 | |
| | ylcyclobutyl)oxy]phenyl}-6,7- | | H), 4.49 (m, 2 H), 3.70 (m, 1 H), 3.56 (m, 2 H), 3.39 (m, 2 H), | |
| | dihydro[1,3]thiazolo[5,4-c]pyridin-5(4H)- | | 3.17 (d, J = 11.5 Hz, 2 H), 2.91 (m, 2 H), 2.54 (m, 4 H), | |
| | yl]methyl}phosphonic acid | | 2.18 (m, 2 H), 1.65 (m, 2 H), 1.42 (m, 3 H), 1.14 (m, 1 H) | |
| 35 | 5-[(5-methyl-2H-1,2,3-triazol-4-yl)carbonyl]-2-{4- | 479 | 10.01 (m, 1 H), 7.57 (m, 2 H), 6.68 (d, J = 8.8 Hz, 2 H), | 150-154 |
| | [(trans-3-piperidin-1-ylcyclobutyl)oxy]phenyl}- | | 4.55 (m, 1 H), 4.48 (m, 2 H), 3.55 (m, 2 H), 2.68 (m, 3 H), 2.14 (m, | |
| | 4,5,6,7-tetrahydro[1,3]thiazolo[5,4-c]pyridine | | 3 H), 2.04 (m, 3 H), 1.93 (m, 5 H), 1.28 (m, 4 H), 1.17 (d, | |
| | | | J = 4.0 Hz, 2 H) | |
| 36 | 5-acetyl-2-{2-fluoro-4-[(trans-3-piperidin-1- | 430 | 7.82 (m, 1 H), 6.63 (m, 2 H), 4.60 (m, 2 H), 4.49 (m, 1 H), | 142.8 |
| | ylcyclobutyl)oxy]phenyl}-4,5,6,7- | | 3.57 (m, 2 H), 2.63 (m, 3 H), 2.16 (m, 2 H), 2.04 (m, 4 H), 1.92 (m, | |
| | tetrahydro[1,3]thiazolo[5,4-c]pyridine | | 4 H), 1.85 (m, 1 H), 1.29 (m, 4 H), 1.15 (m, 2 H) | |
| 37 | 2-[2-{4-[(trans-3-piperidin-1- | 414 | 1.48 (bs, 2 H), 1.58-1.69 (m, 4 H), 2.27-2.54 (m, 8 H), 2.79 (t, | 142 |
| | ylcyclobutyl)oxy]phenyl}-6,7- | | J = 5.3 Hz, 2 H), 2.91-3.00 (m, 4 H), 3.02-3.11 (m, 1 H), | |
| | dihydro[1,3]thiazolo[5,4-c]pyridin-5(4H)-yl]ethanol | | 3.71 (t, J = 5.3 Hz, 2 H), 3.80 (s, 2 H), 4.76-4.83 (m, 1 H), 6.80 (d, | |
| | | | J = 8.6 Hz, 2 H), 7.79 (d, J = 8.6 Hz, 2 H) | |
| 38 | 5-acetyl-2-{2,6-difluoro-4-[(trans-3-piperidin-1- | 448 | 6.43 (m, 2 H), 4.88 (m, 1 H), 4.75 (m, 2 H), 3.91 (m, 2 H), | |
| | ylcyclobutyl)oxy]phenyl}-4,5,6,7- | | 3.03 (m, 3 H), 2.48 (m, 2 H), 2.35 (m, 6 H), 2.23 (m, 3 H), 1.64 (m, | |
| | tetrahydro[1,3]thiazolo[5,4-c]pyridine | | 4 H), 1.48 (s, 2 H) | |
| 39 | 5-acetyl-2-{3-fluoro-4-[(trans-3-piperidin-1- | 430 | 7.66 (m, 1 H), 7.54 (m, 1 H), 6.76 (m, 1 H), 4.91 (m, 1 H), | |
| | ylcyclobutyl)oxy]phenyl}-4,5,6,7- | | 4.83 (m, 1 H), 4.70 (m, 1 H), 3.97 (m, 1 H), 3.80 (m, 1 H), 3.20 (m, | |
| | tetrahydro[1,3]thiazolo[5,4-c]pyridine | | 1 H), 2.96 (m, 2 H), 2.65 (m, 2 H), 2.42 (m, 6 H), 2.21 (m, 3 | |
| | | | H), 1.71 (m, 4 H), 1.51 (m, 2 H) | |
| 40 | 5-acetyl-2-{2,3-difluoro-4-[(trans-3-piperidin-1- | 448 | 7.84 (m, 1 H), 6.96 (m, 1 H), 4.92 (m, 1 H), 4.79 (m, 2 H), | |
| | ylcyclobutyl)oxy]phenyl}-4,5,6,7- | | 3.82 (m, 2 H), 2.84 (m, 3 H), 2.41 (m, 3 H), 2.26 (m, 5 H), 2.09 (m, | |
| | tetrahydro[1,3]thiazolo[5,4-c]pyridine | | 3 H), 1.54 (m, 4 H), 1.35 (m, 2 H) | |
| 41 | 5-[4-(1-oxidothiomorpholin-4-yl)butanoyl]-2-{4- | 557 | 7.78 (m, 2 H), 6.81 (d, J = 8.6 Hz, 2 H), 4.80 (m, 2 H), 4.71 (s, | 165 |
| | [(trans-3-piperidin-1-ylcyclobutyl)oxy]phenyl}- | | 1 H), 3.97 (t, J = 5.5 Hz, 1 H), 3.82 (t, J = 5.7 Hz, 1 H), | |
| | 4,5,6,7-tetrahydro[1,3]thiazolo[5,4-c]pyridine | | 3.01 (m, 5 H), 2.83 (m, 4 H), 2.69 (m, 2 H), 2.47 (m, 7 H), 2.32 (m, | |
| | | | 5 H), 1.89 (m, 2 H), 1.62 (m, 4 H), 1.47 (d, J = 3.7 Hz, 2 H) | |
| 42 | N-{3-oxo-3-[2-{4-[(trans-3-piperidin-1- | 483 | 7.79 (m, 2 H), 6.81 (d, J = 8.8 Hz, 2 H), 6.37 (s, 1 H), 4.81 (m, | 144 |
| | ylcyclobutyl)oxy]phenyl}-6,7- | | 2 H), 4.66 (s, 1 H), 3.97 (t, J = 5.9 Hz, 1 H), 3.77 (t, J = 5.7 Hz, | |
| | dihydro[1,3]thiazolo[5,4-c]pyridin-5(4H)- | | 1 H), 3.59 (m, 2 H), 2.96 (m, 4 H), 2.64 (m, 2 H), 2.44 (m, | |
| | yl]propyl}acetamide | | 2 H), 2.31 (m, 5 H), 1.94 (m, 3 H), 1.62 (m, 4 H), 1.47 (s, 2 H) | |
| 43 | {2-oxo-2-[2-{4-[(trans-3-piperidin-1- | 486 | 7.84 (d, J = 8.5 Hz, 2 H), 6.95 (d, J = 8.8 Hz, 2 H), 4.92 (t, | >200 |
| | ylcyclobutyl)oxy]phenyl}-6,7- | | J = 6.5 Hz, 1 H), 4.74 (s, 2 H), 4.37 (m, 2 H), 4.13 (m, 2 H), | |
| | dihydro[1,3]thiazolo[5,4-c]pyridin-5(4H)- | | 3.95 (m, 1 H), 3.80 (m, 2 H), 3.40 (d, J = 11.8 Hz, 2 H), 2.91 (s, 1 | |
| | yl]ethoxy}acetic acid | | H), 2.78 (m, 5 H), 2.43 (m, 2 H), 1.88 (m, 2 H), 1.65 (m, | |
| 44 | 1,1,1-trifluoro-3-oxo-3-[2-{4-[(trans-3-piperidin-1- | 496 | 7.80 (m, 2 H), 6.90 (m, 2 H), 5.25 (m, 1 H), 4.82 (m, 3 H), | 147-148 |
| | ylcyclobutyl)oxy]phenyl}-6,7- | | 3.90 (m, 2 H), 2.88 (m, 3 H), 2.37 (m, 2 H), 2.21 (m, 4 H), 2.13 (m, | |
| | dihydro[1,3]thiazolo[5,4-c]pyridin-5(4H)-yl]propan- | | 2 H), 1.50 (m, 4 H), 1.36 (m, 2 H) | |
| | 2-ol | | | |
| 45 | 2-{4-[(trans-3-piperidin-1-ylcyclobutyl)oxy]phenyl}- | 482 | 7.83 (d, J = 8.3 Hz, 2 H), 6.95 (d, J = 8.8 Hz, 2 H), 4.90 (m, 2 | 175-176 |
| | 5-(tetrahydro-2H-pyran-4-ylcarbonyl)-4,5,6,7- | | H), 4.73 (s, 1 H), 3.87 (m, 5 H), 3.02 (m, 1 H), 2.80 (m, 6 H), | |
| | tetrahydro[1,3]thiazolo[5,4-c]pyridine | | 2.41 (m, 2 H), 1.86 (d, J = 14.3 Hz, 2 H), 1.64 (m, 7 H), | |
| | | | 1.35 (m, 1 H) | |
| 46 | 1-{[2-{4-[(trans-3-piperidin-1- | 454 | 7.79 (d, J = 8.8 Hz, 2 H), 6.90 (d, J = 8.8 Hz, 2 H), 6.46 (s, 1 | 178 |
| | ylcyclobutyl)oxy]phenyl}-6,7- | | H), 4.84 (m, 3 H), 3.97 (m, 2 H), 2.87 (m, 3 H), 2.36 (m, 2 H), | |
| | dihydro[1,3]thiazolo[5,4-c]pyridin-5(4H)- | | 2.23 (s, 3 H), 2.12 (m, 3 H), 1.50 (m, 4 H), 1.39 (d, J = 4.0 Hz, | |
| | yl]carbonyl}cyclopropanol | | 2 H), 0.97 (s, 2 H), 0.82 (m, 2 H) | |
| 47 | 1-{[2-{4-[(trans-3-piperidin-1- | 481 | 7.79 (d, J = 8.8 Hz, 2 H), 7.21 (m, 2 H), 6.90 (d, J = 8.8 Hz, 2 | 199-200 |
| | ylcyclobutyl)oxy]phenyl}-6,7- | | H), 4.75 (m, 3 H), 3.78 (m, 2 H), 2.88 (m, 3 H), 2.36 (m, 2 H), | |
| | dihydro[1,3]thiazolo[5,4-c]pyridin-5(4H)- | | 2.13 (m, 6 H), 1.49 (m, 4 H), 1.36 (m, 2 H), 1.23 (m, 2 H), | |
| | yl]carbonyl}cyclopropanecarboxamide | | 1.08 (m, 2 H) | |
| 48 | 1-{[2-{4-[(trans-3-piperidin-1- | 481 | 7.78 (d, J = 8.8 Hz, 2 H), 6.91 (d, J = 8.8 Hz, 2 H), 4.72 (s, 3 | |
| | ylcyclobutyl)oxy]phenyl}-6,7- | | H), 3.81 (s, 2 H), 2.88 (d, J = 5.8 Hz, 3 H), 2.37 (m, 2 H), | |
| | dihydro[1,3]thiazolo[5,4-c]pyridin-5(4H)- | | 2.18 (m, 6 H), 1.50 (m, 4 H), 1.39 (d, J = 4.8 Hz, 2 H), 1.26 | |
| | yl]carbonyl}cyclopropanecarboxamide | | (d, J = 2.3 Hz,2 H), 1.11 (m, 2 H) | |
| | trifluoroacetate | | | |
| 49 | ethyl oxo[2-{4-[(trans-3-piperidin-1- | 470 | 7.31 (m, 2 H), 6.34 (d, J = 8.4 Hz, 2 H), 4.29 (m, 3 H), | 145-146 |
| | ylcyclobutyl)oxy]phenyl}-6,7- | | 3.41 (m, 2 H), 2.88 (m, 2 H), 2.51 (m, 3 H), 1.92 (m, 2 H), 1.78 (m, | |
| | dihydro[1,3]thiazolo[5,4-c]pyridin-5(4H)-yl]acetate | | 6 H), 1.13 (m, 4 H), 0.99 (d, J = 3.9 Hz, 2 H) | |
| 50 | 1-{trans-3-[4-(5-acetyl-4,5,6,7- | 441 | 7.78 (m, 2 H), 6.91 (d, J = 8.8 Hz, 2 H), 4.83 (quint, J = 5.8 Hz, | |
| | tetrahydro[1,3]thiazolo[5,4-c]pyridin-2- | | 1 H), 4.73 (m, 2 H), 3.79 (m, 2 H), 2.97 (m, 1 H), 2.89 (t, | |
| | yl)phenoxy]cyclobutyl}-N,N-dimethylpyrrolidin-3- | | J = 5.3 Hz, 1 H), 2.75 (m, 2 H), 2.64 (m, 2 H), 2.43 (m, 2 H), | |
| | amine | | 2.33 (m, 1 H), 2.14 (m, 5 H), 2.09 (s, 7 H), 1.88 (m, 1 H), | |
| | | | 1.62 (m, 1 H) | |
| 51 | 5-acetyl-2-(4-{[trans-3-(4-cyclopentylpiperazin-1- | 481 | 7.84 (m, 2 H), 6.97 (t, J = 9.3 Hz, 2 H), 4.95 (t, J = 6.3 Hz, 1 | 145.7-146.1 |
| | yl)cyclobutyl]oxy}phenyl)-4,5,6,7- | | H), 4.75 (m, 2 H), 4.12 (m, 1 H), 3.50 (m, 12 H), 2.95 (m, 2 H), | |
| | tetrahydro[1,3]thiazolo[5,4-c]pyridine | | 2.82 (m, 2 H), 2.37 (m, 1 H), 2.08 (m, 5 H), 1.71 (m, 4 H), | |
| | | | 1.57 (m, 2 H) | |
| 52 | 1-{2-oxo-2-[2-{4-[(trans-3-piperidin-1- | 470 | 7.79 (d, J = 8.5 Hz, 2 H), 6.91 (d, J = 8.8 Hz, 2 H), 6.13 (s, 1 | 209-210 |
| | ylcyclobutyl)oxy]phenyl}-6,7- | | H), 5.73 (m, 2 H), 4.78 (m, 1 H), 4.74 (s, 2 H), 3.97 (m, 2 H), | |
| | dihydro[1,3]thiazolo[5,4-c]pyridin-5(4H)- | | 3.79 (m, 2 H), 2.84 (m, 3 H), 2.36 (m, 2 H), 2.22 (m, 4 H), | |
| | yl]ethyl}urea | | 2.14 (m, 2 H), 1.50 (m, 4 H), 1.39 (d, J = 4.3 Hz, 2 H) | |
| 53 | 2-oxo-2-[2-{4-[(trans-3-piperidin-1- | 441 | 8.17 (m, 1 H), 7.83 (m, 3 H), 6.94 (m, 2 H), 4.92 (m, 1 H), | |
| | ylcyclobutyl)oxy]phenyl}-6,7- | | 4.78 (m, 2 H), 3.93 (m, 1 H), 3.86 (t, J = 5.8 Hz, 1 H), 3.78 (m, 2 | |
| | dihydro[1,3]thiazolo[5,4-c]pyridin-5(4H)- | | H), 3.41 (m, 3 H), 2.91 (m, 2 H), 2.77 (m, 5 H), 2.42 (m, 2 H), | |
| | yl]acetamide | | 1.87 (m, 2 H), 1.66 (m, 3 H), 1.38 (m, 1 H) | |
| 54 | 3-oxo-3-[2-{4-[(trans-3-piperidin-1- | 458 | 7.79 (d, J = 8.8 Hz, 2 H), 6.91 (d, J = 8.8 Hz, 2 H), | 167-168 |
| | ylcyclobutyl)oxy]phenyl}-6,7- | | 5.13 (m, 1H), 4.81 (m, 4 H), 4.43 (m, 1 H), 3.85 (m, 2 H), 3.53 (m, 2 | |
| | dihydro[1,3]thiazolo[5,4-c]pyridin-5(4H)- | | H), 2.85 (m, 3 H), 2.36 (m, 2 H), 2.24 (m, 4 H), 2.14 (m, 2 H), | |
| | yl]propane-1,2-diol | | 1.49 (m, 4 H), 1.36 (m, 2 H) | |
| 55 | 3-hydroxy-4-[2-{4-[(trans-3-piperidin-1- | 466 | 7.86 (d, J = 8.8 Hz, 2 H), 6.97 (d, J = 8.8 Hz, 2 H), 5.05 (m, 2 | >220 |
| | ylcyclobutyl)oxy]phenyl}-6,7- | | H), 4.94 (m, 1 H), 4.07 (m, 2 H), 3.96 (m, 1 H), 3.41 (d, | |
| | dihydro[1,3]thiazolo[5,4-c]pyridin-5(4H)- | | J = 11.8 Hz, 2 H), 3.00 (m, 2 H), 2.79 (m, 4 H), 2.45 (m, 2 H), | |
| | yl]cyclobut-3-ene-1,2-dione | | 1.89 (m, 2 H), 1.68 (m, 3 H), 1.39 (m, 1 H) | |
| 56 | 3-isopropoxy-4-[2-{4-[(trans-3-piperidin-1- | 508 | 7.80 (d, J = 8.5 Hz, 2 H), 6.91 (d, J = 8.8 Hz, 2 H), 5.29 (m, 1 | 157-159 |
| | ylcyclobutyl)oxy]phenyl}-6,7- | | H), 5.09 (m, 1 H), 4.85 (m, 1 H), 4.79 (m, 1 H), 4.11 (m, 1 H), | |
| | dihydro[1,3]thiazolo[5,4-c]pyridin-5(4H)- | | 3.87 (m, 1 H), 2.99 (m, 2 H), 2.88 (m, 1 H), 2.36 (m, 2 H), | |
| | yl]cyclobut-3-ene-1,2-dione | | 2.24 (m, 4 H), 2.12 (m, 2 H), 1.50 (m, 4 H), 1.36 (m, 8 H | |
| 57 | 5-acetyl-2-[4-({trans-3-[2-methylpyrrolidin-1- | 412 | 7.79 (m, 2 H), 6.91 (d, J = 8.8 Hz, 2 H), 4.81 (m, 1 H), | |
| | yl]cyclobutyl}oxy)phenyl]-4,5,6,7- | | 4.73 (m, 2 H), 3.79 (m, 2 H), 2.89 (m, 2 H), 2.77 (m, 1 H), 2.45 (m, | |
| | tetrahydro[1,3]thiazolo[5,4-c]pyridine, isomer A | | 2 H), 2.29 (m, 2 H), 2.11 (m, 4 H), 1.88 (m, 1 H), 1.65 (m, 2 | |
| | | | H), 1.32 (m, 1 H), 0.99 (m, 3 H) | |
| 58 | 3-amino-4-[2-{4-[(trans-3-piperidin-1- | 465 | 7.85 (m, 4 H), 6.96 (d, J = 8.8 Hz, 2 H), 5.02 (s, 2 H), 4.92 (t, J = | 196-201 |
| | ylcyclobutyl)oxy]phenyl}-6,7- | | 6.8 Hz, 1 H), 4.05 (m, 2 H), 3.93 (m, 1 H), 3.39 (d, J = 11.5 Hz, | |
| | dihydro[1,3]thiazolo[5,4-c]pyridin-5(4H)- | | 2 H), 2.95 (t, J = 5.3 Hz, 2 H), 2.74 (m, 5 H), 2.43 (m, 2 | |
| | yl]cyclobut-3-ene-1,2-dione.1/2 trifluoroacetate | | H), 1.85 (m, 2 H), 1.67 (m, 4 H), 1.38 (m, 1 H) | |
| 59 | 5-acetyl-2-[4-({trans-3-[2-methylpyrrolidin-1- | 412 | 7.79 (m, 2 H), 6.91 (m, 2 H), 4.81 (m, 1 H), 4.71 (m, 2 H), | |
| | yl]cyclobutyl}oxy)phenyl]-4,5,6,7- | | 3.78 (m, 2 H), 2.90 (m, 2 H), 2.77 (m, 1 H), 2.43 (m, 3 H), 2.27 (m, | |
| | tetrahydro[1,3]thiazolo[5,4-c]pyridine, isomer B | | 2 H), 2.13 (m, 4 H), 1.87 (m, 1 H), 1.64 (m, 2 H), 1.33 (m, 1 | |
| | | | H), 0.99 (d, J = 6.3 Hz, 3 H) | |
| 60 | (2S)-3-oxo-3-[2-{4-[(trans-3-piperidin-1- | 458 | 7.79 (d, J = 8.5 Hz, 2 H), 6.91 (d, J = 8.8 Hz, 2 H), 5.11 (m, 1 | |
| | ylcyclobutyl)oxy]phenyl}-6,7- | | H), 4.81 (m, 4 H), 4.44 (m, 1 H), 3.91 (m, 2 H), 3.54 (m, 2 H), | |
| | dihydro[1,3]thiazolo[5,4-c]pyridin-5(4H)- | | 2.84 (m, 3 H), 2.27 (m, 8 H), 1.51 (m, 4 H), 1.40 (d, J = 4.3 Hz, | |
| | yl]propane-1,2-diol | | 2 H) | |
| 61 | 5-acetyl-2-{4-[(cis-3-piperidin-1- | 395 | 7.76 (dd, J = 8.8, 2.5 Hz, 2 H), 6.89 (m, 2 H), 4.47 (m, 3 H), | |
| | ylcyclobutyl)oxy]phenyl}-4,5,6,7-tetrahydro-3H- | | 3.77 (t, J = 5.5 Hz, 1 H), 3.70 (t, J = 5.5 Hz, 1 H), 2.73 (s, 1 | |
| | imidazo[4,5-c]pyridine | | H), 2.65 (m, 3 H), 2.36 (m, 1 H), 2.19 (m, 4 H), 2.11 (m, 3 H), | |
| | | | 1.80 (m, 2 H), 1.47 (m, 4 H), 1.38 (d, J = 4.5 Hz, 2 H) | |
| 62 | 5-acetyl-2-{4-[(trans-3-piperidin-1- | 395 | 12.14 (m, 1 H), 7.76 (m, 2 H), 6.85 (m, 2 H), 4.75 (m, 1 H), | |
| | ylcyclobutyl)oxy]phenyl}-4,5,6,7-tetrahydro-3H- | | 4.47 (m, 2 H), 3.73 (m, 2 H), 2.87 (d, J = 3.8 Hz, 1 H), | |
| | imidazo[4,5-c]pyridine | | 2.68 (m, 2 H), 2.23 (m, 11 H), 1.50 (m, 4 H), 1.39 (d, J = 4.8 Hz, 2 | |
| | | | H) | |
| 63 | 2-oxo-2-[2-{4-[(trans-3-piperidin-1- | 428 | 7.79 (d, J = 8.8 Hz, 2 H), 6.91 (d, J = 8.8 Hz, 2 H), | 155.3 |
| | ylcyclobutyl)oxy]phenyl}-6,7- | | 4.57-4.82 (m, 4 H), 4.21 (m, 2 H), 3.64-3.93 (m, 2 H), 2.89 (m, 3 H), | |
| | dihydro[1,3]thiazolo[4,5-c]pyridin-5(4H)-yl]ethanol | | 2.25 (m, 8 H), 1.49 (m, 4 H), 1.39 (d, J = 4.3 Hz, 2 H) | |
| 64 | 2-{4-[(trans-3-piperidin-1-ylcyclobutyl)oxy]phenyl}- | 413 | 7.78 (d, J = 8.8 Hz, 2 H), 6.91 (d, J = 8.8 Hz, 2 H), 6.17 (s, 2 | 214.3 |
| | 6,7-dihydro[1,3]thiazolo[4,5-c]pyridine-5(4H)- | | H), 4.78 (m, 1 H), 4.53 (m, 2 H), 3.66 (m, 2 H), 2.78-2.92 (m, | |
| | carboxamide | | 3 H), 2.10-2.42 (m, 8 H), 1.52 (m, 4 H), 1.41 (m, 2 H) | |
| 65 | 3-oxo-3-[2-{4-[(trans-3-piperidin-1- | 455 | 7.79 (d, J = 8.8 Hz, 2 H), 7.52 (s, 1 H), 7.05 (m, 1 H), 6.91 (d, | 183.4 |
| | ylcyclobutyl)oxy]phenyl}-6,7- | | J = 8.5 Hz, 2 H), 4.78 (m, 1 H), 4.62-4.73 (m, 2 H), 3.80 (m, 2 | |
| | dihydro[1,3]thiazolo[4,5-c]pyridin-5(4H)- | | H), 3.38-3.41 (m, 2 H), 2.79-2.97 (m, 3 H), 2.36 (m, 2 H), | |
| | yl]propanamide | | 2.17 (m, 6 H), 1.52 (m, 4 H), 1.38 (m, 2 H) | |
| 66 | 2-oxo-2-[2-{4-[(trans-3-piperidin-1- | 428 | 7.77 (d, J = 8.4 Hz, 2 H), 6.82 (d, J = 8.4 Hz, 2 H), 4.80 (m, 3 | |
| | ylcyclobutyl)oxy]phenyl}-6,7- | | H), 4.04 (m, 2 H), 3.83 (m, 1 H), 3.00 (m, 1 H), 2.86 (t, J = 6.2 Hz, | |
| | dihydro[1,3]thiazolo[4,5-b]pyridin-4(5H)-yl]ethanol | | 2 H), 2.42 (m, 2 H), 2.29 (m, 6 H), 2.07 (m, 2 H), 1.61 (m, | |
| | | | 4 H), 1.47 (m, 2 H) | |
| 67 | 4-acetyl-2-{4-[(trans-3-piperidin-1- | 410 | 7.85 (d, J = 8.5 Hz, 2 H), 6.93 (d, J = 8.8 Hz, 2 H), 4.80 (m, 1 | |
| | ylcyclobutyl)oxy]phenyl}-5,6,7,8-tetrahydro-4H- | | H), 3.63 (m, 2 H), 2.91 (m, 1 H), 2.63 (m, 2 H), 2.27 (m, 8 H), | |
| | [1,3]oxazolo[5,4-b]azepine | | 2.08 (s, 3 H), 1.75 (s, 2 H), 1.64 (d, J = 4.5 Hz, 2 H), 1.52 (s, 4 | |
| | | | H), 1.40 (dd, J = 1.8, 1.0 Hz, 2 H) | |
| 68 | 2-{4-[(trans-3-piperidin-1-ylcyclobutyl)oxy]phenyl}- | 464 | 7.80 (d, J = 8.5 Hz, 2 H), 6.97 (d, J = 8.5 Hz, 2 H), 4.80 (s, 1 | |
| | 4-(trifluoroacetyl)-5,6,7,8-tetrahydro-4H- | | H), 3.82 (m, 2 H), 2.88 (m, 1 H), 2.68 (t, J = 5.5 Hz, 2 H), | |
| | [1,3]oxazolo[5,4-b]azepine | | 2.28 (m, 8 H), 1.91 (s, 2 H), 1.70 (m, 2 H), 1.45 (m, 6 H) | |
| 69 | 2-{4-[(trans-3-piperidin-1-ylcyclobutyl)oxy]phenyl}- | 349 | 13.17 (m, 1 H), 8.89 (s, 1 H), 8.28 (d, J = 5.5 Hz, 1 H), | 165-172 |
| | 3H-imidazo[4,5-c]pyridine | | 8.14 (d, J = 8.8 Hz, 2 H), 7.01 (d, J = 8.8 Hz, 2 H), 4.83 (m, 1 H), | |
| | | | 2.89 (t, J = 6.5 Hz, 1 H), 2.40 (m, 2 H), 2.19 (m, 6 H), 1.51 (m, | |
| | | | 4 H), 1.40 (d, J = 4.5 Hz, 2 H) | |

### EXAMPLE 21

### Affinity for the Histamine H₃-Receptor; Inverse Agonism, Antagonism and Agonism Activity: [³⁵S]GTPγS-Binding Assay Human Histamine H₃-Receptor

Material and Methods

Reagents

Reagents and reference compounds are of analytical grade and may be obtained from various commercial sources. [³H]-N-α-methylhistamine (80-85 Ci/mmol) and [³⁵S]-GTPγS (1250 Ci/mmol) are purchased from Perkin Elmer (Belgium). Cell culture reagents are purchased from Cambrex (Belgium).

Test and reference compounds are dissolved in 100% DMSO to give a 1 mM stock solution. Final DMSO concentration in the assay does not exceed 1%.

A CHO cell line expressing the unspliced full length (445 AA) human H₃ histamine receptor may be obtained e.g. from Euroscreen S.A. (Belgium).

Cell Culture

Cells are grown in HAM-F12 culture media containing 10% fetal bovine serum, 100 IU/ml penicillin, 100 μg/ml streptomycin, 1% sodium pyruvate and 400 μg/ml of gentamycin. Cells are maintained at 37° C. in a humidified atmosphere composed of 95% air and 5% CO₂.

Membrane Preparation

Confluent cells are detached by 10 min incubation at 37° C. in PBS/EDTA 0.02%. The cell suspension is centrifuged at 1,500×g for 10 min at 4° C. The pellet is homogenized in a 15 mM Tris-HCl buffer (pH 7.5) containing 2 mM MgCl₂, 0.3 mM EDTA, 1 mM EGTA (buffer A). The crude homogenate is frozen in liquid nitrogen and thawed. DNAse (1 μl/ml) is then added and the homogenate is further incubated for 10 min at 25° C. before being centrifuged at 40,000×g for 25 min at 4° C. The pellet is resuspended in buffer A and washed once more under the same conditions. The final membrane pellet is resuspended, at a protein concentration of 1-3 mg/ml, in a 7.5 mM Tris-HCl buffer (pH 7.5) enriched with 12.5 mM MgCl₂, 0.3 mM EDTA, 1 mM EGTA and 250 mM sucrose and stored in liquid nitrogen until used.

Binding Assays

[³H]-N-α-methylhistamine Binding Assay

Affinity of compounds for human histamine H₃ receptors may be measured by competition with [³H]-N-α-methylhistamine. This binding assay may be performed on any H3 sequence, human or non-human. Briefly, membranes (20-40 μg proteins) expressing human H₃ histamine receptors are incubated at 25° C. in 0.5 ml of a 50 mM Tris-HCl buffer (pH 7.4) containing 2 mM MgCl₂, 0.2 nM [³H]-N-α-methyl-histamine and increasing concentrations of drugs. The non specific binding (NSB) is defined as the residual binding observed in the presence of 10 μM thioperamide or histamine. Membrane-bound and free radioligand are separated by rapid filtration through glass fiber filters presoaked in 0.1% PEI. Samples and filters are rinsed by at least 6 ml of ice-cold 50 mM Tris-HCl buffer (pH 7.4). The entire filtration procedure does not exceed 10 seconds per sample. Radioactivity trapped onto the filters is counted by liquid scintillation in a β-counter.

[³⁵S]-GTPγS Binding Assay

Stimulation (agonist) or inhibition (inverse agonist) of [³⁵S]-GTPγS binding to membrane expressing human H₃ histamine receptors is measured as described by Lorenzen et al. (Mol. Pharmacol. 1993, 44, 115-123) with a few modifications. Briefly, membranes (10-20 μg proteins) expressing human H₃ histamine receptors are incubated at 25° C. in 0.2 ml of a 50 mM Tris-HCl buffer (pH 7.4) containing 3 mM MgCl₂, 50 mM NaCl, 1 μM GDP, 2 μg saponin and increasing concentrations of drugs. After 15 min pre-incubation, 0.2 nM of [³⁵S]-GTPγS are added to the samples. The non specific binding (NSB) is defined as the residual binding observed in the presence of 100 μM Gpp (NH)p. Membrane-bound and free radioligand are separated by rapid filtration through glass fiber filters. Samples and filters are rinsed by at least 6 ml of ice-cold 50 mM Tris-HCl buffer (pH 7.4). The entire filtration procedure does not exceed 10 seconds per sample. Radioactivity trapped onto the filters is counted by liquid scintillation in a β-counter.

Data Analysis

Determination of pIC₅₀/pKi/pEC₅₀/pEC₅₀INV

Analysis

Raw data are analyzed by non-linear regression using XLfit™ (IDBS, United Kingdom) according to the following generic equation

B=MIN+[(MAX−MIN)/(1+(((10^{x})/(10^{-pX50}))^{nH}))]

where:

B is the radioligand bound in the presence of the unlabelled compound (dpm),

MIN is the minimal binding observed (dpm)

MAX is maximal binding observed (dpm),

X is the concentration of unlabelled compound (log M),

pX₅₀ (−log M) is the concentration of unlabelled compound causing 50% of its maximal effect (inhibition or stimulation of radioligand binding). It stands for pIC₅₀ when determining the affinity of a compound for the receptor in binding studies with [3H]-N-α-methylhistamine, for pEC₅₀ for compounds stimulating the binding of [³⁵S]-GTPγS (agonists) and for pEC₅₀INV for compounds inhibiting the binding of [³⁵S]-GTPγS (inverse agonists).

n_{H} is the Hill coefficient.

pKi may be obtained by applying the following equation (Cheng and Prusoff, 1973, Biochem. Pharmacol., 22: 3099-3108):

pKi=pIC₅₀+log(1+L/Kd)

where:

pKi is the unlabelled compound equilibrium dissociation constant (−log M),

L is the radioligand concentration (nM),

Kd is the radioligand equilibrium dissociation constant (nM).

Compounds of formula (I) according to the invention show pIC₅₀ values of at least 6.5, preferably greater than 7.5 for the histamine H₃ receptor.

Compounds of formula (I) according to the invention showed pEC₅₀INV values typically greater than 7.5 for the histamine H₃ receptor.

### EXAMPLE 22

### Antagonism Activity: Paced Isolated Guinea Pig Myenteric Plexus—Electric-Field Stimulation Assay

Material and Methods

Reagents

Stock solutions (10⁻² M) of compounds to be tested and further dilutions are freshly prepared in DMSO (WNR, Leuven, Belgium). All other reagents (R(−)-α-methylhistamine, mepyramine, ranitidine, propranolol, yohimbine and components of the Krebs' solution) are of analytical grade and obtained from conventional commercial sources.

Animals

Four week-old male Dunkin-Hartley guinea pigs (200-300 g) are supplied by Charles River (Sultfeld, Germany). All animals are ordered and used under protocol “orgisol-GP” approved by the UCB Pharma ethical committee. Animals are housed in the UCB animal facility in groups of 12, in stainless steel cages (75×50×30 cm) and allowed to acclimatise for a minimum of one week before inclusion in the study. Room temperature is maintained between 20 and 24° C. with 40 to 70% relative humidity. A light and dark cycle of 12 h is applied. Animals have free access to food and water.

Organ Preparation

The method is adapted from that described by Menkveld et al. in Eur. J. Pharmacol. 1990, 186, 343-347. Longitudinal myenteric plexus is prepared from the isolated guinea pig ileum. Tissues are mounted in 20-ml organ baths containing modified Krebs' solution with 10⁻⁷ M mepyramine, 10⁻⁵ M ranitidine, 10⁻⁵ M propranolol and 10⁻⁶ M yohimbine. The bathing solution is maintained at 37° C. and gassed with 95% O₂-5% CO₂. Tissues are allowed to equilibrate for a 60-min period under a resting tension of 0.5 g and an electrical field stimulation (pulses of 5-20 V, 1 ms and 0.1 Hz is applied during the whole experiment). Such a stimulation induces stable and reproductive twitch contractions. Isometric contractions are measured by force-displacement transducers coupled to an amplifier connected to a computer system (EMKA Technologies) capable of controlling (i) automatic data acquisition, (ii) bath washout by automatic fluid circulation through electrovalves at predetermined times or signal stability and (iii) automatic dilution/injection of drug in the bath at predetermined times or signal stability.

Protocol

After a 60 min-stabilisation period, tissues are stimulated twice with 10⁻⁶ M R(−)-α-methylhistamine at 30-min interval. After a 60-min incubation period in the presence of solvent or antagonist test compound, a cumulative concentration-response to R(−)-α-methylhistamine is elicited (10⁻¹⁰ á 10⁻⁴ M). Only one concentration of antagonist is tested on each tissue.

Data Analysis

An appropriate estimate of interactions between agonist and antagonist can be made by studying the family of curves observed in the absence or presence of increasing antagonist concentrations. The value of each relevant parameter of each concentration-response curve (pD₂ and Eₘₐₓ) is calculated by an iterative computer software (XLfit, IDBS, Guildford, UK) fitting the experimental data to the four parameter logistic equation. Antagonistic activity of the test substance is estimated by the calculation of pD′₂ and/or pA₂ values according to the methods described by Van Rossum et al. in Arch. Int. Pharmacodyn. Ther. 1963, 143, 299 and/or by Arunlakshana & Schild in Br. J. Pharmacol 1959, 14, 48

Results are expressed as the mean±SD. The number of observations is indicated as n.

Compounds of formula (I) according to the invention showed pA₂ values typically greater than or equal to 7.5 for the histamine H₃ receptor.

### EXAMPLE 23

### hERG Study

This is an in vitro electrophysiological patch clamp study to assess the potential effects of test compounds on human ether-a-go-go-related gene (hERG)-encoded channel tail current recorded from HEK293 cells stably transfected with hERG cDNA. Coverslips on which cells are seeded are mounted in a recording chamber and superfused with physiological saline. Recordings of tail current are made in the voltage patch clamp mode. A reference substance e.g. E-4031 is used to confirm that the current observed can be inhibited by a known hERG channel blocker (Zhou, Z. et al., Biophys. J., 1998, 74, 230-241).

Compounds of the current invention typically show weak hERG channel affinities. Generally, the hERG channel affinity of compounds of formula (I) is greater than or equal to 1 μM.

### EXAMPLE 24

### Brain H₃ Receptors Occupancy

Material and Methods

Reagents

[³H]-N-α-methylhistamine (80-85 Ci/mmol) is purchased from Perkin Elmer (Belgium). Reagents and reference compounds used for binding assay on cerebral cortical tissues are of analytical grade and obtained from various commercial sources. Reference compounds are dissolved in 100% dimethylsulfoxide (DMSO) to give a 1 mM stock solution. Final DMSO concentration in the assay does not exceed 1%.

Animals and Treatments

Experimental procedures involving animals are conducted in compliance with the local ethics committee for animal experimentation according to Belgian law. Young male SPF Sprague-Dawley rats (OFA origin, supplied by IFFA CREDO, Belgium) weighting 200-300 g are used. Animals receive vehicle or the test compound by the i.p. route of administration. Compounds are all dissolved in a mixture of methyl cellulose (MC) 1% and DMSO 5%. A dose-volume of 5 ml/kg body weight is used. Control groups receive an equivalent dose-volume of MC 1%/DMSO 5%. Animals are killed one or three hours later. Terminal blood samples are collected and brains rapidly removed. Cerebral cortex are dissected on ice at 4° C.

Membrane Preparation

Cerebral cortex tissues are rapidly homogenized in 2.5 volumes of ice-cold buffer containing 50 mM Tris-HCl and 250 mM sucrose (pH 7.4). Homogenates are frozen in liquid nitrogen and stored at −80° C. until use.

³H]-N-α-Methylhistamine Binding Assay

³H]-N-α-methylhistamine binding assay is carried out in 50 mM Tris-HCl buffer (pH 7.4) containing 2 mM MgCl₂. Briefly, homogenates are thawed and incubated for 15 minutes at room temperature before use. Homogenates (500 μg of proteins) are incubated at 25° C. during 5 minutes in 0.2 ml of buffer and 0.2 nM [³H]-N-α-methylhistamine. Non specific binding (NSB) is defined as the residual binding observed in the presence of 10 μM thioperamide. Membrane-bound and free radioligand are separated by rapid filtration through glass fiber filters (GF/C) (pre-soaked in 0.1% PEI). Samples and filters are rinsed by 8 ml of ice-cold 50 mM Tris-HCl buffer (pH 7.4). The entire filtration procedure does not exceed 10 seconds per sample. Radioactivity trapped onto the filters is counted by liquid scintillation in a β-counter. Protein concentrations are determined using the BCA Pierce method with bovine serum albumin as a standard.

Data Analysis

Percentage of receptor occupancy was defined as:

1−((B-NSB)_{(treated animals)}/(B-NSB)_{(control animals)}))*100

wherein B is the radioligand bound (dpm) and NSB is the non specific binding.

IC₅₀ values (dose required to produce a 50% reduction in ex vivo radioligand binding) are determined by plotting and analyzing the log₁₀ of the i.p. dose against % specific binding by non-linear regression using GraphPad Prism 4 software (GraphPad Inc., San Diego, USA) according to the following generic equation:

Y=MIN+(MAX−MIN)/(1+10^{(LogIC}^{₅₀}^{-X)*nH)})

wherein Y is the response, X is the logarithm of the concentration, MIN is the minimal binding observed (dpm), MAX is maximal binding observed (dpm) and nH is the Hill coefficient.

Preferred compounds of formula (I) according to the present invention typically show a percentage of receptor occupancy generally greater than or equal to 70% at a dose of 1 mg/kg ip.

1. A compound of formula (I), geometrical isomers, enantiomers, diastereoisomers, pharmaceutically acceptable salts and all possible mixtures thereof, 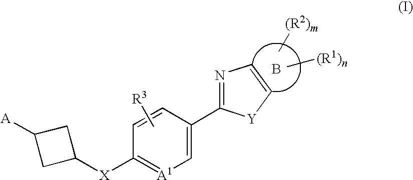
wherein
A is a substituted or unsubstituted aliphatic or cyclic amino group which is linked to the cyclobutyl group via an amino nitrogen;
A¹ is CH, C-halogen or N;
B is selected from the group consisting of heteroaryl, 5-8-membered heterocycloalkyl and 5-8-membered cycloalkyl;
X is O, S, NH or N(C₁₋₄ alkyl);
Y is O, S, or NH;
R¹ is selected from the group consisting of sulfonyl, amino, C₁₋₆ alkyl, C₂₋₆ alkenyl, C₂₋₆ alkynyl, aryl, heteroaryl, C₃₋₈ cycloalkyl, 3-8-membered heterocycloalkyl, acyl, C₁₋₆-alkyl aryl, C₁₋₆-alkyl heteroaryl, C₂₋₆-alkenyl aryl, C₂₋₆-alkenyl heteroaryl, C₂₋₆-alkynyl aryl, C₂₋₆-alkynyl heteroaryl, C₁₋₆-alkyl cycloalkyl, C₁₋₆-alkyl heterocycloalkyl, C₂₋₆-alkenyl cycloalkyl, C₂₋₆-alkenyl heterocycloalkyl, C₂₋₆-alkynyl cycloalkyl, C₂₋₆-alkynyl heterocycloalkyl, alkoxycarbonyl, aminocarbonyl, C₁₋₆-alkyl carboxy, C₁₋₆-alkyl acyl, aryl acyl, heteroaryl acyl, C₃₋₈-(hetero)cycloalkyl acyl, C₁₋₆-alkyl acyloxy, C₁₋₆-alkyl alkoxy, C₁₋₆-alkyl alkoxycarbonyl, C₁₋₆-alkyl aminocarbonyl, C₁₋₆-alkyl acylamino, acylamino, acylaminocarbonyl, ureido, C₁₋₆-alkyl ureido, C₁₋₆-alkyl carbamate, C₁₋₆-alkyl amino, C₁₋₆-alkyl sulfonyloxy, C₁₋₆-alkyl sulfonyl, C₁₋₆-alkyl sulfinyl, C₁₋₆-alkyl sulfanyl, C₁₋₆-alkyl sulfonylamino, aminosulfonyl, C₁₋₆-alkyl aminosulfonyl, hydroxy, C₁₋₆-alkyl hydroxy, phosphonate, C₁₋₆-alkyl phosphonate, C₁₋₆-alkyl phosphono, halogen, cyano, carboxy, oxo, and thioxo;
n is equal to 0, 1, 2 or 3;
R² is selected from the group consisting of hydrogen, sulfonyl, amino, C₁₋₆ alkyl, C₂₋₆ alkenyl, C₂₋₆ alkynyl, aryl, heteroaryl, C₃₋₈ cycloalkyl, 3-8-membered heterocycloalkyl, acyl, C₁₋₆-alkyl aryl, C₁₋₆-alkyl heteroaryl, C₂₋₆-alkenyl aryl, C₂₋₆-alkenyl heteroaryl, C₂₋₆-alkynyl aryl, C₂₋₆-alkynyl heteroaryl, C₁₋₆-alkyl cycloalkyl, C₁₋₆-alkyl heterocycloalkyl, C₂₋₆-alkenyl cycloalkyl, C₂₋₆-alkenyl heterocycloalkyl, C₂₋₆-alkynyl cycloalkyl, C₂₋₆-alkynyl heterocycloalkyl, alkoxycarbonyl, aminocarbonyl, C₁₋₆-alkyl carboxy, C₁₋₆-alkyl acyl, aryl acyl, heteroaryl acyl, C₃₋₈-(hetero)cycloalkyl acyl, C₁₋₆-alkyl acyloxy, C₁₋₆-alkyl alkoxy, C₁₋₆-alkyl alkoxycarbonyl, C₁₋₆-alkyl aminocarbonyl, C₁₋₆-alkyl acylamino, acylamino, acylaminocarbonyl, ureido, C₁₋₆-alkyl ureido, C₁₋₆-alkyl carbamate, C₁₋₆-alkyl amino, C₁₋₆-alkyl sulfonyloxy, C₁₋₆-alkyl sulfonyl, C₁₋₆-alkyl sulfinyl, C₁₋₆-alkyl sulfanyl, C₁₋₆-alkyl sulfonylamino, aminosulfonyl, C₁₋₆-alkyl aminosulfonyl, hydroxy, C₁₋₆-alkyl hydroxy, phosphonate, C₁₋₆-alkyl phosphonate, substituted or unsubstituted C₁₋₆-alkylphosphono, halogen, cyano, carboxy, oxo, and thioxo;
m is equal to 0 or 1; and
R³ is hydrogen or C₁₋₆ alkyl or halogen or C₁₋₆ alkoxy.

2. A compound according to claim 1 wherein A¹ is CH, C—F or N.

3. A compound according to claim 1 wherein n is equal to 0.

4. A compound according to claim 1 wherein R² is selected from the group consisting of hydrogen, carboxy, acyl, substituted or unsubstituted C₃₋₈ cycloalkyl, alkoxycarbonyl, substituted or unsubstituted C₁₋₆-alkyl alkoxycarbonyl, aminocarbonyl, substituted or unsubstituted C₁₋₆-alkyl aminocarbonyl, aminosulfonyl, substituted or unsubstituted C₁₋₆-alkyl hydroxy, substituted or unsubstituted C₁₋₆-alkyl phosphonate, and substituted or unsubstituted C₁₋₆-alkyl phosphono.

5. A compound according to claim 1 wherein X is O.

6. A compound according to claim 1 wherein A is a 3 to 8 membered heterocycloalkyl linked to the cyclobutyl group via a nitrogen atom.

7. A compound according claim 6 wherein A is selected from the group consisting of substituted or unsubstituted piperidin-1-yl, substituted or unsubstituted morpholin-4-yl, substituted or unsubstituted pyrrolidin-1-yl, substituted or unsubstituted piperazin-1-yl, substituted or unsubstituted azepan-1-yl, and substituted or unsubstituted thiomorpholin-4-yl.

8. A compound according to claim 1 wherein B is a substituted or unsubstituted 5, 6 or 7-membered cycloalkyl, a substituted or unsubstituted 5, 6 or 7-membered heterocycloalkyl, or a substituted or unsubstituted heteroaryl selected from the group comprising or consisting of a tetrahydropyridyl, a tetrahydro-1H-azepinyl, a cyclopentenyl, and a pyridyl.

9. A compound according to claim 1 wherein B is a substituted or unsubstituted 5, 6 or 7-membered cycloalkyl, a substituted or unsubstituted 5, 6 or 7-membered heterocycloalkyl, or a substituted or unsubstituted heteroaryl and forms together with the oxazole, the thiazole or the imidazole ring a fused heterocycle selected from the group consisting of 4,5,6,7-tetrahydro[1,3]thiazolo[5,4-c]pyridine, 4,5,6,7-tetrahydro[1,3]thiazolo[4,5-b]pyridine, 4,5,6,7-tetrahydro[1,3]thiazolo[4,5-c]pyridine, 4,5,6,7-tetrahydro[1,3]oxazolo[4,5-c]pyridine, 4,5,6,7-tetrahydro-3H-imidazo[4,5-c]pyridine, 5,6,7,8-tetrahydro-4H-[1,3]thiazolo[5,4-b]azepine, 5,6,7,8-tetrahydro-4H-[1,3]oxazolo[5,4-b]azepine, 5,6-dihydro-4H-cyclopenta[d][1,3]thiazole, 3H-imidazo[4,5-c]pyridine, and [1,3]thiazolo[4,5-c]pyridine.

10. A compound of formula (If) according to claim 1 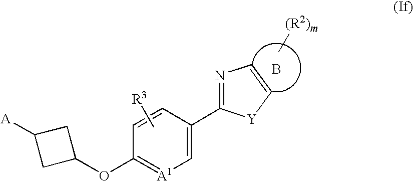
wherein
A is a 3 to 8 membered heterocycloalkyl linked to the cyclobutyl group via a nitrogen atom;
A¹ is CH;
Y is O, S or NH;
B is a substituted or unsubstituted 5, 6 or 7-membered cycloalkyl, a substituted or unsubstituted 5, 6 or 7-membered heterocycloalkyl, or a substituted or unsubstituted heteroaryl selected from the group consisting of a tetrahydropyridyl, a tetrahydro-1H-azepinyl, a cyclopentenyl, and a pyridyl;
R² is selected from the group consisting of hydrogen, carboxy, acyl, substituted or unsubstituted C₃₋₈ cycloalkyl, alkoxycarbonyl, substituted or unsubstituted C₁₋₆-alkyl alkoxycarbonyl, aminocarbonyl, substituted or unsubstituted C₁₋₆-alkyl aminocarbonyl, aminosulfonyl, substituted or unsubstituted C₁₋₆-alkyl hydroxy, substituted or unsubstituted C₁₋₆-alkyl phosphonate, and substituted or unsubstituted C₁₋₆-alkyl phosphono;
m is equal to 0 or 1; and
R³ is hydrogen or halogen.

11. A compound according to claim 1 wherein A is piperidin-1-yl, 2-methylpyrrolidin-1-yl, (2S)-2-methylpyrrolidin-1-yl or (2R)-2-methylpyrrolidin-1-yl.

12. A compound according to claim 1 wherein Y is S.

13. A compound according to claim 1 wherein R² is selected from the group consisting of acetyl, aminocarbonyl, hydroxyacetyl, 2-amino-2-oxoethyl and amino(oxo)acetyl.

14. A compound selected from the group consisting of:
2-{4-[(trans-3-piperidin-1-ylcyclobutyl)oxy]phenyl}-5,6-dihydro-4H-cyclopenta[d][1,3]thiazole-5-carboxylic acid;
5-(methoxyacetyl)-2-{4-[(trans-3-piperidin-1-ylcyclobutyl)oxy]phenyl}-4,5,6,7-tetrahydro[1,3]thiazolo[5,4-c]pyridine;
tert-butyl 2-{4-[(trans-3-piperidin-1-ylcyclobutyl)oxy]phenyl}-6,7-dihydro[1,3]thiazolo[5,4-c]pyridine-5(4H)-carboxylate;
5-acetyl-2-{4-[(trans-3-piperidin-1-ylcyclobutyl)oxy]phenyl}-4,5,6,7-tetrahydro[1,3]thiazolo[5,4-c]pyridine;
2-{4-[(trans-3-piperidin-1-ylcyclobutyl)oxy]phenyl}-4,5,6,7-tetrahydro[1,3]thiazolo[5,4-c]pyridine;
5-(morpholin-4-ylcarbonyl)-2-{4-[(trans-3-piperidin-1-ylcyclobutyl)oxy]phenyl}-4,5,6,7-tetrahydro[1,3]thiazolo[5,4-c]pyridine;
5-(morpholin-4-ylsulfonyl)-2-{4-[(trans-3-piperidin-1-ylcyclobutyl)oxy]phenyl}-4,5,6,7-tetrahydro[1,3]thiazolo[5,4-c]pyridine;
5-acetyl-2-{4-[(trans-3-morpholin-4-ylcyclobutyl)oxy]phenyl}-4,5,6,7-tetrahydro[1,3]thiazolo[5,4-c]pyridine;
2-oxo-2-[2-{4-[(trans-3-piperidin-1-ylcyclobutyl)oxy]phenyl}-6,7-dihydro[1,3]thiazolo[5,4-c]pyridin-5(4H)-yl]ethanamine;
2-{4-[(trans-3-piperidin-1-ylcyclobutyl)oxy]phenyl}-6,7-dihydro[1,3]thiazolo[5,4-c]pyridine-5(4H)-carboxamide;
2-oxo-2-[2-{4-[(trans-3-piperidin-1-ylcyclobutyl)oxy]phenyl}-6,7-dihydro[1,3]thiazolo[5,4-c]pyridin-5(4H)-yl]ethanol;
5-acetyl-2-(4-{[trans-3-(4-isopropylpiperazin-1-yl)cyclobutyl]oxy}phenyl)-4,5,6,7-tetrahydro[1,3]thiazolo[5,4-c]pyridine;
5-acetyl-2-(4-{[trans-3-(4,4-difluoropiperidin-1-yl)cyclobutyl]oxy}phenyl)-4,5,6,7-tetrahydro[1,3]thiazolo[5,4-c]pyridine;
5-acetyl-2-{4-[(trans-3-pyrrolidin-1-ylcyclobutyl)oxy]phenyl}-4,5,6,7-tetrahydro[1,3]thiazolo[5,4-c]pyridine;
3-[2-{4-[(trans-3-piperidin-1-ylcyclobutyl)oxy]phenyl}-6,7-dihydro[1,3]thiazolo[5,4-c]pyridin-5(4H)-yl]propane-1,2-diol;
(2S)-3-[2-{4-[(trans-3-piperidin-1-ylcyclobutyl)oxy]phenyl}-6,7-dihydro[1,3]thiazolo[5,4-c]pyridin-5(4H)-yl]propane-1,2-diol;
(2R)-3-[2-{4-[(trans-3-piperidin-1-ylcyclobutyl)oxy]phenyl}-6,7-dihydro[1,3]thiazolo[5,4-c]pyridin-5(4H)-yl]propane-1,2-diol;
2-[2-{4-[(trans-3-piperidin-1-ylcyclobutyl)oxy]phenyl}-6,7-dihydro[1,3]thiazolo[5,4-c]pyridin-5(4H)-yl]acetamide;
5-acetyl-2-{4-[(trans-3-azepan-1-ylcyclobutyl)oxy]phenyl}-4,5,6,7-tetrahydro[1,3]thiazolo[5,4-c]pyridine;
(3R)-1-{trans-3-[4-(5-acetyl-4,5,6,7-tetrahydro[1,3]thiazolo[5,4-c]pyridin-2-yl)phenoxy]cyclobutyl}-N,N-dimethylpyrrolidin-3-amine;
N-ethyl-2-{4-[(trans-3-piperidin-1-ylcyclobutyl)oxy]phenyl}-6,7-dihydro[1,3]thiazolo[5,4-c]pyridine-5(4H)-carboxamide;
5-acetyl-2-{4-[(trans-3-thiomorpholin-4-ylcyclobutyl)oxy]phenyl}-4,5,6,7-tetrahydro[1,3]thiazolo[5,4-c]pyridine;
5-acetyl-2-{4-[(trans-3-piperidin-1-ylcyclobutyl)thio]phenyl}-4,5,6,7-tetrahydro[1,3]thiazolo[5,4-c]pyridine;
cis-3-[2-{4-[(trans-3-piperidin-1-ylcyclobutyl)oxy]phenyl}-6,7-dihydro[1,3]thiazolo[5,4-c]pyridin-5(4H)-yl]cyclobutanol;
3-oxo-3-[2-{4-[(trans-3-piperidin-1-ylcyclobutyl)oxy]phenyl}-6,7-dihydro[1,3]thiazolo[5,4-c]pyridin-5(4H)-yl]propanamide;
methyl[2-{4-[(trans-3-piperidin-1-ylcyclobutyl)oxy]phenyl}-6,7-dihydro[1,3]thiazolo[5,4-c]pyridin-5(4H)-yl]acetate;
diethyl {[2-{4-[(trans-3-piperidin-1-ylcyclobutyl)oxy]phenyl}-6,7-dihydro[1,3]thiazolo[5,4-c]pyridin-5(4H)-yl]methyl}phosphonate;
5-acetyl-2-{4-[(trans-3-piperidin-1-ylcyclobutyl)oxy]phenyl}-4,5,6,7-tetrahydro[1,3]oxazolo[4,5-c]pyridine;
5-acetyl-2-{4-[(trans-3-piperidin-1-ylcyclobutyl)oxy]phenyl}-4,5,6,7-tetrahydro[1,3]thiazolo[4,5-c]pyridine;
4-acetyl-2-{4-[(trans-3-piperidin-1-ylcyclobutyl)oxy]phenyl}-4,5,6,7-tetrahydro[1,3]thiazolo[4,5-b]pyridine;
4-acetyl-2-{4-[(trans-3-piperidin-1-ylcyclobutyl)oxy]phenyl}-5,6,7,8-tetrahydro-4H-[1,3]thiazolo[5,4-b]azepine;
2-{4-[(trans-3-piperidin-1-ylcyclobutyl)oxy]phenyl}[1,3]thiazolo[4,5-c]pyridine;
2-{4-[(trans-3-piperidin-1-ylcyclobutyl)oxy]phenyl}-5-(3,3,3-trifluoropropanoyl)-4,5,6,7-tetrahydro[1,3]thiazolo[5,4-c]pyridine;
{[2-{4-[(trans-3-piperidin-1-ylcyclobutyl)oxy]phenyl}-6,7-dihydro[1,3]thiazolo[5,4-c]pyridin-5(4H)-yl]methyl}phosphonic acid;
5-[(5-methyl-2H-1,2,3-triazol-4-yl)carbonyl]-2-{4-[(trans-3-piperidin-1-ylcyclobutyl)oxy]phenyl}-4,5,6,7-tetrahydro[1,3]thiazolo[5,4-c]pyridine;
5-acetyl-2-{2-fluoro-4-[(trans-3-piperidin-1-ylcyclobutyl)oxy]phenyl}-4,5,6,7-tetrahydro[1,3]thiazolo[5,4-c]pyridine;
2-[2-{4-[(trans-3-piperidin-1-ylcyclobutyl)oxy]phenyl}-6,7-dihydro[1,3]thiazolo[5,4-c]pyridin-5(4H)-yl]ethanol;
5-acetyl-2-{2,6-difluoro-4-[(trans-3-piperidin-1-ylcyclobutyl)oxy]phenyl}-4,5,6,7-tetrahydro[1,3]thiazolo[5,4-c]pyridine;
5-acetyl-2-{3-fluoro-4-[(trans-3-piperidin-1-ylcyclobutyl)oxy]phenyl}-4,5,6,7-tetrahydro[1,3]thiazolo[5,4-c]pyridine;
5-acetyl-2-{2,3-difluoro-4-[(trans-3-piperidin-1-ylcyclobutyl)oxy]phenyl}-4,5,6,7-tetrahydro[1,3]thiazolo[5,4-c]pyridine;
5-[4-(1-oxidothiomorpholin-4-yl)butanoyl]-2-{4-[(trans-3-piperidin-1-ylcyclobutyl)oxy]phenyl}-4,5,6,7-tetrahydro[1,3]thiazolo[5,4-c]pyridine;
N-{3-oxo-3-[2-{4-[(trans-3-piperidin-1-ylcyclobutyl)oxy]phenyl}-6,7-dihydro[1,3]thiazolo[5,4-c]pyridin-5(4H)-yl]propyl}acetamide;
{2-oxo-2-[2-{4-[(trans-3-piperidin-1-ylcyclobutyl)oxy]phenyl}-6,7-dihydro[1,3]thiazolo[5,4-c]pyridin-5(4H)-yl]ethoxy}acetic acid;
1,1,1-trifluoro-3-oxo-3-[2-{4-[(trans-3-piperidin-1-ylcyclobutyl)oxy]phenyl}-6,7-dihydro[1,3]thiazolo[5,4-c]pyridin-5(4H)-yl]propan-2-ol;
2-{4-[(trans-3-piperidin-1-ylcyclobutyl)oxy]phenyl}-5-(tetrahydro-2H-pyran-4-ylcarbonyl)-4,5,6,7-tetrahydro[1,3]thiazolo[5,4-c]pyridine;
1-{[2-{4-[(trans-3-piperidin-1-ylcyclobutyl)oxy]phenyl}-6,7-dihydro[1,3]thiazolo[5,4-c]pyridin-5(4H)-yl]carbonyl}cyclopropanol;
1-{[2-{4-[(trans-3-piperidin-1-ylcyclobutyl)oxy]phenyl}-6,7-dihydro[1,3]thiazolo[5,4-c]pyridin-5(4H)-yl]carbonyl}cyclopropanecarboxamide;
1-{[2-{4-[(trans-3-piperidin-1-ylcyclobutyl)oxy]phenyl}-6,7-dihydro[1,3]thiazolo[5,4-c]pyridin-5(4H)-yl]carbonyl}cyclopropanecarboxamide trifluoroacetate;
ethyl oxo[2-{4-[(trans-3-piperidin-1-ylcyclobutyl)oxy]phenyl}-6,7-dihydro[1,3]thiazolo[5,4-c]pyridin-5(4H)-yl]acetate;
1-{trans-3-[4-(5-acetyl-4,5,6,7-tetrahydro[1,3]thiazolo[5,4-c]pyridin-2-yl)phenoxy]cyclobutyl}-N,N-dimethylpyrrolidin-3-amine;
5-acetyl-2-(4-{[trans-3-(4-cyclopentylpiperazin-1-yl)cyclobutyl]oxy}phenyl)-4,5,6,7-tetrahydro[1,3]thiazolo[5,4-c]pyridine;
1-{2-oxo-2-[2-{4-[(trans-3-piperidin-1-ylcyclobutyl)oxy]phenyl}-6,7-dihydro[1,3]thiazolo[5,4-c]pyridin-5(4H)-yl]ethyl}urea;
2-oxo-2-[2-{4-[(trans-3-piperidin-1-ylcyclobutyl)oxy]phenyl}-6,7-dihydro[1,3]thiazolo[5,4-c]pyridin-5(4H)-yl]acetamide;
3-oxo-3-[2-{4-[(trans-3-piperidin-1-ylcyclobutyl)oxy]phenyl}-6,7-dihydro[1,3]thiazolo[5,4-c]pyridin-5(4H)-yl]propane-1,2-diol;
3-hydroxy-4-[2-{-4-[(trans-3-piperidin-1-ylcyclobutyl)oxy]phenyl}-6,7-dihydro[1,3]thiazolo[5,4-c]pyridin-5(4H)-yl]cyclobut-3-ene-1,2-dione;
3-isopropoxy-4-[2-{4-[(trans-3-piperidin-1-ylcyclobutyl)oxy]phenyl}-6,7-dihydro[1,3]thiazolo[5,4-c]pyridin-5(4H)-yl]cyclobut-3-ene-1,2-dione;
5-acetyl-2-[4-({trans-3-[2-methylpyrrolidin-1-yl]cyclobutyl}oxy)phenyl]-4,5,6,7-tetrahydro[1,3]thiazolo[5,4-c]pyridine, isomer A;
3-amino-4-[2-{4-[(trans-3-piperidin-1-ylcyclobutyl)oxy]phenyl}-6,7-dihydro[1,3]thiazolo[5,4-c]pyridin-5(4H)-yl]cyclobut-3-ene-1,2-dione.1/2 trifluoroacetate;
5-acetyl-2-[4-({trans-3-[2-methylpyrrolidin-1-yl]cyclobutyl}oxy)phenyl]-4,5,6,7-tetrahydro[1,3]thiazolo[5,4-c]pyridine, isomer B;
(2S)-3-oxo-3-[2-{4-[(trans-3-piperidin-1-ylcyclobutyl)oxy]phenyl}-6,7-dihydro[1,3]thiazolo[5,4-c]pyridin-5(4H)-yl]propane-1,2-diol;
5-acetyl-2-{4-[(cis-3-piperidin-1-ylcyclobutyl)oxy]phenyl}-4,5,6,7-tetrahydro-3H-imidazo[4,5-c]pyridine;
5-acetyl-2-{4-[(trans-3-piperidin-1-ylcyclobutyl)oxy]phenyl}-4,5,6,7-tetrahydro-3H-imidazo[4,5-c]pyridine;
2-oxo-2-[2-{4-[(trans-3-piperidin-1-ylcyclobutyl)oxy]phenyl}-6,7-dihydro[1,3]thiazolo[4,5-c]pyridin-5(4H)-yl]ethanol;
2-{4-[(trans-3-piperidin-1-ylcyclobutyl)oxy]phenyl}-6,7-dihydro[1,3]thiazolo[4,5-c]pyridine-5(4H)-carboxamide;
3-oxo-3-[2-{4-[(trans-3-piperidin-1-ylcyclobutyl)oxy]phenyl}-6,7-dihydro[1,3]thiazolo[4,5-c]pyridin-5(4H)-yl]propanamide;
2-oxo-2-[2-{4-[(trans-3-piperidin-1-ylcyclobutyl)oxy]phenyl}-6,7-dihydro[1,3]thiazolo[4,5-b]pyridin-4(5H)-yl]ethanol;
4-acetyl-2-{4-[(trans-3-piperidin-1-ylcyclobutyl)oxy]phenyl}-5,6,7,8-tetrahydro-4H-[1,3]oxazolo[5,4-b]azepine;
2-{4-[(trans-3-piperidin-1-ylcyclobutyl)oxy]phenyl}-4-(trifluoroacetyl)-5,6,7,8-tetrahydro-4H-[1,3]oxazolo[5,4-b]azepine; and
2-{4-[(trans-3-piperidin-1-ylcyclobutyl)oxy]phenyl}-3H-imidazo[4,5-c]pyridine.

15-17. (canceled)

18. A compound according to claim 10 wherein A is piperidin-1-yl, 2-methylpyrrolidin-1-yl, (2S)-2-methylpyrrolidin-1-yl or (2R)-2-methylpyrrolidin-1-yl.

19. A compound according to claim 10 wherein Y is S.

20. A compound according to claim 10 wherein R² is selected from the group consisting of acetyl, aminocarbonyl, hydroxyacetyl, 2-amino-2-oxoethyl and amino(oxo)acetyl.

21. A pharmaceutical composition comprising a compound according to claim 1 or a pharmaceutically acceptable salt thereof and a pharmaceutically acceptable diluent or carrier.

22. A pharmaceutical composition comprising a compound according to claim 10 or a pharmaceutically acceptable salt thereof and a pharmaceutically acceptable diluent or carrier.

23. A pharmaceutical composition comprising a compound according to claim 14 or a pharmaceutically acceptable salt thereof and a pharmaceutically acceptable diluent or carrier.

24. A method of treating or preventing mild-cognitive impairement, Alzheimer's disease, learning and memory disorders, attention-deficit hyperactivity disorder, Parkinson's disease, schizophrenia, dementia, depression, epilepsy, seizures, convulsions, sleep/wake disorders, cognitive dysfunctions, narcolepsy, hypersomnia, obesity, upper airway allergic disorders, Down's syndrome, anxiety, stress, cardiovascular disorders, inflammation, pain disorders, particularly neuropathic pain, or multiple sclerosis comprising administering an effective amount of a compound according to claim 1.

25. A method of treating or preventing mild-cognitive impairement, Alzheimer's disease, learning and memory disorders, attention-deficit hyperactivity disorder, Parkinson's disease, schizophrenia, dementia, depression, epilepsy, seizures, convulsions, sleep/wake disorders, cognitive dysfunctions, narcolepsy, hypersomnia, obesity, upper airway allergic disorders, Down's syndrome, anxiety, stress, cardiovascular disorders, inflammation, pain disorders, particularly neuropathic pain, or multiple sclerosis comprising administering an effective amount of a pharmaceutical composition according to claim 15.
